SEQUENCE LISTING (55 sequences)

SEQ ID NO: 1 - protein, 562 aa, Homo sapiens
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 2 - DNA, 1689 bp, Homo sapiens
  source 1..1689: mol_type = genomic DNA
atggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtga

SEQ ID NO: 3 - protein, 571 aa, synthetic construct
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRPGGGGSKDEL

SEQ ID NO: 4 - DNA, 1716 bp, synthetic construct
  source 1..1716: mol_type = other DNA
atggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtgagggggtggaggctctaaggatgaactt

SEQ ID NO: 5 - protein, 107 aa, Homo sapiens
STPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFR

SEQ ID NO: 6 - DNA, 321 bp, Homo sapiens
  source 1..321: mol_type = genomic DNA
agcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgc

SEQ ID NO: 7 - protein, 116 aa, synthetic construct
STPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRGGGGSKDEL

SEQ ID NO: 8 - RNA, 348 nt, synthetic construct
  source 1..348: mol_type = other RNA
agcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcgggggtggaggctctaaggatgaactt

SEQ ID NO: 9 - protein, 562 aa, synthetic construct
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDAGGPLVCLNDGRMTLVGIISWGLGCGQKDVPGVYTKVTNYLDWIRDNMRP

SEQ ID NO: 10 - DNA, 1686 bp, synthetic construct
  source 1..1686: mol_type = other DNA
atggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgatgctggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccg

SEQ ID NO: 11 - protein, 4 aa, unidentified
KDEL

SEQ ID NO: 12 - DNA, 12 bp, unidentified
  source 1..12: mol_type = unassigned DNA
aaggatgaactt

SEQ ID NO: 13 - protein, 22 aa, Homo sapiens
NPDGDAKPWCHVLKNRRLTWEY

SEQ ID NO: 14 - protein, 26 aa, synthetic construct
NPDGDAKPWCHVLKNRRLTWEYKDEL

SEQ ID NO: 15 - DNA, 66 bp, Homo sapiens
  source 1..66: mol_type = genomic DNA
aatcctgatggggatgccaagccctggtgccacgtgctgaagaaccgcaggctgacgtgggagtac

SEQ ID NO: 16 - DNA, 78 bp, synthetic construct
  source 1..78: mol_type = other DNA
aatcctgatggggatgccaagccctggtgccacgtgctgaagaaccgcaggctgacgtgggagtacaaggatgaactt

SEQ ID NO: 17 - DNA, 410 bp, synthetic construct
  source 1..410: mol_type = other DNA
tgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataa

SEQ ID NO: 18 - DNA, 130 bp, Adeno-associated virus 2
  source 1..130: mol_type = genomic DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 19 - protein, 568 aa, synthetic construct
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYEDQGISYRGTWSTAESGAECTNWNSSALAQKPYSGRRPDAIRLGLGNHNYCRNPDRDSKPWCYVFKAGKYSSEFCSTPACSEGNSDSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 20 - DNA, 1707 bp, synthetic construct
  source 1..1707: mol_type = other DNA
atggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtga

SEQ ID NO: 21 - protein, 562 aa, synthetic construct
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPNGNAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 22 - DNA, 1689 bp, synthetic construct
  source 1..1689: mol_type = other DNA
atggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctaatgggaacgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtga

SEQ ID NO: 23 - DNA, 7679 bp, synthetic construct
  source 1..7679: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgagtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaactttaatacgactcactataggggccgccaccaagcttggtaccatggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgatgctggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccggggggtggaggctcttatccttacgacgtgcctgactacgcctaaactcgagtctagagcggccgccgaattcgggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgcaggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctctcgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgctggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgagcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaatccgcgcacatttccccgaaaagtgccacctgacgtc

SEQ ID NO: 24 - DNA, 7684 bp, synthetic construct
  source 1..7684: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgagtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaactttaatacgactcactataggggccgccaccaagcttggtaccatggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccggggggtggaggctcttatccttacgacgtgcctgactacgccaaggatgaactttaatctagagcggccgccgaattcgggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgcaggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctctcgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgctggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgagcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaatccgcgcacatttccccgaaaagtgccacctgacgtc

SEQ ID NO: 25 - DNA, 7697 bp, synthetic construct
  source 1..7697: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgagtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaactttaatacgactcactataggggccgccaccaagcttggtaccatggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccggggggtggaggctcttatccttacgacgtgcctgactacgcctaaactcgagtctagagcggccgccgaattcgggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgcaggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctctcgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgctggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgagcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaatccgcgcacatttccccgaaaagtgccacctgacgtc

SEQ ID NO: 26 - DNA, 7679 bp, synthetic construct
  source 1..7679: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgagtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaactttaatacgactcactataggggccgccaccaagcttggtaccatggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctaatgggaacgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccggggggtggaggctcttatccttacgacgtgcctgactacgcctaaactcgagtctagagcggccgccgaattcgggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgcaggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctctcgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgctggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgagcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaatccgcgcacatttccccgaaaagtgccacctgacgtc

SEQ ID NO: 27 - protein, 576 aa, synthetic construct
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRPGGGGSYPYDVPDYA

SEQ ID NO: 28 - DNA, 1731 bp, synthetic construct
  source 1..1731: mol_type = other DNA
atggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtgagggggtggaggctcttatccttacgacgtgcctgactacgcc

SEQ ID NO: 29 - DNA, 7679 bp, synthetic construct
  source 1..7679: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgagtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaactttaatacgactcactataggggccgccaccaagcttggtaccatggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccggggggtggaggctcttatccttacgacgtgcctgactacgcctaaactcgagtctagagcggccgccgaattcgggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgcaggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctctcgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgctggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgagcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaatccgcgcacatttccccgaaaagtgccacctgacgtc

SEQ ID NO: 30 - protein, 310 aa, synthetic construct
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFR

SEQ ID NO: 31 - protein, 125 aa, synthetic construct
STPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRGGGGSYPYDVPDYAKDEL

SEQ ID NO: 32 - RNA, 375 nt, synthetic construct
  source 1..375: mol_type = other RNA
agcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcgggggtggaggctcttatccttacgacgtgcctgactacgccaaggatgaactt

SEQ ID NO: 33 - DNA, 6449 bp, synthetic construct
  source 1..6449: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgagtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaactttaatacgactcactataggggccgccaccaagcttggtaccatggatgcaatgaagagagggctctgctgtgtgctgctgctgtgtggagcagtcttcgtttcgcccagccaggaaatccatgcccgattcagaagaggagccagatcttaccaagtgatctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcgggggtggaggctcttatccttacgacgtgcctgactacgccaaggatgaactttaaactcgagtctagagcggccgccgaattcgggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgcaggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctctcgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgctggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgagcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaatccgcgcacatttccccgaaaagtgccacctgacgtc

SEQ ID NO: 34 - DNA, 348 bp, synthetic construct
  source 1..348: mol_type = other DNA
agcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcgggggtggaggctctaaggatgaactt

SEQ ID NO: 35 - protein, 571 aa, synthetic construct
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDAGGPLVCLNDGRMTLVGIISWGLGCGQKDVPGVYTKVTNYLDWIRDNMRPGGGGSKDEL

SEQ ID NO: 36 - DNA, 1713 bp, synthetic construct
  source 1..1713: mol_type = other DNA
atggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgatgctggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccggggggtggaggctctaaggatgaactt

SEQ ID NO: 37 - protein, 319 aa, synthetic construct
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRGGGGSKDEL

SEQ ID NO: 38 - DNA, 957 bp, synthetic construct
  source 1..957: mol_type = other DNA
atggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcgggggtggaggctctaaggatgaactt

SEQ ID NO: 39 - DNA, 930 bp, synthetic construct
  source 1..930: mol_type = other DNA
atggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgc

SEQ ID NO: 40 - RNA, 756 nt, synthetic construct
  source 1..756: mol_type = other RNA
taatacgactcactataaggacatttgcttctgacacaactgtgttcactagcaacctcaaacagacacccctgcaggcgccgccaccatggacgcaatgaaaagaggactctgctgcgtattgttgctctgcggagcagtcttcgtaagcccatcccaagagatacacgccaggttccgacggggagcaaggtcataccaagtcatatgctcaacacccgcatgctccgagggaaactccgactgctacttcgggaacgggagcgcctacagagggacccacagcctcacagagtcaggggcctcctgcctcccctggaacagcatgatcctgatcggcaaggtgtacaccgcacagaacccgagcgcccaggccctgggcctgggcaaacacaactactgccgcaacccggacggcgacgccaagccgtggtgccacgtgctgaagaaccggaggctcacgtgggaatactgcgacgtgcccagctgctccacctgcggcctgcgccagtactcacagccacagttcagaggaggcggcggcagctacccatacgacgtgccggactacgccaaggacgaactgtgagctcgctttcttgctgtccaatttctattaaaggttcctttgttccctaagtccaactactaaactgggggatattatgaagggccttgagcatctggattctgcctaataaaaaacatttattttcattgcgtcgacacgcgtaaaaaaaaaagaagagcgaattc

SEQ ID NO: 41 - protein, 5 aa, synthetic construct
GGGGS

SEQ ID NO: 42 - protein, 9 aa, synthetic construct
YPYDVPDYA

SEQ ID NO: 43 - DNA, 21 bp, synthetic construct
  source 1..21: mol_type = other DNA
tgctccactcactttaccgtc

SEQ ID NO: 44 - DNA, 21 bp, synthetic construct
  source 1..21: mol_type = other DNA
tagcgtccagtgtgtactgac

SEQ ID NO: 45 - DNA, 23 bp, synthetic construct
  source 1..23: mol_type = other DNA
tgctgtgaaatagataccagggc

SEQ ID NO: 46 - DNA, 22 bp, synthetic construct
  source 1..22: mol_type = other DNA
tgagtctcgatctgggtttctg

SEQ ID NO: 47 - DNA, 19 bp, synthetic construct
  source 1..19: mol_type = other DNA
agcccagaacactggtctc

SEQ ID NO: 48 - DNA, 19 bp, synthetic construct
  source 1..19: mol_type = other DNA
ggtaggctgacccattccc

SEQ ID NO: 49 - DNA, 21 bp, synthetic construct
  source 1..21: mol_type = other DNA
aagcacctccgaaagtacgtg

SEQ ID NO: 50 - DNA, 23 bp, synthetic construct
  source 1..23: mol_type = other DNA
ctccagctctaccttacagttga

SEQ ID NO: 51 - DNA, 20 bp, synthetic construct
  source 1..20: mol_type = other DNA
agcactctcgggacacagaa

SEQ ID NO: 52 - DNA, 20 bp, synthetic construct
  source 1..20: mol_type = other DNA
gtctgcgttggctcatctct

SEQ ID NO: 53 - DNA, 19 bp, synthetic construct
  source 1..19: mol_type = other DNA
gctccaagcagatgcagca

SEQ ID NO: 54 - DNA, 18 bp, synthetic construct
  source 1..18: mol_type = other DNA
ccggatgtgaggcagcag

SEQ ID NO: 55 - protein, 6 aa, synthetic construct
HHHHHH

(19) United States
(12) Patent Application Publication
(10) Pub. No.: US 2026/0327594 A1
(43) Pub. Date: Oct. 22, 2026

(54) RECOMBINANT TISSUE PLASMINOGEN ACTIVATOR (TPA) FRAGMENTS AND USES THEREOF

(71) Applicants: VERSITI BLOOD RESEARCH INSTITUTE FOUNDATION, INC., Milwaukee, WI (US); THE MEDICAL COLLEGE OF WISCONSIN, INC., Milwaukee, WI (US)

(72) Inventors: Ze Zheng, Milwaukee, WI (US); Wen Dai, Milwaukee, WI (US); Hayley Lund, Milwaukee, WI (US); Christian Kastrup, Milwaukee, WI (US)

(73) Assignees: VERSITI BLOOD RESEARCH INSTITUTE FOUNDATION, INC., Milwaukee, WI (US); THE MEDICAL COLLEGE OF WISCONSIN, INC., Milwaukee, WI (US)

(21) Appl. No.: 19/484,995

(22) PCT Filed: May 17, 2024
(86) PCT No.: PCT/US2024/029985

§ 371 Date: Nov. 17, 2025

Related U.S. Application Data
(60) Provisional application No. 63/467,450, filed on May 18, 2023.

(51) Int. Cl. C12N 9/72; A61K 9/51; A61K 35/76; A61K 38/49; A61K 45/06; A61K 48/00; A61P 9/00; C12N 5/071; C12N 7/00; C12N 15/63

(52) U.S. Cl. CPC C12N 9/6459; A61K 9/5123; A61K 35/76; A61K 38/49; A61K 45/06; A61K 48/0058; A61P 9/00; C12N 5/067; C12N 7/00; C12N 15/63; C12Y 304/21068; C12N 2750/14121; C12N 2750/14132; C12N 2830/34

(57) ABSTRACT

The present technology relates to recombinant polypeptides, or nucleotides encoding the same, comprising tissue plasminogen activator (tPA) fragments and uses thereof for treating cardiovascular diseases. In some embodiments, the tPA fragments comprise the kringle 2 domain of tPA.

### CROSS-REFERENCE TO RELATED APPLICATIONS

This application claims the benefit of and priority to U.S. Provisional Patent Application No. 63/467,450, filed May 18, 2023, the contents of which are incorporated by reference in their entirety for any and all purposes.

### STATEMENT REGARDING FEDERALLY SPONSORED RESEARCH OR DEVELOPMENT

This invention was made with government support under HL163516 awarded by the National Institutes of Health. The government has certain rights in the invention.

### TECHNICAL FIELD

The present technology provides recombinant polypeptides, or nucleotides encoding the same, comprising tissue plasminogen activator (tPA) fragments and uses thereof for treating cardiovascular diseases. In some embodiments, the tPA fragments comprise the kringle 2 domain of tPA (tPA-K2).

### SEQUENCE LISTING

An informal sequence listing is provided herein.

### BACKGROUND

Apolipoprotein B (apoB)-containing lipoproteins initiate and promote atherosclerotic cardiovascular disease (CVD) [1,2]. ApoB-containing lipoproteins include hepatocyte-derived very-low-density lipoproteins (VLDL), intermediate-density lipoprotein (IDL), low-density lipoprotein (LDL), and lipoprotein (a) [Lp(a)], as well as enterocyte-derived chylomicrons and chylomicron remnants. Hepatocytes and enterocytes produce VLDL and chylomicrons, respectively, and secrete them into the blood. Circulating VLDL is then progressively hydrolyzed in the blood to form IDL and LDL. Similarly, circulating chylomicrons are hydrolyzed to form chylomicron remnants [2]. Currently recommended lipid intervention therapies to help prevent CVD primarily use statins or protein convertase subtilisin/kexin type 9 (PCSK9) inhibitors, both of which lower LDL by enhancing hepatic LDL receptor (LDLR)-mediated LDL clearance [3]. However, these treatments have only a modest effect on other atherogenic apoB-containing lipoproteins, such as VLDL, IDL, Lp(a), chylomicron and chylomicron remnants [4,5], which contribute to the residual CVD risk in populations with well-controlled LDL cholesterol [6,7]. Thus, therapies that inhibit hepatic VLDL and Lp(a) production and enterocyte chylomicron production might be useful in decreasing CVD risk, as they would lower all atherogenic apoB-lipoproteins. Accordingly, there is a need in the art for new therapeutic approaches to lowering atherogenic apo-B lipoproteins and treating cardiovascular disease.

### SUMMARY

In one aspect, the present disclosure provides an isolated polynucleotide molecule comprising a nucleotide sequence selected from the group consisting of: (a) a nucleotide sequence set forth in SEQ ID NO: 6; (b) a nucleotide sequence set forth in SEQ ID NO: 10; (c) a nucleotide sequence set forth in SEQ ID NO: 15; (d) a nucleotide sequence set forth in SEQ ID NO: 39; (e) a nucleotide sequence set forth in SEQ ID NO: 2; (f) a nucleotide sequence that is at least about 85% identical to the nucleotide sequences of any one of (a)-(f), and which encodes a recombinant tissue plasminogen activator kringle 2 domain (tPA-K2)-containing polypeptide that is capable of binding apolipoprotein B (apoB), and/or inhibiting apoB secretion from a hepatocyte and/or an enterocyte, and/or inhibiting apoB lipoprotein lipidation; (g) a nucleotide sequence that is the complement of any one of (a)-(f); and (h) an RNA sequence encoded by any one of (a)-(g); wherein the nucleotide sequence is operably linked to a heterologous nucleic acid. In some embodiments, the heterologous nucleic acid comprises an endoplasmic reticulum localization sequence. In some embodiments, the endoplasmic reticulum localization sequence encodes for an amino acid having the sequence KDEL (SEQ ID NO: 11). In some embodiments, the nucleotide sequence is set forth in SEQ ID NO: 8. In some embodiments, the nucleotide sequence is set forth in SEQ ID NO: 34. In some embodiments, the nucleotide sequence is set forth in SEQ ID NO: 16. In some embodiments, the nucleotide sequence is set forth in SEQ ID NO: 4. In some embodiments, the nucleotide sequence is set forth in SEQ ID NO: 36. In some embodiments, the nucleotide sequence is set forth in SEQ ID NO: 38. In some embodiments, the nucleotide sequence is set forth in SEQ ID NO: 32. In some embodiments, the nucleotide sequence is set forth in SEQ ID NO: 40. In some embodiments, the nucleotide sequence encodes a polypeptide having the amino acid sequence set forth in SEQ ID NO: 7. In some embodiments, the nucleotide sequence encodes a polypeptide having the amino acid sequence set forth in SEQ ID NO: 14. In some embodiments, the nucleotide sequence encodes a polypeptide having the amino acid sequence set forth in SEQ ID NO: 35. In some embodiments, the nucleotide sequence encodes a polypeptide having the amino acid sequence set forth in SEQ ID NO: 30. In some embodiments, the nucleotide sequence encodes a polypeptide having the amino acid sequence set forth in SEQ ID NO: 3. In some embodiments, the nucleotide sequence encodes a polypeptide having the amino acid sequence set forth in SEQ ID NO: 9. In some embodiments, the nucleotide sequence encodes a polypeptide having the amino acid sequence set forth in SEQ ID NO: 37. In some embodiments, the nucleotide sequence encodes a polypeptide having the amino acid sequence set forth in SEQ ID NO: 31. In some embodiments, the present disclosure provides an expression vector comprising any one of the polynucleotide molecules operably linked to one or more regulatory sequences suitable for directing expression in a eukaryotic cell. In some embodiments, the one or more regulatory sequences comprises a promoter. In some embodiments, the promoter is a thyroxine binding globulin (TBG) promoter or wherein the promoter comprises the nucleic acid sequence set forth in SEQ ID NO: 17. In some embodiments, the present disclosure provides a cell comprising any one of the polynucleotide molecules or the expression vector. In some embodiments, the cell is selected from a hepatocyte or an enterocyte. In some embodiments, the present disclosure provides an infectious particle comprising any one of the polynucleotide molecules. In some embodiments, the infectious particle is a virus. In some embodiments, the virus in an adeno-associated virus (AAV). In some embodiments, the AAV is AAV8. In some embodiments, the present disclosure provides a lipid nanoparticle comprising any one of the polynucleotide molecules. In some embodiments, the lipid nanoparticle is lyophilized, in a suspension, or emulsified. In some embodiments, the present disclosure provides a composition comprising any one of the polynucleotide molecules, the vector, the infectious particle, or the lipid nanoparticle, and a pharmaceutically acceptable carrier. In some embodiments, the present disclosure provides a method for lowering plasma apoB lipoproteins in a subject in need thereof, the method comprising administering a therapeutically effective amount of the composition to the subject. In some embodiments, the plasma apoB lipoproteins are selected from very low density lipoproteins (VLDLs), intermediate density lipoproteins (IDLs), low density lipoproteins (LDLs), lipoprotein a (Lp(a)), chylomicrons, chylomicron remnants, or any combination thereof. In some embodiments, the present disclosure provides a method for treating a disease associated with elevated plasma apoB lipoprotein levels, the method comprising administering a therapeutically effective amount of the composition to the subject. In some embodiments, the disease is selected from atherosclerotic cardiovascular disease, hypercholesterolemia, hyperlipidemia, or type 2 diabetes. In some embodiments, the present disclosure provides a method for lowering plasma triglyceride and/or cholesterol levels in a subject in need thereof, the method comprising administering a therapeutically effective amount of the composition to the subject. In some embodiments, the method further comprises simultaneously, separately, or sequentially administering a therapeutically effective amount of a plasminogen activator inhibitor-1 (PAI-1) inhibitor to the subject. In some embodiments, the PAI-1 inhibitor is selected from the group consisting of: MDI-2268, PAI-039, TM5441, TM5275 sodium, TM5441 sodium, CDE-096, Aleplasinin, Loureirin B, Diaplasinin, Toddalolactone, SK-216, Geodin, Fendosal, AZ3976, TM5007; and any combination thereof. In some embodiments, the composition is administered intravenously, intraperitoneally, subcutaneously, intrabuccally, intradermally, intrahepatically, or intramuscularly to the subject. In some embodiments, the subject is human.

In one aspect, the present disclosure provides a recombinant tissue plasminogen activator kringle 2 domain (tPA-K2)-containing polypeptide, or a pharmaceutically acceptable salt, tautomer, hydrate, and/or solvate thereof, (tPA-K2 polypeptide) comprising: (a) an amino acid sequence selected from the group consisting of: (i) the amino acid sequence set forth in SEQ ID NO: 5; (ii) the amino acid sequence set forth in SEQ ID NO: 13; (iii) the amino acid sequence set forth in SEQ ID NO: 9; (iv) the amino acid sequence set forth in SEQ ID NO: 30; (v) the amino acid sequence set forth in SEQ ID NO: 1; (vi) the amino acid sequence set forth in SEQ ID NO: 37; (vii) an amino acid sequence that is at least about 85% identical to the amino acid sequence of any one of (i)-(vi), and which is capable of binding apolipoprotein B (apoB), and/or inhibiting apoB secretion from a hepatocyte and/or an enterocyte, and/or inhibiting apoB lipoprotein lipidation; and (b) a heterologous amino acid sequence. In some embodiments, the heterologous amino acid sequence comprises an endoplasmic reticulum localization motif. In some embodiments, the endoplasmic reticulum localization motif is KDEL. In some embodiments, the amino acid sequence is set forth in SEQ ID NO: 7. In some embodiments, the amino acid sequence is set forth in SEQ ID NO: 14. In some embodiments, the amino acid sequence is set forth in SEQ ID NO: 35. In some embodiments, the amino acid sequence is set forth in SEQ ID NO: 37. In some embodiments, the amino acid sequence is set forth in SEQ ID NO: 3. In some embodiments, the polypeptide does not comprise serine protease function. In some embodiments, the polypeptide is not fibrinolytic. In some embodiments, the polypeptide binds to apoB. In some embodiments, administration of a therapeutically effective amount of the polypeptide to a subject reduces plasma triglyceride and/or cholesterol levels in the subject. In some embodiments, administration of a therapeutically effective amount of the polypeptide to a subject reduces the plasma level of one or more apoB lipoproteins in the subject. In some embodiments, the one or more apoB lipoproteins is selected from very low density lipoproteins (VLDLs), intermediate density lipoproteins (IDLs), low density lipoproteins (LDLs), lipoprotein a (Lp(a)), chylomicrons, chylomicron remnants, or any combination thereof. In some embodiments, the present disclosure provides a composition comprising any one of the recombinant polypeptides and a pharmaceutically acceptable carrier. In some embodiments, the present disclosure provides a method for lowering plasma apoB lipoproteins in a subject in need thereof, the method comprising administering a therapeutically effective amount of the recombinant polypeptide or the composition of to the subject. In some embodiments, the present disclosure provides a method for treating a disease associated with elevated plasma apoB lipoprotein levels, the method comprising administering a therapeutically effective amount of any one of the recombinant polypeptides or the composition to the subject. In some embodiments, the disease is selected from atherosclerotic cardiovascular disease, hypercholesterolemia, hyperlipidemia, or type 2 diabetes. In some embodiments, the method further comprises simultaneously, separately, or sequentially administering a therapeutically effective amount of a plasminogen activator inhibitor-1 (PAI-1) inhibitor to the subject. In some embodiments, the PAI-1 inhibitor is selected from the group consisting of: MDI-2268, PAI-039, TM5441, TM5275 sodium, TM5441 sodium, CDE-096, Aleplasinin, Loureirin B, Diaplasinin, Toddalolactone, SK-216, Geodin, Fendosal, AZ3976, TM5007; and any combination thereof. In some embodiments, the recombinant polypeptide or composition is administered intravenously, intraperitoneally, subcutaneously, intrabuccally, intradermally, intrahepatically, or intramuscularly to the subject. In some embodiments, the subject is human.

In one aspect, the present disclosure provides a polynucleotide molecule comprising a nucleotide sequence set forth in SEQ ID NO: 8. In some embodiments, the polynucleotide is formulated for delivery to a subject in an infectious particle. In some embodiments, the infectious particle is an adeno-associated virus (AAV). In some embodiments, the polynucleotide is formulated for delivery to a subject in a lipid nanoparticle (LNP). In some embodiments, the present disclosure provides a composition comprising any one of the polynucleotides, and a pharmaceutically acceptable carrier. In some embodiments, the present disclosure provides a method for lowering plasma apoB lipoproteins in a subject in need thereof, the method comprising administering a therapeutically effective amount of the composition to the subject. In some embodiments, the present disclosure provides a method for lowering plasma triglyceride and/or cholesterol levels in a subject in need thereof, the method comprising administering a therapeutically effective amount of the composition to the subject.

In one aspect, the present disclosure provides a polynucleotide molecule comprising a nucleotide sequence selected from the group consisting of: (a) a nucleotide sequence set forth in SEQ ID NO: 34; (b) a nucleotide sequence set forth in SEQ ID NO: 10; and (c) a nucleotide sequence set forth in SEQ ID NO: 36. In some embodiments, the polynucleotide is formulated for delivery to a subject in an infectious particle. In some embodiments, the infectious particle is an adeno-associated virus (AAV). In some embodiments, the polynucleotide is formulated for delivery to a subject in a lipid nanoparticle (LNP). In some embodiments, the present disclosure provides a composition comprising any one of the polynucleotides, and a pharmaceutically acceptable carrier. In some embodiments, the present disclosure provides a method for lowering plasma apoB lipoproteins in a subject in need thereof, the method comprising administering a therapeutically effective amount of the composition to the subject. In some embodiments, the present disclosure provides a method for lowering plasma triglyceride and/or cholesterol levels in a subject in need thereof, the method comprising administering a therapeutically effective amount of the composition to the subject. In some embodiments, the nucleotide sequence is set forth in SEQ ID NO: 34. In some embodiments, the nucleotide sequence is set forth in SEQ ID NO: 10. In some embodiments, the nucleotide sequence is set forth in SEQ ID NO: 36.

In one aspect, the present disclosure provides a recombinant tissue plasminogen activator kringle 2 domain (tPA-K2))-containing polypeptide, or a pharmaceutically acceptable salt, tautomer, hydrate, and/or solvate thereof, (tPA-K2 polypeptide) comprising an amino acid sequence selected from the group consisting of: the amino acid sequence set forth in SEQ ID NO: 9 and SEQ ID NO: 35. In some embodiments, the present disclosure provides a composition comprising the recombinant polypeptide and a pharmaceutically acceptable carrier. In some embodiments, the present disclosure provides a method for lowering plasma apoB lipoproteins in a subject in need thereof, the method comprising administering a therapeutically effective amount of any one of the recombinant polypeptides the composition to the subject. In some embodiments, the present disclosure provides a method for treating a disease associated with elevated plasma apoB lipoprotein levels, the method comprising administering a therapeutically effective amount of any one of the recombinant polypeptides or the composition to the subject. In some embodiments, the disease is selected from atherosclerotic cardiovascular disease, hypercholesterolemia, hyperlipidemia, or type 2 diabetes.

In one aspect, the present disclosure provides a recombinant tissue plasminogen activator kringle 2 domain (tPA-K2) polypeptide, or a fragment thereof. In some embodiments, the recombinant tPA-K2 polypeptide comprises a sequence with at least 80% identity to SEQ ID NO: 5. In some embodiments, the polypeptide does not comprise serine protease function. In some embodiments, the polypeptide is not fibrinolytic. In some embodiments, the recombinant tPA-K2 polypeptide binds to apolipoprotein B (apoB). In some embodiments, the recombinant tPA-K2 polypeptide comprises a sequence selected from SEQ ID NOs: 13-14. In some embodiments, the recombinant tPA-K2 polypeptide comprises SEQ ID NO: 14. In some embodiments, the present disclosure provides a method of making the recombinant tPA-K2 polypeptides. In some embodiments, the present disclosure provides a polynucleotide comprising a nucleotide sequence encoding the recombinant tPA-K2 polypeptides. In some embodiments, the nucleotide sequence encoding the recombinant tPA-K2 polypeptide comprises a nucleotide sequence selected from SEQ ID NOs: 15-16. In some embodiments, the polynucleotides further comprise at least one regulatory sequence operably linked to the nucleotide sequence encoding the recombinant tPA-K2 polypeptide. In some embodiments, the at least one regulatory sequence comprises a promoter, an enhancer, or both a promoter and an enhancer. In some embodiments, the at least one regulatory sequence comprises a promoter. In some embodiments, the promoter is thyroxine binding globulin (TBG) promoter or wherein the promoter comprises SEQ ID NO: 17. In some embodiments, the polynucleotide comprises more than one nucleotide sequence encoding the recombinant tPA-K2 polypeptide. In some embodiments, the present disclosure provides a nanoparticle comprising the recombinant tPA-K2 polypeptide or the polynucleotide. In some embodiments, the present disclosure provides an infectious particle comprising the polynucleotide. In some embodiments, the infectious particle is a virus. In some embodiments, the virus is an adeno-associated virus (AAV). In some embodiments, the AAV is an AAV8. In some embodiments, the present disclosure provides a pharmaceutical composition comprising the recombinant tPA-K2 polypeptide. In some embodiments, the present disclosure provides a pharmaceutical composition comprising the nanoparticle. In some embodiments, the present disclosure provides a pharmaceutical composition comprising the infectious particle. In some embodiments, the present disclosure provides a method comprising administering a therapeutically effective amount of the pharmaceutical composition to a subject. In some embodiments, the subject has hypercholesterolemia or hyperlipidemia. In some embodiments, the subject has been diagnosed with a cardiovascular disease. In some embodiments, the cardiovascular disease comprises atherosclerosis. In some embodiments, the subject has been diagnosed with type 2 diabetes.

In one aspect, the present disclosure provides a method of treating a cardiovascular disease in a subject in need thereof, the method comprising administering tissue plasminogen activator (tPA), or a fragment thereof, to the subject to treat the cardiovascular disease in the subject. In some embodiments, the tPA fragment comprises a tPA-K2 domain.

In one aspect, the present disclosure provides a method of reducing blood cholesterol levels in a subject in need thereof, the method comprising administering tissue plasminogen activator (tPA), or a fragment thereof, to the subject to reduce blood cholesterol levels in the subject. In some embodiments, the tPA fragment comprises a tPA-K2 domain. In some embodiments, administration comprises oral administration or intravenous administration. In some embodiments, the method reduces a level of intermediate density lipoproteins (IDLs), low density lipoproteins (LDLs), very low density lipoproteins (VLDLs), lipoprotein (a) [Lp(a)], chylomicron, or chylomicron remnants in the serum of the subject. In some embodiments, the tPA comprises the pharmaceutical composition.

In one aspect, the present disclosure provides a method for lowering plasma lipids and/or apolipoprotein B (apoB) levels in a subject in need thereof, the method comprising administering a therapeutically effective amount of a composition comprising a polynucleotide molecule comprising a nucleotide sequence selected from the group consisting of: (a) a nucleotide sequence set forth in SEQ ID NO: 2; (b) a nucleotide sequence that encodes for the polypeptide sequence set forth in SEQ ID NO: 1; (c) a nucleotide sequence that is at least about 85% identical to the nucleotide sequences of any one of (a)-(b); (d) a nucleotide sequence that is the complement of any one of (a)-(c); and (e) an RNA sequence encoded by any one of (a)-(d); wherein the nucleotide sequence is operably linked to a heterologous nucleic acid comprising an endoplasmic reticulum localization sequence; and wherein the subject is suffering from or at an increased risk for atherothrombotic events. In some embodiments, the endoplasmic reticulum localization sequence encodes for a polypeptide having the amino acid sequence KDEL (SEQ ID NO: 11). In some embodiments, the polynucleotide molecule comprises a nucleotide sequence set forth in SEQ ID NO: 4. In some embodiments, the nucleotide sequence encodes for a polypeptide sequence set forth in SEQ ID NO: 3. In some embodiments, the composition is formulated for delivery to the subject in a lipid nanoparticle or adeno-associated virus. In some embodiments, the administration of the composition lowers plasma apoB and/or lipid levels in the subject. In some embodiments, the subject is undergoing, will undergo, or has undergone thrombolytic therapy. In some embodiments, the method further comprises simultaneously, sequentially, or separately administering a tissue plasminogen activator to the subject. In some embodiments, the method further comprises simultaneously, sequentially, or separately administering an anticoagulant to the subject.

In some embodiments, for any of the methods of treatment disclosed herein, the subject may be suffering from or at an increased risk for atherothrombotic events. In some embodiments, the subject is undergoing, will undergo, or has undergone treatment with a thrombolytic agent, an anticoagulant, catheter-directed thrombolysis, and/or thrombectomy.

### BRIEF DESCRIPTION OF THE DRAWINGS

FIGS. 1A-1E show that silencing hepatocyte tPA increases atherogenic apoB-lipoprotein cholesterol and apoB independently of LDLR or ApoE. FIG. 1A is a set of charts providing results for Western diet (WD)-fed Ldlr^{-/-} mice treated with AAV8-H1-shPlat (sh-tPA) or AAV8-H1-scrambled control (scr). The Ldlr^{-/-} mice were treated with sh-tPA or scr and then fed a WD for 8 weeks. The mouse livers were assayed for tPA protein, and plasma samples were assayed for total cholesterol and apoB-100 concentrations and for FPLC profiles of cholesterol, triglyceride, and apoB-100 (n=9 to 10 mice per group). FIG. 1B is a set of charts providing results for Western diet (WD)-fed Apoe^{-/-} mice treated with AAV8-shPlat (sh-tPA) or AAV8-scrambled control (scr). The Apoe^{-/-} mice were treated with sh-tPA or scr and then fed the WD for 8 weeks. The livers were assayed for tPA protein, and plasma samples were assayed for total cholesterol and apoB-100 concentrations and for FPLC profiles of cholesterol, triglyceride, and apoB-100 (n=5 mice per group). FIG. 1C is a set of charts providing results for Western diet (WD)-fed Plat^{fl/fl} mice treated with AAV8-TBG-cre (Cre) or AAV8-TBG-GFP (GFP). The Plat^{fl/fl} mice were treated with Cre or GFP and then fed the WD for 8 weeks. The livers were assayed for tPA protein, and plasma samples were assayed for total cholesterol and apoB-100 concentrations and for FPLC profiles of cholesterol, triglyceride, and apoB-100. The cholesterol in the VLDL fractions is shown in a zoomed-in smaller graph (n=6 mice per group). FIG. 1D is a set of charts and immunoblot images showing the results of human primary hepatocytes that were treated with siRNA against tPA mRNA (si-tPA) or scrambled RNA for 24 hours. Cell culture medium apoB was quantified by immunoblot. VLDL fractions were isolated by ultracentrifugation, and cholesterol and triglyceride concentrations in VLDL fractions were assayed. FIG. 1E is a set of charts and immunoblot images showing the results of McA-RH7777 cells that were treated with siRNA against tPA mRNA (si-tPA) or scrambled RNA for 24 hours. VLDL was isolated from the medium by ultracentrifugation, and cholesterol and triglyceride concentrations in VLDL were assayed. Cell culture medium apoB was assayed by immunoblot. Data are shown as means±SEMs; *P<0.05 by two-tailed Student's t test.

FIGS. 2A-2K show tPA limits apoB lipidation in the endoplasmic reticulum (ER). FIG. 2A: Wild-type (WT) mice were treated with AAV8-H1-shPlat (sh-tPA) or AAV8-H1-scrambled control (scr) and then fed the Western diet (WD) for 14 weeks. Mice were injected with P407 intraperitoneally (i.p.) to assess VLDL secretion. Plasma triglyceride concentration was measured (n=4 to 5 mice per group). FIG. 2B: WT mice were treated with AAV8-H1-shPlat (sh-tPA) or AAV8-H1-scrambled control (scr) and then fed the WD for 14 weeks. Mice were injected with P407 i.p. to assess VLDL secretion. Plasma apoB concentration was measured by ELISA (n=4 to 5 mice per group). FIG. 2C: Ldlr^{-/-} mice were treated with AAV8-H1-shPlat (sh-tPA) or AAV8-H1-scrambed control (scr) and then fed the WD for 8 weeks. VLDL was isolated by ultracentrifugation and visualized by transmission electron microscopy. VLDL (n=100 for each group) diameter was measured and analyzed using Image-Pro Plus 10.0. Scale bars, 100 nm. FIG. 2D: Ldlr^{-/-} mice were treated with AAV8-H1-shPlat (sh-tPA) or AAV8-H1-scrambled control (scr) and then fed the WD for 8 weeks (n=9 to 10 mice per group). VLDL was isolated by ultracentrifugation, and VLDL diameter was measured by dynamic light scattering. FIG. 2E: Ldlr^{-/-} mice were treated with AAV8-H1-shPlat (sh-tPA) or AAV8-H1-scrambled control (scr) and then fed the WD for 8 weeks (n=9 to 10 mice per group). VLDL was isolated by ultracentrifugation and assayed for the ratio of triglyceride to apoB-100 (n=9 to 10 mice per group). FIG. 2F: Whole-body tPA knockout mice (holo-tPA-KO) were treated with AAV8-TBG-Plat (tPA) or AAV8-TBG-lacZ (LacZ) and then fed a normal chow diet for 8 weeks. Plasma samples were assayed for VLDL cholesterol, LDL cholesterol, and apoB-100 concentrations (n=6 mice per group). FIG. 2G: Human primary hepatocytes were transduced with a plasmid (SEQ ID NO: 29) encoding tPA and C-terminal HA tag (tPA-HA; SEQ ID NO: 27) or GFP. After 48 hours, apoB secretion was measured using a [³H]-leucine-containing medium and chased for 3 hours in [³H]-leucine-free medium, and then radioactivity associated with apoB in the cell medium was quantified by scintillation counting. FIG. 2H: Human primary hepatocytes were treated with siRNA against tPA mRNA (si-tPA) or scrambled (scr) RNA for 24 hours. apoB secretion was measured using [³H]-labeling, as in FIG. 2G. Radioactivity associated with apoB in cell medium was quantified by scintillation counting. FIG. 2I: Human primary hepatocytes were treated with siRNA against tPA mRNA (si-tPA) or scrambled (scr) RNA for 24 hours. VLDL was isolated by ultracentrifugation and VLDL diameter was measured by dynamic light scattering. FIG. 2J: Human primary hepatocytes were treated with siRNA against tPA mRNA (si-tPA) or scrambled (scr) RNA for 24 hours. VLDL was isolated by ultracentrifugation and assayed for the ratio of triglyceride to apoB-100. FIG. 2K: Human primary hepatocytes were treated with siRNA against tPA mRNA (si-tPA) or scrambled (scr) RNA for 24 hours. The endoplasmic reticulum (ER) fraction was isolated, and proteins from the ER were extracted. apoB-lipoproteins extracted from the ER were further separated by density gradient ultracentrifugation and divided into six fractions of increasing density from fraction 1 to 6. apoB from each fraction was measured by immunoblot. Data are shown as means±SEMs; *P<0.05 by two-tailed Student's t test.

FIGS. 3A-3J show that tPA blocks apoB-VLDL assembly. FIG. 3A: Human primary hepatocytes were treated with siRNA against tPA mRNA (si-tPA) or scrambled RNA for 24 hours. Cell lysates (input) and anti-MTP immunoprecipitates (IP: MTP) were assayed for apoB and MTP by immunoblot. FIG. 3B: Human primary hepatocytes were treated with siRNA against tPA mRNA (si-tPA) or scrambled RNA (group 1) or tPA mRNA (si-tPA) (groups 2 and 3) for 24 hours. The microsomal fraction was isolated and assayed for neutral lipid transfer activity with DMSO (groups 1 and 2) or without with CP-346086 (10 nM), an MTP inhibitor (group 3). FIG. 3C: Human primary hepatocytes were transduced with a plasmid (SEQ ID NO: 29) encoding tPA with C terminal HA tag (tPA-HA; SEQ ID NO: 27) or GFP for 48 hours. Cell lysates (input) and anti-MTP precipitates (IP: MTP) were assayed for apoB and MTP by immunoblot. FIG. 3D: Human primary hepatocytes were transduced with a plasmid (SEQ ID NO: 29) encoding tPA-HA (SEQ ID NO: 27) or GFP for 48 hours. The microsomal fraction was isolated and assayed for neutral lipid transfer activity. FIG. 3E: The effect of recombinant human tPA on lipid transfer from donor vesicles to human LDL was assayed. FIG. 3F: Human primary hepatocytes were transduced with a plasmid (SEQ ID NO: 29) encoding tPA-HA (SEQ ID NO: 27) or GFP for 48 hours. Cell lysates (input) and anti-HA immunoprecipitates (IP: HA) were assayed for apoB and tPA. FIG. 3G: A proximity ligation assay was used to measure apoB-tPA interaction in human primary hepatocytes. Scale bars, 10 μm. FIG. 3H: Confocal microscopy immunofluorescence imaging was used to measure the subcellular localization of tPA and apoB in human primary hepatocytes. Scale bars, 10 μm. FIG. 3I: Human primary hepatocytes were transduced with a plasmid encoding wild-type tPA (tPA-WT (SEQ ID NO: 1)), an enzymatically inactive mutant of tPA (tPA-S513A (SEQ ID NO: 9)), tPA with an endoplasmic reticulum retention signal sequence (tPA-KDEL (SEQ ID NO: 3)), or GFP for 48 hours. ApoB secretion was measured by [³H]-labeling as in FIGS. 2G and 2H. FIG. 3J: The effect of recombinant wild-type tPA (tPA-WT) or enzymatically inactive mutant of tPA (tPA-S513A) on lipid transfer from donor vesicles to human LDL was assayed. Data are shown as mean±SEMs; *P<0.05 by two-tailed Student's t-test (FIG. 3D) or by one-way analysis of variance (ANOVA) followed by Dunnett's test (FIGS. 3B, 3E, 3I, 3J).

FIGS. 4A-4I show that the Kringle 2 (K2) domain of tPA interacts with the N-terminus of apoB. FIG. 4A: A solid-phase binding assay was used to measure the interaction between LDL and recombinant human wild-type tPA (tPA-WT) or enzymatically inactive tPA-S513A. The binding to of tPA-WT or tPA-S513A to wells without LDL is also measured under the same conditions as the control. FIG. 4B: A solid-phase binding assay was used to measure the interaction between recombinant human tPA and purified human MTP complex. FIG. 4C: A solid-phase binding assay was used to measure the ability of tPA to inhibit the binding of MTP to LDL. FIG. 4D: Surface plasmon resonance was used to measure the interaction between human recombinant tPA and LDL. FIG. 4E: A solid-phase binding assay was used to test whether anti-apoB N-terminal antibody (1D1) versus control IgG (raised against the β3 domain of apoB) blocks the binding between human recombinant tPA and LDL. FIG. 4F: Human primary hepatocytes were transduced with the plasmid encoding wild-type tPA (tPA-WT (SEQ ID NO: 1)), a tPA mutant without the K2 domain (tPA-Δ-K2 (SEQ ID NO: 19 and 20)), or tPA mutated in the K2 domain lysine binding site (tPA-D236, 238N (SEQ ID NO: 21 and 22)). ApoB secretion was measured by [³H]-labeling as in FIGS. 2G and 2H. FIG. 4G: A solid-phase binding assay was used to test whether an antibody against tPA-K2 domain interferes with the interaction between human recombinant tPA and purified LDL. FIG. 4H: A solid-phase binding assay was used to measure whether tranexamic acid (TXA) interferes with the interaction between recombinant human tPA and purified LDL. FIG. 4I: A schematic diagram depicting the interaction between the N-terminus of apoB and the K2 domain of tPA. The diagram was generated using biorender.com. Data are shown as mean±SEMs; *P<0.05 by one-way analysis of variance (ANOVA) followed by Dunnett's test. n.s., not significant (P≥0.05).

FIGS. 5A-5J show that PAI-1 sequesters tPA away from apoB, leading to increased VLDL assembly in hepatocytes. FIG. 5A: Human primary hepatocytes lysates (input) and anti-PAI-1 immunoprecipitates (IP: PAI-1) were assayed for tPA and PAI-1 by immunoblot. FIG. 5B: A proximity ligation assay was used to measure tPA-PAI-1 interaction in human primary hepatocytes. Scale bars, 10 μm. FIG. 5C: Human primary hepatocytes were treated for 6 hours with 0.4 mM oleate complexed with fatty acid-free BSA (oleate group) or fatty acid-free BSA alone (vehicle control). Cell lysates were assayed for tPA by immunoblot using the Jess Simple Western system. FIG. 5D: Human primary hepatocytes were treated with 0.4 mM oleate complexed with fatty acid-free BSA (oleate group) or fatty acid-free BSA alone (vehicle control). Cell lysates were assayed for tPA-PA-1 complex and PAI-1-free tPA concentrations by ELISA. FIG. 5E: Human primary hepatocytes were treated with siRNA against PAI-1 mRNA (si-PAI1) or scrambled RNA and then incubated in medium containing either 0.4 mM oleate complexed with fatty acid-free BSA (oleate group) or fatty acid-free BSA alone (vehicle control). Cell lysates were assayed for PAI 1-free tPA concentration by ELISA. FIG. 5F: Human primary hepatocytes were treated with siRNA against PAI-1 mRNA (si-PAI1) or scrambled RNA and then incubated in medium containing either 0.4 mM oleate complexed with fatty acid-free BSA (oleate group) or fatty acid-free BSA alone (vehicle control). ApoB secretion was measured by [³H]-labeling as in FIGS. 2G and 2H. FIG. 5G: Human primary hepatocytes were treated with siRNA against tPA mRNA (si-tPA) or against PAI-1 mRNA (si-PAI1) or scrambled RNA. ApoB secretion was measured by [³H]-labeling as in FIGS. 2G and 2H. FIG. 5H: A solid-phase binding assay was used to measure the interaction between LDL and recombinant human tPA or tPA-PAI-1 complex. FIG. 5I: The effect of recombinant human tPA and tPA-PAI-1 complex on lipid transfer from donor vesicles to human LDL was assayed. FIG. 5J: Normal chow diet-fed wild-type mice had their food withdrawn for 5 hours and then euthanized at 0, 1, 2 and 6 hours after oral gavage with olive oil. Liver lysates were assayed for PAI-1-free tPA concentration by ELISA. Data are shown as mean±s.e.m.; *P<0.05 by one-way analysis of variance (ANOVA) followed by Dunnett's test (FIGS. 5D-5G, 5I, 5J). n.s., not significant (P≥0.05).

FIGS. 6A-6J show that PAI-1 deficiency leads to lower concentrations of plasma apoB and apoB-cholesterol in mice and humans. FIG. 6A: Serpine1fl/fl mice were treated with AAV8-TBG-Cre (Cre) or control AAV8-TBG-LacZ (Ctrl) and then fed a high-fat diet for 8 weeks. Plasma total cholesterol concentration was measured (n=6 mice per group). FIG. 6B: Serpine1fl/fl mice were treated with AAV8-TBG-Cre (Cre) or control AAV8-TBG-LacZ (Ctrl) and then fed a high-fat diet for 8 weeks. Plasma apoB was measured by immunoblot (n=6 mice per group). FIG. 6C: Serpine1fl/fl mice were treated with AAV8-TBG-Cre (Cre) or control AAV8-TBG-LacZ (Ctrl) and then fed a high-fat diet for 8 weeks. Plasma samples were subjected FPLC fractionation and assayed for cholesterol concentration. For the FPLC profile of cholesterol, the cholesterol in VLDL fractions is zoomed in a smaller graph. (n=6 mice per group). FIG. 6D: Serpine1fl/fl mice were treated with AAV8-TBG-Cre (Cre) or control AAV8-TBG-LacZ (Ctrl) and then fed a high-fat diet for 8 weeks. Liver lysates were assayed for PAI-1-free tPA concentration by ELISA (n=6 mice per group). FIG. 6E: Serpine1fl/fl mice were treated with AAV8-TBG-Cre (Cre) or control AAV8-TBG-GFP (Ctrl) and then fed a normal chow diet for 4 weeks. Mice were injected with P407 i.p. to assess VLDL secretion (n=10 mice per group). FIG. 6F: Serpine1fl/fl mice were treated with AAV8-TBG-Cre (Cre) or control AAV8-TBG-GFP (Ctrl) and then fed a normal chow diet for 6 weeks. The mice had their food withdrawn for 5 hours and then were euthanized at 0, 2 and 4 hours after oral gavage with olive oil. Plasma apoB-100 concentration was measured by ELISA. FIG. 6G: Plasma samples from SERPINE1-deficient humans and unaffected age/gender/BMI-matched individuals from the same community were assayed for VLDL cholesterol, LDL cholesterol and apoB-100 concentrations (n=10 per group). FIG. 6H: tPA concentration was measured in VLDL isolated by ultracentrifugation from the plasma of the subjects in panel FIG. 6G (n=10 per group). FIG. 6I: VLDL from the plasma of the subjects in panel FIG. 6G was analyzed by dynamic light scattering (n=10 per group). The correlation between VLDL-associated tPA and VLDL diameter was calculated (n=10 per group). FIG. 6J: A schematic diagram depicting how tPA-PAI-1 interaction in hepatocytes determines VLDL assembly. Without lipid stimulation, tPA interacts with apoB and inhibits MTP-apoB interaction in the ER, thereby limiting MTP-mediated apoB lipidation and VLDL assembly. When hepatocytes are loaded with lipid, PAI-1 sequesters free tPA away from apoB and increases VLDL assembly. The diagram was generated using biorender.com. Data are shown as mean±s.e.m.; P values were calculated by two-tailed Student's t-test (FIGS. 6A, 6D, 6E, 6F), paired Student's t-test (FIGS. 6G, 6H), or Pearson's correlation analysis (FIG. 6I). *P<0.05.

FIGS. 7A-7D show that modulating hepatocyte tPA expression changes plasma tPA levels in mice. FIG. 7A: Ldlr^{-/-} mice were treated with AAV8-H1-shPlat (sh-tPA) or AAV8-H1-scrambled control (scr) and then fed the Western diet for 8 weeks. Plasma tPA were measured by ELISA (n=9-10 mice per group). FIG. 7B: Apoe^{-/-} mice were treated with AAV8-H1-shPlat (sh-tPA) or AAV8-H1-scrambled control (scr) and then fed the Western diet for 8 weeks. Plasma tPA were measured by ELISA (n=5 mice per group). FIG. 7C: C57BL/6J mice were treated with AAV8-H1-shPlat (sh-tPA) or AAV8-H1-scrambled control (scr) and then fed the Western diet for 8 weeks. FIG. 7D: Plat^{fl/fl} mice were treated with AAV8-TBG-cre (Cre) or AAV8-TBG-GFP (GFP) and then fed the Western diet for 8 weeks. Plasma tPA were measured by ELISA (n=6 mice per group). Plasma tPA were measured by ELISA (n=9-10 mice per group). Data are shown as mean±s.e.m.; *P<0.05 by two-tailed Student's t-test.

FIG. 8 shows that silencing hepatocyte tPA increases plasma apoB-lipoprotein cholesterol and apoB in mice. Wild-type mice were treated with AAV8-H1-sh-tPA (sh-tPA) or AAV8-H1-scramble (scr) and then fed the Western diet for 8 weeks. The livers were assayed for tPA protein, and plasma samples were assayed for tPA, total cholesterol and apoB-100 concentrations and for FPLC profiles of cholesterol, triglyceride, and apoB-100. The cholesterol in VLDL fractions is zoomed in a smaller graph. (n=6 mice per group). Data are shown as mean±s.e.m; *P<0.05 by two-tailed Student's t-test.

FIGS. 9A-9C show that silencing hepatocyte tPA does not change hepatic apoB mRNA levels in mice. FIG. 9A: Ldlr^{-/-} mice were treated with AAV8-H1-shPlat (sh-tPA) or AAV8-H1-scrambled control (scr) and then fed the Western diet for 8 weeks. Liver apoB mRNA were measured by real-time PCR. FIG. 9B: Apoe^{-/-} mice were treated with AAV8-H1-shPlat (sh-tPA) or AAV8-H1-scrambled control (scr) and then fed the Western diet for 8 weeks. Liver Apob mRNA were measured by real-time PCR. FIG. 9C: C57BL/6J mice were treated with AAV8-H1-shPlat (sh-tPA) or AAV8-H1-scrambled control (scr) and then fed the Western diet for 8 weeks. Liver Apob mRNA were measured by real-time PCR. Data are shown as mean±s.e.m.; Statistical analysis was performed by two-tailed Student's t-test. n.s., not significant (P≥0.05).

FIGS. 10A-10B show that silencing hepatocyte tPA does not change plasma apoE and liver LDLR levels. FIG. 10A: C57BL/6J mice were treated with AAV8-H1-shPlat (sh-tPA) or AAV8-H1-scrambled control (scr) and then fed the Western diet for 8 weeks. Plasma apoE concentration were measured by ELISA. FIG. 10B: C57BL/6J mice were treated with AAV8-H1-shPlat (sh-tPA) or AAV8-H1-scrambled control (scr) and then fed the Western diet for 8 weeks. Liver LDLR was measured by immunoblot. Data are shown as mean±s.e.m.; Statistical analysis was performed by two-tailed Student's t-test. n.s., not significant (P≥0.05).

FIGS. 11A-11D show that silencing hepatocyte tPA does not change apoB mRNA levels. FIG. 11A: Human primary hepatocytes were treated with siRNA against tPA mRNA (si-tPA) or scrambled RNA for 24 hours. Cell tPA mRNA was measured by real-time PCR. FIG. 11B: Human primary hepatocytes were treated with siRNA against tPA (si-tPA) or scrambled RNA for 24 hours. Cell apoB mRNA was measured by real-time PCR. FIG. 11C: McA-RH7777 cells were treated with siRNA against tPA (si-tPA) or scrambled RNA for 24 hours. Cell tPA mRNA was measured by real-time PCR. FIG. 11D: McA-RH7777 cells were treated with siRNA against tPA (si-tPA) or scrambled RNA for 24 hours. Cell apoB mRNA was measured by real-time PCR. Data are shown as mean±s.e.m.; *P<0.05 by two-tailed Student's t-test. n.s., not significant (P≥0.05).

FIG. 12 shows that silencing tPA increases apoB secretion in McA-RH7777 cells. McA-RH7777 cells were treated with siRNA against tPA mRNA (si-tPA) or scrambled RNA for 24 hours. ApoB secretion was measured by [³H]-labeling as in FIGS. 2G and 2H. Data are shown as mean±s.e.m.; *P<0.05 by two-tailed Student's t-test or two-way analysis of variance (ANOVA) followed by Dunnett's test. n.s., not significant (P≥0.05).

FIG. 13 shows that incubation of human primary hepatocytes with recombinant tPA does not change cell medium apoB levels. Human primary hepatocytes were incubated in a medium containing recombinant human tPA (10 ng/ml) for 1, 6, and 24 hours, respectively. Hepatocytes incubated in medium without adding recombinant tPA were used as controls (Ctrl). ApoB-100 in cell medium were measured by ELISA. VLDL were isolated by ultracentrifugation, and cholesterol and triglyceride in VLDL were measured. Data are shown as mean±s.e.m.; * Statistical analysis was performed by two-way analysis of variance (ANOVA) followed by Dunnett's test. n.s., not significant (P≥0.05).

FIG. 14 shows that silencing tPA increases MTP-dependent lipid transfer in McA-RH7777 cell microsomal fraction. McA-RH7777 cells were treated with siRNA against tPA mRNA (si-tPA) or scrambled RNA for 24 hours. The microsomal fraction was assayed for neutral lipid transfer activity with or without 10 nM CP-346086, an MTP inhibitor. Data are shown as mean±s.e.m.; *P<0.05 by two-tailed Student's t-test or one-way analysis of variance (ANOVA) followed by Dunnett's test.

FIGS. 15A-15B show purification of human recombinant tPA and LDL by size-exclusion chromatography. FIG. 15A: Further purification of ultracentrifugation-isolated human LDL by size-exclusion chromatography. FIG. 15B: Further purification of affinity chromatography-purified recombinant human tPA by size-exclusion chromatography.

FIG. 16 shows that tPA interacts with delipidated apoB-100. Purification of human recombinant tPA and LDL by size-exclusion chromatography. A solid-binding was used to assay the interaction between delipidated apoB-100 and recombinant tPA. Data are shown as mean±s.e.m.

FIG. 17 shows that tranexamic acid partially reduces the interaction between tPA and solid-phase-bound LDL. Solid-phase binding assays were used to measure the interaction between LDL and recombinant human tPA in the presence of tranexamic acid. Data are shown as mean±s.e.m; *P<0.05 by one-way analysis of variance (ANOVA) followed by Dunnett's test. n.s., not significant (P≥0.05).

FIG. 18 shows that silencing tPA PAI-1 reduces apoB secretion in McA-RH7777 cells. McA-RH7777 cells were treated with siRNA against PAI-1 mRNA (si-PAI1) or scrambled RNA for 24 hours. Cells were treated with 0.4 mM oleate complexed with fatty acid-free BSA (oleate group) or fatty acid-free BSA alone (without oleate group). ApoB secretion was measured by [³H]-labeling as in FIG. 2. Data are shown as mean±s.e.m; *P<0.05 by two-tailed Student's t-test. n.s., not significant (P≥0.05).

FIG. 19 shows that oral gavage with olive oil does not change liver total tPA and total PAI-1 levels in mice. Normal chow diet-fed C57BL/6 mice has their food withdrawn for 5 hours and sacrificed at 0, 1, 2 and 6 hours after oral gavage with olive oil. Liver lysates were assayed for tPA and PAI-1 concentrations by ELISA. Data are shown as mean±s.e.m; *P<0.05 by two-tailed Student's t-test or two-way analysis of variance (ANOVA) followed by Dunnett's test. n.s., not significant (P≥0.05).

FIGS. 20A-20B show that silencing tPA in hepatocytes increases apo(a) levels in cell medium. Cell medium apo(a) levels were measured by ELISA. FIG. 20A is a chart showing apo(a) levels in scramble siRNA- or si-tPA-treated human primary hepatocytes. FIG. 20B is a chart showing apo(a) levels in HepG2 cells transduced with apo(a)-expressing plasmids and then treated with scramble siRNA or si-tPA.

FIGS. 21A-21C show that knocking out enterocyte tPA in mice increased plasma cholesterol levels. Enterocyte tPA knockout mice (e-tPA-KO) or littermate controls (ctrl) were processed to measure: FIG. 21A: tPA mRNA levels in enterocytes and liver normalized to Rplp0 mRNA; FIG. 21B: plasma total cholesterol; and FIG. 21C: plasma cholesterol profiled by FPLC (Ctrl, pooled plasma from 8 mice; e-tPA-KO, pooled plasma from 11 mice).

FIGS. 22A-22B show that knocking out enterocyte tPA in mice promoted chylomicron production. Enterocyte tPA knockout mice (e-tPA-KO) or littermate controls (ctrl) were challenged with oral olive oil gavage. FIG. 22A: time-course of plasma triglyceride levels post-gavage (n=9 for ctrl (bottom line in chart) and n=11 for e-tPA-KO (top line in chart)); FIG. 22B: Immunoblot analysis of apoB-48 levels in isolated chylomicron fractions and of albumin in plasma collected 2 hours post-gavage.

FIGS. 23A-23B show that expression of tPA-K2 domain reduces apoB secretion in cultured hepatocytes. Cultured McA-RH7777 cells were treated with a plasmid (SEQ ID NO: 33) encoding a tPA-K2 (SEQ ID NO: 31) or with GFP control. FIG. 23A: the expression of tPA-K2 domain was detected by immunoblot. FIG. 23B: the apoB levels in cell medium were detected by immunoblot.

FIGS. 24A-24B show that expression of a tPA-K2 domain reduces plasma lipids in mice. Male C57 wild-type mice were fed a high-fat diet for 4 weeks. Mice were i.v. injected with LNP carrying mRNA encoding a tPA-K2 domain with an endoplasmic reticulum localization motif or control Luciferase (1 mg/kg body weight). Blood was collected at 6-, 24- and 48-hours post-injection and just before injection. Plasma triglyceride (FIG. 24A) and cholesterol (FIG. 24B) levels were measured.

FIG. 25 shows that expression of a tPA-K2 domain does not change bleeding time in mice. Male C57 wild-type mice were fed a high-fat diet for 5 weeks. Mice were i.v. injected with LNP carrying mRNA encoding a tPA-K2 domain with an endoplasmic reticulum localization motif or control Luciferase (1 mg/kg body weight). Tail bleeding tests were conducted 24 hrs post-injection of LNP.

### DETAILED DESCRIPTION

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs.

### Overview

Statins and protein convertase subtilisin/kexin type 9 (PCSK9) inhibitors, both of which lower low-density lipoprotein (LDL) by enhancing hepatic LDL receptor (LDLR)-mediated LDL clearance, are currently used to prevent or treat cardiovascular disease. However, despite being able to reduce LDL, these treatments only mildly alter the levels of other atherogenic apolipoprotein B (apoB) containing apolipoproteins, e.g., intermediate density lipoproteins (IDLs), very low density lipoproteins (VLDLs), lipoprotein (a) [Lp(a)], chylomicron and chylomicron remnant.

ApoB, a large amphipathic protein, is the structural scaffold for the formation of VLDL and chylomicron [8]. VLDL and chylomicron are assembled in hepatocytes and enterocytes, respectively, through incorporating triglyceride, cholesteryl esters, and phospholipids onto apoB to form spherical particles [9]. This process, known as apoB lipidation, is dependent on both lipid availability, and a neutral lipid transporter, microsomal triglyceride transfer protein (MTP) [10, 11]. MTP binds to apoB in the hepatocyte endoplasmic reticulum (ER) and transfers lipids onto apoB [10]. When MTP and/or lipids are not available, VLDL and chylomicron cannot be synthesized, and newly translated apoB is targeted for degradation [12, 13]. Although the essential role of MTP in apoB lipidation has been well-established, much less is known about the regulation of apoB-MTP interaction and MTP-mediated lipid transfer to apoB.

Tissue plasminogen activator (tPA) is a serine protease that plays a critical role in fibrinolysis, a process that lyses blood clots [14]. Previous studies have shown that low plasma tPA activity is associated with a higher risk of atherosclerotic CVD [15-17], but whether decreased fibrinolysis contributes to CVD in this setting remains unknown. Another plausible mechanism linking low tPA to CVD is elevated plasma cholesterol, as this is seen in humans with decreased tPA activity [18-20]. However, very little is known about how tPA might affect circulating atherogenic lipoproteins. Given the central role of hepatocytes in apoB-lipoprotein production and the inventors' recent study showing that hepatocytes are an important source of tPA [21, 22], the inventors sought to identify possible links between tPA and apoB-lipoprotein assembly and secretion in hepatocyte and enterocyte. The inventors' investigation revealed that endogenous hepatocyte tPA limits VLDL and chylomicron production by directly interacting with apoB, interrupting the interaction between apoB and MTP, and therefore impairing the MTP-dependent neutral lipid transfer and apoB lipidation.

Plasminogen activator inhibitor 1 (PAI-1), encoded by SERPINE1 gene, is the key serine protease inhibitor of tPA, and is also expressed in hepatocytes. In this disclosure, the inventors demonstrate that PAI-1 binds to tPA within hepatocytes and abolishes the effect of tPA on limiting VLDL assembly. Moreover, the regulatory pathway of VLDL assembly by the hepatocyte PA1-1/tPA-axis is of physiologic relevance in post-prandial lipid-loading-associated VLDL production. These findings suggest novel therapeutic strategies to lower atherogenic apoB-lipoproteins production.

The inventors of the present technology discovered that tPA directly interacts with apoB and prevents transfer of neutral lipids to apoB by MTP, leading to degradation of apoB and thereby reducing levels of atherogenic apolipoproteins.

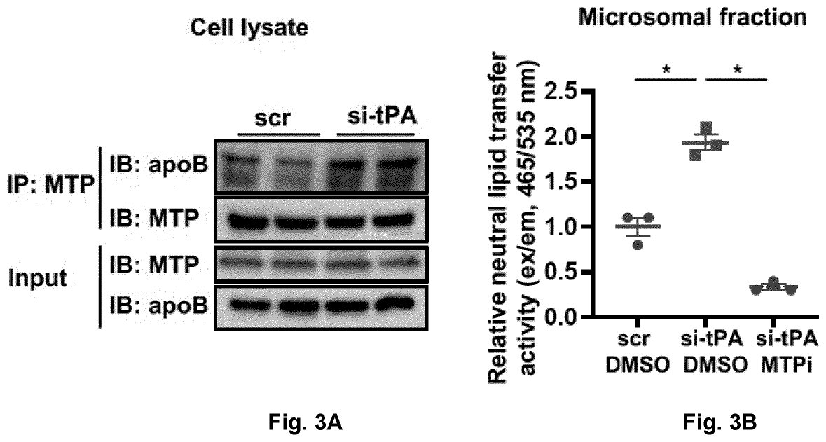

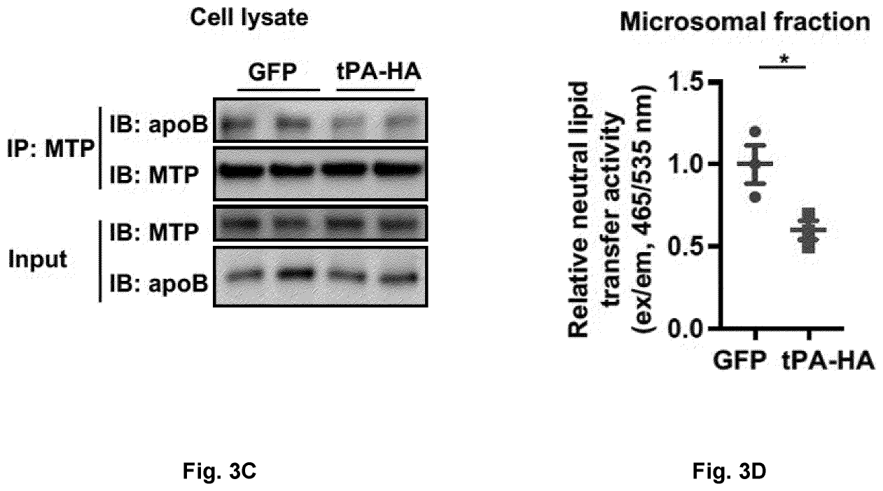

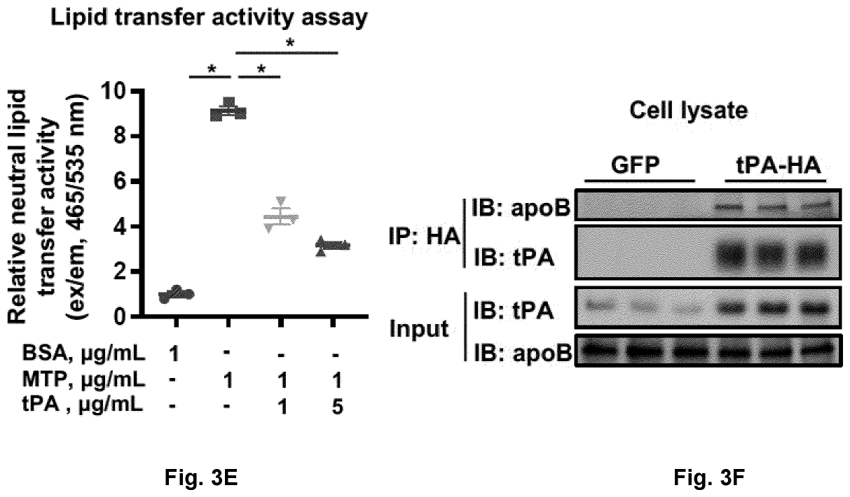

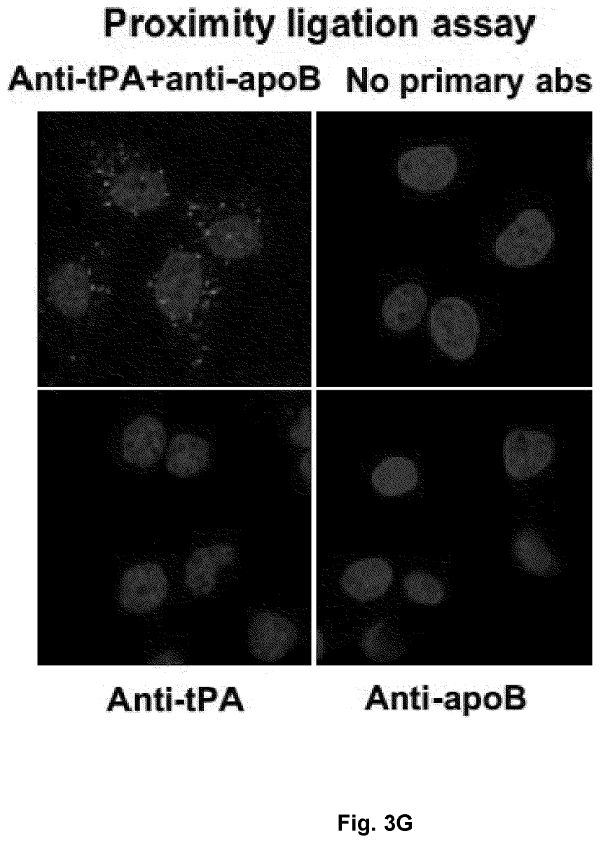

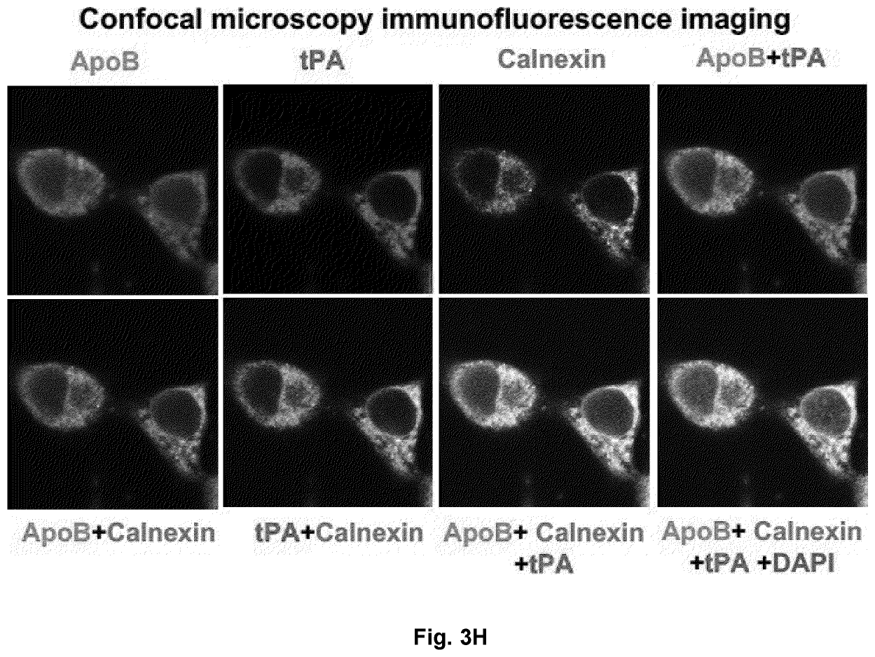

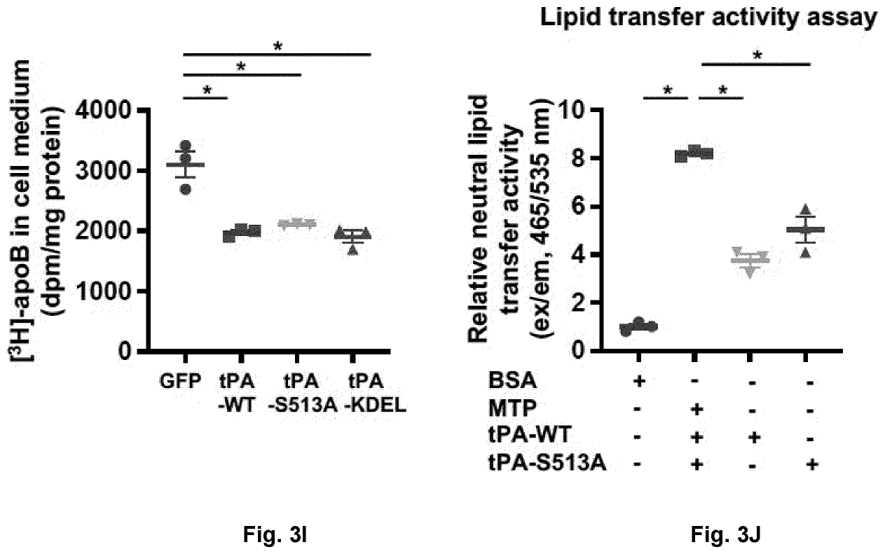

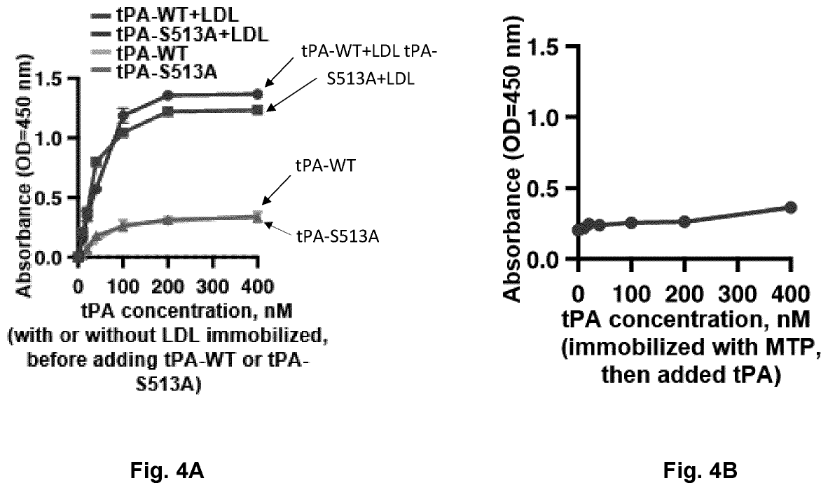

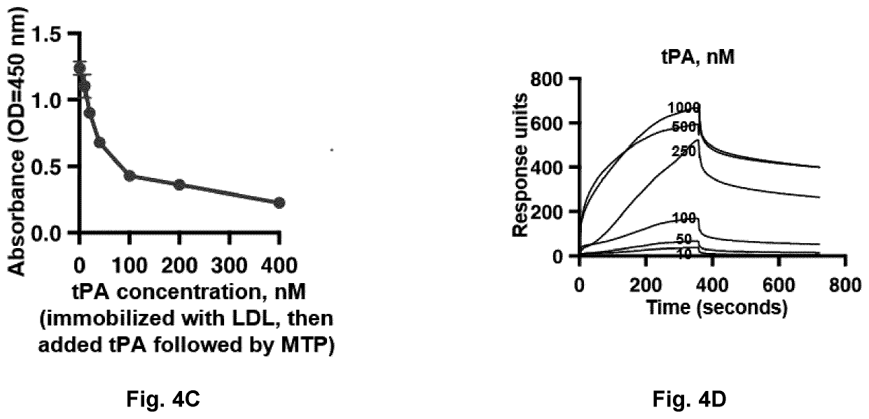

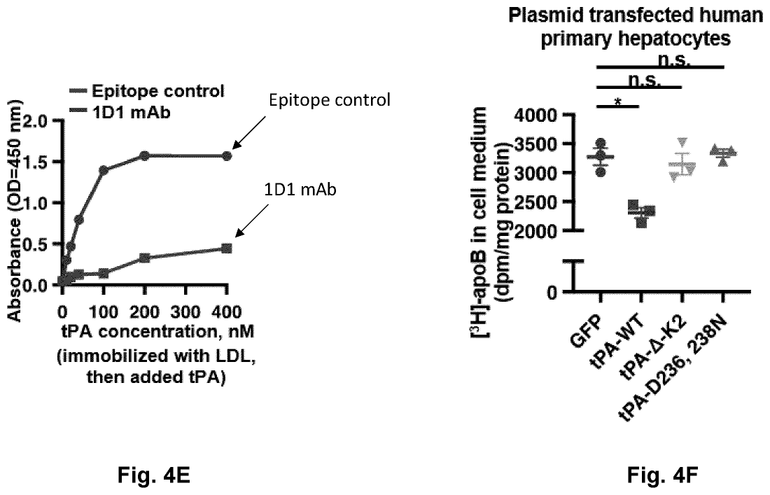

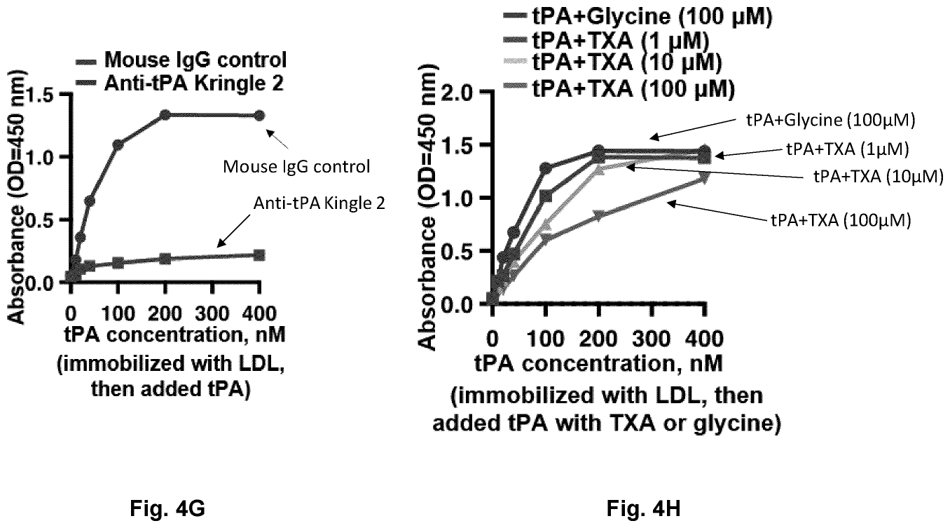

Further, the inventors of the present technology discovered that the serine protease function of tPA (specific cleavage of Arg-Val bond in plasminogen to form plasmin: EC: 3.4.21.68) is not required for tPA to reduce apoB secretion by hepatocytes. See, e.g., FIGS. 3I, 3J, 4A, which demonstrate that the expression of protease deficient tPA S513A mutant in hepatocytes still leads to reduced apoB secretion in conditional tPA knockout animals.

Recombinant tPA (also known as rtPA and ACTIVASE (alteplase)) is used for its “clot busting” activity, which helps dissolve blood clots and is used to treat heart attacks, strokes, and clots in the lungs. However, these functions are dependent on the serine protease activity of tPA.

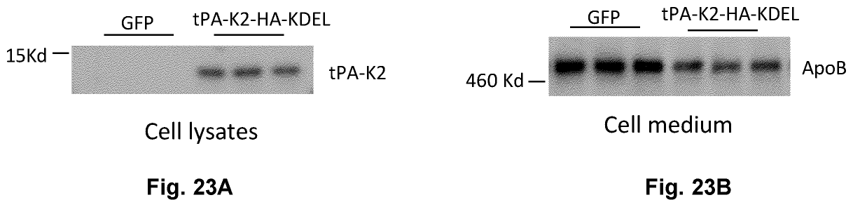

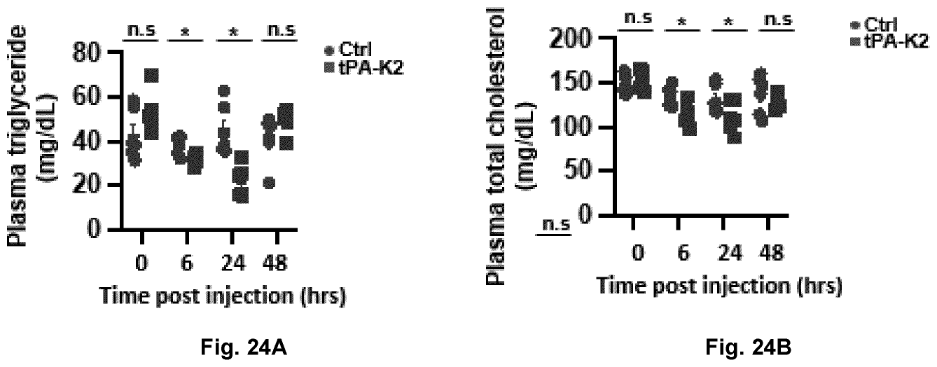

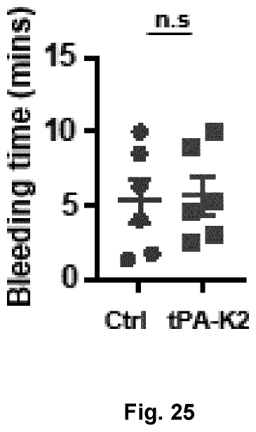

The disclosure of the present technology is based, at least in part, on the discovery that the tPA-K2 polypeptides of the present technology—although lacking serine protease and proteolytic capabilities—are effective in methods for reducing apoB secretion and lowering plasma triglyceride and total cholesterol levels when administered to a subject in need thereof. Moreover, because the tPA-K2 fragments of the present technology (e.g., comprising an amino acid sequence as set forth in SEQ ID NO. 7), do not have serine protease and proteolytic activity, administration of the peptides of the present technology will not increase the bleeding risk in a subject. The examples provided herein demonstrate that the expression of recombinant tPA-K2 peptides comprising the tPA Kringle 2 domain alone and an endoplasmic reticulum localization sequence are able to reduce apoB secretion from hepatocytes (FIGS. 23A-23B) and lower plasma lipid levels in an in vivo mouse model (FIGS. 24A-24B), but do not alter the bleeding time in the mice (FIG. 25). These data support that compositions comprising the recombinant tPA-K2 polypeptides (or nucleic acids (e.g., cDNA, mRNA) encoding the same) are useful in methods for the treatment of diseases or conditions associated with elevated plasma apoB-lipoprotein levels. In some embodiments, the compositions of the present technology are useful in methods for reducing any one or more of plasma VLDL, IDL, LDL, Lp(a), chylomicron, chylomicron remnant, triglyceride, or total cholesterol levels.

### Compositions

In an aspect of the current disclosure, recombinant tissue plasminogen activator kringle 2 domain (tPA-K2) polypeptides, or nucleic acids (e.g., mRNA, cDNA) encoding the same, are provided. As used herein, “recombinant tissue plasminogen activator kringle 2 domain (tPA-K2) polypeptide” or “recombinant tPA-K2 polypeptide,” also referred to as the “recombinant polypeptides” of the instant disclosure, refers to a recombinant polypeptide comprising the kringle 2 domain of tissue plasminogen activator, a fragment of the kringle 2 domain, fragments thereof, or nucleic acids encoding the same, wherein the recombinant polypeptide has less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, or less than about 10% sequence identity to the full length wild-type human tPA (SEQ ID NO: 1). Accordingly, the present technology provides a recombinant tPA-K2 polypeptide having an amino acid sequence that is at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% identical to an amino acid sequence described in any of SEQ ID NOs: 1, 3, 5, 7, 9, 13, 14, 30, 31, 35, or 37. Recombinant tPA-K2 polypeptides suitable for use in the methods described herein also include variants, including polypeptides having amino acid changes, e.g., amino acid substitutions, deletions, or additions, compared to the amino acid sequences of any tPA-K2 polypeptide described herein. Such sequence-variant proteins are suitable for the methods described herein provided the altered amino acid sequence retains sufficient biological activity to be functional in the compositions and methods described herein. Where amino acid substitutions are made, the substitutions can be conservative amino acid substitutions. Among the common, naturally occurring amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

The present technology also provides nucleic acids (e.g., mRNA, cDNA) encoding the recombinant tPA-K2 polypeptides, such as a polynucleotide having a nucleic acid sequence that is at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or 100% identical to a nucleic acid sequence described in any of SEQ ID NOs: 2, 4, 6, 8, 10, 15, 16, 32, 34, 36, 38, 39, or 40, or a complement thereof, or to a nucleic acid sequence encoding a polypeptide described in any of SEQ ID Nos: 1, 3, 5, 7, 9, 13, 14, 30, 31, 35, or 37. In some embodiments, the present technology provides RNA transcripts encoded by any of SEQ ID NOs: 2, 4, 6, 10, 15, 16, 34, 36, 38, or 39. In some embodiments, the nucleic acid is capable of reducing apoB-lipoprotein blood (e.g., plasma or serum) levels when administered to a subject.

Exemplary recombinant tPA-K2 polypeptides of the present technology (or nucleic acids encoding the same) include, for example, SEQ ID NOs: 1, 3, 5, 7, 9, 13, 14, 30, 31, 35, or 37. In some embodiments, the recombinant tPA-K2 polypeptides of the present technology (or nucleic acids encoding the same) comprise an endoplasmic reticulum (ER) localization sequence. One non-limiting example of an ER localization sequence is KKXX (where “K” stands for lysine and “X” can be any amino acid). Another non-limiting example of an ER localization sequence is KDEL (SEQ ID NO: 11). Accordingly, for example, SEQ ID NO: 7 is a recombinant tPA-K2 polypeptide, which comprises SEQ ID NO: 5 and a KDEL (SEQ ID NO: 11) sequence. In some embodiments, the recombinant tPA-K2 polypeptides of the present technology (or nucleic acids encoding the same) include, for example, SEQ ID NO: 9, which is a tPA S513A mutant lacking serine protease function. In some embodiments, the tPA S513A mutant further comprises an ER localization sequence, such as KDEL (SEQ ID NO: 11), and comprises the amino acid sequence as set forth in SEQ ID NO: 35. In some embodiments, the recombinant tPA-K2 polypeptides of the present technology (or nucleic acids encoding the same) include, for example, SEQ ID NO: 7, which comprises SEQ ID NO: 5 and a KDEL (SEQ ID NO: 11). In some embodiments, the recombinant tPA-K2 polypeptides may comprise a sequence with 100% identity, at least about 99% identity, at least about 98% identity, at least about 97% identity, at least about 96% identity, or at least about 95% identity to the full length wild-type tPA sequence (SEQ ID NO: 1), but further comprises one or more additional heterologous amino acid sequences.

The recombinant tPA-K2 polypeptides of the present technology may be constructed by methods well known in the art. By way of example, but not by way of limitation, a nucleotide sequence encoding a recombinant tPA-K2 polypeptide can be generated by PCR. In some embodiments, the tPA-K2 sequence comprises an in-frame C-terminal endoplasmic reticulum localization sequence, such as a sequence encoding KDEL (e.g., SEQ ID NO: 12). In some embodiments, the recombinant tPA-K2 polypeptides of the present technology may be referred to as “fusion proteins,” comprising a tPA-K2 polypeptide, or fragment thereof, a linker, and an endoplasmic reticulum localization motif. In some embodiments, the recombinant tPA-K2 polypeptides of the present technology have the following, non-limiting, formula: tPA-K2 polypeptide (or fragment thereof) -X- endoplasmic reticulum localization motif (Formula I), wherein -X- is a linker. In some embodiments, -X- is one or more amino acids. In some embodiments, the amino acid sequence of the linker comprises GGGGS (SEQ ID NO: 41).

In some embodiments, the recombinant tPA-K2 polypeptides of the present technology comprise a protein tag domain that comprises one or more amino acid sequences that facilitates immunoprecipitation, purification, and/or detection of the exogenously expressed fusion protein. In some embodiments, the protein tag domain comprises an epitope tag and/or a polyhistidine tag. By way of example, but not by way of limitation, exemplary tags include one or more of an HA (hemagglutinin) tag, a histidine-tag (e.g., a 6-histidine tag), a FLAG tag, a CBP (calmodulin binding peptide), a CYD (covalent yet dissociable NorpD peptide), Strepll, or HPC (heavy chain of protein C). In some embodiments, the protein tag domain comprises about 10 to 20 amino acids in length. In some embodiments, the protein tag domain comprises 2 to 40 amino acids in length, for example 6-20 amino acids in length. In some embodiments, the epitope tag is an HA tag. In some embodiments, the HA tag comprises the amino acid sequence of YPYDVPDYA (SEQ ID NO: 42). One of skill in the art can appreciate that the addition of an epitope tag, such as an HA tag, can be employed to facilitate immunoprecipitation, purification, and/or detection of an expressed protein and that the inclusion of a tag is in no way meant to be limiting to the recombinant tPA-K2 polypeptides or nucleic acids encoding the same or their function as described herein.

In some embodiments, the recombinant tPA-K2 polypeptides of the present technology (or nucleic acids encoding the same) are therapeutic polypeptides (or nucleic acids) and are useful in inhibiting the interaction of MTP and apoB, blocking PAI-I binding to endogenous tPA, inhibiting the formation of VLDL and chylomicron, enhancing degradation of apoB, and reducing the likelihood of CVD and related diseases and conditions associated with VLDL, IDL, LDL, Lp(a), chylomicron, and chylomicron remnants, such as but not limited to hyperlipidemia, atherosclerosis, increased risk of blood clots, angina, heart attack, heart failure, stroke, transient ischemic attack(s) (TIA), peripheral artery disease, or high blood pressure. The recombinant tPA-K2 polypeptides of the instant disclosure comprise a tissue plasminogen kringle 2 domain, e.g., SEQ ID NO: 5, or a fragment thereof. The tPA kringle 2 domain may comprise a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to SEQ ID NO: 5. Without wishing to be bound by theory, the disclosed compositions block the interaction of MTP and apoB. The tPA kringle 2 domain (tPA-K2) may comprise the sequence NPDGDAKPWCHVLKNRRLTWEY (SEQ ID NO: 13). The tPA-K2 may further comprise a KDEL (SEQ ID NO: 11) sequence, which localizes the tPA-K2 to the endoplasmic reticulum, e.g., NPDGDAKPWCHVLKNRRLTWEYKDEL (SEQ ID NO: 14).

The recombinant polypeptides of the instant disclosure may also comprise tPA with no serine protease activity, i.e., the tPA is not fibrinolytic. As discussed above, serine protease activity is required for the “clot busting” or fibrinolytic activity tPA. Loss of serine protease activity may be accomplished in the recombinant polypeptides by mutation of amino acid residues that are critical to the serine protease function of tPA, e.g., serine 513 may be mutated to alanine (S513A), wherein the 513 position is relative to SEQ ID NO: 1. Alternatively, the entire peptidase domain of tPA, or a critical portion of the peptidase domain of tPA may be removed from the recombinant polypeptide. The peptidase domain of tPA comprises amino acids 311-561, with reference to SEQ ID NO: 1. Therefore, the recombinant polypeptides may comprise SEQ ID NO: 1, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to SEQ ID NO: 1, with a suitable mutation, e.g., S513A, to destroy serine protease function of tPA, or with a truncation of SEQ ID NO: 1 to remove the peptidase domain, i.e., amino acids 311-561, with reference to SEQ ID NO: 1.

The recombinant polypeptides may comprise SEQ ID NO: 9 (tPA-S513A), or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to SEQ ID NO: 9.

The recombinant polypeptides may comprise SEQ ID NO: 35 (tPA-S513A-KDEL), or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to SEQ ID NO: 35.

The recombinant polypeptides may comprise SEQ ID NO: 7 (tPA-K2 KDEL), or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to SEQ ID NO: 7.

The recombinant polypeptides may comprise SEQ ID NO: 5 (tPA kringle 2 domain), or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to SEQ ID NO: 5.

tPA lacking the peptidase domain, i.e., lacking amino acids 311-516 with reference to SEQ ID NO: 1, may comprise SEQ ID NO: 30, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to SEQ ID NO: 30.

In some embodiments, delivery of the disclosed polypeptides, or nucleic acids (mRNA, cDNA) encoding the same, may be efficiently facilitated by nanoparticles. Therefore, the present disclosure further provides nanoparticles comprising the recombinant polypeptides, or nucleic acids encoding the same. The nanoparticles may comprise one or more polymers, e.g., poly(lactide) (PLA), poly(lactide-co-glycolide) (PLGA) copolymers, poly (ε-caprolactone) (PCL), and poly(amino acids), alginate, chitosan, gelatin, and albumin.

In some embodiments, the nanoparticles may comprise, e.g., one or more lipids, e.g., 1,2-di-(9Z-octadecenoyl)-sn-glycero-3-phosphocholine (DOPC), which may suitably be formed into a liposome. Additional or alternative exemplary lipids for incorporation into nanoparticles are known in the art and include, but are not limited to, 1,2-dipalmitoyl-sn-glycero-3-phosphoethanolamine-N-[3-(2-pyridyldithio) propionate] (PDP-PE), 306Oᵢ₁₀, tetrakis(8-methylnonyl) 3,3′,3″,3″-(((methylazanediyl)bis(propane-3,1 diyl))bis (azanetriyl))tetrapropionate; 9A1P9, decyl(2-(dioctylammonio)ethyl)phosphate; A2-Iso5-2DC18, ethyl 5,5-di((Z)-heptadec-8-en-1-yl)-1-(3-(pyrrolidin-1-yl) propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; C12-200, 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol); cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino)butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DOTAP, 1,2-dioleoyl-3-trimethylammonium-propane; DOTMA, 1,2-di-O-octadecenyl-3-trimethylammonium-propane; DSPC, 1,2-distearoyl-sn-glycero-3-phosphocholine; ePC, ethylphosphatidylcholine; FTT5, hexa (octan-3-yl) 9,9′,9″,9′″,9″″,9′″″-((((benzene-1,3,5-tricarbonyl)yris(azanediyl))tris(propane-3,1-diyl))tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl)tetrakis(ethane-2,1-diyl)(9Z,9′Z,9″Z,9′″Z,12Z,12′Z,12″Z,12′″Z)-tetrakis (octadeca-9,12-dienoate); PEG2000-DMG, 1,2-dimyristoyl-rac-glycero-3-methoxypolyethylene glycol-2000; TT3, N¹,N³,N⁵-tris(3-(didodecylamino) propyl)benzene-1,3,5-tricarboxamide.

### Polynucleotides

As described above, the present disclosure also provides polynucleotides encoding the recombinant polypeptides of the instant disclosure. The polynucleotides of the present disclosure may comprise a nucleotide sequence set forth in any one or more of SEQ ID NOs: 2, 4, 6, 8, 10, 15, 16, 32, 34, 36, 38, 39, or 40, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to SEQ ID NOs: 2, 4, 6, 8, 10, 15, 16, 32, 34, 36, 38, 39, or 40, or nucleotide sequences encoding a polypeptide described in any of SEQ ID Nos: 1, 3, 5, 7, 9, 13, 14, 30, 31, 35, or 37. The polynucleotides of the present disclosure also encompass RNA sequences encoded by any one of SEQ ID NOs: 2, 4, 6, 10, 15, 16, 34, 36, 38, or 39. In some embodiments, the polynucleotide comprises the mRNA sequence set forth in SEQ ID NO: 8. In some embodiments, the polynucleotide comprises the mRNA sequence set forth in SEQ ID NO: 40. In some embodiments, the polynucleotide comprises the mRNA sequence set forth in SEQ ID NO: 32.

In some embodiments, the polynucleotide comprises the tPA kringle 2-KDEL encoding nucleic acid sequence as set forth in SEQ ID NO: 34, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to SEQ ID NO: 34.

In some embodiments, the polynucleotide comprises the tPA kringle 2 domain encoding nucleic acid sequence as set forth in SEQ ID NO: 6, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to SEQ ID NO: 6.

In some embodiments, the polynucleotide comprises the tPA S513A encoding nucleic acid sequence as set forth in SEQ ID NO: 10, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to SEQ ID NO: 10.

In some embodiments, the polynucleotide comprises the tPA-S513A-KDEL encoding nucleic acid sequence encoding nucleic acid sequence as set forth in SEQ ID NO: 36, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to SEQ ID NO: 36.

In some embodiments, the polynucleotides of the present technology comprise a nucleotide sequence encoding a protein tag (e.g., an epitope tag) that facilitates immunoprecipitation, purification, and/or detection of an exogenously expressed protein. One of skill in the art can appreciate that the inclusion of a nucleotide sequence encoding an epitope tag, such as an HA tag, can be employed to facilitate immunoprecipitation, purification, and/or detection of an expressed protein and that the inclusion of a tag is in no way meant to be limiting to nucleic acids encoding recombinant tPA-K2 polypeptides or to their function as described herein.

The polynucleotides of the instant disclosure may further comprise at least one regulatory sequence operably linked to a nucleotide sequence encoding a recombinant polypeptide of the instant disclosure. The at least one regulatory region may comprise, e.g., an enhancer, a promoter, or both an enhancer and promoter. The regulatory region may comprise a tissue-specific promoter, e.g., a hepatocyte-specific promoter, such as a thyroxine binding globulin (TBG) promoter (SEQ ID NO: 17). As used herein, a polynucleotide is “operably linked” or “operably connected” when it is placed into a functional relationship with a second polynucleotide sequence.

It is envisioned that the disclosed polynucleotides may be utilized to express the disclosed polypeptides in cells, e.g., human cells, e.g., hepatocytes, or in a human subject. Accordingly, the disclosed polynucleotides may be, e.g., a plasmid, a minicircle, and may comprise regulatory regions allowing for viral delivery of the polynucleotides.

For example, the disclosed polynucleotides may comprise viral regulatory regions, e.g., adeno-associated virus (AAV) inverted terminal repeat sequences (ITR).

As used herein, the term “inverted terminal repeat” (ITR) sequences refers to sequences of DNA that flank the portion of the AAV genome that allows insertion of the genome into the host cell. ITRs are the only cis-acting element required for AAV formation. Therefore, the additional elements of the AAV genome may be added in trans to facilitate formation of a complete viral particle. Accordingly, the only portion of the viral genome that is required to be included in the DNA carried by the AAV are the 5′ and 3′ ITRs. Suitable ITRs are known in the art and may include, for example, a 5′ or 3′ ITR with the sequence SEQ ID NO: 18.

### Infectious Particles

Suitable infectious particles are known in the art. For example, see Deverman et al. (Cre-dependent selection yields AAV variants for widespread gene transfer to the adult brain, Nature Biotechnology, 34(2): 204-209, 2016) and Chan et al. (Engineered AAVs for efficient noninvasive gene delivery to the central and peripheral nervous system, Nature Neuroscience, 20(8): 1172-1179, 2017), which are incorporated herein by reference in their entirety. A skilled artisan will be familiar with the elements and configurations necessary for vector construction to encode the constructs described herein. Exemplary infectious particles comprise adeno-associated virus (AAV) particles, adenovirus particles, herpesvirus particles, lentiviral particles, baculovirus particles, virus-like particles (VLPs), or any other suitable virus particles that may be used to deliver the polypeptides of the present technology and/or nucleic acids (e.g., mRNA, DNA) encoding the same to a cell or tissue.

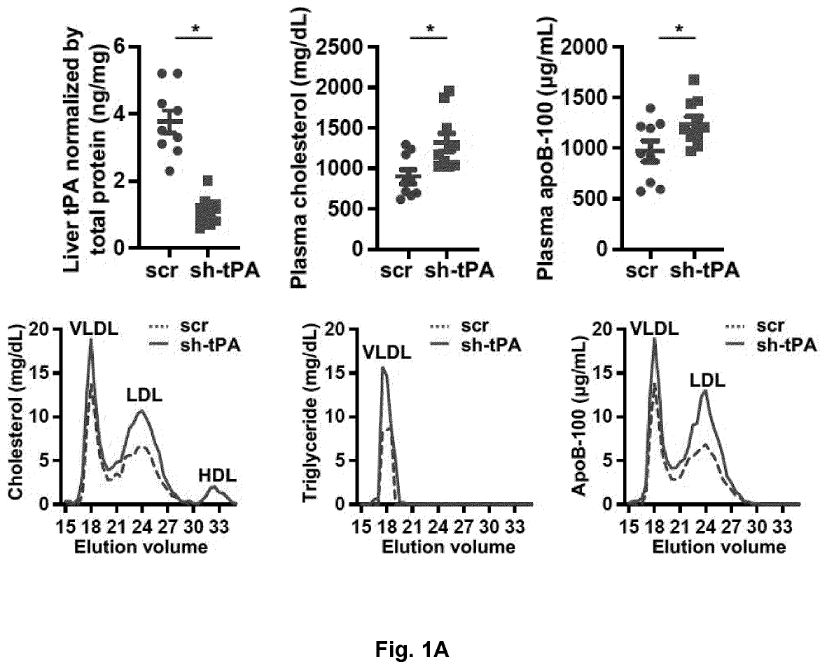

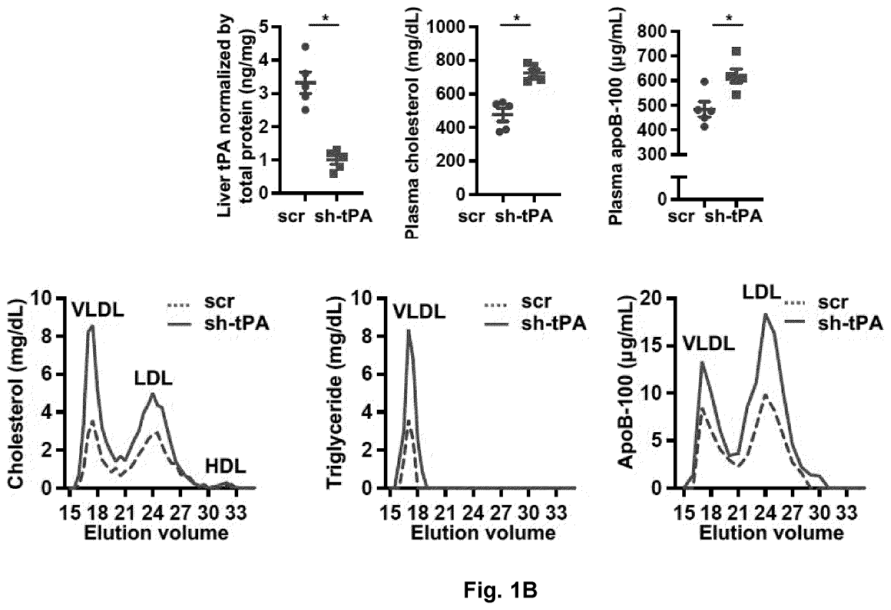

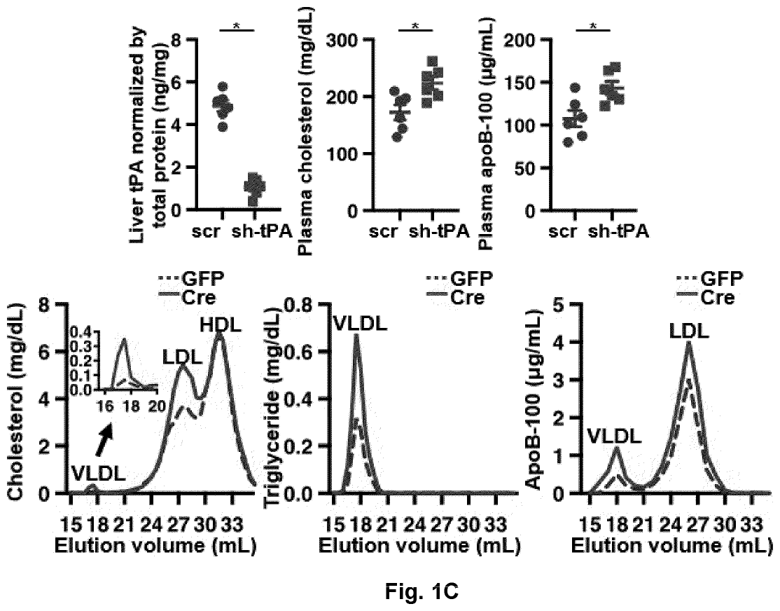

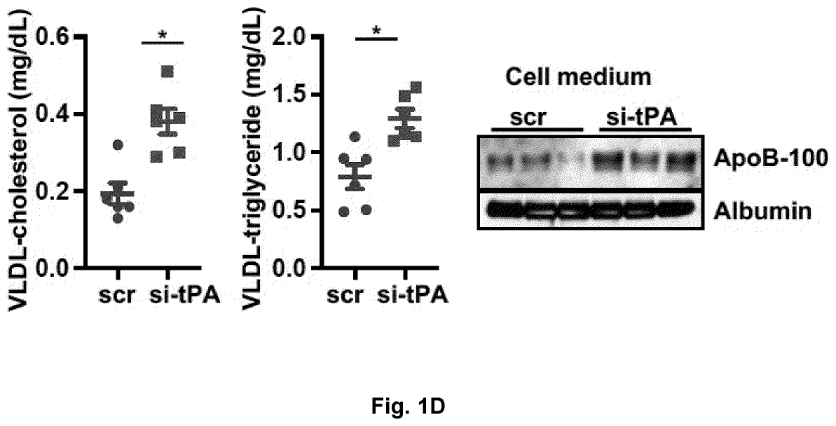

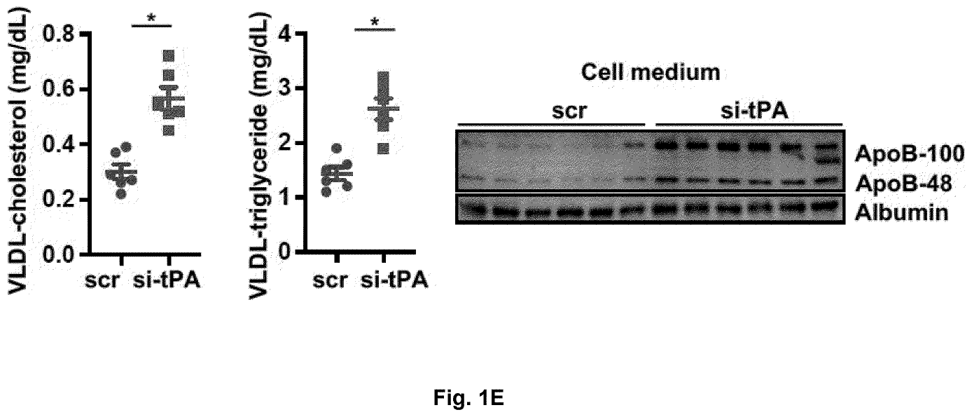

The disclosed infectious particles may comprise adeno-associated viruses AAVs comprising the aforementioned viral regulatory regions, which direct expression of a product from the disclosed polynucleotides, which are contained within the virus or associated with the virus, in a host cell, where the host cell expresses the polynucleotides to generate the disclosed recombinant polypeptides. In some cases, the virus is selected from AAV type 1, AAV type 2, AAV type 3 (including types 3A and 3B), AAV type 4, AAV type 5, AAV type 6, AAV type 7, AAV type 8, AAV type 9, AAV type 10, AAV type 11, among others. In some embodiments, the virus is an AAV type 8 (AAV8) virus. See, e.g., FIGS. 1A, 1B; 2A-2F; 6A-6F.

### Pharmaceutical Compositions

It is envisioned that the disclosed polynucleotides and recombinant polypeptides and/or nucleic acids encoding the same may be administered to a subject. Therefore, in an aspect of the current disclosure, pharmaceutical compositions are provided. In some embodiments, the pharmaceutical compositions comprise one or more of the disclosed polynucleotides, polypeptides and/or nucleic acids encoding the same, and a pharmaceutically acceptable carrier or excipient or the disclosed infectious particles and a pharmaceutically acceptable carrier or excipient. In some embodiments, the polynucleotides of the present technology are formulated for delivery to a subject via infectious particle (e.g., via adeno-associated virus (AAV)) or a lipid nanoparticle (LNP).

As used herein, “an effective amount” or a “therapeutically effective amount” refers to the amount or dose of the disclosed compositions, upon single or multiple dose administration to a subject, which provides the desired effect in the subject under diagnosis or treatment, e.g., reduction of apoB lipoprotein in the serum, including one or more of reduction of LDL in the serum, reduction of IDL in the serum, reduction of VLDL in the serum, reduction of Lp(a) in the serum, reduction of chylomicron in the serum, reduction of chylomicron remnant in the serum, or improvement in one or more metrics related to cardiovascular disease including, e.g., reduction in total cholesterol, reduction in blood pressure, reduction in fasting blood glucose, reduction in hemoglobin A1C. An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances. The compositions can also be administered in combination with one or more additional therapeutic compounds/agents (a “co-administration” where, for example, the additional or other therapeutic agent(s) could be administered simultaneously, sequentially, or by separate administration).

As is apparent to those skilled in the art, a therapeutically effective amount of one or more of the recombinant tPA-K2 polypeptides, and/or nucleic acids (e.g., mRNA, cDNA) encoding the same, of the present technology may vary according to factors such as the disease state, age, sex, weight, and general condition of the subject, and the ability of the recombinant tPA-K2 polypeptides, and/or nucleic acids (e.g., mRNA, cDNA) encoding the same, of the present technology to elicit a desired response in the particular subject (the subject's response to therapy). In delivering the recombinant tPA-K2 polypeptides, or nucleic acids (e.g., mRNA, cDNA) encoding the same, to a subject, the dosage will also vary depending upon such factors as the general medical condition, previous medical history, disease type and progression, and the like.

In general, a typical dose of a therapeutically effective amount of the recombinant tPA-K2 polypeptides, or nucleic acids (e.g., mRNA, cDNA) encoding the same, of the present technology may contain from about 0.01 mg/kg to about 100 mg/kg (such as from about 0.05 mg/kg to about 50 mg/kg, and/or from about 0.1 mg/kg to about 25 mg/kg, and/or from about 1 mg/kg to about 10 mg/kg) of the disclosed recombinant polypeptides, or nucleic acids encoding the same.

A typical dose of the disclosed pharmaceutical compositions comprising viral particles, may comprise an amount from about 5 μg of viral DNA to about 100 μg viral DNA, about 10 μg viral DNA to about 50 μg viral DNA. The dose of the disclosed viral particles administered to a subject may comprise about 1×10¹⁰ viral particles to about 1×10²⁰ viral particles, e.g., 1×10¹⁴ to 1×10¹⁶ viral particles, or about 1×10¹⁴, about 2×10¹⁴, about 3×10¹⁴, about 4×10¹⁴, about 5×10¹⁴, about 6×10¹⁴, about 7×10¹⁴, about 8×10¹⁴, about 9×10¹⁴, about 1×10¹⁵, about 2×10¹⁵, about 3×10¹⁵, about 4×10¹⁵, about 5×10¹⁵, about 6×10¹⁵, about 7×10¹⁵, about 8×10¹⁵, about 9×10¹⁵, about 1×10¹⁶ administered in a single dose to a subject. Compositions comprising the disclosed recombinant polypeptides can be formulated in a unit dosage form, each dosage containing from about 1 to about 500 mg of each polypeptide individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

In some embodiments, the present disclosure provides a composition comprising one or more of the recombinant tPA-K2 polypeptides of the present technology as a pharmaceutically acceptable salt, tautomer, hydrate, and/or solvate thereof. In some embodiments, mixtures of two or more of the recombinant tPA-K2 polypeptides may be used as a therapeutic agent. The polypeptides may be synthesized by any of the methods well known in the art. In some embodiments, the polypeptide may be formulated as a pharmaceutically acceptable salt. The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when a polypeptide contains both a basic moiety, such as an amine, pyridine or imidazole, and an acidic moiety such as a carboxylic acid or tetrazole, zwitterions may be formed and are included within the term “salt” as used herein. Certain compound(s)/polypeptide(s) disclosed in the present disclosure can exist in unsolvated forms as well as solvated forms, including hydrated forms. Solvated forms can exist, for example, because it is difficult or impossible to remove all the solvent from the polypeptide post synthesis. In general, the solvated forms are equivalent to unsolvated forms and are encompassed within the scope of the present disclosure.

Certain compound(s)/polypeptide(s) of the present disclosure may exist in crystalline form, multiple crystalline forms, amorphous forms or any combination of the foregoing. Certain compound(s)/polypeptide(s) of the present disclosure may exist in various tautomeric forms. Certain compound(s)/polypeptide(s) of the present disclosure may exist in various salt forms or mixtures of salt forms. In general, all physical forms of the compound(s)/polypeptide(s) disclosed herein are equivalent for the uses contemplated by the present disclosure and are intended to be within the scope of the present disclosure.

Combination therapy is also disclosed herein. The disclosed compositions, e.g., recombinant polypeptides and/or nucleic acids encoding the same, nanoparticles, infectious particles, or pharmaceutical compositions may be administered in combination with a statin, a PCSK9 inhibitor, a plasminogen activator inhibitor-1 (PAI-1) inhibitor, and ACE inhibitor, an insulin-sensitizing agent, hormone replacement therapy in women, or any combination thereof, or in combination with the standard of care currently known or unknown for the treatment of dyslipidemia or cholesterolemia. Exemplary, non-limiting PAI-1 inhibitors may include MDI-2268, PAI-039, TM5441, TM5275 sodium, TM5441 sodium, CDE-096, Aleplasinin, Loureirin B, Diaplasinin, Toddalolactone, SK-216, Geodin, Fendosal, AZ3976, and TM5007. Other PAI-1 inhibitors have been described in U.S. Pat. Nos. 8,759,327; 9,096,501, 9,230,744, and 9,527,878, which are each incorporated by reference in their entireties. Intravenous administration is an illustrative route of administering the compounds employed in the compositions and methods disclosed herein. Other illustrative routes of administration include transdermal, percutaneous, intravenous, intramuscular, intranasal, buccal, intrathecal, intracerebral, oral, or intrarectal routes. The route of administration may be varied in any way, limited by the physical properties of the compounds being employed and/or the convenience of the subject and/or the caregiver.

As one skilled in the art will appreciate, suitable formulations include those that are suitable for more than one route of administration. For example, the formulation can be one that is suitable for both intravenous and intramuscular administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions may be conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount,” that is, the amount of the compound that provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Pharmaceutical compositions and preparations comprising the recombinant tPA-K2 polypeptides of the present technology, or nucleic acids (e.g., mRNA, cDNA) encoding the same, may be manufactured by means of conventional mixing, dissolving, granulating, emulsifying, encapsulating, entrapping or lyophilizing processes. Pharmaceutical compositions may be formulated in conventional manner using one or more physiologically acceptable carriers, diluents, excipients or auxiliaries that facilitate formulating preparations suitable for in vitro, in vivo, or ex vivo use. The compositions can be combined with one or more additional active agents and may be formulated with a pharmaceutically acceptable carrier, diluent or excipient to generate pharmaceutical (including biologic) or veterinary compositions of the instant disclosure suitable for parenteral administration.

Many types of formulation are possible as is appreciated by those skilled in the art. The particular type chosen is dependent upon the route of administration chosen, as is well-recognized in the art. For example, systemic formulations will generally be designed for administration by injection, e.g., intravenous. In some embodiments, the systemic formulation is sterile.

Sterile injectable solutions are prepared by incorporating recombinant tPA-K2 polypeptides, or nucleic acids (e.g., mRNA, cDNA) encoding the same, in the required amount of the appropriate solvent with various other ingredients enumerated herein, as required, followed by suitable sterilization means. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuum drying and freeze-drying techniques, which yield a powder of plus any additional desired ingredient from a previously sterile-filtered solution thereof.

In some embodiments, compositions comprising the recombinant tPA-K2 polypeptides, or nucleic acids (e.g., mRNA, cDNA) encoding the same, may be formulated in aqueous solutions, or in physiologically compatible solutions or buffers such as Hanks's solution, Ringer's solution, mannitol solutions or physiological saline buffer. In certain embodiments, any of the recombinant tPA-K2 polypeptides, or nucleic acids (e.g., mRNA, cDNA) encoding the same, may contain formulator agents, such as suspending, stabilizing, penetrating or dispersing agents, buffers, lyoprotectants or preservatives such as polyethylene glycol, polysorbate 80, 1-dodecylhexahydro-2H-azepin-2-one (laurocapran), oleic acid, sodium citrate, Tris HCl, dextrose, propylene glycol, mannitol, polysorbate polyethylenesorbitan monolaurate (Tween®-20), isopropyl myristate, benzyl alcohol, isopropyl alcohol, ethanol sucrose, trehalose and other such generally known in the art may be used in any of the compositions of the instant disclosure. (Pramanick et al., Pharma Times 45(3):65-76 (2013)).

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol, and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As a further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acidic contents of the stomach. Such formulations can be created by coating a solid dosage form with a film of a polymer that is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Methods

As demonstrated herein, the disclosed polynucleotides, recombinant tPA-K2 fragments or nucleic acids encoding the same are effective in methods for reducing the assembly and secretion of atherogenic apoB-lipoproteins from hepatocytes (FIGS. 23A-23B and FIGS. 24A-24B). Therefore, in another aspect of the current disclosure, methods are provided. The methods comprise administering a therapeutically effective amount of any one or more of the disclosed polynucleotides, recombinant polypeptides or nucleic acids encoding the same to a subject in need thereof. The subject may have hypercholesterolemia or hyperlipidemia, may be diagnosed with a cardiovascular disease, e.g., atherosclerosis or arteriosclerosis, or the subject may be diagnosed with type 2 diabetes.

The disclosed methods may reduce total cholesterol in a subject, or a level of intermediate density lipoproteins (IDLs), low density lipoproteins (LDLs), very low density lipoproteins (VLDLs), lipoprotein (a) [Lp(a)], chylomicron or chylomicron remnant in the blood (e.g., serum or plasma) of the subject. Methods of measuring lipoproteins, e.g., VLDL, LDL, IDL, Lp(a), and HDL are known in the art and are considered to be routine medical laboratory tests. The disclosed methods may be used to treat hyperlipidemia in a subject in need thereof.

In some embodiments, methods include lowering plasma lipids and/or apoB levels in subjects suffering from or at an increased risk for atherothrombotic events. In some embodiments, the methods comprise administering a composition comprising a tPA polypeptide (e.g., full-length tPA and/or any of the recombinant tPA-K2 polypeptides disclosed herein or nucleic acids encoding the same) or polynucleotide comprising an endoplasmic reticulum localization motif (e.g., KDEL) to the subject. In some embodiments, the composition is formulated for delivery to the subject in an LNP or an infectious particle, such as an adeno-associated virus. In some embodiments, the subject suffers from or is at risk of suffering from severe atherothrombotic events. In some embodiments, administration of the composition to the subject lowers plasma lipid levels in the subject. In some embodiments, the subject is undergoing or has undergone the standard of care treatment for atherothrombotic therapy or blood clots. In some embodiments, the standard of care treatment for atherothrombotic therapy or blood clots may include any one or more of anticoagulants, thrombolytics, catheter-directed thrombolysis, or thrombectomy. In some embodiments, the subject is receiving or has received tissue plasminogen activator therapy. In some embodiments, the subject is receiving or has received an anticoagulant. In some embodiments, the subject is undergoing, will undergo, or has undergone treatment with a thrombolytic agent, an anticoagulant, catheter-directed thrombolysis, and/or thrombectomy.

As used herein, “hyperlipidemia” refers to abnormally elevated levels of any or all lipids or lipoproteins, e.g., fats, cholesterol or triglycerides, in the blood. Hyperlipidemia encompasses hypercholesterolemia.

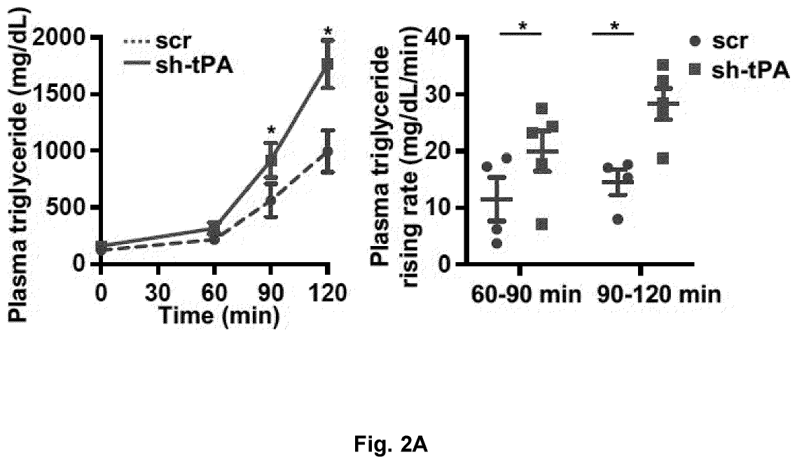

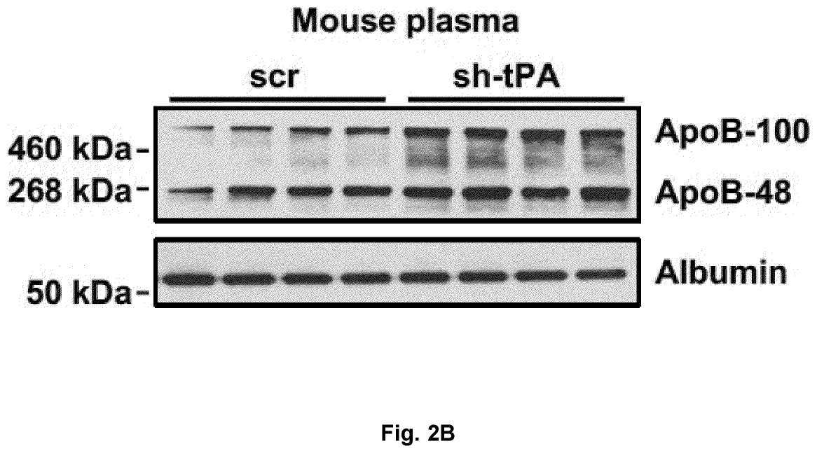

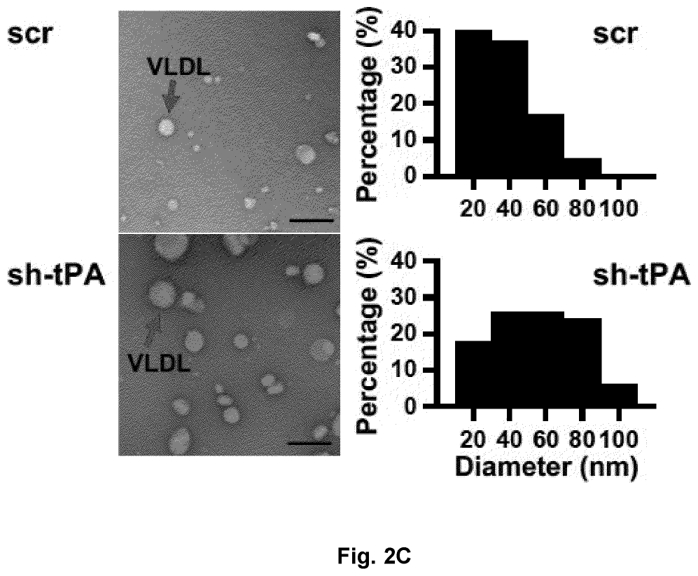

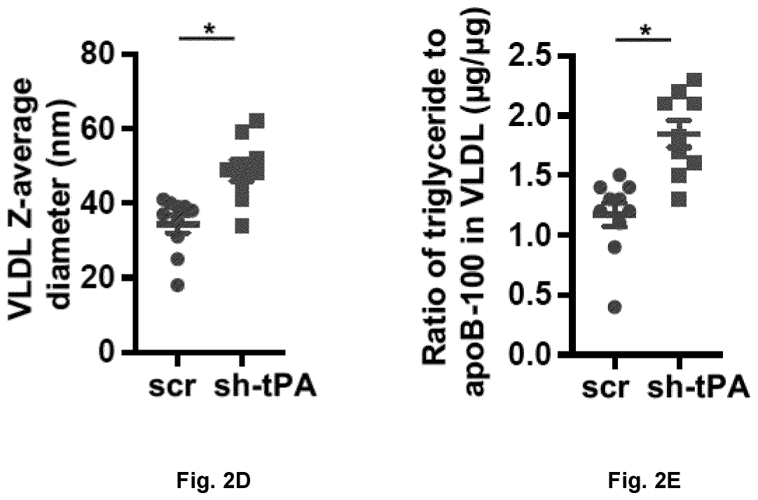

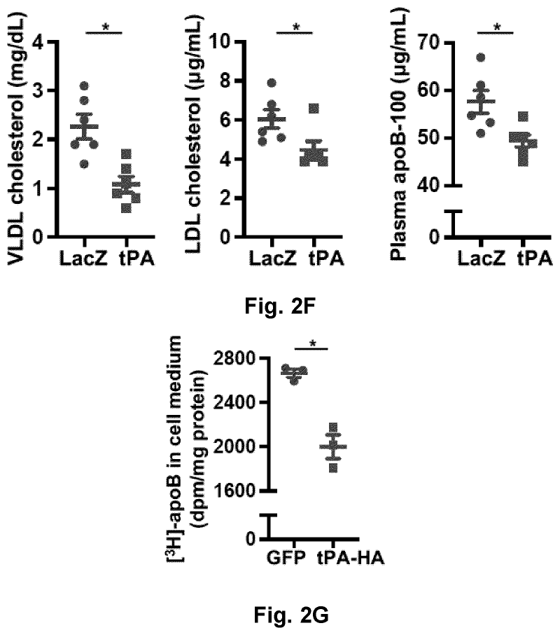

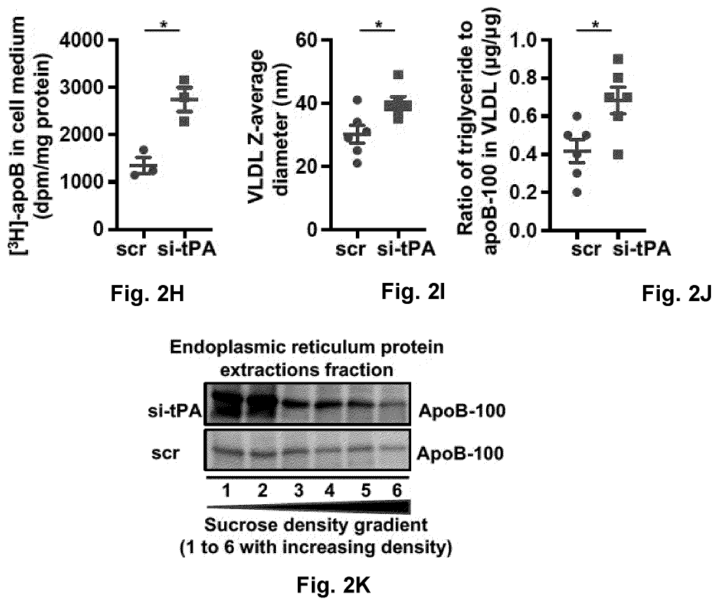

The inventors demonstrated that administering AAVs with viral genomes encoding the disclosed recombinant polypeptides, i.e., comprising the disclosed polynucleotides, was effective in reducing VLDL and LDL in animals lacking expression of tPA. See, FIG. 2F (mice treated with AAV8-TBG-Plat encoding full-length murine tPA). Therefore, in other aspects of the disclosure, the methods comprise administering the disclosed pharmaceutical compositions comprising infectious particles to a subject in need thereof.

The disclosed polynucleotides may be administered using any appropriate delivery vehicle. For example, in some cases, a nucleic acid encoding a recombinant polypeptide of the present technology may be incorporated into a delivery vehicle that can drive expression of the nucleic acid. Examples of delivery vehicles include, without limitation, non-viral vectors (e.g., plasmids (e.g., expression plasmids), liposomes, and polymersomes) and viral vectors (e.g., adeno-associated virus (AAV) vectors, HSV vectors, and lentiviral vectors). In some embodiments, the disclosed polynucleotides may be delivered using an AAV vector. As used herein, the term “adeno-associated virus” (AAV) includes, without limitation, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered. The genomic sequences of various AAV and autonomous parvoviruses, as well as the sequences of the ITRs, Rep proteins, and capsid subunits are known in the art. Such sequences may be found in the literature or in public databases such as the GenBank database.

By way of example, but not by way of limitation, in some embodiments, a subject administered a therapeutically effective amount will show a reduction in total cholesterol in a subject, or a level of intermediate density lipoproteins (IDLs), low density lipoproteins (LDLs), very low density lipoproteins (VLDLs), Lp(a), chylomicrons, or chylomicron remnants in the serum of the subject, compared to an untreated control subject within about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 7 months, about 8 months, about 9 months, about 10 months, about 11 months, or about 12 months.

Treatment may be administered multiple times daily, multiple times per week, on a monthly basis, or, in some cases, the treatment may only need to be performed once, e.g., in the case of infectious particle-mediated delivery of the disclosed tPA-K2 polypeptides.

Methods of Making Recombinant tPA-K2 Polypeptides

Methods of making the recombinant tPA-K2 polypeptides are also disclosed herein. The disclosed polypeptides may be made according to known methods for producing recombinant polypeptides. For example, the polynucleotides of the instant disclosure may be introduced into a suitable cell, e.g., an animal cell, a human cell line according to standard procedures, e.g., transfection, whereby the polynucleotide is expressed to produce recombinant tPA-K2 polypeptides. The recombinant tPA-K2 polypeptides may be isolated and further purified according to methods known in the art, e.g., column purification, liquid chromatography, etc.

Further, methods of generating rAAV virions are well known. See, e.g., K. Fisher et al, J. Virol., 70:520-532 (1993) and U.S. Pat. No. 5,478,745, which are incorporated by reference herein in their entireties.

### Kits, Systems, and Platforms

In another aspect of the instant disclosure, kits, systems, and platforms are provided. The kits, systems, and platforms may comprise, e.g., one or more of the disclosed recombinant polypeptides, one or more of the disclosed polynucleotides, and/or one or more of the disclosed infectious particles. In some embodiments, the kit also provides instructions for use.

The present technology is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

As used herein, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about,” “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, “administering” or the “administration” of an agent (i.e., a therapeutic agent) or compound/drug product (including a composition (i.e., a formulation or medicament)) to a subject includes any route of introducing or delivering to a subject a compound/drug product to perform its intended function. Administration may be carried out by any suitable route, such as oral administration. Administration can be carried out subcutaneously. Administration can be carried out intravenously. Administration can be carried out intraocularly. Administration can be carried out systemically. Alternatively, administration may be carried out topically, intranasally, intraperitoneally, intradermally, ophthalmically, intrathecally, intracerebroventricularly, iontophoretically, transmucosally, intravitreally, or intramuscularly. Administration includes self-administration, the administration by another or administration by use of a device (e.g., an infusion pump).

As used herein, the term “excipient” refers to a natural or synthetic substance formulated alongside the active ingredient of a medication, included for the purpose of long-term stabilization, bulking up solid formulations, or to confer a therapeutic enhancement on the active ingredient in the final dosage form, such as facilitating drug absorption, reducing viscosity, or enhancing solubility.

As used herein, “heterologous” refers to a sequence, e.g., an amino acid or a nucleotide sequence that does not occur naturally as part of the genome in which it is present or which is found in a location or locations in a genome or vector that differ from that in which it occurs naturally.

“Homology” or “identity” or “percent identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

As used herein, “operably linked” with reference to nucleic acid sequences, regions, elements or domains means that the nucleic acid regions are functionally related to each other. For example, a nucleic acid encoding a leader peptide can be operably linked to a nucleic acid encoding a polypeptide, whereby the nucleic acids can be transcribed and translated to express a functional fusion protein, wherein the leader peptide affects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, the term “pharmaceutically acceptable carriers” refers to any diluents, excipients, or carriers that may be used in the compositions disclosed herein. In some embodiments, a pharmaceutically acceptable carrier comprises, or consists essentially of, or yet further consists of a nanoparticle, such as a polymeric nanoparticle carrier or a lipid nanoparticle (LNP). Additionally or alternatively, pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They can be selected with respect to the intended form of administration, that is, oral tablets, capsules, elixirs, syrups and the like, and consistent with conventional pharmaceutical practices.

As used herein, “prevention” or “preventing” of a disease, disorder, or condition refers to results that, in a statistical sample, exhibit a reduction in the occurrence of the disease, disorder, or condition in a sample or subject administered a therapeutic agent or agents relative to a control sample or subject. Such prevention is sometimes referred to as a prophylactic treatment.

As used herein, “recombinant,” with respect to polynucleotides or polypeptides, refers to polynucleotides or polypeptides that have been modified in vitro by techniques known in the art. For example, expression vectors or expression plasmids, or expression products thereof are considered to be recombinant. In some embodiments, the polynucleotide or polypeptide is modified by the introduction of a heterologous nucleic acid or protein or the alteration of a native nucleic acid or protein, or the material is derived from a cell so modified. Thus, for example, recombinant cells express genes that are not found within the native (non-recombinant) form of the cell or express native genes that are otherwise abnormally expressed, under expressed or not expressed at all.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, a “synergistic therapeutic effect” refers to a greater-than-additive therapeutic effect which is produced by a combination of at least two agents, and which exceeds that which would otherwise result from the individual administration of the agents.

As used herein, the terms “treating” or “treatment” refer to therapeutic treatment, wherein the object is to reduce, alleviate or slow down (lessen) a pre-existing disease or disorder, or its related signs, symptoms, or conditions. By way of example, but not by way of limitation, a subject is successfully “treated” for a disease if, after receiving an effective amount of the composition, the subject shows observable and/or measurable reduction in or absence of one or more signs, symptoms or conditions associated with the disease, disorder or condition. It is also to be appreciated that the various modes of treatment of medical conditions as described are intended to mean “substantial,” which includes total alleviation of conditions, signs or symptoms of the disease or disorder, as well as “partial,” where some biologically or medically relevant result is achieved.

The phrase “such as” should be interpreted as “for example, including.” Moreover, the use of any and all exemplary language, including but not limited to “such as,” is intended merely to better illuminate the present technology and does not pose a limitation on the scope of the present technology unless otherwise claimed.

Furthermore, in those instances where a convention analogous to “at least one of A, B, and C, etc.” is used, in general such a construction is intended in the sense of one having ordinary skill in the art would understand the convention (e.g., “a system having at least one of A, B, and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 members refers to groups having 1, 2, or 3 members. Similarly, a group having 1-5 members refers to groups having 1, 2, 3, 4, or 5 members, and so forth. The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### EXAMPLES

The following Examples are illustrative and should not be interpreted to limit the scope of the claimed subject matter.

### Example 1—Intracellular tPA-PAI-1 Interaction Determines apoB Lipidation and VLDL Assembly in Hepatocytes

### Methods and Materials

Plasma from humans with PAI-1 deficiency. Plasma samples were collected from members of the Berne Amish community who harbor a frameshift mutation in SERPINE1 (SERPINE1^{-/-}, n=10) (55), and their age, gender and BMI-matched control individuals from the same community (n=10). The age and gender information of both subjects with PAI-1 deficiency and the controls were listed in Table 2. The institutional review boards at the Indiana Center for Hemophilia and Thrombosis and Medical College of Wisconsin (MCW) approved the study protocols. The study participants provided written informed consent.

Mice. Plat^{fl/fl} mice were generated using wild type C57BL6/J mice through the homologous recombination in embryonic stem cell-based approach (Biocytogen, Wakefield, MA). Briefly, a targeting construct is designed to insert loxP sites into the intron 3 and 6, to flox the exon 4-6 of Plat gene. This design is to conditionally knockout the exon 4-6 of Plat by Cre-loxP system. Hepatocyte tPA knockout mice were generated by administering Plat^{fl/fl} mice with an AAV8 expressing a Cre recombinase driven by the thyroxine-binding globulin (TBG) promoter, AAV8-TBG-cre (Cre), and Plat^{fl/fl} mice receiving AAV8-TBG-GFP (GFP) were used as controls. To silence tPA expression in heptocytes, mice were intravenously injected with AAV8 virus containing shPlat (AAV8-H1-shPlat) (19). Age-matched control mice were injected with AAV8-H1-scramble silencing control. Ldlr^{-/-}, Apoe^{-/-} and WT C57BL/6J mice, used for silencing hepatocyte tPA, were purchased from Jackson Laboratory (JAX) (Cat #002207, 002052 and 000664, respectively). Ldlr^{-/-} and Apoe^{-/-} mice were fed with WD (Teklad, Cat #TD 88137). WT mice were on a standard chow (Lab diet, Cat #5053), DIO diet (Research Diets, Cat #12492), or WD (Teklad, Cat #TD 88137). To express tPA in hepatocytes, WT C57BL/6J mice and holo-tPA-KO mice (Jax, Cat #002508), on a standard chow diet, received intravenous injection of AAV8-TBG-Plat. For all experiments, mice were maintained on a 12-hour light/12-hour dark cycle with free access to normal chow, WD or DIO diet and water. Mice of the same age and similar weight were randomly assigned to experimental and control groups. Plasma lipids were assayed in blood collected after a 5-hour withdrawal of food. We use power calculations to determine the number of mice for each experiment. We include both male and female mice. Mice of the same age and weight are randomly assigned to groups; exclusion criteria are death, injury requiring euthanasia, or weight loss >10%, assuming these are rare events that are not statistically different between groups. All endpoint assays and analyses are conducted by researchers who are blinded to the identity of the cohort. All mouse experiments were performed with the approval of the Institutional Animal Care and Use Committee of Biomedical Resource Center at MCW and Institutional Animal Care and Use Committee of Columbia University Irving Medical Center.

Vector constructs. AAV8-TBG-cre and AAV8-TBG-GFP were purchased from Addgene. As previously described (19, 20), AAV8-H1-short hairpin RNA (shRNA) construct targeting murine Plat was made by annealing complementary oligonucleotides and then ligating them into the pAAV-RSV-GFPH1 vector. AAV8-TBG-Plat was purchased from Vector Biolabs. Plasmid expressing wild-type human tPA, pCMV3-tPA-HA, was purchased from Sino Biologic (Beijing, China). The plasmid constructs to express human tPA mutants were generated by Versiti BRI Core based on the pCMV3-tPA-HA. Constructed tPA mutants include tPA-S513A, tPA-KDEL, tPA-Δ-K2-HA and tPA-D236, 238N. In particular, tPA-Δ-K2 refers to the tPA mutant with the replacement of K2 with K1, leading to the presence of 2 copies of K1 but no K2. The purpose of this design is to create a mutant tPA that lacks K2 but mimic normal tPA structure.

Mouse plasma collection and analyses. Blood obtained by cardiac puncture into 10% volume of sodium citrate (3.8%, w/v) was centrifuged at room temperature for 15 minutes at 2300 g, and plasma was carefully collected from the supernatant fraction. Plasma samples were divided into aliquots, snap frozen, and stored at −80° C. until analyses. Plasma total antigen levels of tPA and apoB-100 were measured by ELISA using kits according to the manufacturer's instructions.

Human primary hepatocyte experiments. Human primary hepatocytes were obtained from the Liver Tissue Cell Distribution System at the University of Pittsburgh (Pittsburgh, Pennsylvania, USA). All cells were cultured in Williams' Medium E supplemented with Hepatocyte Maintenance Supplement Pack (Thermo Fisher Scientific, Cat #CM4000). Experiments were conducted as described in the figure legends. Cells were harvested and culture media were collected, snap-frozen in liquid nitrogen, and stored at −80° C. until processing. The age and gender information of human donors were listed in Table 3.

McA-RH7777 cell experiments. Rat hepatoma McA-RH7777 cells were obtained from American Type Culture Collection (Manassas, Virginia, USA). Cells were grown in Dulbecco's Modified Eagle Medium (DMEM) (Thermo Fisher Scientific, Cat #12430054) containing 10% Fetal Bovine Serum (FBS). Experiments were conducted as described in the figure legends. Cells were harvested and culture media were collected, snap-frozen in liquid nitrogen, and stored at −80° C. until processing.

Transfection of cultured hepatocytes with plasmid encoding tPA-K2-HA-KDEL. McA-RH7777 cells were plated in culture plates. At approximately 30-40% confluency, the transfection mixture was prepared by preincubating plasmids coding for tPA-K2-HA-KDEL or GFP control with the transfection reagent Lipofectamine 3000 in Opti-MEM for 10 minutes. Then, the transfection mixture was added to the culture medium. Seventy-two hours post-transfection, hepatocytes and culture medium were harvested and collected. The expression efficiency of tPA-K2-HA-KDEL in cell lysates and apoB in the cell medium were detected by immunoblot (FIGS. 23A-23B).

LNP injection in mice. Mice were intravenously injected with LNP carrying mRNA encoding tPA-K2-HA-KDEL (SEQ ID NO: 32) or control Luciferase (1 mg/kg body weight). Similar experiments will be performed with LNP carrying mRNA encoding tPA-K2-KDEL (SEQ ID NO: 8). Blood was collected at 6-, 24-, and 48-hours post-injection and just before injection. Plasma triglyceride and cholesterol levels were measured (FIGS. 24A-24B).

Protein extraction and immunoblotting. Liver tissue samples and cultured hepatocytes were homogenized in RIPA buffer (Thermo Fisher Scientific, Cat #89900) supplemented with Halt™ Protease and Phosphatase Inhibitor Cocktail (Thermo Fisher Scientific, Cat #78444). Protein extracts were electrophoresed on SDS-PAGE gels and transferred to PVDF membranes. The membranes were blocked in tris-buffered saline with 0.1% Tween 20 (TBST) containing 5% (w/v) BSA. The membranes were then incubated at 4° C. overnight with primary antibodies in TBST containing 5% BSA, followed by incubation with the appropriate secondary antibodies coupled to horseradish peroxidase. Proteins were detected by ECL chemiluminescence.

Quantitative RT-PCR. Total RNA was extracted using RNeasy kit (QIAGNE, Cat #74004). cDNA was synthesized using iScript™ cDNA Synthesis Kit (BIO-RAD, Cat #1708891). Quantitative RT-PCR was performed using the Quant Studio 6 system (Applied Biosystems). To normalize the relative expression, the expression level of each gene was normalized to the 36B4 (housekeeping). The primer sequences used for quantitative RT-PCR were listed in Table 4.

Mouse hepatic VLDL production rate. To measure the hepatic VLDL secretion rate, mice received an intraperitoneal injection of Poloxamer-407 (1,000 mg/kg body weight) (96). 60, 90 and 120 minutes after administration of Poloxamer-407, tail vein blood samples were taken, and plasma triglyceride levels were measured by assays according to manufacturer's instructions (Wako, Fujifilm). Plasma triglyceride rising rate, between 60-90 minutes, or between 90-120 minutes, was calculated as the increased triglyceride level divided by the corresponding time, which reflects VLDL secretion rate within this time frame.

Pulse-chase assay for apoB secretion. Hepatocyte apoB-100 secretion was assayed using the ³[H] labeling method as previously (31). Human primary hepatocytes or McA-RH7777 cells were washed with leucine-free media and pulsed with ³[H] leucine (80 uCi/ml; 160 Ci/mmol, Perkin Elmer, Cat #NET1166005MC) for 20 minutes. The ³[H] leucine-containing medium was removed, and cells were incubated with fresh DMEM for an additional 0.5, 1 or 3 hours. ApoB was immunoprecipitated from cell homogenates and media using anti-apoB antibodies (Sigma-Aldrich, Cat #AB742). Nonlabelled apoB-100 standards were added to the precipitates, and the samples were separated by SDS-PAGE gel. Gels were silver stained, and the bands corresponding to apoB-100 were excised, and radioactivity associated with apoB-100 was quantified by a scintillation counter.

Endoplasmic reticulum (ER) isolation and protein extraction. ER fractions from hepatocytes were isolated as previously (97). Cells were washed in ice cold PBS and then collected in ER extraction buffer (20 mM HEPES, 250 mM sucrose, pH=7.4). Cell membrane was sheared by passing through cell through 29-gauge needles. Cell debris was pelleted by two rounds of centrifugation at 3000 g for 10 minutes. The supernatant was layered on top of a discontinuous sucrose gradient of 580-, 880-, and 1100-mM sucrose in ER extraction buffer and centrifuged at 100,000 g for 2 hours at 4° C. This pellet contained purified ER membrane. Extraction of proteins from ER was conducted as described (21). The ER pellets were dissolved with 1 M sodium carbonate, pH 11.5, containing 250 mM sucrose and 2 ml of 0.0625% deoxycholate in 3 M KCl. The mixture was incubated for 30 minutes at room temperature. After centrifugation, the supernatant contains the extracted proteins from ER.

Sucrose Gradient Ultracentrifugation of apoB-lipoprotein extracted from ER. The ER protein extractions were adjusted to 12.5% sucrose. The sucrose gradient was formed by layering from the bottom of the tube: 1 ml of 47% sucrose, 1 ml of 25% sucrose, 2.5 ml of sample in 12.5% sucrose, and 1.5 ml of phosphate-buffered saline. The gradients were spun at 35,000 rpm in a Beckman SW40 rotor for 65 hours at 12° C., and were unloaded into 6 fractions from the top to the bottom (21, 98).

MTP expression and purification. As previously (99), the plasmid carrying the complementary DNA (cDNA) of the hMTP cDNA (pcDNA3-hMTP-FLAG) (Addgene, Cat #138335) was transfected into Cos-7 cells. At 48 hours after transfection, cells were collected in 1 ml buffer K (10 mM Tris-Cl, 1 mM MgCl2, and 1 mM EGTA, pH 7.4) containing 150 mM NaCl and lysed by sonication on ice. The cell lysates were spun at 13,500 g for 10 minutes at 4° C. to remove unbroken cells and cell debris. The FLAG-tagged MTP was purified with columns packed with anti-FLAG M2 affinity agarose beads. The removal of FLAG peptide and concentration of the purified protein was done by ultrafiltration with cutoff centrifugal filter (Am Amicon Ultra, Merck Millipore, Cat #UFC9010).

Neutral lipid transfer activity assay. Neutral lipid transfer activity in cultured hepatocyte microsomal fractions was measured using a commercially available kit (Sigma-Aldrich, MAK110) as previously (36). Specifically, hepatocytes were homogenized in low hypotonic buffer (10 mM Tris-HCl, 1 mM EGTA, and 1 mM MgCl2, pH 7.4) using a Polytron homogenizer. Microsomes were isolated by ultracentrifugation (SW55 Ti rotor, 50,000 rpm, 1 hour). Neutral lipid transfer activity was assayed using the kit according to the manufacturer's manual. Isolated microsomes were incubated with fluorescent lipids-containing “donor vesicles” and the “acceptor vesicles”-LDL. The fluorescence signal was self-quenched when labeled lipids existing within the “donor vesicles” but would be detected after lipids transferred to “acceptor vesicles”.

Immunofluorescence imaging and proximity ligation assay (PLA). Human primary hepatocytes were plated on collagen-coated coverslips. After rinsing with PBS, cells were fixed with 4% paraformaldehyde for 15 minutes, permeabilized with 0.1% TritonX-100 in PBS for 5 minutes, and then blocked for 1 hour at room temperature using PBS containing 5% BSA. Blocked slides were incubated with the appropriate primary antibody overnight at 4° C., followed by species-specific fluorophore-conjugated secondary antibody for 1 hour at room temperature. For primary antibodies, we used rabbit anti-tPA antibody (ProteinTech, Cat #10147-1-AP), goat polyclonal anti-apoB (Sigma-Aldrich, Cat #AB742), and rabbit antibodies against each intracellular organelle marker-Calnexin for the endoplasmic reticulum (ER), TGN46 for the trans-Golgi network. For secondary antibodies, we used Alexa Fluor 488-conjugated donkey anti-goat IgG (Thermo Fisher Scientific, Cat #A21206), Alexa Fluor 568-conjugated donkey anti-rabbit IgG (Thermo Fisher Scientific, Cat #A11057) and Alexa Fluor 647-conjugated donkey anti-mouse IgG (Thermo Fisher Scientific, Cat #A31571)). Coverslips were washed 3 times for 5 minutes each with PBS and then incubated with DAPI nuclear stain (Invitrogen) for 2 minutes. Coverslips were then subjected to a final rinse in PBS and mounted with a slow anti-fade Mount.

For the PLA, the Duolink in situ kit (Sigma-Aldrich) was used following the manufacturer's protocol. Primary anti-tPA and anti-apoB antibodies were the same as those used for the immunofluorescence experiment described above. All reactions were performed in a humidified chamber at 37° C. After fixation and permeation, hepatocytes were incubated with primary antibodies for 40 minutes and a pair of PLA probes (anti-rabbit Minus and anti-goat Plus) for 60 minutes; ligase was added for 30 minutes, followed by amplification of signal for 100 minutes using red detection reagents. Coverslips were then mounted using the Duolink in situ mounting medium with DAPI and sealed with clear nail polish.

Fluorescent images were captured using a Nikon A1R Confocal Laser Scanning at ×40 magnification and digital zoom using the NIS-Elements analysis software. To detect Alexa fluorophores, respective lasers and filters were used for blue, green, red, and far-red. For PLA detection, the Texas Red setting was used.

Solid-phase protein binding assay. Solid-phase binding was performed in polystyrene microtiter plates using an enzyme-linked immunosorbent assay. Microtiter plate wells were coated with LDL at 5 μg/ml in coating buffer (TBS) overnight at 4° C. Unbound sites were blocked with 3% nonfat milk in TBS for 1 hour at 37° C. After washing with TBS containing 0.05% Tween 20 (TBS-Tween), tPA was added to the wells at concentrations from 0-20 μg/ml in TBS-Tween. Following a 1-hour incubation at 37° C. the wells were washed with TBS-Tween. Bound proteins were reacted with anti-tPA (1 μg/mL IgG in TBS-Tween in the presence of 3% nonfat milk) followed by goat anti-rabbit IgG conjugated to horseradish peroxidase. TMB substrates were added. After stopping the reaction, the absorbance at 450 nm was measured.

Surface plasmon resonance (SPR). Studies of the binding of recombinant tPA to purified LDL were performed with a Biacore S200 SPR instrument (Biacore) using a CM5 sensor chip (Cytiva, Cat #29149603) (94). LDL was attached to the chip using amine coupling chemistry, according to the manufacturer's instructions. In brief, the chip surface was prepared by exposing the carboxylated dextran matrix to an aqueous solution containing 0.4 M 1-ethyl-3-(3-dimethylaminopropyl) carbodiimide and 0.1 M N-hydroxysuccinimide (10 μl/min for 7 minutes). Then LDL (500 μg/ml in 10 mM sodium acetate buffer, pH 5.5) flowed across the chip surface at the same rate for 7 minutes, followed by 1 M ethanolamine-HCl (pH 8.5) at 19 μl/min for 7 minutes to deactivate excessive reactive groups and remove any noncovalently bound LDL. This procedure led to approximately 6,000 response units (RU) of LDL immobilized. To monitor LDL association with tPA, tPA solutions in HBS-E buffer (Biacore) (0.01 M Hepes, 0.15 M NaCl, 3 mM EDTA, pH 7.4) in the concentration range of 10 to 500 g/mL were flowed across the chip at 20 μl/min for 6 minutes at room temperature. The dissociation of tPA was then monitored by washing the surface for 6 minutes with HES buffer alone. tPA flowed across an activated, but uncoated CM5 chip under the same conditions as the control for nonspecific binding.

Plasma lipoprotein isolation by FPLC. FPLC was conducted at 4° C. using AKTA purifier 10 with two columns of Superose 6 increase 10/300 GL (Cytiva, Cat #29091596) in tandem. 300 μl (1.5 times of the volume of the sample loop) of pooled plasma was injected. Tris-buffered saline (25 mM Tris, 150 mM NaCl, 2 mM EDTA, pH=7.4) was used as the running buffer. The eluant was collected in 1-mL fractions from fractions 12-ml to 47-mL. Cholesterol, triglyceride (Wako, Fujifilm) and apoB-100 (Abcam, Cat #ab230932; Mabtech, Cat #3715-1HP) levels from fractions were measured according to manuals.

Plasma lipoprotein isolation by sequential ultracentrifugation. Plasma lipoproteins were separated by KBr (potassium bromide) density ultracentrifugation as described previously (95, 100). Equal amounts of mouse plasma were used for sequential density ultracentrifugation to separate very-low-density lipoprotein (d<1.006 g/mL), low-density lipoprotein (d=1.006-1.063 g/mL), and high-density lipoprotein (d=1.063-1.21 g/mL) in a TLA 100 rotor.

VLDL particle diameters analysis. VLDL particles (d<1.006 g/mL) were isolated by KBr density ultracentrifugation (95), followed by two methods to measure their diameters. First, isolated VLDL particles were negatively stained with 20 g/L phosphotungstic acid (pH 7.0) for 2 minutes and then viewed under a Philips CM10 electron microscope. The mean diameters of VLDL particles were determined using Image-Pro Plus 5.0 image analysis software. Second, the hydrodynamic diameters of isolated VLDL particles were measured using a Zetasizer μV dynamic laser light scattering instrument (Malvern Instruments) at 633 nm. VLDL samples were transferred to a quartz cuvette, and light scatter readings were performed at 20° C. (25-27).

Mouse tail bleeding assay. Mice were anesthetized with isoflurane and positioned horizontally on a platform that allowed the tail to descend ~2 cm from the top of the platform. A segment of tail on the distal tail tip was transected with a no. 11 surgical scalpel to induce wounds ~2 mm in diameter. Bleeding was monitored by gently dabbing the tail tip on Whatman paper at 10-second intervals until the cessation of bleeding. The time to stable cessation of bleeding was defined as the time interval between the tail incision and cessation of bleeding, with no evidence of rebleeding for 60 seconds. Bleeding exceeding 15 minutes was stopped by applying pressure.

Statistics analysis. The data described in the study were generated from biological replicates. The number for human participants research and mouse experiments were described in the figure legend where appliable. The in vitro cell experiments were repeated at least 3 times. All results are presented as mean±SEM. P values were calculated using 2-tailed Student's t test for data that passed the normality test or the Mann-Whitney rank-sum U test for data that were not normally distributed. One-way ANOVA with post hoc Tukey's test was used to evaluate differences among groups when 3 or more groups were analyzed.

Study approval. All mouse experiments were conducted with the approval of the IACUC and Institutional Biosafety Committee of MCW, and the IACUC of Columbia University Medical Center. The use of human cells and plasma samples in this study was approved by the IRB at the MCW and Indiana Hemophilia and Thrombosis Center. All participants provided written informed consent.

TABLE 1
Key resources.

| Reagent or resource | Source | Identifier |
|---|---|---|
| Antibody | | |
| Rabbit anti-tPA | Proteintech | 10147-1-AP |
| Rabbit anti-tPA | Sigma | HPA003412 |
| Rabbit anti-PAI-1 | Abcam | 66705 |
| Goat anti-apoB | Sigma | AB742 |
| Mouse anti-MTP | Santa Cruz | sc-515742 |
| Mouse anti-Calnexin | Proteintech | 66903-1-Ig |
| Rabbit anti-albumin | Cell Signaling | 4929S |
| Rabbit anti-β actin | Cell Signaling | 5125 |
| Rabbit anti-HA | Cell Signaling | 3724 |
| Horse anti-rabbit IgG, | Cell signaling | 7074 |
| HRP-linked | | |
| Horse anti-mouse IgG, | Cell signaling | 7076 |
| HRP-linked | | |
| Donkey anti-Rabbit IgG, | Thermo | A21206 |
| Alexa Fluor ™ 488 | | |
| Donkey anti-Goat IgG, | Thermo | A11057 |
| Alexa Fluor ™ 568 | | |
| Donkey anti-Mouse IgG, | Thermo | A31571 |
| Alexa Fluor ™ 647 | | |
| Elisa | | |
| Human apoB-100 | Mabtech | 3715-1HP |
| Mouse apoB-100 | Abcam | ab230932 |
| Human tPA | Innovative research | IHUTPAKTT |
| Human PAI-1 free tPA | Innovative research | IHUTPAKT |
| Human PAI-1/tPA complex | Innovative research | IHUPAI1TPACXKT |
| Human PAI-1 | Innovative research | IHUPAI1KTT |
| Mouse tPA | Innovative research | IMSTPAKTT |
| Mouse PAI-1 free tPA | Innovative research | IMSTPAKTA |
| Mouse PAI-1 | Innovative research | IMSPAI1KTA |
| Recombinant protein | | |
| Human tPA | Innovative research | IHUTPA85SC |
| Human serine protease | Innovative research | IHUTPANE |
| negative tPA | | |
| Human tPA/PAI-1 complex | Innovative research | IHUPAI1TPACX |
| Plasmid | | |
| tPA cDNA ORF Clone, | Sino Biologic | HG10157-CY |
| Human, C-HA tag | | |
| hMTP-flag | Addgene | 138335 |
| Chemicals and reagents | | |
| Sodium citrate (3.8%, w/v) | Fisher scientific | 72284 |
| RIPA buffer | Thermo fisher | 89900 |
| HaltTM Protease and | Thermo fisher | 78444 |
| Phosphatase Inhibitor | | |
| Cocktail | | |
| Dynabeads ™ Protein G | Thermo fisher | 10004D |
| CP-346086 | Sigma | PZ0103 |
| tranexamic acid | Sigma | PHR1812 |
| Duolink ® In Situ Detection | Sigma | DUO92013-100RXN |
| Reagents FarRed | | |
| Duolink ® In Situ | Sigma Aldrich | DUO92002 |
| PLA ® Probe Anti-Rabbit | | |
| PLUS | | |
| Duolink ® In Situ | Sigma Aldrich | DUO92004 |
| PLA ® Probe Anti-Mouse | | |
| MINUS | | |
| Duolink ® In Situ | Sigma Aldrich | DUO92006 |
| PLA ® Probe Anti-Goat | | |
| MINUS | | |
| Leucine, L-[4,5-3H], 1 mCi | Perkin Elmer | NET1166001MC |
| OptiPrep ™ Density | Sigma | D1556 |
| Gradient Medium | | |
| Oleate | Sigma | O7501 |
| Albumin, Fatty Acid-Free | Sigma | 126575 |

TABLE 2
Age and gender information of the homozygote for
PAI-1 deficiency and matched control individuals.

| | | | Age and gender-matched | | |
|---|---|---|---|---|---|
| Homozygote for PAI-1 deficiency | | | normal control | | |
| ID # | Age, years | Gender | ID # | Age, years | Gender |
| 1 | 21 | F | 11 | 21 | F |
| 2 | 24 | F | 12 | 23 | F |
| 3 | 30 | F | 13 | 30 | F |
| 4 | 33 | F | 14 | 32 | F |
| 5 | 34 | F | 15 | 34 | F |
| 6 | 19 | M | 16 | 19 | M |
| 7 | 32 | M | 17 | 32 | M |
| 8 | 25 | F | 18 | 26 | F |
| 9 | 24 | M | 19 | 24 | M |
| 10 | 11 | M | 20 | 18 | M |

TABLE 3
Age and gender information of the human participants
donating their primary hepatocyte.

| Donor # | Age, years | Gender |
|---|---|---|
| 1 | 69 | female |
| 2 | 65 | male |
| 3 | 66 | female |
| 4 | 57 | female |
| 5 | 82 | male |
| 6 | 68 | male |
| 7 | 35 | female |
| 8 | 63 | male |
| 9 | 47 | female |
| 10 | 45 | female |
| 11 | 61 | female |
| 12 | 29 | female |

TABLE 4
Primers for quantitative PCR.

| Genomic | | Primer sequence (5′-3′) | |
|---|---|---|---|
| region | Organism | Forward | Reverse |
| APOB | Human | TGCTCCACTCAC | TAGCGTCCAGTG |
| | | TTTACCGTC | TGTACTGAC |
| PLAT | Human | TGCTGTGAAATA | TGAGTCTCGATC |
| | | GATACCAGGGC | TGGGTTTCTG |
| RPLP0 | Human | AGCCCAGAACAC | GGTAGGCTGACC |
| | | TGGTCTC | CATTCCC |
| Apob | Mice | AAGCACCTCCGA | CTCCAGCTCTAC |
| | | AAGTACGTG | CTTACAGTTGA |
| Plat | Mice | AGCACTCTCGGG | GTCTGCGTTGGC |
| | | ACACAGAA | TCATCTCT |
| Rplp0 | Mice | GCTCCAAGCAGA | CCGGATGTGAGG |
| | | TGCAGCA | CAGCAG |

### Results

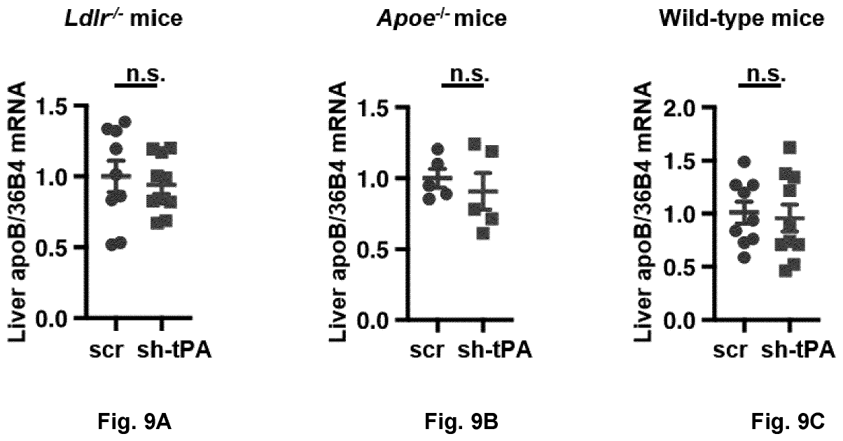

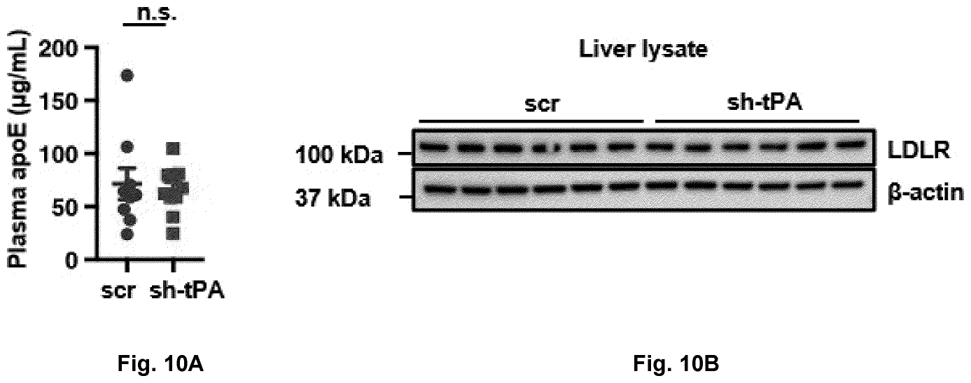

Silencing hepatocyte tPA increases atherogenic apoB-lipoprotein-cholesterol and apoB independent of LDLR or ApoE. An adenovirus-associated virus-8 (AAV8) expressing a hairpin RNA against Plat mRNA (encodes tPA) driven by H1 promoter (AAV8-H1-shPlat, short as sh-tPA) was administrated to Ldlr^{-/-} mice fed the Western Diet (WD), an established hypercholesterolemic mouse model, to silence tPA expression specifically in the hepatocytes [21, 22]. The hepatocyte tPA-silenced mice showed a 47% higher plasma total cholesterol level (p<0.01) and 28% higher apoB-100 (p<0.05) (FIG. 1A) than mice receiving AAV8-H1-scrambled RNA (scr). Plasma lipoprotein fraction profiling by fast protein liquid chromatography (FPLC) revealed higher cholesterol and apoB in the VLDL and LDL fractions, and higher triglyceride in VLDL (FIG. 1A) of the hepatocyte-tPA-silenced mice. Similarly, silencing hepatocyte tPA in WD-fed Apoe knockout (Apoe^{-/-}) mice led to 30% higher plasma total cholesterol (p<0.05), 25% higher apoB-100 (p<0.05), and 27% higher triglyceride (p<0.05) with consistent distribution in lipoprotein fractions compared to scramble-silencing controls (FIG. 1B). Similar results were observed in WD-fed C57BL/6J wild-type (WT) mice without changing in liver Apob mRNA (FIGS. 9A-9C), suggesting that increased apoB level was not due to increased apoB synthesis. Moreover, silencing hepatocyte tPA did not change liver LDLR and plasma apoE levels in WT mice (FIGS. 10A-10B). Collectively, silencing hepatocyte tPA leads to a higher plasma apoB lipoprotein-cholesterol through a mechanism independent of LDLR or apoE.

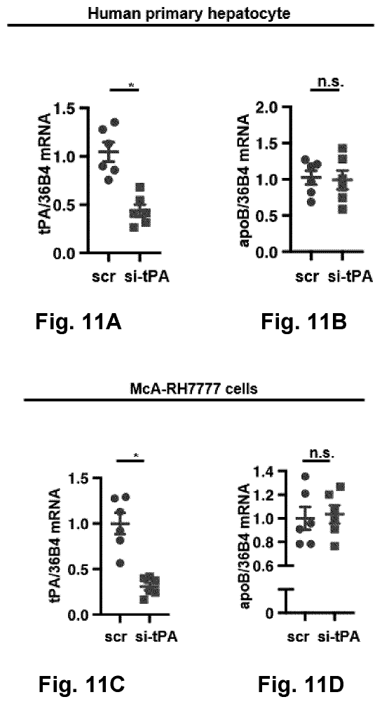

Consistently, silencing tPA in primary human hepatocytes using siRNA against PLAT mRNA (si-tPA) led to higher apoB-100 levels in serum-free culture medium (FIG. 1D), without altering hepatocyte APOB mRNA levels (FIG. 11B). The cholesterol and triglyceride in isolated VLDL were higher in tPA-silenced human hepatocytes (FIG. 1D). Similar findings were observed in cultured McA-RH7777 cells (FIG. 1E; FIGS. 11C-11D), a rat hepatoma cell line that is an established model system to study VLDL production, as it synthesizes VLDL having a size that is similar to human VLDL particles [23, 24].

Silencing hepatocyte tPA increases VLDL production and apoB lipidation in ER. After injecting a non-ionic surfactant detergent poloxamer 407 (P407), which inhibits lipoprotein lipase activity and VLDL lipolysis [25, 26], hepatocyte tPA-silenced mice have a faster triglyceride rising rate (FIG. 2A), suggesting silencing tPA in hepatocytes increases apoB-VLDL production. Moreover, plasma apoB-100, derived only from hepatocytes, increased to a lager degree upon hepatocyte tPA silencing compared to plasma apoB-48, produced by both hepatocytes and intestine in mice [27] (FIG. 2B).

Lipidation is a key factor determining the fate of intrahepatic apoB. Poorly lipidated apoB undergoes intracellular degradation, while fully lipidated apoB is efficiently secreted as particles with larger size and lower density [28]. Electron microscopy scanning of VLDL particles isolated by density ultracentrifugation showed a shifted distribution of VLDL to larger diameters in the hepatocyte tPA silenced Ldlr^{-/-} mice (FIG. 2C). Consistent larger hydrodynamic diameter of isolated VLDL was observed by dynamic light-scattering (DLS) method for their slower dispersed particle movement speed (Brownian motion) [29-32] (FIG. 2D), suggesting higher lipid content, which was validated by the higher TG/apoB ratio (FIG. 2E).

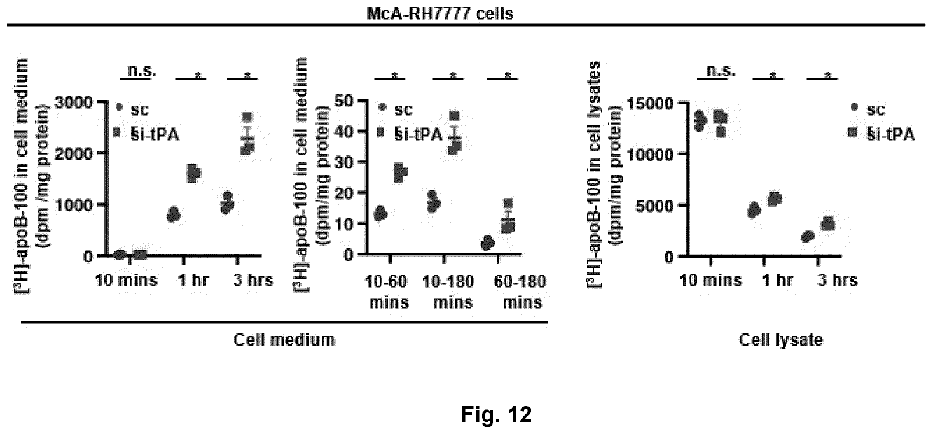

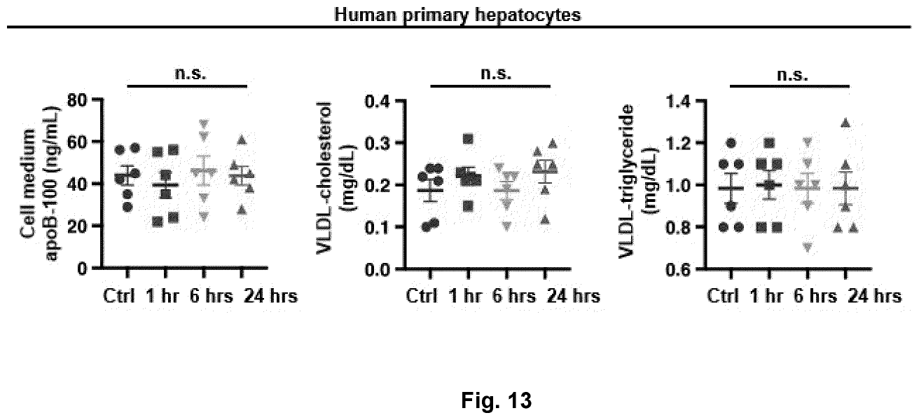

Consistently, silencing tPA in human primary hepatocytes led to higher apoB-associated radioactivity in the cell medium, indicating higher ³[H]-leucine-labelled apoB [33] secretion (FIG. 2H). Similar results were observed in McA-RH7777 cells (FIG. 12). Silencing tPA increased the diameter and TG/apoB ratio of VLDL isolated from the cell medium (FIGS. 2I, 2J). The ER-associated apoB levels were higher in tPA-silenced versus control human primary hepatocytes (FIG. 2K), whereas tPA-silenced hepatocytes have more apoB accumulated in the less dense ER fractions (fractions 1 and 2, FIG. 2K). As density and lipidation are inversely related, the findings were consistent with the hypothesis that tPA limits apoB lipidation.

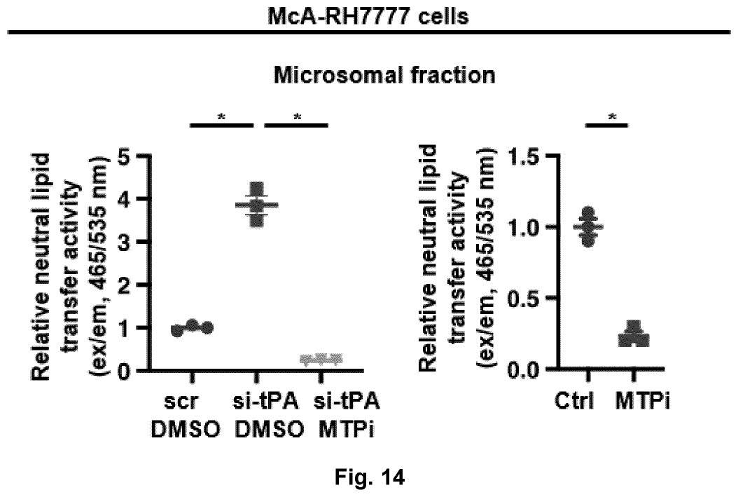

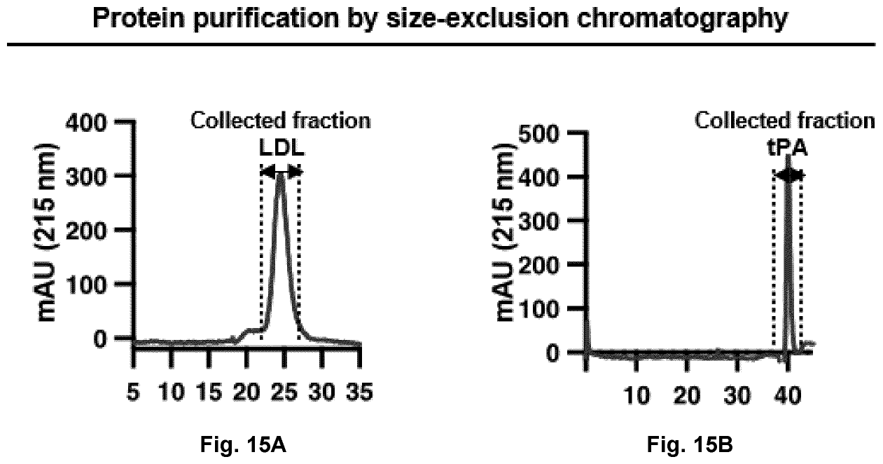

Hepatocyte tPA disrupts MTP-apoB interaction and inhibits MTP-dependent neutral lipid transfer. MTP is a critical chaperone that promotes intrahepatic apoB lipidation by transferring and incorporating neutral lipids, notably triglyceride and cholesteryl esters, to apoB to assemble VLDL [10, 11]. Although tPA silencing in human primary hepatocytes did not alter the level of MTP protein (FIG. 3A, input), anti-MTP immuno-precipitated from tPA-silenced cells showed higher apoB compared to control hepatocytes (FIG. 3A), suggesting that silencing tPA increased apoB-MTP interaction. Consistently, microsomal fractions isolated from tPA-silenced hepatocytes showed two-fold higher neutral lipid transfer activity than the microsomes from control cells (FIG. 3B, groups 1-2). The transfer activity was indeed due to MTP, as the MTP inhibitor, CP-346086 [34], [35], completely abolished the elevated neutral lipid transfer activity in tPA-silenced hepatocytes (FIG. 3B, group 3). Similar findings were observed in McA-RH7777 cells (FIG. 14). MTP-mediated lipid transfer to apoB involves its direct binding to apoB [10], as inhibiting apoB-MTP interaction decreased apoB lipidation and secretion [11].

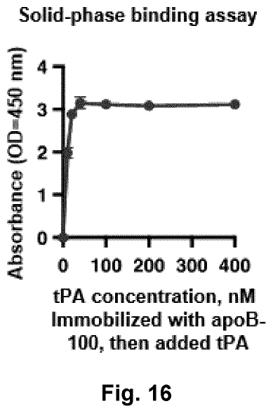

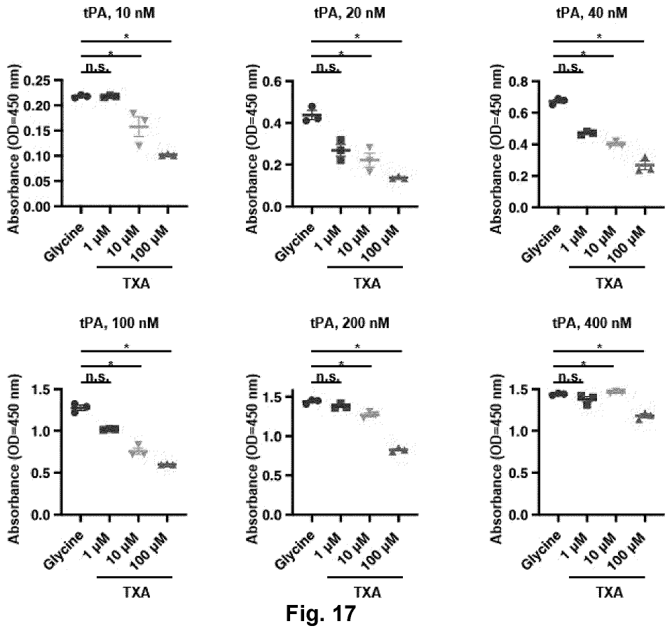

In primary human hepatocytes transfected with plasmids expressing tPA with a C-terminal HA-tag, apoB was detected in the anti-HA-precipitated elute (FIG. 3F). Proximity ligation assay (PLA) showing punctate fluorescent signal (FIG. 3G;) indicates the proximity between tPA and apoB in hepatocytes, suggesting the intracellular interaction between endogenous tPA and apoB in hepatocytes, further supported by the colocalization of tPA and apoB in ER by Immunofluorescence staining (FIG. 3H). The purified recombinant tPA directly interacts with microtiter plate surface-bound purified apoB-containing LDL dose-dependently in a solid-phase protein-binding assay (FIG. 4A). Consistent interaction was observed between purified recombinant tPA and purified apoB-100 (FIG. 16). By contrast, purified tPA did not interact with surface-bound MTP (FIG. 3B). Pre-incubating LDL with tPA inhibited the binding between MTP and surface-bound LDL (FIG. 3C), and reduced the MTP-mediated neutral lipid transfer to the LDL (FIG. 3J). Surface plasmon resonance (SPR) studies suggested a non-covalent bind between tPA and LDL particles (FIG. 4D). Quantitative analysis of the sensorgrams using a 2-state binding model yielded a Kd of approximately 260 nM for the LDL-tPA interaction. Taken together, tPA directly interacts with apoB containing lipoproteins to reduce the availability of apoB to MTP for incorporating neutral lipids, and thereby inhibits VLDL assembly.

Transduction of hepatocytes with tPA lowers VLDL assembly in hepatocytes ER and apoB secretion independent of tPA's serine protease activity. Increasing tPA expression only in hepatocytes with AAV8 virus under the TBG promoter (AAV8-TBG-tPA) in tPA knockout mice (holo-tPA-KO) [21, 22, 36-38] lowered plasma apoB-100, VLDL- and LDL-cholesterol vs. AAV8-TBG-LacZ controls (FIG. 2F). Consistently, expressing tPA by plasmids (e.g., pCMV3-tPA-HA) in human primary hepatocytes reduced newly synthesized apoB secretion using pulse-chase assay, neutral lipid transfer activity and apoB-MTP interaction (FIGS. 2G, 3C, 3D). Taken together, tPA limits apoB lipidation and its subsequent secretion by reducing the accessibility of apoB to MTP.

MTP-apoB interaction occurs in hepatocyte ER [39]. Using confocal immunofluorescence microscopy, tPA and apoB colocalize in ER (FIG. 3H), where apoB interacts with MTP for its lipidation and VLDL assembly. Retaining tPA in the ER by adding a KDEL sequence [40-43] at the C-terminal of human tPA protein (tPA-KDEL; SEQ ID NO: SEQ ID NO: 3) reduced newly synthesized apoB secretion by pulse-chase assay in human primary hepatocytes transduced with a plasmid (pCMV3-tPA-KDEL set forth in SEQ ID NO: 24) encoding tPA-KDEL (SEQ ID NO: 3) (FIG. 3I). Collectively, these results demonstrate that tPA interacts with apoB in ER and limits apoB-VLDL assembly and secretion.

The protease activity of tPA depends on the serine at position 513, and substitution of serine to alanine (S513A) completely abolish the serine proteases activity of tPA [44]. Similar to WT tPA, the serine protease mutant tPA (S513A; SEQ ID NO: 9) reduced apoB secretion by pulse-chase assay in human primary hepatocytes transduced with a plasmid (pCMV3-tPA-S513A; SEQ ID NO: 23) encoding tPA S513A (FIG. 3I). The recombinant tPA-S513A protein bound to surface-bound LDL with similar affinity to WT tPA (FIG. 4A) and impaired MTP-mediated lipid transfer activity (FIG. 3J), suggesting tPA binds to apoB and decreases apoB lipidation and secretion independent of tPA serine protease activity.

The lysine-binding site at Kringle 2 domain of tPA interacts with the lysine-rich region at N-terminus of apoB. The Kringle 2 domain of tPA has the lysine binding site [45], and is required for its interaction with fibrin. The negatively charged residues, aspartic acid-236 and -238, in the Kringle 2 domain of human tPA are responsible for the binding of tPA to positively charged lysine [46]. The surface exposing N-terminus of apoB [47] has a lysine-rich region that is responsible for its binding with MTP and the MTP-mediated lipidation activity [48]. Expressing tPA mutants lacking either Kringle 2 domain (tPA-Δ-K2) or substituting the aspartic acids at positions 236 and 238 with asparagine (tPA-D236, 238N) did not alter apoB secretion compared to wild type tPA by pulse-chase assay (FIG. 4F) Similarly, in the solid-phase protein binding assay, the antibody against tPA Kringle 2 domain (sigma, HPA003412) inhibited the binding between surface-bound tPA and LDL (FIG. 4G) compared to rabbit IgG control. Tranexamic acid (TXA), a lysine analogue, reduced the interaction between tPA and surface-bound LDL (FIG. 4H). Therefore, tPA blocks apoB-MTP interaction by competitively binding to the lysine-rich region of apoB through the lysine binding site at aspartic acid-236 and -238 on Kringle 2 domain of tPA, and thereby reduces the accessibility of MTP to the apoB for lipidation.

PAI-1 sequesters tPA from apoB, leading to increased VLDL assembly within hepatocytes. Work by many groups have characterized the lipid-loading-induced prolonged postprandial lipidemia enhances hepatic production of VLDL, yet the underlying mechanism is not clearly understood. Obesity, often associated with dyslipidemia, increases tPA synthesis in hepatocytes [21], which is then overcompensated by a larger increase in its serpin inhibitor, PAI-1, resulting in decreased net free functional tPA in the livers and plasma [21]. PAI-1 covalently binds to tPA, forming a stable complex, and inactivates tPA's serine protease function [49]. Therefore, the inventors hypothesize that postprandial lipid-loading increases the intracellular interaction of tPA and PAI-1, leading to reduced free tPA which restrains the availability of apoB to be accessed by MTP for lipidation, and eventually causes increased VLDL assembly and secretion to blood circulation.

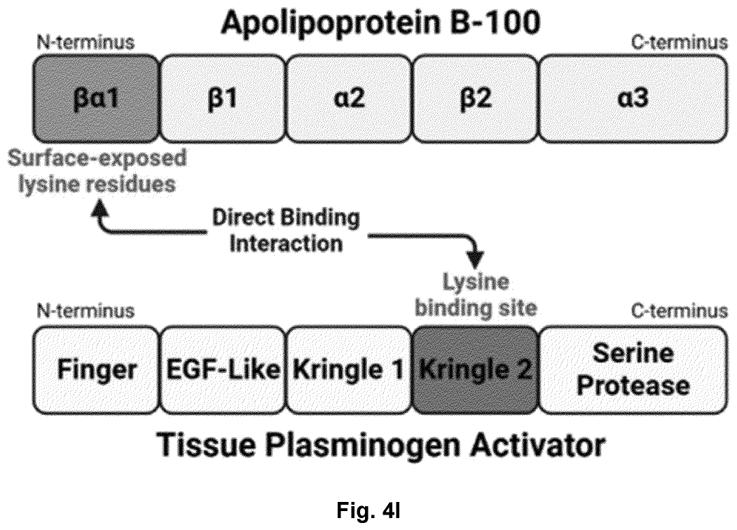

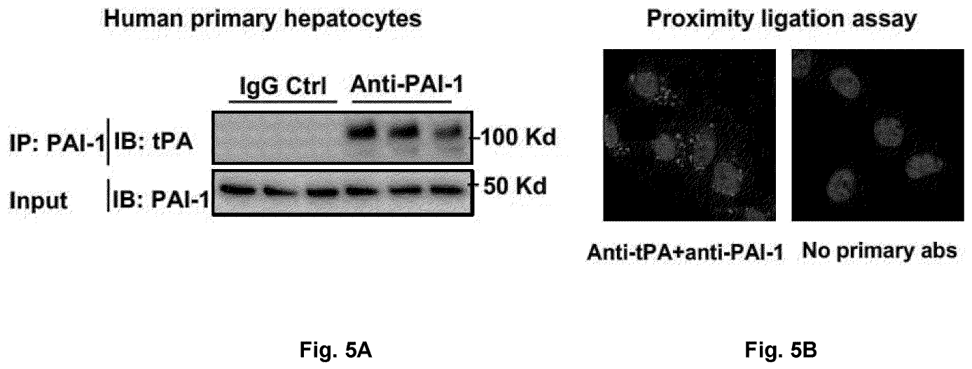

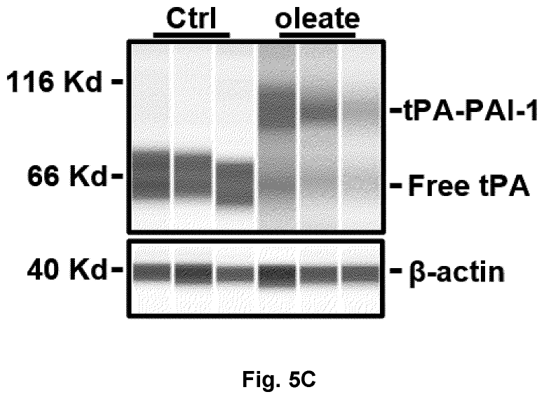

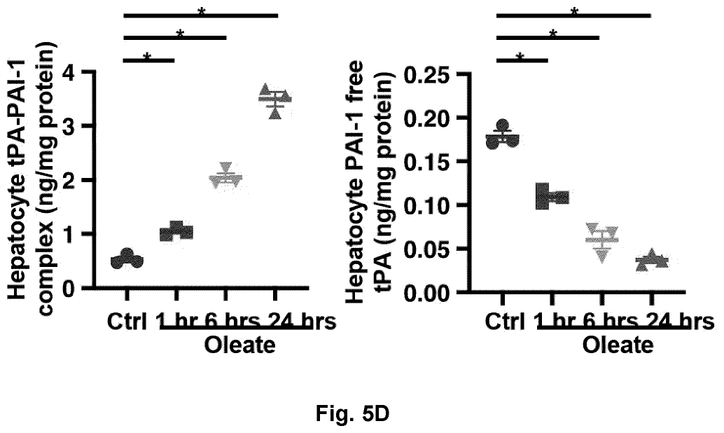

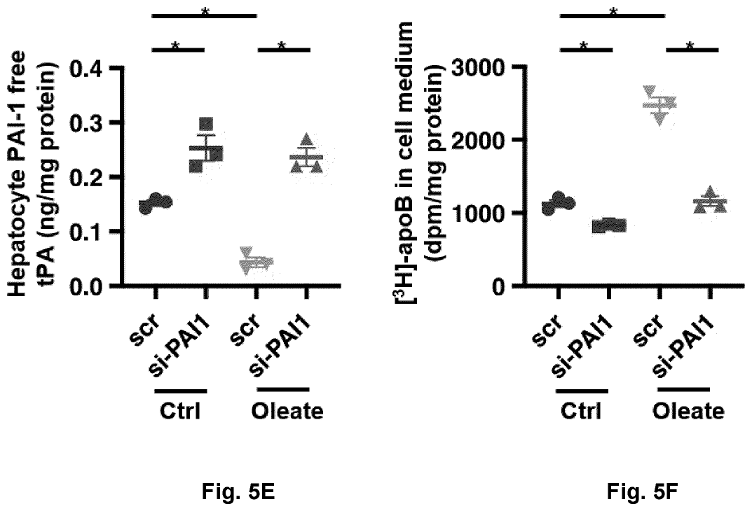

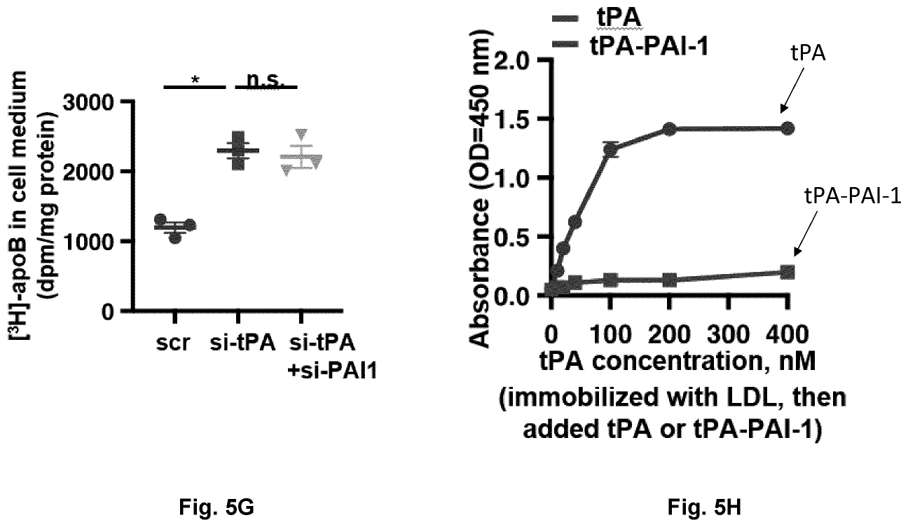

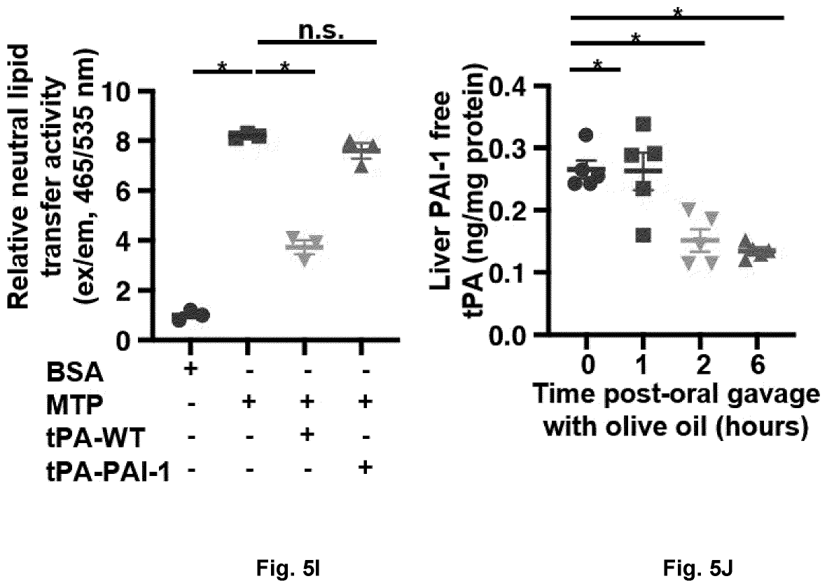

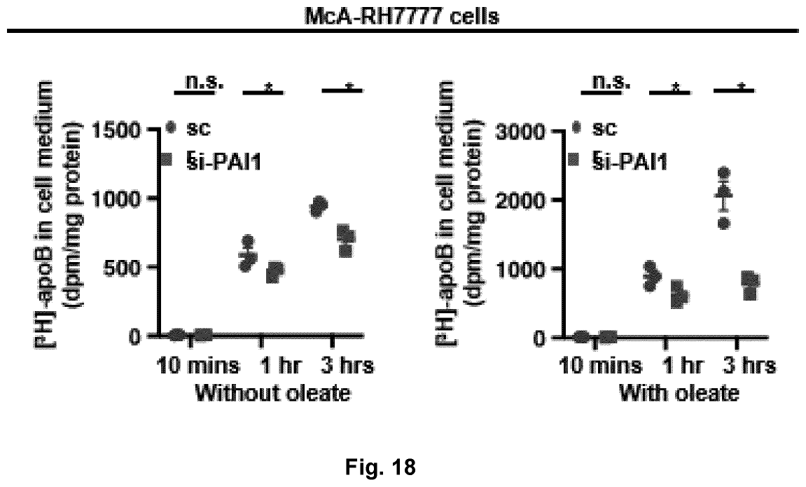

Probing tPA (~70 Kd) after running the immunoprecipitated PAI-1 (~50 Kd) elute from human primary hepatocytes through SDS-PAGE gel showed a band located at ~120 Kd, representing a covalently-bound, SDS-stable tPA-PAI-1 complex, further validated by PLA for their endogenous interaction within proximity in live human primary hepatocytes (FIGS. 5A-5B). As early as one-hour post-treating human primary hepatocytes with oleate, a well-established stimulus for apoB lipidation and VLDL production [50], the tPA-PAI-1 complexation increased while free tPA reduced (FIG. 5C-5D), similar to the time for stimulatory effect of oleate on VLDL production [51]. This rapid complexation of PAI-1 to tPA without requiring newly synthesized tPA or PAI-1 proteins, and the sequestration of tPA by PAI-1 suggests a timely and fine-tune regulation of apoB lipidation and VLDL production when the hepatocytes are loaded with lipids. With the prolonged oleate treatment for 6 and 24 hours, the free tPA is further decreased due to more PAI-1-tPA complex formation (FIG. 5D). Consistent results were observed in McA-RH7777 cells (FIG. 18).

Silencing PAI-1 in the human primary hepatocytes led to higher free tPA and lower apoB secretion by pulse-chase (FIGS. 5E-5F). The inhibitory impact of si-PAI-1 on apoB secretion is more prominent after oleate-treatment with ~400% increase in free tPA and ~60% decrease in apoB secretion, while only ~66% increase in free tPA and 25% decrease in apoB secretion without oleate. These observations are consistent with the hypothesis that under basal condition, most of tPA are free and not bound by PAI-1; silencing PAI-1 under this basal condition only leads to moderate increase in free tPA and the subsequent mild reduction in apoB secretion. However, under the oleate over-load condition, more tPA are PAI-1-bound, and silencing PAI1 leads to a robust increase in free tPA and reduction in apoB secretion. Consistently, compared to silencing tPA alone, silencing both tPA and PAI-1 at the same time did not further lower apoB secretion, supporting the idea that PAI-1 facilitates apoB lipidation through sequestering tPA from apoB, rather than functions through PAI-1 itself (FIG. 5G). Consistent results were observed in the McA-RH7777 hepatocytes (FIG. 18).

The complexation of tPA and PAI-1 causes conformation change in tPA protein structure [52], and loses its fibrin binding ability mediated by the lysine binding site in Kringle 2 domain [53], suggesting that the binding of PAI-1 to tPA blocks the lysine binding site in Kringle 2 domain potentially mediated by the steric hindrance effect or the conformational change of Kringle 2 domain. Unlike purified tPA alone, the purified PAI-1-tPA complex was unable to bind to LDL, or to inhibit MTP-mediated neutral lipid transfer activity (FIGS. 5H-5I), indicating the complexation of PAI-1 with tPA prevents interaction between tPA and apoB.

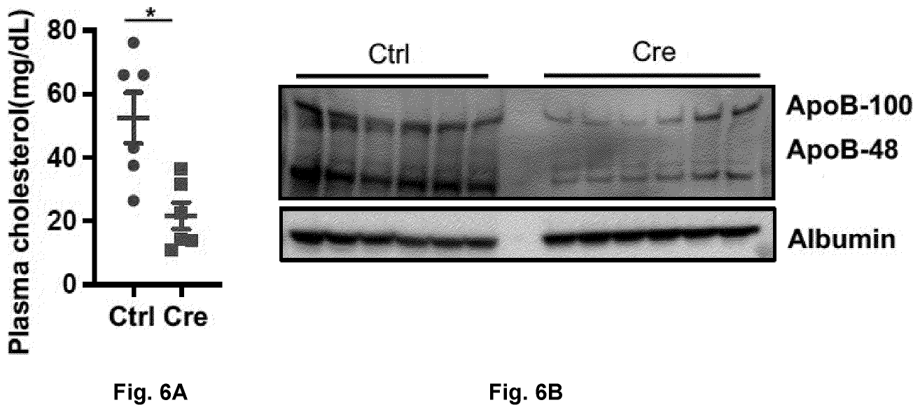

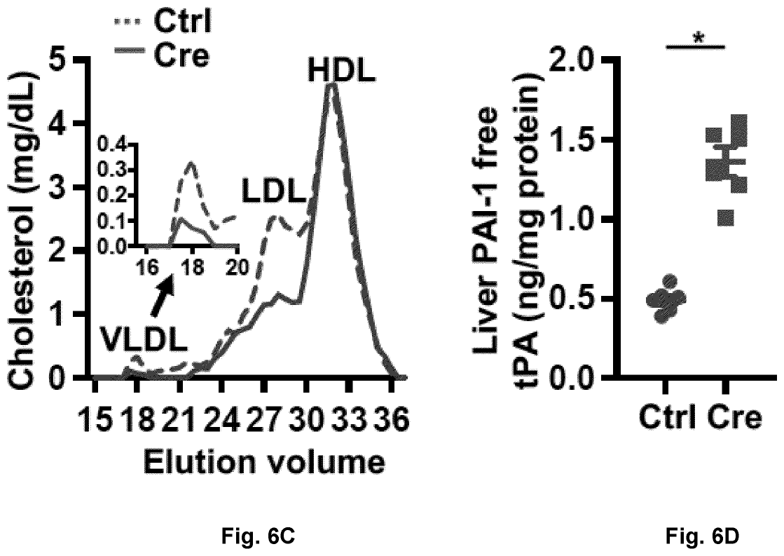

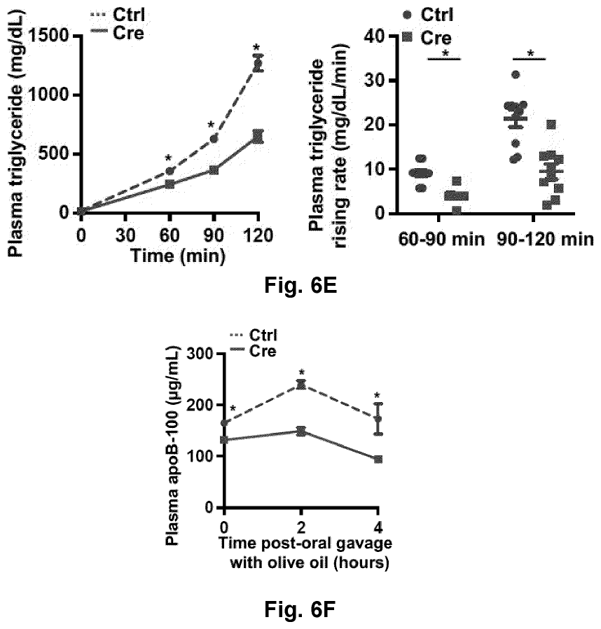

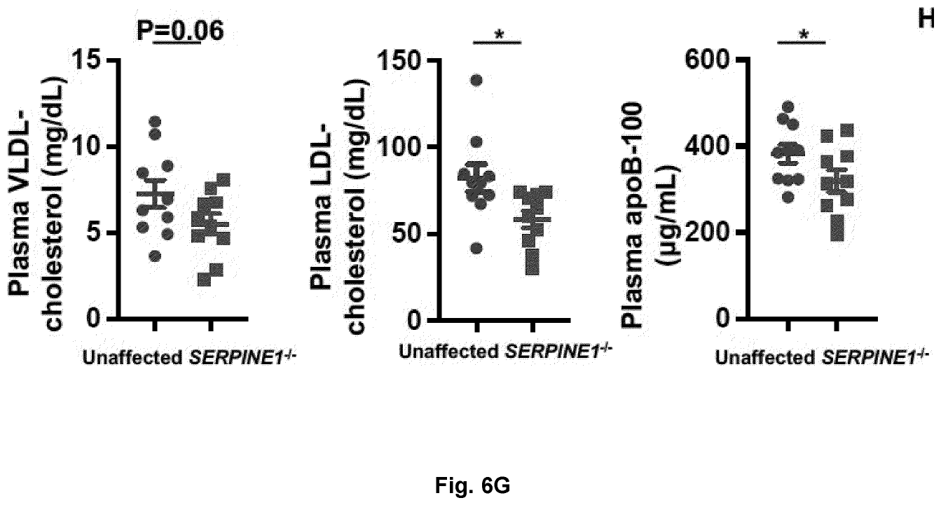

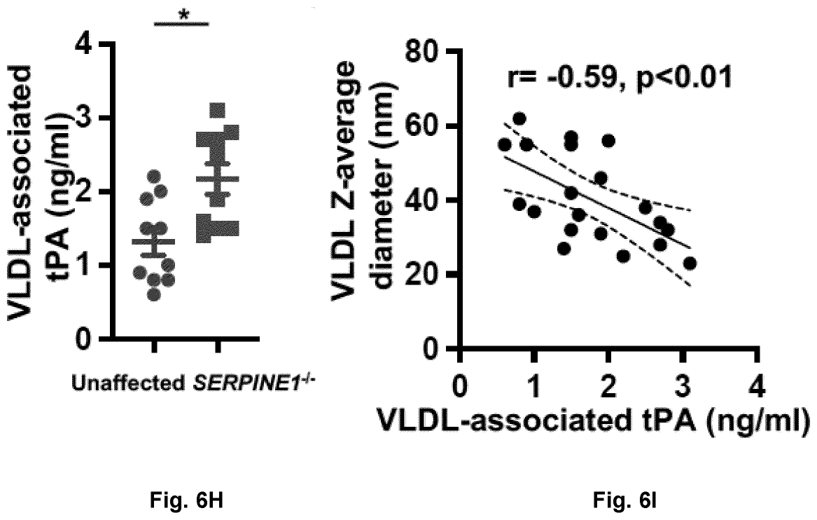

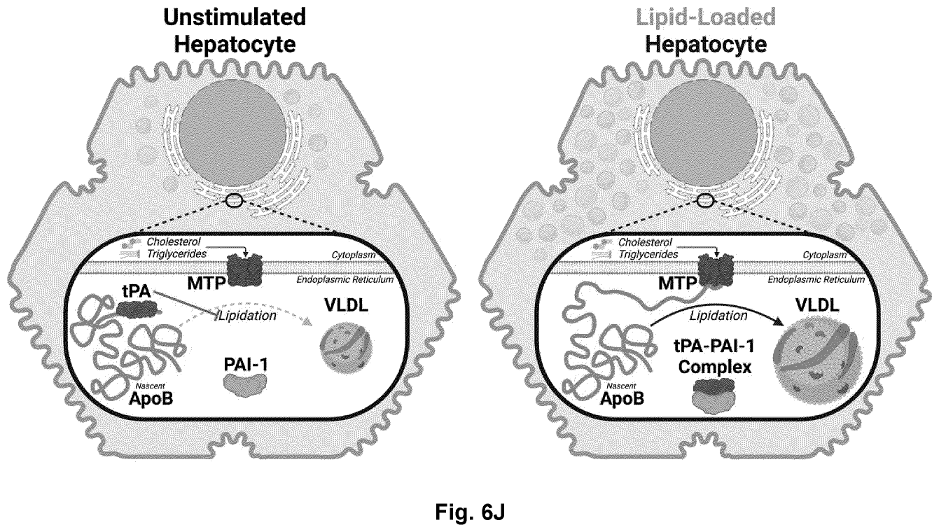

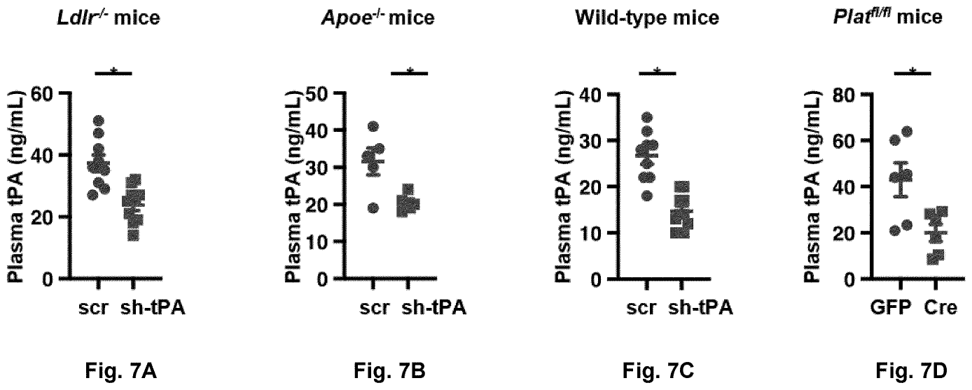

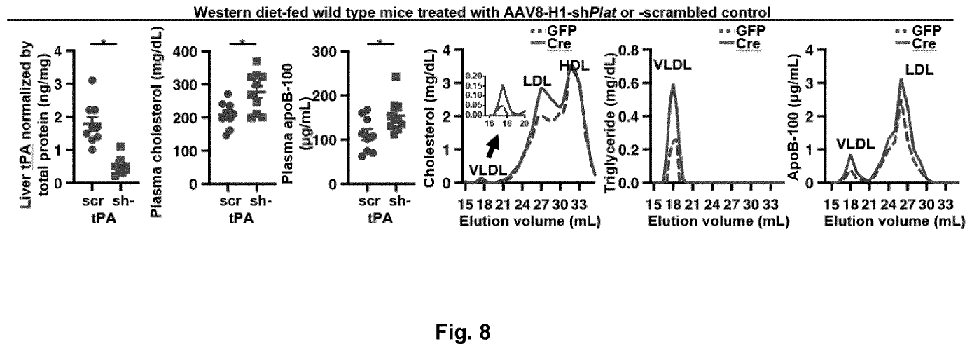

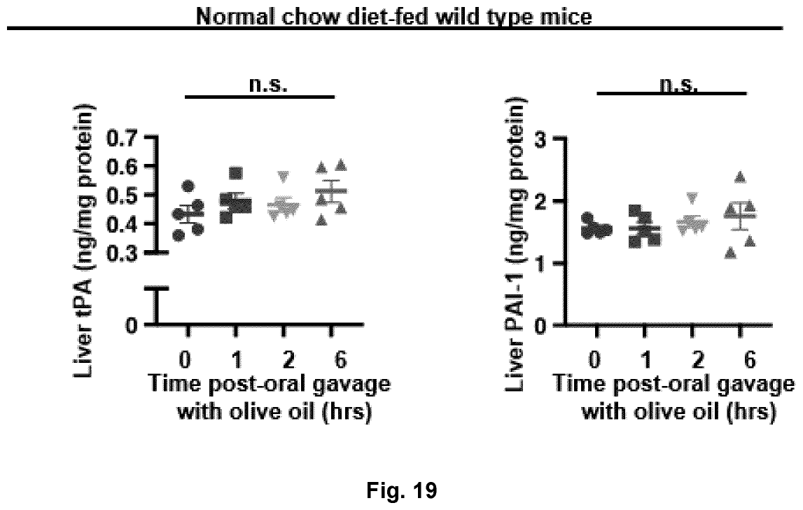

After 2 hours or 6 hours post olive oil gavage to C57BL/6J mice to increase dietary fatty acid intake which subsequently increases blood fatty acid and lipid loading to hepatocytes, the liver free tPA was decreased compared to baseline (FIG. 5J, FIG. 19), without altering liver total tPA and PAI-1 levels. As expected, the obese hepatocyte-specific PAI-1 knockout mice (H-PAI-1 KO) [21, 54] had higher free tPA in both livers and plasma, lower plasma apoB, total cholesterol, and cholesterol in VLDL and LDL fractions than their littermate controls (FIGS. 6A-6F).

Taken together, the PAI1-tPA intracellular interaction maintains the balance of VLDL production. When fatty acids are loaded to the hepatocytes, PAI-1 rapidly complexed with tPA within the hepatocytes, reducing available free tPA to directly interacts with apoB and therefore limiting its lipidation, leading to the eventual increased VLDL assembly and secretion to the blood circulation.

PAI-1 deficiency in humans leads to lower plasma apoB and apoB-cholesterol levels. Humans with homozygous PAI-1 deficiency, due to a unique loss-of-function mutation in SERPINE1 (SERPINE1^{-/-}, n=10), had 22% lower LDL cholesterol (p<0.05), and borderline significant 21% lower apoB (p=0.07), 18% lower cholesterol (p=0.06) and 16% lower triglyceride (p=0.07) on VLDL fractions than the unaffected individuals from the same community (age, gender and BMI matched controls, n=10) (FIG. 6G). None of these individuals were taking lipid-lowering agents or had known history of cardiovascular diseases. Compared to unaffected control individuals, the PAI-1 deficient individuals had higher tPA on the isolated VLDL particles (FIG. 6H). Moreover, the VLDL-associated tPA levels were inversely associated with VLDL diameter (r=−0.59, p<0.01) (FIG. 6I). Together with data from hepatocyte-PAI-1 deficient mice and PAI-1-silenced human primary hepatocytes (FIGS. 5F and 6D), the deficiency of PAI-1 leads to higher free-tPA interacting with apoB and limits apoB lipidation in hepatocytes.

Taken together, the results described above demonstrate that tPA directly interacts with apoB, via tPA's K2 domain, within the ER of hepatocytes and that this interaction reduces MPT-mediated VLDL assembly. Lipid loading to hepatocytes induces tPA-PAI-1 complex formation, sequestering tPA from apoB, and thereby facilitating apoB lipidation and VLDL assembly (FIG. 6J).

Notably, in cultured hepatocytes, exogenous expression of only the tPA-K2 domain by transducing cells with plasmids encoding a tPA-K2 domain operably linked to an endoplasmic reticulum localization motif reduces apoB secretion (FIGS. 23A-23B). Moreover, intravenous injection with LNP encompassing the mRNA of tPA-K2 reduces plasma lipids in mice (FIGS. 24A-24B). These findings demonstrate that the recombinant tPA-K2 polypeptides of the present technology are useful in compositions and methods for treating hyperlipidemia.

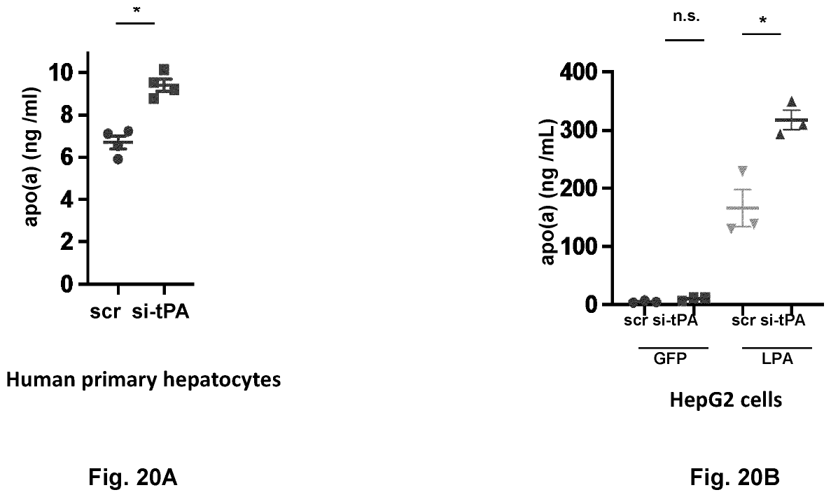

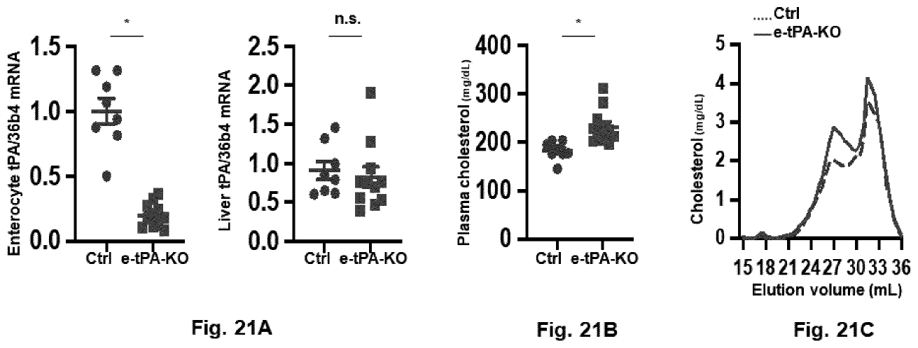

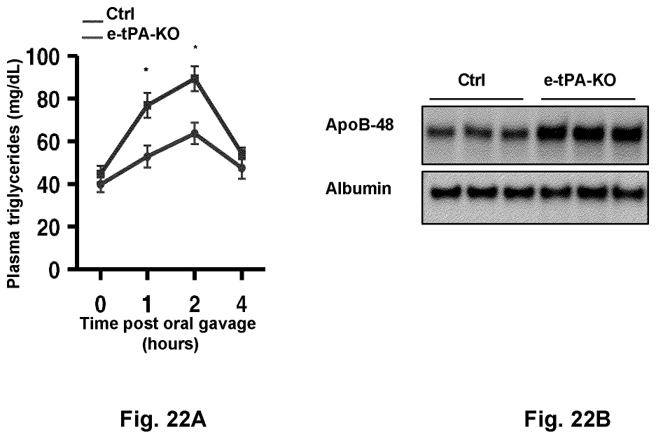

Silencing tPA leads to reduced Lp(a) in cell medium from both cultured human primary hepatocytes (FIG. 20A) and HepG2 cells (FIG. 20B), indicating tPA also limits the production of Lp(a), another atherogenic apoB-containing lipoprotein, in hepatocytes. Moreover, knocking out enterocyte tPA increases plasma cholesterol levels (FIGS. 21A-21C) and chylomicron production (FIGS. 22A-22B) in mice, indicating that the regulatory effect of tPA in apoB-lipoprotein production also exist in enterocytes, which absorb and transport dietary lipids into bodies. These findings demonstrate that the recombinant tPA-K2 polypeptides of the present technology would be useful in compositions and methods for reducing plasma Lp(a) and chylomicron levels, further reducing cardiovascular risk.

### DISCUSSION

The inventors' findings reveal a novel mechanism that fine-tunes the rate of apoB-lipoprotein assembly by tPA in hepatocytes. For example, without wishing to be bound by theory, assembly of VLDL in ER of hepatocytes is accomplished in two steps [9, 55]. In the first step, apoB co-translational lipidation allows MTP transferring lipids to the growing apoB polypeptides, including its surface-exposing N-terminus, on ER membrane to form a primordial VLDL particle, that are approximately the same size as plasma HDL [11]. Then the primoridial VLDL detaches from the ER membrane and becomes lumenal particles [9], which undergoes further lipidation into mature VLDL particles in the second step of VLDL assembly [55]. MTP also facilitates the fusion of primordial VLDL with ER lumenal lipid droplet [56]. MTP inhibitor disrupts apoB lipidation and reduces VLDL production [57]. Within hepatocytes, tPA, similar as the MTP inhibitor, competes with MTP for interaction with the apoB (FIG. 6J). Kringle domains are autonomous protein domains that fold into large loops stabilized by 3 disulfide bonds, which are responsible for protein-protein interactions [58]. The Kringle 2 domain of tPA has the lysine binding site [46], which interacts with lysine-rich region [45, 59]. The N-terminal of apoB contains a lysine-rich region that is required for its interaction with MTP for lipidation [10]. The inventors' competitive binding assays showed an antibody against tPA Kringle 2 domain, or the lysine analogue, TXA, inhibited the interaction between tPA and LDL, suggesting the interaction is potentially mediated by the lysine binding site in Kringle 2 domain of tPA. The newly-synthesized apoB takes ~40 minutes to be secreted from hepatocyte [51], and the apoB-associated radioactivity at the early chasing stage (5-10 minutes) is used to represent the newly-synthesized apoB quantity after pulse labeling [51]. The inventors found no difference in apoB-associated radioactivity in cell lysate after chasing for 10 mins (FIG. 18). This suggested that silencing tPA did not increase apoB protein synthesis rate, which is also supported by the observations that silencing tPA did not alter apoB mRNA level in WT mouse liver, cultured human primary hepatocytes and McA-RH7777 cells (FIGS. 9A-9C and 11A-11D).

Oleic acid influx to hepatocyte stimulates apoB lipidation and VLDL assembly [51], with the mechanisms not fully understood. The inventors' findings that oleate treatment increases intracellular tPA-PAI-1 complex formation, leading to decreased free tPA in hepatocyte, provide new insights in the molecular mechanism of the stimulatory effect of oleic acid on apoB production. The tPA-PAI-1 complex increased only 1 hour post oleate treatment, in which both the total tPA and PAI-1 level unaffected. In contrast, prolonged oleatetreament more than 6 hours also increased the total PAI-1 protein levels in culture human primary hepatocyte. Consistently, two-hour post oral gavage mouse with olive oil, in which around 70% percent of fatty acid is oleic acid, the liver tPA-PAI-1 decreased while no change in total tPA and PAI-1 (FIG. 19). These results suggest oleate influx to hepatocyte quickly induces the complexation between tPA and PAI-1 within one hour in vitro or two hours in vivo. In summary, lipid loading to hepatocyte induces PAI-1 compexation with tPA, sequesters tPA away from apoB, making apoB available for MTP to bind, and therefore augments apoB lipidation and VLDL production, revealing the physiological significance of tPA-PAI-1 interaction in fine-tuning apoB lipidation after lipid-loading to hepatocytes.

Increased blood PAI-1 level and decreased tPA activity, resulting in impaired fibrinolysis, are independent risk factors of atherothrombotic diseases [60]. Clinical observational data indicate that the plasma concentration of PAI-1 is positively while tPA activity is negatively associated with apoB cholesterol [57, 60, 61].

### SUMMARY

In summary, these results demonstrate that the tPA-K2 polypeptides of the present technology—although lacking serine protease and proteolytic capabilities—are effective in methods for reducing apoB secretion and lowering plasma triglyceride and total cholesterol levels when administered to a subject. Moreover, because the tPA-K2 fragments of the present technology (e.g., comprising an amino acid sequence as set forth in SEQ ID NO. 7), do not have serine protease and proteolytic activity, administration of the peptides of the present technology will not increase the bleeding risk in a subject. This Example demonstrates that the expression of a recombinant tPA-K2 peptide comprising the tPA Kringle 2 domain alone and an endoplasmic reticulum localization sequence reduces apoB secretion from hepatocytes (FIGS. 23A-23B) and lowers plasma lipid levels in an in vivo mouse model (FIGS. 24A-24B), but does not alter the bleeding time in the mice (FIG. 25). Accordingly, these data support that compositions comprising the recombinant tPA-K2 polypeptides (or nucleic acids (e.g., cDNA, mRNA) encoding the same) are useful in methods for the treatment of diseases or conditions associated with elevated plasma apoB-lipoprotein levels, and are useful in methods of reducing any one or more of plasma VLDL, IDL, LDL, Lp(a), chylomicron, chylomicron remnant, triglyceride, or total cholesterol levels. These data also demonstrate that the compositions of the present technology are useful in methods for lowering plasma lipids and/or apoB levels in subjects in need of and/or undergoing thrombolytic therapy.

### Example 2—Treatment of Subjects with Recombinant Polypeptides of the Instant Disclosure

In one example, a subject suffering from cardiovascular disease, e.g., hyperlipidemia, atherosclerosis, increased risk of blood clots or atherothrombotic events, angina, heart attack, heart failure, stroke, transient ischemic attack(s) (TIA), peripheral artery disease, or high blood pressure, is administered a therapeutically effective amount of a pharmaceutical composition comprising the disclosed recombinant tPA-K2 polypeptides or nucleic acids encoding the same. The subject may be undergoing a standard of care for thrombolytic therapy (e.g., receiving a tissue plasminogen activator therapy). The disclosed recombinant tPA-K2 polypeptides or nucleic acids encoding the same (e.g., polypeptides comprising amino acid sequences as set forth in SEQ ID Nos. 3, 5 and 7 and/or polynucleotide molecules comprising nucleic acid sequences as set forth in SEQ ID Nos. 4, 6, 8, or 34) may be administered by any route that is indicated by the particular treatment needs of the subject, e.g., oral, transdermal, percutaneous, intravenous, intramuscular, intranasal, buccal, intrathecal, intracerebral, or intrarectal routes. Signs and symptoms of cardiovascular disease may be reduced by the administration of the pharmaceutical compositions comprising the disclosed recombinant tPA-K2 polypeptides or nucleic acids encoding the same. Treatment may be administered daily, every other day, every third day, or on a schedule as determined by the patient's progress, pursuant to a physician's decision. It is anticipated that the subject will experience a decrease in triglycerides, total cholesterol, VLDL, LDL, IDL, Lp(a), chylomicrons, chylomicron remnants, a decrease in blood pressure or other metrics associated with reduction in signs or symptoms of cardiovascular disease, as compared to an untreated subject. Methods of measuring reductions in signs and symptoms of cardiovascular disease are known in the art.

It is anticipated that these results will demonstrate that compositions comprising the recombinant tPA-K2 polypeptides of the present technology or nucleic acids encoding the same are useful in methods for treating subjects with cardiovascular disease.

### Example 3—Treatment of High Cholesterol in Subjects with Recombinant Tissue Plasminogen Activator

In one example, a subject suffering from hyperlipidemia is administered a therapeutically effective amount of a pharmaceutical composition comprising one or more of the recombinant tPA-K2 polypeptides of the present technology or a nucleic acid encoding the same. The subject may be at risk for or suffering from atherothrombotic events and undergoing a standard of care for thrombolytic therapy (e.g., receiving a tissue plasminogen activator therapy). The recombinant tPA-K2 or nucleic acid encoding the same (e.g., polypeptides comprising amino acid sequences as set forth in SEQ ID Nos. 3, 5 and 7 and/or polynucleotide molecules comprising nucleic acid sequences as set forth in SEQ ID Nos. 4, 6, 8, or 34) may be administered by any route that is indicated by the particular treatment needs of the subject, e.g., oral, transdermal, percutaneous, intravenous, intramuscular, intranasal, buccal, intrathecal, intracerebral, or intrarectal routes. Levels of triglycerides, total cholesterol, VLDL, LDL, IDL, Lp(a), chylomicrons, or chylomicron remnants may be reduced by the administration of the recombinant tPA-K2 or nucleic acid encoding the same. Treatment may be administered daily, every other day, every third day, or on a schedule as determined by the patient's progress, pursuant to a physician's decision. It is anticipated that the subject will experience a decrease in triglycerides, total cholesterol, VLDL, LDL, IDL, Lp(a), chylomicrons, chylomicron remnants, or other metrics associated with reduction in signs or symptoms of hyperlipidemia, as compared to an untreated subject. Methods of measuring reductions in signs and symptoms of hyperlipidemia are known in the art.

It is anticipated that these results will demonstrate that compositions comprising the recombinant tPA-K2 polypeptides of the present technology or nucleic acids encoding the same are useful in methods for treating subjects with hyperlipidemia.

### Example 4—Treatment with Recombinant tPA-K2 Reduces Lp(a) Assembly and Production in Hepatocytes

This experiment will demonstrate the efficacy of the recombinant tPA-K2 polypeptides of the present technology or nucleic acids encoding the same (e.g., polypeptides comprising amino acid sequences as set forth in SEQ ID Nos. 5 and 7 and/or polynucleotide molecules comprising nucleic acid sequences as set forth in SEQ ID Nos. 6, 8, or 34) to reduce Lp(a) assembly and production in hepatocytes. Briefly, human primary hepatocytes will be treated with plasmid, cDNA, and/or mRNA encoding tPA-K2 (e.g., tPA-K2-HA-KDEL). Cells and cell medium will be harvest and collected. Lp(a) levels in cell lysate and cell medium will be measured by immunoblot.

It is anticipated that these results will demonstrate that compositions comprising the recombinant tPA-K2 polypeptides of the present technology or nucleic acids encoding the same are useful in methods for lowering plasma Lp(a) levels in a subject in need thereof.

### Example 5—Treatment with Recombinant tPA-K2 Reduces Chylomicron Assembly and Production in Enterocytes

This experiment will demonstrate the efficacy of the recombinant tPA-K2 polypeptides of the present technology or nucleic acids encoding the same (e.g., polypeptides comprising amino acid sequences as set forth in SEQ ID Nos. 5 and 7 and/or polynucleotide molecules comprising nucleic acid sequences as set forth in SEQ ID Nos. 6, 8, or 34) to reduce chylomicron assembly and production in enterocytes. Briefly, Caco-2 cells, an intestinal epithelial cell line, will be treated with plasmid, cDNA, and/or mRNA encoding tPA-K2 (e.g., tPA-K2-HA-KDEL). Cells and cell medium will be harvest and collected. ApoB48 levels in cell lysate and cell medium will be measured by immunoblot.

It is anticipated that these results will demonstrate that compositions comprising recombinant tPA-K2 polypeptides of the present technology or nucleic acids encoding the same are useful in methods for reducing chylomicron production.

### Example 6—Treatment with Recombinant tPA-K2 does not Significantly Increase the Risk of Liver Steatosis or Liver Injuries in Subjects

This experiment will demonstrate that treatment of subjects with the recombinant tPA-K2 polypeptides of the present technology or nucleic acids encoding the same (e.g., polypeptides comprising amino acid sequences as set forth in SEQ ID Nos. 5 and 7 and/or polynucleotide molecules comprising nucleic acid sequences as set forth in SEQ ID Nos. 6, 8, or 34) will not increase their risk of liver steatosis or injury. Briefly, mice are intravenously injected with LNP carrying mRNA encoding tPA-K2-HA-KDEL (SEQ ID NO: 32) or control Luciferase (1 mg/kg body weight). Blood will be collected 24 hours post-injection and just before injection. Mice will be euthanized 24 hours post-injection, and liver will be collected Blood liver injury markers will be measured. Liver histology analysis will be conducted.

It is anticipated that these results will demonstrate that treatment of a subject with recombinant tPA-K2 of the present technology or nucleic acids encoding the same will not increase the risk of liver steatosis or liver injury in the subject.

### REFERENCES

1. Brunner, F. J., et al., Application of non-HDL cholesterol for population-based cardiovascular risk stratification: results from the Multinational Cardiovascular Risk Consortium. Lancet, 2019. 394(10215): p. 2173-2183.
2. Marston, N. A., et al., Association of Apolipoprotein B-Containing Lipoproteins and Risk of Myocardial Infarction in Individuals With and Without Atherosclerosis: Distinguishing Between Particle Concentration, Type, and Content. JAMA Cardiol, 2022. 7(3): p. 250-256.
3. Stone, N.J., et al., 2013 ACC/AHA guideline on the treatment of blood cholesterol to reduce atherosclerotic cardiovascular risk in adults: a report of the American College of Cardiology/American Heart Association Task Force on Practice Guidelines. J Am Coll Cardiol, 2014. 63(25 Pt B): p. 2889-934.
4. Mabuchi, H., et al., Effect of an inhibitor of 3-hydroxy-3-methyglutaryl coenzyme A reductase on serum lipoproteins and ubiquinone-10-levels in patients with familial hypercholesterolemia. N Engl J Med, 1981. 305(9): p. 478-82.
5. Horton, J. D., J. C. Cohen, and H. H. Hobbs, PCSK9: a convertase that coordinates LDL catabolismj Lipid Res, 2009. 50 Suppl: p. S172-7.
6. Reith, C. and J. Armitage, Management of residual risk after statin therapy. Atherosclerosis, 2016. 245: p. 161-70.
7. Wong, N. D., Residual Risk After Treatment of Patients With Atherosclerotic Cardiovascular Disease With Proprotein Convertase Subtilisin-Kexin Type 9 Monoclonal Antibody Therapy (from the Further Cardiovascular Outcomes Research With PCSK9 Inhibition in Subjects With Elevated Risk Trial). Am J Cardiol, 2017. 120(7): p. 1220-1222.
8. Sniderman, A. D., et al., Apolipoprotein B Particles and Cardiovascular Disease: A Narrative Review. JAMA Cardiol, 2019. 4(12): p. 1287-1295.
9. Sirwi, A. and M. M. Hussain, Lipid transfer proteins in the assembly of apoB-containing lipoproteins. J Lipid Res, 2018. 59(7): p. 1094-1102.
10. Davidson, N. O. and G. S. Shelness, APOLIPOPROTEIN B: mRNA editing, lipoprotein assembly, and presecretory degradation. Annu Rev Nutr, 2000. 20: p. 169-93.
11. Hussain, M. M., J. Shi, and P. Dreizen, Microsomal triglyceride transfer protein and its role in apoB-lipoprotein assembly. J Lipid Res, 2003. 44(1): p. 22-32.
12. Rutledge, A. C., Q. Su, and K. Adeli, Apolipoprotein B100 biogenesis: a complex array of intracellular mechanisms regulating folding, stability, and lipoprotein assembly. Biochem Cell Biol, 2010. 88(2): p. 251-67.
13. Fisher, E. A., The degradation of apolipoprotein B100: multiple opportunities to regulate VLDL triglyceride production by different proteolytic pathways. Biochim Biophys Acta, 2012. 1821(5): p. 778-81.
14. Cesarman-Maus, G. and K. A. Hajjar, Molecular mechanisms of fibrinolysis. Br J Haematol, 2005. 129(3): p. 307-21.
15. Munkvad, S., J. Gram, and J. Jespersen, A depression of active tissue plasminogen activator in plasma characterizes patients with unstable angina pectoris who develop myocardial infarction. Eur Heart J, 1990. 11(6): p. 525-8.
16. Gram, J. and J. Jespersen, A selective depression of tissue plasminogen activator (t-PA) activity in euglobulins characterises a risk group among survivors of acute myocardial infarction. Thromb Haemost, 1987. 57(2): p. 137-9.
17. Gram, J., et al., On the usefulness of fibrinolysis variables in the characterization of a risk group for myocardial reinfarction. Acta Med Scand, 1987. 221(2): p. 149-53.
18. Jansson, J. H., et al., Hypo-fibrinolysis in patients with hypertension and elevated cholesterol. J Intern Med, 1991. 229(4): p. 309-16.
19. Yamada, R., et al., Association between tissue plasminogen activator and serum lipids in healthy volunteers. Ann Med, 1990. 22(5): p. 313-8.
20. Glueck, C. J., et al., Endogenous testosterone, fibrinolysis, and coronary heart disease risk in hyperlipidemic men. J Lab Clin Med, 1993. 122(4): p. 412-20.
21. Zheng, Z., et al., Interacting hepatic PAI-1/tPA gene regulatory pathways influence impaired fibrinolysis severity in obesity. J Clin Invest, 2020. 130(8): p. 4348-4359.
22. Zheng, Z., et al., An ATF6-tPA pathway in hepatocytes contributes to systemic fibrinolysis and is repressed by DACH1. Blood, 2019. 133(7): p. 743-753.
23. Tran, K., et al., Intracellular assembly of very low density lipoproteins containing apolipoprotein B100 in rat hepatoma McA-RH7777 cells. J Biol Chem, 2002. 277(34): p. 31187-200.
24. Yamaguchi, J., et al., The conversion of apoB100 low density lipoprotein/high density lipoprotein particles to apoB100 very low density lipoproteins in response to oleic acid occurs in the endoplasmic reticulum and not in the Golgi in McA RH7777 cells. J Biol Chem, 2003. 278(43): p. 42643-51.
25. Millar, J. S., et al., Determining hepatic triglyceride production in mice: comparison of poloxamer 407 with Triton WR-1339. J Lipid Res, 2005. 46(9): p. 2023-8.
26. Sehayek, E. and S. Eisenberg, The role of native apolipoprotein B-containing lipoproteins in atherosclerosis: cellular mechanisms. Curr Opin Lipidol, 1994. 5(5): p. 350-3.
27. Hinsdale, M. E., et al., ApoB-48 and apoB-100 differentially influence the expression of type-III hyperlipoproteinemia in APOE*2 mice. J Lipid Res, 2002. 43(9): p. 1520-8.
28. Tiwari, S. and S. A. Siddiqi, Intracellular trafficking and secretion of VLDL. Arterioscler Thromb Vasc Biol, 2012. 32(5): p. 1079-86.
29. Cheng, D., et al., Very Low Density Lipoprotein Assembly Is Required for cAMP-responsive Element-binding Protein H Processing and Hepatic Apolipoprotein A-IV Expression. J Biol Chem, 2016. 291(45): p. 23793-23803.
30. Weinberg, R. B., et al., ApoA-IV modulates the secretory trafficking of apoB and the size of triglyceride-rich lipoproteins. J Lipid Res, 2012. 53(4): p. 736-43.
31. Blade, A. M., et al., Biogenesis of apolipoprotein A-V and its impact on VLDL triglyceride secretion. J Lipid Res, 2011. 52(2): p. 237-44.
32. Sakurai, T., et al., Measurement of lipoprotein particle sizes using dynamic light scattering. Ann Clin Biochem, 2010. 47(Pt 5): p. 476-81.
33. Sahoo, D., et al., ABCA1-dependent lipid efflux to apolipoprotein A-I mediates HDL particle formation and decreases VLDL secretion from murine hepatocytes. J Lipid Res, 2004. 45(6): p. 1122-31.
34. Athar, H., et al., A simple, rapid, and sensitive fluorescence assay for microsomal triglyceride transfer protein. J Lipid Res, 2004. 45(4): p. 764-72.
35. Chandler, C. E., et al., CP-346086: an MTP inhibitor that lowers plasma cholesterol and triglycerides in experimental animals and in humans. J Lipid Res, 2003. 44(10): p. 1887-901.
36. Ozcan, L., et al., Hepatocyte DACH1 Is Increased in Obesity via Nuclear Exclusion of HDAC4 and Promotes Hepatic Insulin Resistance. Cell Rep, 2016. 15(10): p. 2214-2225.
37. Ghorpade, D. S., et al., Hepatocyte-secreted DPP4 in obesity promotes adipose inflammation and insulin resistance. Nature, 2018. 555(7698): p. 673-677.
38. Wang, X., et al., Cholesterol Stabilizes TAZ in Hepatocytes to Promote Experimental Non-alcoholic Steatohepatitis. Cell Metab, 2020. 31(5): p. 969-986 e7.
39. Shelness, G. S., et al., Apolipoprotein B in the rough endoplasmic reticulum: translation, translocation and the initiation of lipoprotein assembly. J Nutr, 1999. 129(2S Suppl): p. 456S-462S.
40. Andres, D. A., I. M. Dickerson, and J. E. Dixon, Variants of the carboxyl-terminal KDEL sequence direct intracellular retention. J Biol Chem, 1990. 265(11): p. 5952-5.
41. Gerondopoulos, A., et al., A signal capture and proofreading mechanism for the KDEL-receptor explains selectivity and dynamic range in ER retrieval. Elife, 2021. 10.
42. Newstead, S. and F. Barr, Molecular basis for KDEL-mediated retrieval of escaped ER-resident proteins—SWEET talking the COPs. J Cell Sci, 2020. 133(19).
43. Raykhel, I., et al., A molecular specificity code for the three mammalian KDEL receptors. J Cell Biol, 2007. 179(6): p. 1193-204.
44. Pu, H., et al., Protease-independent action of tissue plasminogen activator in brain plasticity and neurological recovery after ischemic stroke. Proc Natl Acad Sci USA, 2019. 116(18): p. 9115-9124.
45. de Munk, G. A., et al., Binding of tissue-type plasminogen activator to lysine, lysine analogues, and fibrin fragments. Biochemistry, 1989. 28(18): p. 7318-25.
46. Weening-Verhoeff, E. J., et al., Involvement of aspartic and glutamic residues in kringle-2 of tissue-type plasminogen activator in lysine binding, fibrin binding and stimulation of activity as revealed by chemical modification and oligonucleotide-directed mutagenesis. Protein Eng, 1990. 4(2): p. 191-8.
47. Segrest, J. P., et al., Structure of apolipoprotein B-100 in low density lipoproteins. J Lipid Res, 2001. 42(9): p. 1346-67.
48. Hussain, M. M., A. Bakillah, and H. Jamil, Apolipoprotein B binding to microsomal triglyceride transfer protein decreases with increases in length and lipidation: implications in lipoprotein biosynthesis. Biochemistry, 1997. 36(42): p. 13060-7.
49. Lin, H., et al., Therapeutics targeting the fibrinolytic system. Exp Mol Med, 2020. 52(3): p. 367-379.
50. White, A. L., et al., Oleate-mediated stimulation of apolipoprotein B secretion from rat hepatoma cells. A function of the ability of apolipoprotein B to direct lipoprotein assembly and escape presecretory degradation. J Biol Chem, 1992. 267(22): p. 15657-64.
51. Dixon, J. L., S. Furukawa, and H. N. Ginsberg, Oleate stimulates secretion of apolipoprotein B-containing lipoproteins from Hep G2 cells by inhibiting early intracellular degradation of apolipoprotein B. J Biol Chem, 1991. 266(8): p. 5080-6.
52. Gong, L., et al., Crystal Structure of the Michaelis Complex between Tissue-type Plasminogen Activator and Plasminogen Activators Inhibitor-1. J Biol Chem, 2015. 290(43): p. 25795-804.
53. Kaneko, M., et al., Interactions between the finger and kringle-2 domains of tissue-type plasminogen activator and plasminogen activator inhibitor-1. J Biochem, 1992. 111(2): p. 244-8.
54. Jiang, C., et al., Serpine 1 induces alveolar type II cell senescence through activating p53-p21-Rb pathway in fibrotic lung disease. Aging Cell, 2017. 16(5): p. 1114-1124.
55. Rustaeus, S., et al., Assembly of very low density lipoprotein: a two-step process of apolipoprotein B core lipidation. J Nutr, 1999. 129(2S Suppl): p. 463S-466S.
56. Read, J., et al., A mechanism of membrane neutral lipid acquisition by the microsomal triglyceride transfer protein. J Biol Chem, 2000. 275(39): p. 30372-7.
57. Jovin, I. S. and G. Muller-Berghaus, Interrelationships between the fibrinolytic system and lipoproteins in the pathogenesis of coronary atherosclerosis. Atherosclerosis, 2004. 174(2): p. 225-33.
58. Wu, W., J. D. Bancroft, and J. W. Suttie, Structural features of the kringle domain determine the intracellular degradation of under-gamma-carboxylated prothrombin: studies of chimeric rat/human prothrombin. Proc Natl Acad Sci USA, 1997. 94(25): p. 13654-60.
59. Novokhatny, V., et al., Tissue-type plasminogen activator (tPA) interacts with urokinase-type plasminogen activator (uPA) via tPA's lysine binding site. An explanation of the poor fibrin affinity of recombinant tPA/uPA chimeric molecules. J Biol Chem, 1995. 270(15): p. 8680-5.
60. Puccetti, L., et al., Different mechanisms of fibrinolysis impairment among dyslipidemic subjects. Int J Clin Pharmacol Res, 2001. 21(3-4): p. 147-55.
61. Aso, Y., et al., Metabolic syndrome accompanied by hypercholesterolemia is strongly associated with proinflammatory state and impairment of fibrinolysis in patients with type 2 diabetes: synergistic effects of plasminogen activator inhibitor-1 and thrombin-activatable fibrinolysis inhibitor. Diabetes Care, 2005. 28(9): p. 2211-6.
62. Khan, S. S., et al., A null mutation in SERPINE1 protects against biological aging in humans. Sci Adv, 2017. 3(11): p. eaao1617.
63. Ai, D., et al., Activation of ER stress and mTORCI suppresses hepatic sortilin-1 levels in obese mice. J Clin Invest, 2012. 122(5): p. 1677-87.
64. Widenmaier, S. B., et al., NRF1 Is an ER Membrane Sensor that Is Central to Cholesterol Homeostasis. Cell, 2017. 171(5): p. 1094-1109 e15.
65. Boren, J., S. Rustaeus, and S. O. Olofsson, Studies on the assembly of apolipoprotein B-100-and B-48-containing very low density lipoproteins in McA-RH7777 cells. J Biol Chem, 1994. 269(41): p. 25879-88.
66. Anaganti, N., S. Rajan, and M. M. Hussain, An improved assay to measure the phospholipid transfer activity of microsomal triglyceride transport protein. J Lipid Res, 2021. 62: p. 100136.
67. Basu, D., et al., Novel Reversible Model of Atherosclerosis and Regression Using Oligonucleotide Regulation of the LDL Receptor. Circ Res, 2018. 122(4): p. 560-567.

In the foregoing description, it will be readily apparent to one skilled in the art that varying substitutions and modifications may be made to the technology disclosed herein without departing from the scope and spirit of the present technology. The technology illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the present technology. Thus, it should be understood that although the present technology has been illustrated by specific embodiments and optional features, modification and/or variation of the concepts herein disclosed may be resorted to by those skilled in the art, and that such modifications and variations are considered to be within the scope of the present technology.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. As will also be understood by one skilled in the art all language such as “up to,” “at least,” and the like, include the number recited.

SEQUENCE LISTING

| SEQ | | |
|---|---|---|
| ID | | |
| NO. | Sequence | Description |
| 1 | MDAMKRGLCCVLLLCGAVFVSPSQEIHARFRRGARSY | WT human tPA |
| | QVICRDEKTQMIYQQHQSWLRPVLRSNRVEYCWCNSG | amino acid |
| | RAQCHSVPVKSCSEPRCFNGGTCQQALYFSDFVCQCPE | sequence |
| | GFAGKCCEIDTRATCYEDQGISYRGTWSTAESGAECTN | (kringle 2 |
| | WNSSALAQKPYSGRRPDAIRLGLGNHNYCRNPDRDSKP | domain |
| | WCYVFKAGKYSSEFCSTPACSEGNSDCYFGNGSAYRGT | underlined) |
| | HSLTESGASCLPWNSMILIGKVYTAQNPSAQALGLGKH | |
| | NYCRNPDGDAKPWCHVLKNRRLTWEYCDVPSCSTCGL | |
| | RQYSQPQFRIKGGLFADIASHPWQAAIFAKHRRSPGERF | |
| | LCGGILISSCWILSAAHCFQERFPPHHLTVILGRTYRVVP | |
| | GEEEQKFEVEKYIVHKEFDDDTYDNDIALLQLKSDSSR | |
| | CAQESSVVRTVCLPPADLQLPDWTECELSGYGKHEALS | |
| | PFYSERLKEAHVRLYPSSRCTSQHLLNRTVTDNMLCAG | |
| | DTRSGGPQANLHDACQGDSGGPLVCLNDGRMTLVGIIS | |
| | WGLGCGQKDVPGVYTKVTNYLDWIRDNMRP | |
| 2 | ATGGATGCAATGAAGAGAGGGCTCTGCTGTGTGCTG | WT human tPA |
| | CTGCTGTGTGGAGCAGTCTTCGTTTCGCCCAGCC | coding nucleic |
| | AGGAAATCCATGCCCGATTCAGAAGAGGAGCCAGAT | acid sequence |
| | CTTACCAAGTGATCTGCAGAGATGAAAAAACGCA | |
| | GATGATATACCAGCAACATCAGTCATGGCTGCGCCCT | |
| | GTGCTCAGAAGCAACCGGGTGGAATATTGCTGG | |
| | TGCAACAGTGGCAGGGCACAGTGCCACTCAGTGCCT | |
| | GTCAAAAGTTGCAGCGAGCCAAGGTGTTTCAACG | |
| | GGGGCACCTGCCAGCAGGCCCTGTACTTCTCAGATTT | |
| | CGTGTGCCAGTGCCCCGAAGGATTTGCTGGGAA | |
| | GTGCTGTGAAATAGATACCAGGGCCACGTGCTACGA | |
| | GGACCAGGGCATCAGCTACAGGGGCACGTGGAGC | |
| | ACAGCGGAGAGTGGCGCCGAGTGCACCAACTGGAAC | |
| | AGCAGCGCGTTGGCCCAGAAGCCCTACAGCGGGC | |
| | GGAGGCCAGACGCCATCAGGCTGGGCCTGGGGAACC | |
| | ACAACTACTGCAGAAACCCAGATCGAGACTCAAA | |
| | GCCCTGGTGCTACGTCTTTAAGGCGGGGAAGTACAG | |
| | CTCAGAGTTCTGCAGCACCCCTGCCTGCTCTGAG | |
| | GGAAACAGTGACTGCTACTTTGGGAATGGGTCAGCC | |
| | TACCGTGGCACGCACAGCCTCACCGAGTCGGGTG | |
| | CCTCCTGCCTCCCGTGGAATTCCATGATCCTGATAGG | |
| | CAAGGTTTACACAGCACAGAACCCCAGTGCCCA | |
| | GGCACTGGGCCTGGGCAAACATAATTACTGCCGGAA | |
| | TCCTGATGGGGATGCCAAGCCCTGGTGCCACGTG | |
| | CTGAAGAACCGCAGGCTGACGTGGGAGTACTGTGAT | |
| | GTGCCCTCCTGCTCCACCTGCGGCCTGAGACAGT | |
| | ACAGCCAGCCTCAGTTTCGCATCAAAGGAGGGCTCTT | |
| | CGCCGACATCGCCTCCCACCCCTGGCAGGCTGC | |
| | CATCTTTGCCAAGCACAGGAGGTCGCCCGGAGAGCG | |
| | GTTCCTGTGCGGGGGCATACTCATCAGCTCCTGC | |
| | TGGATTCTCTCTGCCGCCCACTGCTTCCAGGAGAGGT | |
| | TTCCGCCCCACCACCTGACGGTGATCTTGGGCA | |
| | GAACATACCGGGTGGTCCCTGGCGAGGAGGAGCAGA | |
| | AATTTGAAGTCGAAAAATACATTGTCCATAAGGA | |
| | ATTCGATGATGACACTTACGACAATGACATTGCGCTG | |
| | CTGCAGCTGAAATCGGATTCGTCCCGCTGTGCC | |
| | CAGGAGAGCAGCGTGGTCCGCACTGTGTGCCTTCCCC | |
| | CGGCGGACCTGCAGCTGCCGGACTGGACGGAGT | |
| | GTGAGCTCTCCGGCTACGGCAAGCATGAGGCCTTGTC | |
| | TCCTTTCTATTCGGAGCGGCTGAAGGAGGCTCA | |
| | TGTCAGACTGTACCCATCCAGCCGCTGCACATCACAA | |
| | CATTTACTTAACAGAACAGTCACCGACAACATG | |
| | CTGTGTGCTGGAGACACTCGGAGCGGCGGGCCCCAG | |
| | GCAAACTTGCACGACGCCTGCCAGGGCGATTCGG | |
| | GAGGCCCCCTGGTGTGTCTGAACGATGGCCGCATGA | |
| | CTTTGGTGGGCATCATCAGCTGGGGCCTGGGCTG | |
| | TGGACAGAAGGATGTCCCGGGTGTGTACACCAAGGT | |
| | TACCAACTACCTAGACTGGATTCGTGACAACATG | |
| | CGACCGTGA | |
| 3 | MDAMKRGLCCVLLLCGAVFVSPSQEIHARFRRGARSY | tPA KDEL |
| | QVICRDEKTQMIYQQHQSWLRPVLRSNRVEYCWCNSG | amino acid |
| | RAQCHSVPVKSCSEPRCFNGGTCQQALYFSDFVCQCPE | sequence |
| | GFAGKCCEIDTRATCYEDQGISYRGTWSTAESGAECTN | (kringle 2 |
| | WNSSALAQKPYSGRRPDAIRLGLGNHNYCRNPDRDSKP | domain |
| | WCYVFKAGKYSSEFCSTPACSEGNSDCYFGNGSAYRGT | underlined; |
| | HSLTESGASCLPWNSMILIGKVYTAQNPSAQALGLGKH | GGGGS linker |
| | NYCRNPDGDAKPWCHVLKNRRLTWEYCDVPSCSTCGL | sequence in |
| | RQYSQPQFRIKGGLFADIASHPWQAAIFAKHRRSPGERF | bold) |
| | LCGGILISSCWILSAAHCFQERFPPHHLTVILGRTYRVVP | |
| | GEEEQKFEVEKYIVHKEFDDDTYDNDIALLQLKSDSSR | |
| | CAQESSVVRTVCLPPADLQLPDWTECELSGYGKHEALS | |
| | PFYSERLKEAHVRLYPSSRCTSQHLLNRTVTDNMLCAG | |
| | DTRSGGPQANLHDACQGDSGGPLVCLNDGRMTLVGIIS | |
| | WGLGCGQKDVPGVYTKVTNYLDWIRDNMRP | |
| | GGGGSKDEL | |
| 4 | ATGGATGCAATGAAGAGAGGGCTCTGCTGTGTGCTG | tPA KDEL |
| | CTGCTGTGTGGAGCAGTCTTCGTTTCGCCCAGCC | encoding nucleic |
| | AGGAAATCCATGCCCGATTCAGAAGAGGAGCCAGAT | acid sequence |
| | CTTACCAAGTGATCTGCAGAGATGAAAAAACGCA | |
| | GATGATATACCAGCAACATCAGTCATGGCTGCGCCCT | |
| | GTGCTCAGAAGCAACCGGGTGGAATATTGCTGG | |
| | TGCAACAGTGGCAGGGCACAGTGCCACTCAGTGCCT | |
| | GTCAAAAGTTGCAGCGAGCCAAGGTGTTTCAACG | |
| | GGGGCACCTGCCAGCAGGCCCTGTACTTCTCAGATTT | |
| | CGTGTGCCAGTGCCCCGAAGGATTTGCTGGGAA | |
| | GTGCTGTGAAATAGATACCAGGGCCACGTGCTACGA | |
| | GGACCAGGGCATCAGCTACAGGGGCACGTGGAGC | |
| | ACAGCGGAGAGTGGCGCCGAGTGCACCAACTGGAAC | |
| | AGCAGCGCGTTGGCCCAGAAGCCCTACAGCGGGC | |
| | GGAGGCCAGACGCCATCAGGCTGGGCCTGGGGAACC | |
| | ACAACTACTGCAGAAACCCAGATCGAGACTCAAA | |
| | GCCCTGGTGCTACGTCTTTAAGGCGGGGAAGTACAG | |
| | CTCAGAGTTCTGCAGCACCCCTGCCTGCTCTGAG | |
| | GGAAACAGTGACTGCTACTTTGGGAATGGGTCAGCC | |
| | TACCGTGGCACGCACAGCCTCACCGAGTCGGGTG | |
| | CCTCCTGCCTCCCGTGGAATTCCATGATCCTGATAGG | |
| | CAAGGTTTACACAGCACAGAACCCCAGTGCCCA | |
| | GGCACTGGGCCTGGGCAAACATAATTACTGCCGGAA | |
| | TCCTGATGGGGATGCCAAGCCCTGGTGCCACGTG | |
| | CTGAAGAACCGCAGGCTGACGTGGGAGTACTGTGAT | |
| | GTGCCCTCCTGCTCCACCTGCGGCCTGAGACAGT | |
| | ACAGCCAGCCTCAGTTTCGCATCAAAGGAGGGCTCTT | |
| | CGCCGACATCGCCTCCCACCCCTGGCAGGCTGC | |
| | CATCTTTGCCAAGCACAGGAGGTCGCCCGGAGAGCG | |
| | GTTCCTGTGCGGGGGCATACTCATCAGCTCCTGC | |
| | TGGATTCTCTCTGCCGCCCACTGCTTCCAGGAGAGGT | |
| | TTCCGCCCCACCACCTGACGGTGATCTTGGGCA | |
| | GAACATACCGGGTGGTCCCTGGCGAGGAGGAGCAGA | |
| | AATTTGAAGTCGAAAAATACATTGTCCATAAGGA | |
| | ATTCGATGATGACACTTACGACAATGACATTGCGCTG | |
| | CTGCAGCTGAAATCGGATTCGTCCCGCTGTGCC | |
| | CAGGAGAGCAGCGTGGTCCGCACTGTGTGCCTTCCCC | |
| | CGGCGGACCTGCAGCTGCCGGACTGGACGGAGT | |
| | GTGAGCTCTCCGGCTACGGCAAGCATGAGGCCTTGTC | |
| | TCCTTTCTATTCGGAGCGGCTGAAGGAGGCTCA | |
| | TGTCAGACTGTACCCATCCAGCCGCTGCACATCACAA | |
| | CATTTACTTAACAGAACAGTCACCGACAACATG | |
| | CTGTGTGCTGGAGACACTCGGAGCGGCGGGCCCCAG | |
| | GCAAACTTGCACGACGCCTGCCAGGGCGATTCGG | |
| | GAGGCCCCCTGGTGTGTCTGAACGATGGCCGCATGA | |
| | CTTTGGTGGGCATCATCAGCTGGGGCCTGGGCTG | |
| | TGGACAGAAGGATGTCCCGGGTGTGTACACCAAGGT | |
| | TACCAACTACCTAGACTGGATTCGTGACAACATG | |
| | CGACCGTGA | |
| | GGGGGTGGAGGCTCTAAGGATGAACTT | |
| 5 | STPACSEGNSDCYFGNGSAYRGTHSLTESGASCLPWNS | tPA kringle 2 |
| | MILIGKVYTAQNPSAQALGLGKHNYCRNPDGDAKPWC | domain amino |
| | HVLKNRRLTWEYCDVPSCSTCGLRQYSQPQFR | acid sequence |
| 6 | Agcacccctgcctgctctgagggaaacagtgactgctactttgggaatgggtcagccta | tPA kringle 2 |
| | ccgtggcacgcacagcctcaccgagtcgggtgcctcctgcctcccgtggaattccatga | domain encoding |
| | tcctgataggcaaggtttacacagcacagaaccccagtgcccaggcactgggcctggg | nucleic acid |
| | caaacataattactgccggaatcctgatggggatgccaagccctggtgccacgtgctga | sequence |
| | agaaccgcaggctgacgtgggagtactgtgatgtgccctcctgctccacctgcggcctg | |
| | agacagtacagccagcctcagtttcgc | |
| 7 | STPACSEGNSDCYFGNGSAYRGTHSLTESGASCLPWNS | tPA kringle 2 |
| | MILIGKVYTAQNPSAQALGLGKHNYCRNPDGDAKPWC | KDEL amino |
| | HVLKNRRLTWEYCDVPSCSTCGLRQYSQPQFR | acid sequence |
| | GGGGSKDEL | (GGGGS linker |
| | | sequence in |
| | | bold) |
| 8 | Agcaccccugccugcucugagggaaacagugacugcuacuuugggaaugggu | tPA kringle 2 |
| | cagccuaccguggcacgcacagccucaccgagucgggugccuccugccucccgu | KDEL encoding |
| | ggaauuccaugauccugauaggcaagguuuacacagcacagaaccccagugccc | sequence in the |
| | aggcacugggccugggcaaacauaauuacugccggaauccugauggggaugcc | mRNA |
| | aagcccuggugccacgugcugaagaaccgcaggcugacgugggaguacuguga | |
| | ugugcccuccugcuccaccugcggccugagacaguacagccagccucaguuuc | |
| | gcGGGGGUGGAGGCUCUAAGGAUGAACUU | |
| 9 | MDAMKRGLCCVLLLCGAVFVSPSQEIHARFRRGARSY | tPA S513A |
| | QVICRDEKTQMIYQQHQSWLRPVLRSNRVEYCWCNSG | mutant amino |
| | RAQCHSVPVKSCSEPRCFNGGTCQQALYFSDFVCQCPE | acid sequence |
| | GFAGKCCEIDTRATCYEDQGISYRGTWSTAESGAECTN | lacking serine |
| | WNSSALAQKPYSGRRPDAIRLGLGNHNYCRNPDRDSKP | protease function |
| | WCYVFKAGKYSSEFCSTPACSEGNSDCYFGNGSAYRGT | (kringle 2 |
| | HSLTESGASCLPWNSMILIGKVYTAQNPSAQALGLGKH | domain |
| | NYCRNPDGDAKPWCHVLKNRRLTWEYCDVPSCSTCGL | underlined; |
| | RQYSQPQFRIKGGLFADIASHPWQAAIFAKHRRSPGERF | S513A bold |
| | LCGGILISSCWILSAAHCFQERFPPHHLTVILGRTYRVVP | underlined) |
| | GEEEQKFEVEKYIVHKEFDDDTYDNDIALLQLKSDSSR | |
| | CAQESSVVRTVCLPPADLQLPDWTECELSGYGKHEALS | |
| | PFYSERLKEAHVRLYPSSRCTSQHLLNRTVTDNMLCAG | |
| | DTRSGGPQANLHDACQGDAGGPLVCLNDGRMTLVGIIS | |
| | WGLGCGQKDVPGVYTKVTNYLDWIRDNMRP | |
| 10 | ATGGATGCAATGAAGAGAGGGCtctgctgtgtgctgctgctgtgtg | tPA S513A |
| | gagcagtcttcgtttcgcccagccaggaaatccatgcccgattcagaagaggagccag | encoding nucleic |
| | atcttaccaagtgatctgcagagatgaaaaaacgcagatgatataccagcaacatcagtc | acid sequence |
| | atggctgcgccctgtgctcagaagcaaccgggtggaatattgctggtgcaacagtggca | |
| | gggcacagtgccactcagtgcctgtcaaaagttgcagcgagccaaggtgtttcaacgg | |
| | gggcacctgccagcaggccctgtacttctcagatttcgtgtgccagtgccccgaaggatt | |
| | tgctgggaagtgctgtgaaatagataccagggccacgtgctacgaggaccagggcatc | |
| | agctacaggggcacgtggagcacagcggagagtggcgccgagtgcaccaactggaa | |
| | cagcagcgcgttggcccagaagccctacagcgggcggaggccagacgccatcaggc | |
| | tgggcctggggaaccacaactactgcagaaacccagatcgagactcaaagccctggtg | |
| | ctacgtctttaaggcggggaagtacagctcagagttctgcagcacccctgcctgctctga | |
| | gggaaacagtgactgctactttgggaatgggtcagcctaccgtggcacgcacagcctc | |
| | accgagtcgggtgcctcctgcctcccgtggaattccatgatcctgataggcaaggtttac | |
| | acagcacagaaccccagtgcccaggcactgggcctgggcaaacataattactgccgg | |
| | aatcctgatggggatgccaagccctggtgccacgtgctgaagaaccgcaggctgacgt | |
| | gggagtactgtgatgtgccctcctgctccacctgcggcctgagacagtacagccagcct | |
| | cagtttcgcatcaaaggagggctcttcgccgacatcgcctcccacccctggcaggctgc | |
| | catctttgccaagcacaggaggtcgcccggagagcggttcctgtgcgggggcatactc | |
| | atcagctcctgctggattctctctgccgcccactgcttccaggagaggtttccgccccacc | |
| | acctgacggtgatcttgggcagaacataccgggtggtccctggcgaggaggagcaga | |
| | aatttgaagtcgaaaaatacattgtccataaggaattcgatgatgacacttacgacaatga | |
| | cattgcgctgctgcagctgaaatcggattcgtcccgctgtgcccaggagagcagcgtgg | |
| | tccgcactgtgtgccttcccccggcggacctgcagctgccggactggacggagtgtga | |
| | gctctccggctacggcaagcatgaggccttgtctcctttctattcggagcggctgaagga | |
| | ggctcatgtcagactgtacccatccagccgctgcacatcacaacatttacttaacagaac | |
| | agtcaccgacaacatgctgtgtgctggagacactcggagcggcgggccccaggcaaa | |
| | cttgcacgacgcCTGCCAGGGCGATGCTGGAGGCCCCCTGG | |
| | Tgtgtctgaacgatggccgcatgactttggtgggcatcatcagctggggcctgggctgt | |
| | ggacagaaggatgtcccgggtgtgtacaccaaggttaccaactacctagactggattcg | |
| | tgacaacatgcgaccg | |
| 11 | KDEL | KDEL amino |
| | | acid sequence |
| | | ER localization |
| 12 | AAGGATGAACTT | KDEL encoding |
| | | nucleic acid |
| | | sequence |
| 13 | NPDGDAKPWCHVLKNRRLTWEY | tPA-K2 lysine |
| | | binding site |
| | | (LBS) peptide |
| 14 | NPDGDAKPWCHVLKNRRLTWEYKDEL | tPA-K2 LBS |
| | | KDEL peptide |
| 15 | aatcctgatggggatgccaagccctggtgccacgtgctgaagaaccgcaggctgacgt | tPA-K2 LBS |
| | gggagtac | nucleotide |
| 16 | aatcctgatggggatgccaagccctggtgccacgtgctgaagaaccgcaggctgacgt | tPA-K2 LBS |
| | gggagtacaaggatgaactt | KDEL |
| | | nucleotide |
| 17 | Tgcatgtataatttctacagaacctattagaaaggatcacccagcctctgcttttgtacaac | TBG promoter |
| | tttcccttaaaaaactgccaattccactgctgtttggcccaatagtgagaactttttcctgct | |
| | gcctcttggtgcttttgcctatggcccctattctgcctgctgaagacactcttgccagcatgg | |
| | acttaaacccctccagctctgacaatcctctttctcttttgttttacatgaagggtctggcag | |
| | ccaaagcaatcactcaaagttcaaaccttatcattttttgctttgttcctcttggccttggtt | |
| | ttgtacatcagctttgaaaataccatcccagggttaatgctggggttaatttataactaagag | |
| | tgctctagttttgcaatacaggacatgctataa | |
| 18 | ctgcgcgctcgctcgctcactgaggccgcccgggcaaagcccgggcgtcgggcgac | AAV2 ITR |
| | ctttggtcgcccggcctcagtgagcgagcgagcgcgcagagagggagtggccaactc | |
| | catcactaggggttcct | |
| 19 | MDAMKRGLCCVLLLCGAVFVSPSQEIHARFRRGARSY | WT human tPA |
| | QVICRDEKTQMIYQQHQSWLRPVLRSNRVEYCWCNSG | amino acid |
| | RAQCHSVPVKSCSEPRCFNGGTCQQALYFSDFVCQCPE | sequence without |
| | GFAGKCCEIDTRAT | the K2 domain |
| | CYEDQGISYRGTWSTAESGAECTNWNSSALAQKPYSG | (tPA-A-K2) |
| | RRPDAIRLGLGNHNYCRNPDRDSKPWCYVFKAGKYSS | |
| | EFCSTPACSEGNSD | |
| | CYEDQGISYRGTWSTAESGAECTNWNSSALAQKPYSG | |
| | RRPDAIRLGLGNHNYCRNPDRDSKPWCYVFKAGKYSS | |
| | EFCSTPACSEGNSD | |
| 20 | STCGLRQYSQPQFRIKGGLFADIASHPWQAAIFAKHRRS | WT human tPA |
| | PGERFLCGGILISSCWILSAAHCFQERFPPHHLTVILGRT | without the K2 |
| | YRVVPGEEEQKFEVEKYIVHKEFDDDTYDNDIALLQLK | domain (tPA-A- |
| | SDSSRCAQESSVVRTVCLPPADLQLPDWTECELSGYGK | K2) encoding |
| | HEALSPFYSERLKEAHVRLYPSSRCTSQHLLNRTVTDN | nucleotide |
| | MLCAGDTRSGGPQANLHDACQGDSGGPLVCLNDGRM | sequence |
| | TLVGIISWGLGCGQKDVPGVYTKVTNYLDWIRDNMRP | |
| | ATGGATGCAATGAAGAGAGGGCTCTGCTGTGTGC | |
| | TGCTGCTGTGTGGAGCAGTCTTCGTTTCGCCCAG | |
| | CCAGGAAATCCATGCCCGATTCAGAAGAGGAGC | |
| | CAGATCTTACCAAGTGATCTGCAGAGATGAAAA | |
| | AACGCAGATGATATACCAGCAACATCAGTCATG | |
| | GCTGCGCCCTGTGCTCAGAAGCAACCGGGTGGA | |
| | ATATTGCTGGTGCAACAGTGGCAGGGCACAGTGC | |
| | CACTCAGTGCCTGTCAAAAGTTGCAGCGAGCCAA | |
| | GGTGTTTCAACGGGGGCACCTGCCAGCAGGCCCT | |
| | GTACTTCTCAGATTTCGTGTGCCAGTGCCCCGAA | |
| | GGATTTGCTGGGAAGTGCTGTGAAATAGATACCA | |
| | GGGCCACG | |
| | TGCTACGAGGACCAGGGCATCAGCTACAGGGGC | |
| | ACGTGGAGCACAGCGGAGAGTGGCGCCGAGTGC | |
| | ACCAACTGGAACAGCAGCGCGTTGGCCCAGAAG | |
| | CCCTACAGCGGGCGGAGGCCAGACGCCATCAGG | |
| | CTGGGCCTGGGGAACCACAACTACTGCAGAAAC | |
| | CCAGATCGAGACTCAAAGCCCTGGTGCTACGTCT | |
| | TTAAGGCGGGGAAGTACAGCTCAGAGTTCTGCA | |
| | GCACCCCTGCCTGCTCTGAGGGAAACAGTGAC | |
| | TGCTACGAGGACCAGGGCATCAGCTACAGGGGC | |
| | ACGTGGAGCACAGCGGAGAGTGGCGCCGAGTGC | |
| | ACCAACTGGAACAGCAGCGCGTTGGCCCAGAAG | |
| | CCCTACAGCGGGCGGAGGCCAGACGCCATCAGG | |
| | CTGGGCCTGGGGAACCACAACTACTGCAGAAAC | |
| | CCAGATCGAGACTCAAAGCCCTGGTGCTACGTCT | |
| | TTAAGGCGGGGAAGTACAGCTCAGAGTTCTGCA | |
| | GCACCCCTGCCTGCTCTGAGGGAAACAGTGAC | |
| | TCCACCTGCGGCCTGAGACAGTACAGCCAGCCTC | |
| | AGTTTCGCATCAAAGGAGGGCTCTTCGCCGACAT | |
| | CGCCTCCCACCCCTGGCAGGCTGCCATCTTTGCC | |
| | AAGCACAGGAGGTCGCCCGGAGAGCGGTTCCTG | |
| | TGCGGGGGCATACTCATCAGCTCCTGCTGGATTC | |
| | TCTCTGCCGCCCACTGCTTCCAGGAGAGGTTTCC | |
| | GCCCCACCACCTGACGGTGATCTTGGGCAGAACA | |
| | TACCGGGTGGTCCCTGGCGAGGAGGAGCAGAAA | |
| | TTTGAAGTCGAAAAATACATTGTCCATAAGGAAT | |
| | TCGATGATGACACTTACGACAATGACATTGCGCT | |
| | GCTGCAGCTGAAATCGGATTCGTCCCGCTGTGCC | |
| | CAGGAGAGCAGCGTGGTCCGCACTGTGTGCCTTC | |
| | CCCCGGCGGACCTGCAGCTGCCGGACTGGACGG | |
| | AGTGTGAGCTCTCCGGCTACGGCAAGCATGAGGC | |
| | CTTGTCTCCTTTCTATTCGGAGCGGCTGAAGGAG | |
| | GCTCATGTCAGACTGTACCCATCCAGCCGCTGCA | |
| | CATCACAACATTTACTTAACAGAACAGTCACCGA | |
| | CAACATGCTGTGTGCTGGAGACACTCGGAGCGGC | |
| | GGGCCCCAGGCAAACTTGCACGACGCCTGCCAG | |
| | GGCGATTCGGGAGGCCCCCTGGTGTGTCTGAACG | |
| | ATGGCCGCATGACTTTGGTGGGCATCATCAGCTG | |
| | GGGCCTGGGCTGTGGACAGAAGGATGTCCCGGG | |
| | TGTGTACACCAAGGTTACCAACTACCTAGACTGG | |
| | ATTCGTGACAACATGCGACCGTGA | |
| 21 | MDAMKRGLCCVLLLCGAVFVSPSQEIHARFRRGARSY | tPA-D236, |
| | QVICRDEKTQMIYQQHQSWLRPVLRSNRVEYCWCNSG | 238N amino |
| | RAQCHSVPVKSCSEPRCFNGGTCQQALYFSDFVCQCPE | acid sequence |
| | GFAGKCCEIDTRATCYEDQGISYRGTWSTAESGAECTN | (D236, 238N |
| | WNSSALAQKPYSGRRPDAIRLGLGNHNYCRNPDRDSKP | bold |
| | WCYVFKAGKYSSEFCSTPACSEGNSDCYFGNGSAYRGT | underlined) |
| | HSLTESGASCLPWNSMILIGKVYTAQNPSAQALGLGKH | |
| | NYCRNPNGNAKPWCHVLKNRRLTWEYCDVPSCSTCGL | |
| | RQYSQPQFRIKGGLFADIASHPWQAAIFAKHRRSPGERF | |
| | LCGGILISSCWILSAAHCFQERFPPHHLTVILGRTYRVVP | |
| | GEEEQKFEVEKYIVHKEFDDDTYDNDIALLQLKSDSSRC | |
| | AQESSVVRTVCLPPADLQLPDWTECELSGYGKHEALSP | |
| | FYSERLKEAHVRLYPSSRCTSQHLLNRTVTDNMLCAGD | |
| | TRSGGPQANLHDACQGDSGGPLVCLNDGRMTLVGIISW | |
| | GLGCGQKDVPGVYTKVTNYLDWIRDNMRP | |
| 22 | ATGGATGCAATGAAGAGAGGGCTCTGCTGTGTGC | tPA-D236, |
| | TGCTGCTGTGTGGAGCAGTCTTCGTTTCGCCCAG | 238N encoding |
| | CCAGGAAATCCATGCCCGATTCAGAAGAGGAGC | nucleotide |
| | CAGATCTTACCAAGTGATCTGCAGAGATGAAAA | sequence |
| | AACGCAGATGATATACCAGCAACATCAGTCATG | (D236, 238N |
| | GCTGCGCCCTGTGCTCAGAAGCAACCGGGTGGA | bold |
| | ATATTGCTGGTGCAACAGTGGCAGGGCACAGTGC | underlined) |
| | CACTCAGTGCCTGTCAAAAGTTGCAGCGAGCCAA | |
| | GGTGTTTCAACGGGGGCACCTGCCAGCAGGCCCT | |
| | GTACTTCTCAGATTTCGTGTGCCAGTGCCCCGAA | |
| | GGATTTGCTGGGAAGTGCTGTGAAATAGATACCA | |
| | GGGCCACGTGCTACGAGGACCAGGGCATCAGCT | |
| | ACAGGGGCACGTGGAGCACAGCGGAGAGTGGCG | |
| | CCGAGTGCACCAACTGGAACAGCAGCGCGTTGG | |
| | CCCAGAAGCCCTACAGCGGGCGGAGGCCAGACG | |
| | CCATCAGGCTGGGCCTGGGGAACCACAACTACTG | |
| | CAGAAACCCAGATCGAGACTCAAAGCCCTGGTG | |
| | CTACGTCTTTAAGGCGGGGAAGTACAGCTCAGAG | |
| | TTCTGCAGCACCCCTGCCTGCTCTGAGGGAAACA | |
| | GTGACTGCTACTTTGGGAATGGGTCAGCCTACCG | |
| | TGGCACGCACAGCCTCACCGAGTCGGGTGCCTCC | |
| | TGCCTCCCGTGGAATTCCATGATCCTGATAGGCA | |
| | AGGTTTACACAGCACAGAACCCCAGTGCCCAGG | |
| | CACTGGGCCTGGGCAAACATAATTACTGCCGGAA | |
| | TCCTAATGGGAACGCCAAGCCCTGGTGCCACGTG | |
| | CTGAAGAACCGCAGGCTGACGTGGGAGTACTGT | |
| | GATGTGCCCTCCTGCTCCACCTGCGGCCTGAGAC | |
| | AGTACAGCCAGCCTCAGTTTCGCATCAAAGGAGG | |
| | GCTCTTCGCCGACATCGCCTCCCACCCCTGGCAG | |
| | GCTGCCATCTTTGCCAAGCACAGGAGGTCGCCCG | |
| | GAGAGCGGTTCCTGTGCGGGGGCATACTCATCAG | |
| | CTCCTGCTGGATTCTCTCTGCCGCCCACTGCTTCC | |
| | AGGAGAGGTTTCCGCCCCACCACCTGACGGTGAT | |
| | CTTGGGCAGAACATACCGGGTGGTCCCTGGCGAG | |
| | GAGGAGCAGAAATTTGAAGTCGAAAAATACATT | |
| | GTCCATAAGGAATTCGATGATGACACTTACGACA | |
| | ATGACATTGCGCTGCTGCAGCTGAAATCGGATTC | |
| | GTCCCGCTGTGCCCAGGAGAGCAGCGTGGTCCGC | |
| | ACTGTGTGCCTTCCCCCGGCGGACCTGCAGCTGC | |
| | CGGACTGGACGGAGTGTGAGCTCTCCGGCTACGG | |
| | CAAGCATGAGGCCTTGTCTCCTTTCTATTCGGAG | |
| | CGGCTGAAGGAGGCTCATGTCAGACTGTACCCAT | |
| | CCAGCCGCTGCACATCACAACATTTACTTAACAG | |
| | AACAGTCACCGACAACATGCTGTGTGCTGGAGAC | |
| | ACTCGGAGCGGCGGGCCCCAGGCAAACTTGCAC | |
| | GACGCCTGCCAGGGCGATTCGGGAGGCCCCCTG | |
| | GTGTGTCTGAACGATGGCCGCATGACTTTGGTGG | |
| | GCATCATCAGCTGGGGCCTGGGCTGTGGACAGA | |
| | AGGATGTCCCGGGTGTGTACACCAAGGTTACCAA | |
| | CTACCTAGACTGGATTCGTGACAACATGCGACCG | |
| | TGA | |
| 23 | GACGGATCGGGAGATCTCCCGATCCCCTATGGTG | pCMV3-tPA- |
| | CACTCTCAGTACAATCTGCTCTGATGCCGCATAG | S513A |
| | TTAAGCCAGTATCTGCTCCCTGCTTGTGTGTTGGA | (plasmid |
| | GGTCGCTGAGTAGTGCGCGAGCAAAATTTAAGCT | sequence for |
| | ACAACAAGGCAAGGCTTGACCGACAATTGCATG | tPA-S513A) |
| | AAGAATCTGCTTAGGGTTAGGCGTTTTGCGCTGC | |
| | TTCGCGAGTACATTTATATTGGCTCATGTCCAAT | |
| | ATGACCGCCATGTTGACATTGATTATTGACTAGT | |
| | TATTAATAGTAATCAATTACGGGGTCATTAGTTC | |
| | ATAGCCCATATATGGAGTTCCGCGTTACATAACT | |
| | TACGGTAAATGGCCCGCCTGGCTGACCGCCCAAC | |
| | GACCCCCGCCCATTGACGTCAATAATGACGTATG | |
| | TTCCCATAGTAACGCCAATAGGGACTTTCCATTG | |
| | ACGTCAATGGGTGGAGTATTTACGGTAAACTGCC | |
| | CACTTGGCAGTACATCAAGTGTATCATATGCCAA | |
| | GTCCGCCCCCTATTGACGTCAATGACGGTAAATG | |
| | GCCCGCCTGGCATTATGCCCAGTACATGACCTTA | |
| | CGGGACTTTCCTACTTGGCAGTACATCTACGTAT | |
| | TAGTCATCGCTATTACCATGGTGATGCGGTTTTG | |
| | GCAGTACACCAATGGGCGTGGATAGCGGTTTGAC | |
| | TCACGGGGATTTCCAAGTCTCCACCCCATTGACG | |
| | TCAATGGGAGTTTGTTTTGGCACCAAAATCAACG | |
| | GGACTTTCCAAAATGTCGTAATAACCCCGCCCCG | |
| | TTGACGCAAATGGGCGGTAGGCGTGTACGGTGG | |
| | GAGGTCTATATAAGCAGAGCTCGTTTAGTGAACC | |
| | GTCAGATCCTCACTCTCTTCCGCATCGCTGTCTGC | |
| | GAGGGCCAGCTGTTGGGCTCGCGGTTGAGGACA | |
| | AACTCTTCGCGGTCTTTCCAGTACTCTTGGATCGG | |
| | AAACCCGTCGGCCTCCGAACGGTACTCCGCCACC | |
| | GAGGGACCTGAGCGAGTCCGCATCGACCGGATC | |
| | GGAAAACCTCTCGAGAAAGGCGTCTAACCAGTC | |
| | ACAGTCGCAAGGTAGGCTGAGCACCGTGGCGGG | |
| | CGGCAGCGGGTGGCGGTCGGGGTTGTTTCTGGCG | |
| | GAGGTGCTGCTGATGATGTAATTAAAGTAGGCGG | |
| | TCTTGAGACGGCGGATGGTCGAGGTGAGGTGTG | |
| | GCAGGCTTGAGATCCAGCTGTTGGGGTGAGTACT | |
| | CCCTCTCAAAAGCGGGCATTACTTCTGCGCTAAG | |
| | ATTGTCAGTTTCCAAAAACGAGGAGGATTTGATA | |
| | TTCACCTGGCCCGATCTGGCCATACACTTGAGTG | |
| | ACAATGACATCCACTTTGCCTTTCTCTCCACAGGT | |
| | GTCCACTCCCAGGTCCAAGTTTAAACTTTAATAC | |
| | GACTCACTATAGGGGCCGCCACCAAGCTTGGTAC | |
| | CATGGATGCAATGAAGAGAGGGCtctgctgtgtgctgctgc | |
| | tgtgtggagcagtcttcgtttcgcccagccaggaaatccatgcccgattcagaag | |
| | aggagccagatcttaccaagtgatctgcagagatgaaaaaacgcagatgatata | |
| | ccagcaacatcagtcatggctgcgccctgtgctcagaagcaaccgggtggaata | |
| | ttgctggtgcaacagtggcagggcacagtgccactcagtgcctgtcaaaagttgc | |
| | agcgagccaaggtgtttcaacgggggcacctgccagcaggccctgtacttctca | |
| | gatttcgtgtgccagtgccccgaaggatttgctgggaagtgctgtgaaatagatac | |
| | cagggccacgtgctacgaggaccagggcatcagctacaggggcacgtggagc | |
| | acagcggagagtggcgccgagtgcaccaactggaacagcagcgcgttggccc | |
| | agaagccctacagcgggcggaggccagacgccatcaggctgggcctgggga | |
| | accacaactactgcagaaacccagatcgagactcaaagccctggtgctacgtctt | |
| | taaggggggaagtacagctcagagttctgcagcacccctgcctgctctgaggg | |
| | aaacagtgactgctactttgggaatgggtcagcctaccgtggcacgcacagcctc | |
| | accgagtcgggtgcctcctgcctcccgtggaattccatgatcctgataggcaagg | |
| | tttacacagcacagaaccccagtgcccaggcactgggcctgggcaaacataatt | |
| | actgccggaatcctgatggggatgccaagccctggtgccacgtgctgaagaacc | |
| | gcaggctgacgtgggagtactgtgatgtgccctcctgctccacctgcggcctga | |
| | gacagtacagccagcctcagtttcgcatcaaaggagggctcttcgccgacatcg | |
| | cctcccacccctggcaggctgccatctttgccaagcacaggaggtcgcccgga | |
| | gagcggttcctgtgcgggggcatactcatcagctcctgctggattctctctgccgc | |
| | ccactgcttccaggagaggtttccgccccaccacctgacggtgatcttgggcaga | |
| | acataccgggtggtccctggcgaggaggagcagaaatttgaagtcgaaaaatac | |
| | attgtccataaggaattcgatgatgacacttacgacaatgacattgcgctgctgca | |
| | gctgaaatcggattcgtcccgctgtgcccaggagagcagcgtggtccgcactgt | |
| | gtgccttcccccggcggacctgcagctgccggactggacggagtgtgagctctc | |
| | cggctacggcaagcatgaggccttgtctcctttctattcggagcggctgaaggag | |
| | gctcatgtcagactgtacccatccagccgctgcacatcacaacatttacttaacag | |
| | aacagtcaccgacaacatgctgtgtgctggagacactcggagcgggggcccc | |
| | aggcaaacttgcacgacgcCTGCCAGGGCGATGCTGGAGG | |
| | CCCCCTGGTgtgtctgaacgatggccgcatgactttggtgggcatcatca | |
| | gctggggcctgggctgtggacagaaggatgtcccgggtgtgtacaccaaggtta | |
| | ccaactacctagactggattcgtgacaacatgcgaccgGGGGGTGGAG | |
| | GCTCTTATCCTTACGACGTGCCTGACTACGCCTA | |
| | AACTCGAGTCTAGAGCGGCCGCCGAATTCGGGCC | |
| | CGTTTAAACCCGCTGATCAGCCTCGACTGTGCCT | |
| | TCTAGTTGCCAGCCATCTGTTGTTTGCCCCTCCCC | |
| | CGTGCCTTCCTTGACCCTGGAAGGTGCCACTCCC | |
| | ACTGTCCTTTCCTAATAAAATGAGGAAATTGCAT | |
| | CGCATTGTCTGAGTAGGTGTCATTCTATTCTGGG | |
| | GGGTGGGGGGGGCAGGACAGCAAGGGGGAGG | |
| | ATTGGGAAGACAATAGCAGGCATGCTGGGGATG | |
| | CGGTGGGCTCTATGGCTTCTGAGGCGGAAAGAAC | |
| | CAGCTGGGGCTCTAGGGGGTATCCCCACGCGCCC | |
| | TGTAGCGGCGCATTAAGCGCGGCGGGTGTGGTG | |
| | GTTACGCGCAGCGTGACCGCTACACTTGCCAGCG | |
| | CCCTAGCGCCCGCTCCTTTCGCTTTCTTCCCTTCC | |
| | TTTCTCGCCACGTTCGCAGGCTTTCCCCGTCAAGC | |
| | TCTAAATCGGGGGCTCCCTTTAGGGTTCCGATTT | |
| | AGTGCTTTACGGCACCTCGACCCCAAAAAACTTG | |
| | ATTAGGGTGATGGTTCACGTAGTGGGCCATCGCC | |
| | CTGATAGACGGTTTTTCGCCCTTTGACGTTGGAG | |
| | TCCACGTTCTTTAATAGTGGACTCTTGTTCCAAAC | |
| | TGGAACAACACTCAACCCTATCTCGGTCTATTCT | |
| | TTTGATTTATAAGGGATTTTGCCGATTTCGGCCTA | |
| | TTGGTTAAAAAATGAGCTGATTTAACAAAAATTT | |
| | AACGCGAATTAATTCTGTGGAATGTGTGTCAGTT | |
| | AGGGTGTGGAAAGTCCCCAGGCTCCCCAGCAGG | |
| | CAGAAGTATGCAAAGCATGCATCTCAATTAGTCA | |
| | GCAACCAGGTGTGGAAAGTCCCCAGGCTCCCCA | |
| | GCAGGCAGAAGTATGCAAAGCATGCATCTCAATT | |
| | AGTCAGCAACCATAGTCCCGCCCCTAACTCCGCC | |
| | CATCCCGCCCCTAACTCCGCCCAGTTCCGCCCAT | |
| | TCTCCGCCCCATGGCTGACTAATTTTTTTTATTTA | |
| | TGCAGAGGCCGAGGCCGCCTCTGCCTCTGAGCTA | |
| | TTCCAGAAGTAGTGAGGAGGCTTTTTTGGAGGCC | |
| | TAGGCTTTTGCAAAAAGCTCTCGGGAGCTTGTAT | |
| | ATCCATTTTCGGATCTGATCAGCACGTGATGAAA | |
| | AAGCCTGAACTCACCGCGACGTCTGTCGAGAAGT | |
| | TTCTGATCGAAAAGTTCGACAGCGTCTCCGACCT | |
| | GATGCAGCTCTCGGAGGGCGAAGAATCTCGTGCT | |
| | TTCAGCTTCGATGTAGGAGGGCGTGGATATGTCC | |
| | TGCGGGTAAATAGCTGCGCCGATGGTTTCTACAA | |
| | AGATCGTTATGTTTATCGGCACTTTGCATCGGCC | |
| | GCGCTCCCGATTCCGGAAGTGCTTGACATTGGGG | |
| | AATTCAGCGAGAGCCTGACCTATTGCATCTCCCG | |
| | CCGTGCACAGGGTGTCACGTTGCAAGACCTGCCT | |
| | GAAACCGAACTGCCCGCTGTTCTGCAGCCGGTCG | |
| | CGGAGGCCATGGATGCGATCGCTGCGGCCGATCT | |
| | TAGCCAGACGAGCGGGTTCGGCCCATTCGGACCG | |
| | CAAGGAATCGGTCAATACACTACATGGCGTGATT | |
| | TCATATGCGCGATTGCTGATCCCCATGTGTATCA | |
| | CTGGCAAACTGTGATGGACGACACCGTCAGTGCG | |
| | TCCGTCGCGCAGGCTCTCGATGAGCTGATGCTTT | |
| | GGGCCGAGGACTGCCCCGAAGTCCGGCACCTCGT | |
| | GCACGCGGATTTCGGCTCCAACAATGTCCTGACG | |
| | GACAATGGCCGCATAACAGCGGTCATTGACTGG | |
| | AGCGAGGCGATGTTCGGGGATTCCCAATACGAG | |
| | GTCGCCAACATCTTCTTCTGGAGGCCGTGGTTGG | |
| | CTTGTATGGAGCAGCAGACGCGCTACTTCGAGCG | |
| | GAGGCATCCGGAGCTTGCAGGATCGCCGCGGCTC | |
| | CGGGCGTATATGCTCCGCATTGGTCTTGACCAAC | |
| | TCTATCAGAGCTTGGTTGACGGCAATTTCGATGA | |
| | TGCAGCTTGGGCGCAGGGTCGATGCGACGCAATC | |
| | GTCCGATCCGGAGCCGGGACTGTCGGGCGTACAC | |
| | AAATCGCCCGCAGAAGCGCGGCCGTCTGGACCG | |
| | ATGGCTGTGTAGAAGTACTCGCCGATAGTGGAAA | |
| | CCGACGCCCCAGCACTCGTCCGAGGGCAAAGGA | |
| | ATAGCACGTGCTACGAGATTTCGATTCCACCGCC | |
| | GCCTTCTATGAAAGGTTGGGCTTCGGAATCGTTT | |
| | TCCGGGACGCTGGCTGGATGATCCTCCAGCGCGG | |
| | GGATCTCATGCTGGAGTTCTTCGCCCACCCCAAC | |
| | TTGTTTATTGCAGCTTATAATGGTTACAAATAAA | |
| | GCAATAGCATCACAAATTTCACAAATAAAGCATT | |
| | TTTTTCACTGCATTCTAGTTGTGGTTTGTCCAAAC | |
| | TCATCAATGTATCTTATCATGTCTGTATACCGTCG | |
| | ACCTCTAGCTAGAGCTTGGCGTAATCATGGTCAT | |
| | AGCTGTTTCCTGTGTGAAATTGTTATCCGCTCACA | |
| | ATTCCACACAACATACGAGCCGGAAGCATAAAG | |
| | TGTAAAGCCTGGGGTGCCTAATGAGTGAGCTAAC | |
| | TCACATTAATTGCGTTGCGCTCACTGCCCGCTTTC | |
| | CAGTCGGGAAACCTGTCGTGCCAGCTGCATTAAT | |
| | GAATCGGCCAACGCGCGGGGAGAGGCGGTTTGC | |
| | GTATTGGGCGCTCTTCCGCTTCCTCGCTCACTGAC | |
| | TCGCTGCGCTCGGTCGTTCGGCTGCGGCGAGCGG | |
| | TATCAGCTCACTCAAAGGCGGTAATACGGTTATC | |
| | CACAGAATCAGGGGATAACGCAGGAAAGAACAT | |
| | GTGAGCAAAAGGCCAGCAAAAGGCCAGGAACCG | |
| | TAAAAAGGCCGCGTTGCTGGCGTTTTTCCATAGG | |
| | CTCCGCCCCCCTGACGAGCATCACAAAAATCGAC | |
| | GCTCAAGTCAGAGGTGGCGAAACCCGACAGGAC | |
| | TATAAAGATACCAGGCGTTTCCCCCTGGAAGCTC | |
| | CCTCGTGCGCTCTCCTGTTCCGACCCTGCCGCTTA | |
| | CCGGATACCTGTCCGCCTTTCTCCCTTCGGGAAG | |
| | CGTGGCGCTTTCTCATAGCTCACGCTGTAGGTAT | |
| | CTCAGTTCGGTGTAGGTCGTTCGCTCCAAGCTGG | |
| | GCTGTGTGCACGAACCCCCCGTTCAGCCCGACCG | |
| | CTGCGCCTTATCCGGTAACTATCGTCTTGAGTCC | |
| | AACCCGGTAAGACACGACTTATCGCCACTGGCAG | |
| | CAGCCACTGGTAACAGGATTAGCAGAGCGAGGT | |
| | ATGTAGGCGGTGCTACAGAGTTCTTGAAGTGGTG | |
| | GCCTAACTACGGCTACACTAGAAGAACAGTATTT | |
| | GGTATCTGCGCTCTGCTGAAGCCAGTTACCTTCG | |
| | GAAAAAGAGTTGGTAGCTCTTGATCCGGCAAAC | |
| | AAACCACCGCTGGTAGCGGTGGTTTTTTTGTTTG | |
| | CAAGCAGCAGATTACGCGCAGAAAAAAAGGATC | |
| | TCAAGAAGATCCTTTGATCTTTTCTACGGGGTCT | |
| | GAGCGCGGAACCCCTATTTGTTTATTTTTCTAAAT | |
| | ACATTCAAATATGTATCCGCTCATGAATTAATTC | |
| | TTAGAAAAACTCATCGAGCATCAAATGAAACTGC | |
| | AATTTATTCATATCAGGATTATCAATACCATATTT | |
| | TTGAAAAAGCCGTTTCTGTAATGAAGGAGAAAA | |
| | CTCACCGAGGCAGTTCCATAGGATGGCAAGATCC | |
| | TGGTATCGGTCTGCGATTCCGACTCGTCCAACAT | |
| | CAATACAACCTATTAATTTCCCCTCGTCAAAAAT | |
| | AAGGTTATCAAGTGAGAAATCACCATGAGTGAC | |
| | GACTGAATCCGGTGAGAATGGCAAAAGTTTATGC | |
| | ATTTCTTTCCAGACTTGTTCAACAGGCCAGCCATT | |
| | ACGCTCGTCATCAAAATCACTCGCATCAACCAAA | |
| | CCGTTATTCATTCGTGATTGCGCCTGAGCGAGAC | |
| | GAAATACGCGATCGCTGTTAAAAGGACAATTAC | |
| | AAACAGGAATCGAATGCAACCGGCGCAGGAACA | |
| | CTGCCAGCGCATCAACAATATTTTCACCTGAATC | |
| | AGGATATTCTTCTAATACCTGGAATGCTGTTTTCC | |
| | CAGGGATCGCAGTGGTGAGTAACCATGCATCATC | |
| | AGGAGTACGGATAAAATGCTTGATGGTCGGAAG | |
| | AGGCATAAATTCCGTCAGCCAGTTTAGTCTGACC | |
| | ATCTCATCTGTAACATCATTGGCAACGCTACCTTT | |
| | GCCATGTTTCAGAAACAACTCTGGCGCATCGGGC | |
| | TTCCCATACAATCGATAGATTGTCGCACCTGATT | |
| | GCCCGACATTATCGCGAGCCCATTTATACCCATA | |
| | TAAATCAGCATCCATGTTGGAATTTAATCGCGGC | |
| | CTAGAGCAAGACGTTTCCCGTTGAATATGGCTCA | |
| | TAACACCCCTTGTATTACTGTTTATGTAAGCAGA | |
| | CAGTTTTATTGTTCATGACCAAAATCCCTTAACGT | |
| | GAGTTTTCGTTCCACTGAGCGTCAGACCCCGTAG | |
| | AAATCCGCGCACATTTCCCCGAAAAGTGCCACCT | |
| | GACGTC | |
| 24 | GACGGATCGGGAGATCTCCCGATCCCCTATGGTG | pCMV3-tPA- |
| | CACTCTCAGTACAATCTGCTCTGATGCCGCATAGT | HA-KDEL |
| | TAAGCCAGTATCTGCTCCCTGCTTGTGTGTTGGAG | (plasmid |
| | GTCGCTGAGTAGTGCGCGAGCAAAATTTAAGCTA | encoding tPA- |
| | CAACAAGGCAAGGCTTGACCGACAATTGCATGAA | HA-KDEL) |
| | GAATCTGCTTAGGGTTAGGCGTTTTGCGCTGCTTC | |
| | GCGAGTACATTTATATTGGCTCATGTCCAATATGA | |
| | CCGCCATGTTGACATTGATTATTGACTAGTTATTA | |
| | ATAGTAATCAATTACGGGGTCATTAGTTCATAGCC | |
| | CATATATGGAGTTCCGCGTTACATAACTTACGGTA | |
| | AATGGCCCGCCTGGCTGACCGCCCAACGACCCCC | |
| | GCCCATTGACGTCAATAATGACGTATGTTCCCATA | |
| | GTAACGCCAATAGGGACTTTCCATTGACGTCAATG | |
| | GGTGGAGTATTTACGGTAAACTGCCCACTTGGCA | |
| | GTACATCAAGTGTATCATATGCCAAGTCCGCCCCC | |
| | TATTGACGTCAATGACGGTAAATGGCCCGCCTGG | |
| | CATTATGCCCAGTACATGACCTTACGGGACTTTCC | |
| | TACTTGGCAGTACATCTACGTATTAGTCATCGCTA | |
| | TTACCATGGTGATGCGGTTTTGGCAGTACACCAAT | |
| | GGGCGTGGATAGCGGTTTGACTCACGGGGATTTC | |
| | CAAGTCTCCACCCCATTGACGTCAATGGGAGTTTG | |
| | TTTTGGCACCAAAATCAACGGGACTTTCCAAAATG | |
| | TCGTAATAACCCCGCCCCGTTGACGCAAATGGGC | |
| | GGTAGGCGTGTACGGTGGGAGGTCTATATAAGCA | |
| | GAGCTCGTTTAGTGAACCGTCAGATCCTCACTCTC | |
| | TTCCGCATCGCTGTCTGCGAGGGCCAGCTGTTGG | |
| | GCTCGCGGTTGAGGACAAACTCTTCGCGGTCTTTC | |
| | CAGTACTCTTGGATCGGAAACCCGTCGGCCTCCG | |
| | AACGGTACTCCGCCACCGAGGGACCTGAGCGAGT | |
| | CCGCATCGACCGGATCGGAAAACCTCTCGAGAAA | |
| | GGCGTCTAACCAGTCACAGTCGCAAGGTAGGCTG | |
| | AGCACCGTGGCGGGCGGCAGCGGGTGGCGGTCG | |
| | GGGTTGTTTCTGGCGGAGGTGCTGCTGATGATGT | |
| | AATTAAAGTAGGCGGTCTTGAGACGGCGGATGGT | |
| | CGAGGTGAGGTGTGGCAGGCTTGAGATCCAGCTG | |
| | TTGGGGTGAGTACTCCCTCTCAAAAGCGGGCATT | |
| | ACTTCTGCGCTAAGATTGTCAGTTTCCAAAAACGA | |
| | GGAGGATTTGATATTCACCTGGCCCGATCTGGCC | |
| | ATACACTTGAGTGACAATGACATCCACTTTGCCTT | |
| | TCTCTCCACAGGTGTCCACTCCCAGGTCCAAGTTT | |
| | AAACTTTAATACGACTCACTATAGGGGCCGCCACC | |
| | AAGCTTGGTACCATGGATGCAATGAAGAGAGGGCt | |
| | ctgctgtgtgctgctgctgtgtggagcagtcttcgtttcgcccagccaggaaatccat | |
| | gcccgattcagaagaggagccagatcttaccaagtgatctgcagagatgaaaaa | |
| | acgcagatgatataccagcaacatcagtcatggctgcgccctgtgctcagaagca | |
| | accgggtggaatattgctggtgcaacagtggcagggcacagtgccactcagtgcc | |
| | tgtcaaaagttgcagcgagccaaggtgtttcaacgggggcacctgccagcaggcc | |
| | ctgtacttctcagatttcgtgtgccagtgccccgaaggatttgctgggaagtgctgtg | |
| | aaatagataccagggccacgtgctacgaggaccagggcatcagctacaggggca | |
| | cgtggagcacagcggagagtggcgccgagtgcaccaactggaacagcagcgcgt | |
| | tggcccagaagccctacagcgggcggaggccagacgccatcaggctgggcctgg | |
| | ggaaccacaactactgcagaaacccagatcgagactcaaagccctggtgctacgt | |
| | ctttaaggcggggaagtacagctcagagttctgcagcacccctgcctgctctgagg | |
| | gaaacagtgactgctactttgggaatgggtcagcctaccgtggcacgcacagcctc | |
| | accgagtcgggtgcctcctgcctcccgtggaattccatgatcctgataggcaaggtt | |
| | tacacagcacagaaccccagtgcccaggcactgggcctgggcaaacataattact | |
| | gccggaatcctgatggggatgccaagccctggtgccacgtgctgaagaaccgcag | |
| | gctgacgtgggagtactgtgatgtgccctcctgctccacctgcggcctgagacagt | |
| | acagccagcctcagtttcgcatcaaaggagggctcttcgccgacatcgcctcccac | |
| | ccctggcaggctgccatctttgccaagcacaggaggtcgcccggagagcggttcct | |
| | gtgcgggggcatactcatcagctcctgctggattctctctgccgcccactgcttccag | |
| | gagaggtttccgccccaccacctgacggtgatcttgggcagaacataccgggtggt | |
| | ccctggcgaggaggagcagaaatttgaagtcgaaaaatacattgtccataaggaa | |
| | ttcgatgatgacacttacgacaatgacattgcgctgctgcagctgaaatcggattcg | |
| | tcccgctgtgcccaggagagcagcgtggtccgcactgtgtgccttcccccggcgga | |
| | cctgcagctgccggactggacggagtgtgagctctccggctacggcaagcatgag | |
| | gccttgtctcctttctattcggagcggctgaaggaggctcatgtcagactgtacccat | |
| | ccagccgctgcacatcacaacatttacttaacagaacagtcaccgacaacatgctg | |
| | tgtgctggagacactcggagcggcgggccccaggcaaacttgcacgacgcctgcc | |
| | agggcgattcgggaggccccctggtgtgtctgaacgatggccgcatgactttggtg | |
| | ggcatcatcagctggggcctgggctgtggacagaaggatgtcccgggtgtgtaca | |
| | ccaaggttaccaactacctagactggattcgtgacaacatgcgaccgGGGGG | |
| | TGGAGGCTCTTATCCTTACGACGTGCCTGACTACG | |
| | CCAAGGATGAACTTTAATCTAGAGCGGCCGCCGA | |
| | ATTCGGGCCCGTTTAAACCCGCTGATCAGCCTCGA | |
| | CTGTGCCTTCTAGTTGCCAGCCATCTGTTGTTTGC | |
| | CCCTCCCCCGTGCCTTCCTTGACCCTGGAAGGTGC | |
| | CACTCCCACTGTCCTTTCCTAATAAAATGAGGAAA | |
| | TTGCATCGCATTGTCTGAGTAGGTGTCATTCTATT | |
| | CTGGGGGGTGGGGTGGGGCAGGACAGCAAGGGG | |
| | GAGGATTGGGAAGACAATAGCAGGCATGCTGGGG | |
| | ATGCGGTGGGCTCTATGGCTTCTGAGGCGGAAAG | |
| | AACCAGCTGGGGCTCTAGGGGGTATCCCCACGCG | |
| | CCCTGTAGCGGCGCATTAAGCGCGGCGGGTGTGG | |
| | TGGTTACGCGCAGCGTGACCGCTACACTTGCCAG | |
| | CGCCCTAGCGCCCGCTCCTTTCGCTTTCTTCCCTT | |
| | CCTTTCTCGCCACGTTCGCAGGCTTTCCCCGTCAA | |
| | GCTCTAAATCGGGGGCTCCCTTTAGGGTTCCGATT | |
| | TAGTGCTTTACGGCACCTCGACCCCAAAAAACTTG | |
| | ATTAGGGTGATGGTTCACGTAGTGGGCCATCGCC | |
| | CTGATAGACGGTTTTTCGCCCTTTGACGTTGGAGT | |
| | CCACGTTCTTTAATAGTGGACTCTTGTTCCAAACT | |
| | GGAACAACACTCAACCCTATCTCGGTCTATTCTTT | |
| | TGATTTATAAGGGATTTTGCCGATTTCGGCCTATT | |
| | GGTTAAAAAATGAGCTGATTTAACAAAAATTTAAC | |
| | GCGAATTAATTCTGTGGAATGTGTGTCAGTTAGGG | |
| | TGTGGAAAGTCCCCAGGCTCCCCAGCAGGCAGAA | |
| | GTATGCAAAGCATGCATCTCAATTAGTCAGCAACC | |
| | AGGTGTGGAAAGTCCCCAGGCTCCCCAGCAGGCA | |
| | GAAGTATGCAAAGCATGCATCTCAATTAGTCAGCA | |
| | ACCATAGTCCCGCCCCTAACTCCGCCCATCCCGCC | |
| | CCTAACTCCGCCCAGTTCCGCCCATTCTCCGCCCC | |
| | ATGGCTGACTAATTTTTTTTATTTATGCAGAGGCC | |
| | GAGGCCGCCTCTGCCTCTGAGCTATTCCAGAAGT | |
| | AGTGAGGAGGCTTTTTTGGAGGCCTAGGCTTTTG | |
| | CAAAAAGCTCTCGGGAGCTTGTATATCCATTTTCG | |
| | GATCTGATCAGCACGTGATGAAAAAGCCTGAACT | |
| | CACCGCGACGTCTGTCGAGAAGTTTCTGATCGAA | |
| | AAGTTCGACAGCGTCTCCGACCTGATGCAGCTCTC | |
| | GGAGGGCGAAGAATCTCGTGCTTTCAGCTTCGAT | |
| | GTAGGAGGGCGTGGATATGTCCTGCGGGTAAATA | |
| | GCTGCGCCGATGGTTTCTACAAAGATCGTTATGTT | |
| | TATCGGCACTTTGCATCGGCCGCGCTCCCGATTCC | |
| | GGAAGTGCTTGACATTGGGGAATTCAGCGAGAGC | |
| | CTGACCTATTGCATCTCCCGCCGTGCACAGGGTGT | |
| | CACGTTGCAAGACCTGCCTGAAACCGAACTGCCC | |
| | GCTGTTCTGCAGCCGGTCGCGGAGGCCATGGATG | |
| | CGATCGCTGCGGCCGATCTTAGCCAGACGAGCGG | |
| | GTTCGGCCCATTCGGACCGCAAGGAATCGGTCAA | |
| | TACACTACATGGCGTGATTTCATATGCGCGATTGC | |
| | TGATCCCCATGTGTATCACTGGCAAACTGTGATGG | |
| | ACGACACCGTCAGTGCGTCCGTCGCGCAGGCTCT | |
| | CGATGAGCTGATGCTTTGGGCCGAGGACTGCCCC | |
| | GAAGTCCGGCACCTCGTGCACGCGGATTTCGGCT | |
| | CCAACAATGTCCTGACGGACAATGGCCGCATAAC | |
| | AGCGGTCATTGACTGGAGCGAGGCGATGTTCGGG | |
| | GATTCCCAATACGAGGTCGCCAACATCTTCTTCTG | |
| | GAGGCCGTGGTTGGCTTGTATGGAGCAGCAGACG | |
| | CGCTACTTCGAGCGGAGGCATCCGGAGCTTGCAG | |
| | GATCGCCGCGGCTCCGGGCGTATATGCTCCGCAT | |
| | TGGTCTTGACCAACTCTATCAGAGCTTGGTTGACG | |
| | GCAATTTCGATGATGCAGCTTGGGCGCAGGGTCG | |
| | ATGCGACGCAATCGTCCGATCCGGAGCCGGGACT | |
| | GTCGGGCGTACACAAATCGCCCGCAGAAGCGCGG | |
| | CCGTCTGGACCGATGGCTGTGTAGAAGTACTCGC | |
| | CGATAGTGGAAACCGACGCCCCAGCACTCGTCCG | |
| | AGGGCAAAGGAATAGCACGTGCTACGAGATTTCG | |
| | ATTCCACCGCCGCCTTCTATGAAAGGTTGGGCTTC | |
| | GGAATCGTTTTCCGGGACGCTGGCTGGATGATCC | |
| | TCCAGCGCGGGGATCTCATGCTGGAGTTCTTCGC | |
| | CCACCCCAACTTGTTTATTGCAGCTTATAATGGTT | |
| | ACAAATAAAGCAATAGCATCACAAATTTCACAAAT | |
| | AAAGCATTTTTTTCACTGCATTCTAGTTGTGGTTT | |
| | GTCCAAACTCATCAATGTATCTTATCATGTCTGTA | |
| | TACCGTCGACCTCTAGCTAGAGCTTGGCGTAATCA | |
| | TGGTCATAGCTGTTTCCTGTGTGAAATTGTTATCC | |
| | GCTCACAATTCCACACAACATACGAGCCGGAAGC | |
| | ATAAAGTGTAAAGCCTGGGGTGCCTAATGAGTGA | |
| | GCTAACTCACATTAATTGCGTTGCGCTCACTGCCC | |
| | GCTTTCCAGTCGGGAAACCTGTCGTGCCAGCTGC | |
| | ATTAATGAATCGGCCAACGCGCGGGGAGAGGCGG | |
| | TTTGCGTATTGGGCGCTCTTCCGCTTCCTCGCTCA | |
| | CTGACTCGCTGCGCTCGGTCGTTCGGCTGCGGCG | |
| | AGCGGTATCAGCTCACTCAAAGGCGGTAATACGG | |
| | TTATCCACAGAATCAGGGGATAACGCAGGAAAGA | |
| | ACATGTGAGCAAAAGGCCAGCAAAAGGCCAGGAA | |
| | CCGTAAAAAGGCCGCGTTGCTGGCGTTTTTCCATA | |
| | GGCTCCGCCCCCCTGACGAGCATCACAAAAATCG | |
| | ACGCTCAAGTCAGAGGTGGCGAAACCCGACAGGA | |
| | CTATAAAGATACCAGGCGTTTCCCCCTGGAAGCTC | |
| | CCTCGTGCGCTCTCCTGTTCCGACCCTGCCGCTTA | |
| | CCGGATACCTGTCCGCCTTTCTCCCTTCGGGAAGC | |
| | GTGGCGCTTTCTCATAGCTCACGCTGTAGGTATCT | |
| | CAGTTCGGTGTAGGTCGTTCGCTCCAAGCTGGGC | |
| | TGTGTGCACGAACCCCCCGTTCAGCCCGACCGCT | |
| | GCGCCTTATCCGGTAACTATCGTCTTGAGTCCAAC | |
| | CCGGTAAGACACGACTTATCGCCACTGGCAGCAG | |
| | CCACTGGTAACAGGATTAGCAGAGCGAGGTATGT | |
| | AGGCGGTGCTACAGAGTTCTTGAAGTGGTGGCCT | |
| | AACTACGGCTACACTAGAAGAACAGTATTTGGTAT | |
| | CTGCGCTCTGCTGAAGCCAGTTACCTTCGGAAAAA | |
| | GAGTTGGTAGCTCTTGATCCGGCAAACAAACCAC | |
| | CGCTGGTAGCGGTGGTTTTTTTGTTTGCAAGCAGC | |
| | AGATTACGCGCAGAAAAAAAGGATCTCAAGAAGA | |
| | TCCTTTGATCTTTTCTACGGGGTCTGAGCGCGGAA | |
| | CCCCTATTTGTTTATTTTTCTAAATACATTCAAATA | |
| | TGTATCCGCTCATGAATTAATTCTTAGAAAAACTC | |
| | ATCGAGCATCAAATGAAACTGCAATTTATTCATAT | |
| | CAGGATTATCAATACCATATTTTTGAAAAAGCCGT | |
| | TTCTGTAATGAAGGAGAAAACTCACCGAGGCAGT | |
| | TCCATAGGATGGCAAGATCCTGGTATCGGTCTGC | |
| | GATTCCGACTCGTCCAACATCAATACAACCTATTA | |
| | ATTTCCCCTCGTCAAAAATAAGGTTATCAAGTGAG | |
| | AAATCACCATGAGTGACGACTGAATCCGGTGAGA | |
| | ATGGCAAAAGTTTATGCATTTCTTTCCAGACTTGT | |
| | TCAACAGGCCAGCCATTACGCTCGTCATCAAAATC | |
| | ACTCGCATCAACCAAACCGTTATTCATTCGTGATT | |
| | GCGCCTGAGCGAGACGAAATACGCGATCGCTGTT | |
| | AAAAGGACAATTACAAACAGGAATCGAATGCAAC | |
| | CGGCGCAGGAACACTGCCAGCGCATCAACAATAT | |
| | TTTCACCTGAATCAGGATATTCTTCTAATACCTGG | |
| | AATGCTGTTTTCCCAGGGATCGCAGTGGTGAGTA | |
| | ACCATGCATCATCAGGAGTACGGATAAAATGCTTG | |
| | ATGGTCGGAAGAGGCATAAATTCCGTCAGCCAGT | |
| | TTAGTCTGACCATCTCATCTGTAACATCATTGGCA | |
| | ACGCTACCTTTGCCATGTTTCAGAAACAACTCTGG | |
| | CGCATCGGGCTTCCCATACAATCGATAGATTGTCG | |
| | CACCTGATTGCCCGACATTATCGCGAGCCCATTTA | |
| | TACCCATATAAATCAGCATCCATGTTGGAATTTAA | |
| | TCGCGGCCTAGAGCAAGACGTTTCCCGTTGAATAT | |
| | GGCTCATAACACCCCTTGTATTACTGTTTATGTAA | |
| | GCAGACAGTTTTATTGTTCATGACCAAAATCCCTT | |
| | AACGTGAGTTTTCGTTCCACTGAGCGTCAGACCCC | |
| | GTAGAAATCCGCGCACATTTCCCCGAAAAGTGCC | |
| | ACCTGACGTC | |
| 25 | GACGGATCGGGAGATCTCCCGATCCCCTATGGTG | pCMV3-tPA- |
| | CACTCTCAGTACAATCTGCTCTGATGCCGCATAGT | Δ-K2-HA |
| | TAAGCCAGTATCTGCTCCCTGCTTGTGTGTTGGAG | (plasmid |
| | CAACAAGGCAAGGCTTGACCGACAATTGCATGAA | encoding tPA- |
| | GAATCTGCTTAGGGTTAGGCGTTTTGCGCTGCTTC | A-K2-HA) |
| | GTCGCTGAGTAGTGCGCGAGCAAAATTTAAGCTA | |
| | GCGAGTACATTTATATTGGCTCATGTCCAATATGA | |
| | CCGCCATGTTGACATTGATTATTGACTAGTTATTA | |
| | ATAGTAATCAATTACGGGGTCATTAGTTCATAGCC | |
| | CATATATGGAGTTCCGCGTTACATAACTTACGGTA | |
| | AATGGCCCGCCTGGCTGACCGCCCAACGACCCCC | |
| | GCCCATTGACGTCAATAATGACGTATGTTCCCATA | |
| | GTAACGCCAATAGGGACTTTCCATTGACGTCAATG | |
| | GGTGGAGTATTTACGGTAAACTGCCCACTTGGCA | |
| | GTACATCAAGTGTATCATATGCCAAGTCCGCCCCC | |
| | TATTGACGTCAATGACGGTAAATGGCCCGCCTGG | |
| | CATTATGCCCAGTACATGACCTTACGGGACTTTCC | |
| | TACTTGGCAGTACATCTACGTATTAGTCATCGCTA | |
| | TTACCATGGTGATGCGGTTTTGGCAGTACACCAAT | |
| | GGGCGTGGATAGCGGTTTGACTCACGGGGATTTC | |
| | CAAGTCTCCACCCCATTGACGTCAATGGGAGTTTG | |
| | TTTTGGCACCAAAATCAACGGGACTTTCCAAAATG | |
| | TCGTAATAACCCCGCCCCGTTGACGCAAATGGGC | |
| | GGTAGGCGTGTACGGTGGGAGGTCTATATAAGCA | |
| | GAGCTCGTTTAGTGAACCGTCAGATCCTCACTCTC | |
| | TTCCGCATCGCTGTCTGCGAGGGCCAGCTGTTGG | |
| | GCTCGCGGTTGAGGACAAACTCTTCGCGGTCTTTC | |
| | CAGTACTCTTGGATCGGAAACCCGTCGGCCTCCG | |
| | AACGGTACTCCGCCACCGAGGGACCTGAGCGAGT | |
| | CCGCATCGACCGGATCGGAAAACCTCTCGAGAAA | |
| | GGCGTCTAACCAGTCACAGTCGCAAGGTAGGCTG | |
| | AGCACCGTGGCGGGCGGCAGCGGGTGGCGGTCG | |
| | GGGTTGTTTCTGGCGGAGGTGCTGCTGATGATGT | |
| | AATTAAAGTAGGCGGTCTTGAGACGGCGGATGGT | |
| | CGAGGTGAGGTGTGGCAGGCTTGAGATCCAGCTG | |
| | TTGGGGTGAGTACTCCCTCTCAAAAGCGGGCATT | |
| | ACTTCTGCGCTAAGATTGTCAGTTTCCAAAAACGA | |
| | GGAGGATTTGATATTCACCTGGCCCGATCTGGCC | |
| | ATACACTTGAGTGACAATGACATCCACTTTGCCTT | |
| | TCTCTCCACAGGTGTCCACTCCCAGGTCCAAGTTT | |
| | AAACTTTAATACGACTCACTATAGGGGCCGCCACC | |
| | AAGCTTGGTACCatggatgcaatgaagagagggctctgctgtgtgctgc | |
| | tgctgtgtggagcagtcttcgtttcgcccagccaggaaatccatgcccgattcagaa | |
| | gaggagccagatcttaccaagtgatctgcagagatgaaaaaacgcagatgatata | |
| | ccagcaacatcagtcatggctgcgccctgtgctcagaagcaaccgggtggaatatt | |
| | gctggtgcaacagtggcagggcacagtgccactcagtgcctgtcaaaagttgcag | |
| | cgagccaaggtgtttcaacgggggcacctgccagcaggccctgtacttctcagatt | |
| | tcgtgtgccagtgccccgaaggatttgctgggaagtgctgtgaaatagataccagg | |
| | gccacgtgctacgaggaccagggcatcagctacaggggcacgtggagcacagcg | |
| | gagagtggcgccgagtgcaccaactggaacagcagcgcgttggcccagaagccc | |
| | tacagcgggcggaggccagacgccatcaggctgggcctggggaaccacaactac | |
| | tgcagaaacccagatcgagactcaaagccctggtgctacgtctttaaggcgggga | |
| | agtacagctcagagttctgcagcacccctgccTGCTCTGAGGGAAAC | |
| | AGTGACTGCTACGAGGACCAGGGcatcagctacaggggcac | |
| | gtggagcacagcggagagtggcgccgagtgcaccaactggaacagcagcgcgtt | |
| | ggcccagaagccctacagcgggcggaggccagacgccatcaggctgggcctggg | |
| | gaaccacaactactgcagaaacccagatcgagactcaaagccctggtgctacgtc | |
| | tttaaggggggaagtacagctcagagttctgcagcacccctgccTGCTCTG | |
| | AGGGAAACAGTGACTCCACCTGCGGCCTGagacagta | |
| | cagccagcctcagtttcgcatcaaaggagggctcttcgccgacatcgcctcccacc | |
| | cctggcaggctgccatctttgccaagcacaggaggtcgcccggagagcggttcctg | |
| | tgcgggggcatactcatcagctcctgctggattctctctgccgcccactgcttccagg | |
| | agaggtttccgccccaccacctgacggtgatcttgggcagaacataccgggtggtc | |
| | cctggcgaggaggagcagaaatttgaagtcgaaaaatacattgtccataaggaat | |
| | tcgatgatgacacttacgacaatgacattgcgctgctgcagctgaaatcggattcgt | |
| | cccgctgtgcccaggagagcagcgtggtccgcactgtgtgccttcccccggcggac | |
| | ctgcagctgccggactggacggagtgtgagctctccggctacggcaagcatgagg | |
| | ccttgtctcctttctattcggagcggctgaaggaggctcatgtcagactgtacccatc | |
| | cagccgctgcacatcacaacatttacttaacagaacagtcaccgacaacatgctgt | |
| | gtgctggagacactcggagcggcgggccccaggcaaacttgcacgacgcctgcc | |
| | agggcgattcgggaggccccctggtgtgtctgaacgatggccgcatgactttggtg | |
| | ggcatcatcagctggggcctgggctgtggacagaaggatgtcccgggtgtgtaca | |
| | ccaaggttaccaactacctagactggattcgtgacaacatgcgaccgGGGGG | |
| | TGGAGGCTCTTATCCTTACGACGTGCCTGACTACG | |
| | CCTAAACTCGAGTCTAGAGCGGCCGCCGAATTCG | |
| | GGCCCGTTTAAACCCGCTGATCAGCCTCGACTGT | |
| | GCCTTCTAGTTGCCAGCCATCTGTTGTTTGCCCCT | |
| | CCCCCGTGCCTTCCTTGACCCTGGAAGGTGCCACT | |
| | CCCACTGTCCTTTCCTAATAAAATGAGGAAATTGC | |
| | ATCGCATTGTCTGAGTAGGTGTCATTCTATTCTGG | |
| | GGGGTGGGGTGGGGCAGGACAGCAAGGGGGAGG | |
| | ATTGGGAAGACAATAGCAGGCATGCTGGGGATGC | |
| | GGTGGGCTCTATGGCTTCTGAGGCGGAAAGAACC | |
| | AGCTGGGGCTCTAGGGGGTATCCCCACGCGCCCT | |
| | GTAGCGGCGCATTAAGCGCGGCGGGTGTGGTGGT | |
| | TACGCGCAGCGTGACCGCTACACTTGCCAGCGCC | |
| | CTAGCGCCCGCTCCTTTCGCTTTCTTCCCTTCCTT | |
| | TCTCGCCACGTTCGCAGGCTTTCCCCGTCAAGCTC | |
| | TAAATCGGGGGCTCCCTTTAGGGTTCCGATTTAGT | |
| | GCTTTACGGCACCTCGACCCCAAAAAACTTGATTA | |
| | GGGTGATGGTTCACGTAGTGGGCCATCGCCCTGA | |
| | TAGACGGTTTTTCGCCCTTTGACGTTGGAGTCCAC | |
| | GTTCTTTAATAGTGGACTCTTGTTCCAAACTGGAA | |
| | CAACACTCAACCCTATCTCGGTCTATTCTTTTGAT | |
| | TTATAAGGGATTTTGCCGATTTCGGCCTATTGGTT | |
| | AAAAAATGAGCTGATTTAACAAAAATTTAACGCGA | |
| | ATTAATTCTGTGGAATGTGTGTCAGTTAGGGTGTG | |
| | GAAAGTCCCCAGGCTCCCCAGCAGGCAGAAGTAT | |
| | GCAAAGCATGCATCTCAATTAGTCAGCAACCAGGT | |
| | GTGGAAAGTCCCCAGGCTCCCCAGCAGGCAGAAG | |
| | TATGCAAAGCATGCATCTCAATTAGTCAGCAACCA | |
| | TAGTCCCGCCCCTAACTCCGCCCATCCCGCCCCTA | |
| | ACTCCGCCCAGTTCCGCCCATTCTCCGCCCCATGG | |
| | CTGACTAATTTTTTTTATTTATGCAGAGGCCGAGG | |
| | CCGCCTCTGCCTCTGAGCTATTCCAGAAGTAGTGA | |
| | GGAGGCTTTTTTGGAGGCCTAGGCTTTTGCAAAAA | |
| | GCTCTCGGGAGCTTGTATATCCATTTTCGGATCTG | |
| | ATCAGCACGTGATGAAAAAGCCTGAACTCACCGC | |
| | GACGTCTGTCGAGAAGTTTCTGATCGAAAAGTTCG | |
| | ACAGCGTCTCCGACCTGATGCAGCTCTCGGAGGG | |
| | CGAAGAATCTCGTGCTTTCAGCTTCGATGTAGGAG | |
| | GGCGTGGATATGTCCTGCGGGTAAATAGCTGCGC | |
| | CGATGGTTTCTACAAAGATCGTTATGTTTATCGGC | |
| | ACTTTGCATCGGCCGCGCTCCCGATTCCGGAAGT | |
| | GCTTGACATTGGGGAATTCAGCGAGAGCCTGACC | |
| | TATTGCATCTCCCGCCGTGCACAGGGTGTCACGTT | |
| | GCAAGACCTGCCTGAAACCGAACTGCCCGCTGTT | |
| | CTGCAGCCGGTCGCGGAGGCCATGGATGCGATCG | |
| | CTGCGGCCGATCTTAGCCAGACGAGCGGGTTCGG | |
| | CCCATTCGGACCGCAAGGAATCGGTCAATACACT | |
| | ACATGGCGTGATTTCATATGCGCGATTGCTGATCC | |
| | CCATGTGTATCACTGGCAAACTGTGATGGACGAC | |
| | ACCGTCAGTGCGTCCGTCGCGCAGGCTCTCGATG | |
| | AGCTGATGCTTTGGGCCGAGGACTGCCCCGAAGT | |
| | CCGGCACCTCGTGCACGCGGATTTCGGCTCCAAC | |
| | AATGTCCTGACGGACAATGGCCGCATAACAGCGG | |
| | TCATTGACTGGAGCGAGGCGATGTTCGGGGATTC | |
| | CCAATACGAGGTCGCCAACATCTTCTTCTGGAGGC | |
| | CGTGGTTGGCTTGTATGGAGCAGCAGACGCGCTA | |
| | CTTCGAGCGGAGGCATCCGGAGCTTGCAGGATCG | |
| | CCGCGGCTCCGGGCGTATATGCTCCGCATTGGTC | |
| | TTGACCAACTCTATCAGAGCTTGGTTGACGGCAAT | |
| | TTCGATGATGCAGCTTGGGCGCAGGGTCGATGCG | |
| | ACGCAATCGTCCGATCCGGAGCCGGGACTGTCGG | |
| | GCGTACACAAATCGCCCGCAGAAGCGCGGCCGTC | |
| | TGGACCGATGGCTGTGTAGAAGTACTCGCCGATA | |
| | GTGGAAACCGACGCCCCAGCACTCGTCCGAGGGC | |
| | AAAGGAATAGCACGTGCTACGAGATTTCGATTCCA | |
| | CCGCCGCCTTCTATGAAAGGTTGGGCTTCGGAAT | |
| | CGTTTTCCGGGACGCTGGCTGGATGATCCTCCAG | |
| | CGCGGGGATCTCATGCTGGAGTTCTTCGCCCACC | |
| | CCAACTTGTTTATTGCAGCTTATAATGGTTACAAA | |
| | TAAAGCAATAGCATCACAAATTTCACAAATAAAGC | |
| | ATTTTTTTCACTGCATTCTAGTTGTGGTTTGTCCA | |
| | AACTCATCAATGTATCTTATCATGTCTGTATACCG | |
| | TCGACCTCTAGCTAGAGCTTGGCGTAATCATGGTC | |
| | ATAGCTGTTTCCTGTGTGAAATTGTTATCCGCTCA | |
| | CAATTCCACACAACATACGAGCCGGAAGCATAAA | |
| | GTGTAAAGCCTGGGGTGCCTAATGAGTGAGCTAA | |
| | CTCACATTAATTGCGTTGCGCTCACTGCCCGCTTT | |
| | CCAGTCGGGAAACCTGTCGTGCCAGCTGCATTAA | |
| | TGAATCGGCCAACGCGCGGGGAGAGGCGGTTTGC | |
| | GTATTGGGCGCTCTTCCGCTTCCTCGCTCACTGAC | |
| | TCGCTGCGCTCGGTCGTTCGGCTGCGGCGAGCGG | |
| | TATCAGCTCACTCAAAGGCGGTAATACGGTTATCC | |
| | ACAGAATCAGGGGATAACGCAGGAAAGAACATGT | |
| | GAGCAAAAGGCCAGCAAAAGGCCAGGAACCGTAA | |
| | AAAGGCCGCGTTGCTGGCGTTTTTCCATAGGCTCC | |
| | GCCCCCCTGACGAGCATCACAAAAATCGACGCTC | |
| | AAGTCAGAGGTGGCGAAACCCGACAGGACTATAA | |
| | AGATACCAGGCGTTTCCCCCTGGAAGCTCCCTCGT | |
| | GCGCTCTCCTGTTCCGACCCTGCCGCTTACCGGAT | |
| | ACCTGTCCGCCTTTCTCCCTTCGGGAAGCGTGGC | |
| | GCTTTCTCATAGCTCACGCTGTAGGTATCTCAGTT | |
| | CGGTGTAGGTCGTTCGCTCCAAGCTGGGCTGTGT | |
| | GCACGAACCCCCCGTTCAGCCCGACCGCTGCGCC | |
| | TTATCCGGTAACTATCGTCTTGAGTCCAACCCGGT | |
| | AAGACACGACTTATCGCCACTGGCAGCAGCCACT | |
| | GGTAACAGGATTAGCAGAGCGAGGTATGTAGGCG | |
| | GTGCTACAGAGTTCTTGAAGTGGTGGCCTAACTAC | |
| | GGCTACACTAGAAGAACAGTATTTGGTATCTGCGC | |
| | TCTGCTGAAGCCAGTTACCTTCGGAAAAAGAGTTG | |
| | GTAGCTCTTGATCCGGCAAACAAACCACCGCTGG | |
| | TAGCGGTGGTTTTTTTGTTTGCAAGCAGCAGATTA | |
| | CGCGCAGAAAAAAAGGATCTCAAGAAGATCCTTT | |
| | GATCTTTTCTACGGGGTCTGAGCGCGGAACCCCT | |
| | ATTTGTTTATTTTTCTAAATACATTCAAATATGTAT | |
| | CCGCTCATGAATTAATTCTTAGAAAAACTCATCGA | |
| | GCATCAAATGAAACTGCAATTTATTCATATCAGGA | |
| | TTATCAATACCATATTTTTGAAAAAGCCGTTTCTG | |
| | TAATGAAGGAGAAAACTCACCGAGGCAGTTCCAT | |
| | AGGATGGCAAGATCCTGGTATCGGTCTGCGATTC | |
| | CGACTCGTCCAACATCAATACAACCTATTAATTTC | |
| | CCCTCGTCAAAAATAAGGTTATCAAGTGAGAAATC | |
| | ACCATGAGTGACGACTGAATCCGGTGAGAATGGC | |
| | AAAAGTTTATGCATTTCTTTCCAGACTTGTTCAAC | |
| | AGGCCAGCCATTACGCTCGTCATCAAAATCACTCG | |
| | CATCAACCAAACCGTTATTCATTCGTGATTGCGCC | |
| | TGAGCGAGACGAAATACGCGATCGCTGTTAAAAG | |
| | GACAATTACAAACAGGAATCGAATGCAACCGGCG | |
| | CAGGAACACTGCCAGCGCATCAACAATATTTTCAC | |
| | CTGAATCAGGATATTCTTCTAATACCTGGAATGCT | |
| | GTTTTCCCAGGGATCGCAGTGGTGAGTAACCATG | |
| | CATCATCAGGAGTACGGATAAAATGCTTGATGGTC | |
| | GGAAGAGGCATAAATTCCGTCAGCCAGTTTAGTCT | |
| | GACCATCTCATCTGTAACATCATTGGCAACGCTAC | |
| | CTTTGCCATGTTTCAGAAACAACTCTGGCGCATCG | |
| | GGCTTCCCATACAATCGATAGATTGTCGCACCTGA | |
| | TTGCCCGACATTATCGCGAGCCCATTTATACCCAT | |
| | ATAAATCAGCATCCATGTTGGAATTTAATCGCGGC | |
| | CTAGAGCAAGACGTTTCCCGTTGAATATGGCTCAT | |
| | AACACCCCTTGTATTACTGTTTATGTAAGCAGACA | |
| | GTTTTATTGTTCATGACCAAAATCCCTTAACGTGA | |
| | GTTTTCGTTCCACTGAGCGTCAGACCCCGTAGAAA | |
| | TCCGCGCACATTTCCCCGAAAAGTGCCACCTGAC | |
| | GTC | |
| 26 | GACGGATCGGGAGATCTCCCGATCCCCTATGGTG | pCMV3-tPA- |
| | CACTCTCAGTACAATCTGCTCTGATGCCGCATAGT | D236, 238N- |
| | TAAGCCAGTATCTGCTCCCTGCTTGTGTGTTGGAG | HA (plasmid |
| | GAATCTGCTTAGGGTTAGGCGTTTTGCGCTGCTTC | for the tPA- |
| | GTCGCTGAGTAGTGCGCGAGCAAAATTTAAGCTA | D236, 238N-HA |
| | CAACAAGGCAAGGCTTGACCGACAATTGCATGAA | mutant) |
| | GCGAGTACATTTATATTGGCTCATGTCCAATATGA | |
| | CCGCCATGTTGACATTGATTATTGACTAGTTATTA | |
| | ATAGTAATCAATTACGGGGTCATTAGTTCATAGCC | |
| | CATATATGGAGTTCCGCGTTACATAACTTACGGTA | |
| | AATGGCCCGCCTGGCTGACCGCCCAACGACCCCC | |
| | GCCCATTGACGTCAATAATGACGTATGTTCCCATA | |
| | GTAACGCCAATAGGGACTTTCCATTGACGTCAATG | |
| | GGTGGAGTATTTACGGTAAACTGCCCACTTGGCA | |
| | GTACATCAAGTGTATCATATGCCAAGTCCGCCCCC | |
| | TATTGACGTCAATGACGGTAAATGGCCCGCCTGG | |
| | CATTATGCCCAGTACATGACCTTACGGGACTTTCC | |
| | TACTTGGCAGTACATCTACGTATTAGTCATCGCTA | |
| | TTACCATGGTGATGCGGTTTTGGCAGTACACCAAT | |
| | GGGCGTGGATAGCGGTTTGACTCACGGGGATTTC | |
| | CAAGTCTCCACCCCATTGACGTCAATGGGAGTTTG | |
| | TTTTGGCACCAAAATCAACGGGACTTTCCAAAATG | |
| | TCGTAATAACCCCGCCCCGTTGACGCAAATGGGC | |
| | GGTAGGCGTGTACGGTGGGAGGTCTATATAAGCA | |
| | GAGCTCGTTTAGTGAACCGTCAGATCCTCACTCTC | |
| | TTCCGCATCGCTGTCTGCGAGGGCCAGCTGTTGG | |
| | GCTCGCGGTTGAGGACAAACTCTTCGCGGTCTTTC | |
| | CAGTACTCTTGGATCGGAAACCCGTCGGCCTCCG | |
| | AACGGTACTCCGCCACCGAGGGACCTGAGCGAGT | |
| | CCGCATCGACCGGATCGGAAAACCTCTCGAGAAA | |
| | GGCGTCTAACCAGTCACAGTCGCAAGGTAGGCTG | |
| | AGCACCGTGGCGGGCGGCAGCGGGTGGCGGTCG | |
| | GGGTTGTTTCTGGCGGAGGTGCTGCTGATGATGT | |
| | AATTAAAGTAGGCGGTCTTGAGACGGCGGATGGT | |
| | CGAGGTGAGGTGTGGCAGGCTTGAGATCCAGCTG | |
| | TTGGGGTGAGTACTCCCTCTCAAAAGCGGGCATT | |
| | ACTTCTGCGCTAAGATTGTCAGTTTCCAAAAACGA | |
| | GGAGGATTTGATATTCACCTGGCCCGATCTGGCC | |
| | ATACACTTGAGTGACAATGACATCCACTTTGCCTT | |
| | TCTCTCCACAGGTGTCCACTCCCAGGTCCAAGTTT | |
| | AAACTTTAATACGACTCACTATAGGGGCCGCCACC | |
| | AAGCTTGGTACCatggatgcaatgaagagagggctctgctgtgtgctgc | |
| | tgctgtgtggagcagtcttcgtttcgcccagccaggaaatccatgcccgattcagaa | |
| | gaggagccagatcttaccaagtgatctgcagagatgaaaaaacgcagatgatata | |
| | ccagcaacatcagtcatggctgcgccctgtgctcagaagcaaccgggtggaatatt | |
| | gctggtgcaacagtggcagggcacagtgccactcagtgcctgtcaaaagttgcag | |
| | cgagccaaggtgtttcaacgggggcacctgccagcaggccctgtacttctcagatt | |
| | tcgtgtgccagtgccccgaaggatttgctgggaagtgctgtgaaatagataccagg | |
| | gccacgtgctacgaggaccagggcatcagctacaggggcacgtggagcacagcg | |
| | gagagtggcgccgagtgcaccaactggaacagcagcgcgttggcccagaagccc | |
| | tacagcgggcggaggccagacgccatcaggctgggcctggggaaccacaactac | |
| | tgcagaaacccagatcgagactcaaagccctggtgctacgtctttaaggcgggga | |
| | agtacagctcagagttctgcagcacccctgcctgctctgagggaaacagtgactgc | |
| | tactttgggaatgggtcagcctaccgtggcacgcacagcctcaccgagtcgggtgc | |
| | ctcctgcctcccgtggaattccatgatcctgataggcaaggtttacacagcacaga | |
| | accccagtgcccaggcactgggcctggGCAAACATAATTACTGCC | |
| | GGAATCCTAATGGGAACGCCAAGCCCTGGTGCcacg | |
| | tgctgaagaaccgcaggctgacgtgggagtactgtgatgtgccctcctgctccacc | |
| | tgcggcctgagacagtacagccagcctcagtttcgcatcaaaggagggctcttcgc | |
| | cgacatcgcctcccacccctggcaggctgccatctttgccaagcacaggaggtcgc | |
| | ccggagagcggttcctgtgcgggggcatactcatcagctcctgctggattctctctg | |
| | ccgcccactgcttccaggagaggtttccgccccaccacctgacggtgatcttgggc | |
| | agaacataccgggtggtccctggcgaggaggagcagaaatttgaagtcgaaaaa | |
| | tacattgtccataaggaattcgatgatgacacttacgacaatgacattgcgctgctg | |
| | cagctgaaatcggattcgtcccgctgtgcccaggagagcagcgtggtccgcactgt | |
| | gtgccttcccccggcggacctgcagctgccggactggacggagtgtgagctctccg | |
| | gctacggcaagcatgaggccttgtctcctttctattcggagcggctgaaggaggctc | |
| | atgtcagactgtacccatccagccgctgcacatcacaacatttacttaacagaaca | |
| | gtcaccgacaacatgctgtgtgctggagacactcggagcggcgggccccaggca | |
| | aacttgcacgacgcctgccagggcgattcgggaggccccctggtgtgtctgaacg | |
| | atggccgcatgactttggtgggcatcatcagctggggcctgggctgtggacagaag | |
| | gatgtcccgggtgtgtacaccaaggttaccaactacctagactggattcgtgacaa | |
| | catgcgaccgGGGGGTGGAGGCTCTTATCCTTACGACG | |
| | TGCCTGACTACGCCTAAACTCGAGTCTAGAGCGG | |
| | CCGCCGAATTCGGGCCCGTTTAAACCCGCTGATC | |
| | AGCCTCGACTGTGCCTTCTAGTTGCCAGCCATCTG | |
| | TTGTTTGCCCCTCCCCCGTGCCTTCCTTGACCCTG | |
| | GAAGGTGCCACTCCCACTGTCCTTTCCTAATAAAA | |
| | TGAGGAAATTGCATCGCATTGTCTGAGTAGGTGTC | |
| | ATTCTATTCTGGGGGGTGGGGTGGGGCAGGACAG | |
| | CAAGGGGGAGGATTGGGAAGACAATAGCAGGCAT | |
| | GCTGGGGATGCGGTGGGCTCTATGGCTTCTGAGG | |
| | CGGAAAGAACCAGCTGGGGCTCTAGGGGGTATCC | |
| | CCACGCGCCCTGTAGCGGCGCATTAAGCGCGGCG | |
| | GGTGTGGTGGTTACGCGCAGCGTGACCGCTACAC | |
| | TTGCCAGCGCCCTAGCGCCCGCTCCTTTCGCTTTC | |
| | TTCCCTTCCTTTCTCGCCACGTTCGCAGGCTTTCC | |
| | CCGTCAAGCTCTAAATCGGGGGCTCCCTTTAGGG | |
| | TTCCGATTTAGTGCTTTACGGCACCTCGACCCCAA | |
| | AAAACTTGATTAGGGTGATGGTTCACGTAGTGGG | |
| | CCATCGCCCTGATAGACGGTTTTTCGCCCTTTGAC | |
| | GTTGGAGTCCACGTTCTTTAATAGTGGACTCTTGT | |
| | TCCAAACTGGAACAACACTCAACCCTATCTCGGTC | |
| | TATTCTTTTGATTTATAAGGGATTTTGCCGATTTC | |
| | GGCCTATTGGTTAAAAAATGAGCTGATTTAACAAA | |
| | AATTTAACGCGAATTAATTCTGTGGAATGTGTGTC | |
| | AGTTAGGGTGTGGAAAGTCCCCAGGCTCCCCAGC | |
| | AGGCAGAAGTATGCAAAGCATGCATCTCAATTAGT | |
| | CAGCAACCAGGTGTGGAAAGTCCCCAGGCTCCCC | |
| | AGCAGGCAGAAGTATGCAAAGCATGCATCTCAAT | |
| | TAGTCAGCAACCATAGTCCCGCCCCTAACTCCGCC | |
| | CATCCCGCCCCTAACTCCGCCCAGTTCCGCCCATT | |
| | CTCCGCCCCATGGCTGACTAATTTTTTTTATTTAT | |
| | GCAGAGGCCGAGGCCGCCTCTGCCTCTGAGCTAT | |
| | TCCAGAAGTAGTGAGGAGGCTTTTTTGGAGGCCT | |
| | AGGCTTTTGCAAAAAGCTCTCGGGAGCTTGTATAT | |
| | CCATTTTCGGATCTGATCAGCACGTGATGAAAAAG | |
| | CCTGAACTCACCGCGACGTCTGTCGAGAAGTTTCT | |
| | GATCGAAAAGTTCGACAGCGTCTCCGACCTGATG | |
| | CAGCTCTCGGAGGGCGAAGAATCTCGTGCTTTCA | |
| | GCTTCGATGTAGGAGGGCGTGGATATGTCCTGCG | |
| | GGTAAATAGCTGCGCCGATGGTTTCTACAAAGATC | |
| | GTTATGTTTATCGGCACTTTGCATCGGCCGCGCTC | |
| | CCGATTCCGGAAGTGCTTGACATTGGGGAATTCA | |
| | GCGAGAGCCTGACCTATTGCATCTCCCGCCGTGC | |
| | ACAGGGTGTCACGTTGCAAGACCTGCCTGAAACC | |
| | GAACTGCCCGCTGTTCTGCAGCCGGTCGCGGAGG | |
| | CCATGGATGCGATCGCTGCGGCCGATCTTAGCCA | |
| | GACGAGCGGGTTCGGCCCATTCGGACCGCAAGGA | |
| | ATCGGTCAATACACTACATGGCGTGATTTCATATG | |
| | CGCGATTGCTGATCCCCATGTGTATCACTGGCAAA | |
| | CTGTGATGGACGACACCGTCAGTGCGTCCGTCGC | |
| | GCAGGCTCTCGATGAGCTGATGCTTTGGGCCGAG | |
| | GACTGCCCCGAAGTCCGGCACCTCGTGCACGCGG | |
| | ATTTCGGCTCCAACAATGTCCTGACGGACAATGGC | |
| | CGCATAACAGCGGTCATTGACTGGAGCGAGGCGA | |
| | TGTTCGGGGATTCCCAATACGAGGTCGCCAACAT | |
| | CTTCTTCTGGAGGCCGTGGTTGGCTTGTATGGAG | |
| | CAGCAGACGCGCTACTTCGAGCGGAGGCATCCGG | |
| | AGCTTGCAGGATCGCCGCGGCTCCGGGCGTATAT | |
| | GCTCCGCATTGGTCTTGACCAACTCTATCAGAGCT | |
| | TGGTTGACGGCAATTTCGATGATGCAGCTTGGGC | |
| | GCAGGGTCGATGCGACGCAATCGTCCGATCCGGA | |
| | GCCGGGACTGTCGGGCGTACACAAATCGCCCGCA | |
| | GAAGCGCGGCCGTCTGGACCGATGGCTGTGTAGA | |
| | AGTACTCGCCGATAGTGGAAACCGACGCCCCAGC | |
| | ACTCGTCCGAGGGCAAAGGAATAGCACGTGCTAC | |
| | GAGATTTCGATTCCACCGCCGCCTTCTATGAAAGG | |
| | TTGGGCTTCGGAATCGTTTTCCGGGACGCTGGCT | |
| | GGATGATCCTCCAGCGCGGGGATCTCATGCTGGA | |
| | GTTCTTCGCCCACCCCAACTTGTTTATTGCAGCTT | |
| | ATAATGGTTACAAATAAAGCAATAGCATCACAAAT | |
| | TTCACAAATAAAGCATTTTTTTCACTGCATTCTAG | |
| | TTGTGGTTTGTCCAAACTCATCAATGTATCTTATC | |
| | ATGTCTGTATACCGTCGACCTCTAGCTAGAGCTTG | |
| | GCGTAATCATGGTCATAGCTGTTTCCTGTGTGAAA | |
| | TTGTTATCCGCTCACAATTCCACACAACATACGAG | |
| | CCGGAAGCATAAAGTGTAAAGCCTGGGGTGCCTA | |
| | ATGAGTGAGCTAACTCACATTAATTGCGTTGCGCT | |
| | CACTGCCCGCTTTCCAGTCGGGAAACCTGTCGTG | |
| | CCAGCTGCATTAATGAATCGGCCAACGCGCGGGG | |
| | AGAGGCGGTTTGCGTATTGGGCGCTCTTCCGCTT | |
| | CCTCGCTCACTGACTCGCTGCGCTCGGTCGTTCG | |
| | GCTGCGGCGAGCGGTATCAGCTCACTCAAAGGCG | |
| | GTAATACGGTTATCCACAGAATCAGGGGATAACG | |
| | CAGGAAAGAACATGTGAGCAAAAGGCCAGCAAAA | |
| | GGCCAGGAACCGTAAAAAGGCCGCGTTGCTGGCG | |
| | TTTTTCCATAGGCTCCGCCCCCCTGACGAGCATCA | |
| | CAAAAATCGACGCTCAAGTCAGAGGTGGCGAAAC | |
| | CCGACAGGACTATAAAGATACCAGGCGTTTCCCC | |
| | CTGGAAGCTCCCTCGTGCGCTCTCCTGTTCCGACC | |
| | CTGCCGCTTACCGGATACCTGTCCGCCTTTCTCCC | |
| | TTCGGGAAGCGTGGCGCTTTCTCATAGCTCACGCT | |
| | GTAGGTATCTCAGTTCGGTGTAGGTCGTTCGCTCC | |
| | AAGCTGGGCTGTGTGCACGAACCCCCCGTTCAGC | |
| | CCGACCGCTGCGCCTTATCCGGTAACTATCGTCTT | |
| | GAGTCCAACCCGGTAAGACACGACTTATCGCCAC | |
| | TGGCAGCAGCCACTGGTAACAGGATTAGCAGAGC | |
| | GAGGTATGTAGGCGGTGCTACAGAGTTCTTGAAG | |
| | TGGTGGCCTAACTACGGCTACACTAGAAGAACAG | |
| | TATTTGGTATCTGCGCTCTGCTGAAGCCAGTTACC | |
| | TTCGGAAAAAGAGTTGGTAGCTCTTGATCCGGCA | |
| | AACAAACCACCGCTGGTAGCGGTGGTTTTTTTGTT | |
| | TGCAAGCAGCAGATTACGCGCAGAAAAAAAGGAT | |
| | CTCAAGAAGATCCTTTGATCTTTTCTACGGGGTCT | |
| | GAGCGCGGAACCCCTATTTGTTTATTTTTCTAAAT | |
| | ACATTCAAATATGTATCCGCTCATGAATTAATTCT | |
| | TAGAAAAACTCATCGAGCATCAAATGAAACTGCAA | |
| | TTTATTCATATCAGGATTATCAATACCATATTTTTG | |
| | AAAAAGCCGTTTCTGTAATGAAGGAGAAAACTCAC | |
| | CGAGGCAGTTCCATAGGATGGCAAGATCCTGGTA | |
| | TCGGTCTGCGATTCCGACTCGTCCAACATCAATAC | |
| | AACCTATTAATTTCCCCTCGTCAAAAATAAGGTTA | |
| | TCAAGTGAGAAATCACCATGAGTGACGACTGAAT | |
| | CCGGTGAGAATGGCAAAAGTTTATGCATTTCTTTC | |
| | CAGACTTGTTCAACAGGCCAGCCATTACGCTCGTC | |
| | ATCAAAATCACTCGCATCAACCAAACCGTTATTCA | |
| | TTCGTGATTGCGCCTGAGCGAGACGAAATACGCG | |
| | ATCGCTGTTAAAAGGACAATTACAAACAGGAATCG | |
| | AATGCAACCGGCGCAGGAACACTGCCAGCGCATC | |
| | AACAATATTTTCACCTGAATCAGGATATTCTTCTA | |
| | ATACCTGGAATGCTGTTTTCCCAGGGATCGCAGTG | |
| | GTGAGTAACCATGCATCATCAGGAGTACGGATAA | |
| | AATGCTTGATGGTCGGAAGAGGCATAAATTCCGT | |
| | CAGCCAGTTTAGTCTGACCATCTCATCTGTAACAT | |
| | CATTGGCAACGCTACCTTTGCCATGTTTCAGAAAC | |
| | AACTCTGGCGCATCGGGCTTCCCATACAATCGATA | |
| | GATTGTCGCACCTGATTGCCCGACATTATCGCGAG | |
| | CCCATTTATACCCATATAAATCAGCATCCATGTTG | |
| | GAATTTAATCGCGGCCTAGAGCAAGACGTTTCCC | |
| | GTTGAATATGGCTCATAACACCCCTTGTATTACTG | |
| | TTTATGTAAGCAGACAGTTTTATTGTTCATGACCA | |
| | AAATCCCTTAACGTGAGTTTTCGTTCCACTGAGCG | |
| | TCAGACCCCGTAGAAATCCGCGCACATTTCCCCGA | |
| | AAAGTGCCACCTGACGTC | |
| 27 | MDAMKRGLCCVLLLCGAVFVSPSQEIHARFRRGARSY | Human tPA- |
| | QVICRDEKTQMIYQQHQSWLRPVLRSNRVEYCWCNSG | HA amino acid |
| | RAQCHSVPVKSCSEPRCFNGGTCQQALYFSDFVCQCPE | sequence |
| | GFAGKCCEIDTRATCYEDQGISYRGTWSTAESGAECTN | (kringle 2 |
| | WNSSALAQKPYSGRRPDAIRLGLGNHNYCRNPDRDSKP | domain |
| | WCYVFKAGKYSSEFCSTPACSEGNSDCYFGNGSAYRGT | underlined; |
| | HSLTESGASCLPWNSMILIGKVYTAQNPSAQALGLGKH | GGGGS linker |
| | NYCRNPDGDAKPWCHVLKNRRLTWEYCDVPSCSTCGL | in bold; HA tag |
| | RQYSQPQFRIKGGLFADIASHPWQAAIFAKHRRSPGERF | sequence bold |
| | LCGGILISSCWILSAAHCFQERFPPHHLTVILGRTYRVVP | underlined) |
| | GEEEQKFEVEKYIVHKEFDDDTYDNDIALLQLKSDSSR | |
| | CAQESSVVRTVCLPPADLQLPDWTECELSGYGKHEALS | |
| | PFYSERLKEAHVRLYPSSRCTSQHLLNRTVTDNMLCAG | |
| | DTRSGGPQANLHDACQGDSGGPLVCLNDGRMTLVGIIS | |
| | WGLGCGQKDVPGVYTKVTNYLDWIRDNMRPGGGGS | |
| | YPYDVPDYA | |
| 28 | ATGGATGCAATGAAGAGAGGGCTCTGCTGTGTGCTG | Human tPA- |
| | AGGAAATCCATGCCCGATTCAGAAGAGGAGCCAGAT | HA encoding |
| | CTGCTGTGTGGAGCAGTCTTCGTTTCGCCCAGCC | nucleic acid |
| | GATGATATACCAGCAACATCAGTCATGGCTGCGCCCT | sequence |
| | GTGCTCAGAAGCAACCGGGTGGAATATTGCTGG | (linker |
| | CGTGTGCCAGTGCCCCGAAGGATTTGCTGGGAA | sequence bold; |
| | CTTACCAAGTGATCTGCAGAGATGAAAAAACGCA | HA tag |
| | TGCAACAGTGGCAGGGCACAGTGCCACTCAGTGCCT | sequence bold |
| | GTCAAAAGTTGCAGCGAGCCAAGGTGTTTCAACG | underlined) |
| | GGGGCACCTGCCAGCAGGCCCTGTACTTCTCAGATTT | |
| | GTGCTGTGAAATAGATACCAGGGCCACGTGCTACGA | |
| | GGACCAGGGCATCAGCTACAGGGGCACGTGGAGC | |
| | ACAGCGGAGAGTGGCGCCGAGTGCACCAACTGGAAC | |
| | AGCAGCGCGTTGGCCCAGAAGCCCTACAGCGGGC | |
| | GGAGGCCAGACGCCATCAGGCTGGGCCTGGGGAACC | |
| | ACAACTACTGCAGAAACCCAGATCGAGACTCAAA | |
| | GCCCTGGTGCTACGTCTTTAAGGCGGGGAAGTACAG | |
| | CTCAGAGTTCTGCAGCACCCCTGCCTGCTCTGAG | |
| | GGAAACAGTGACTGCTACTTTGGGAATGGGTCAGCC | |
| | TACCGTGGCACGCACAGCCTCACCGAGTCGGGTG | |
| | CCTCCTGCCTCCCGTGGAATTCCATGATCCTGATAGG | |
| | CAAGGTTTACACAGCACAGAACCCCAGTGCCCA | |
| | GGCACTGGGCCTGGGCAAACATAATTACTGCCGGAA | |
| | TCCTGATGGGGATGCCAAGCCCTGGTGCCACGTG | |
| | CTGAAGAACCGCAGGCTGACGTGGGAGTACTGTGAT | |
| | GTGCCCTCCTGCTCCACCTGCGGCCTGAGACAGT | |
| | ACAGCCAGCCTCAGTTTCGCATCAAAGGAGGGCTCTT | |
| | CGCCGACATCGCCTCCCACCCCTGGCAGGCTGC | |
| | CATCTTTGCCAAGCACAGGAGGTCGCCCGGAGAGCG | |
| | GTTCCTGTGCGGGGGCATACTCATCAGCTCCTGC | |
| | TGGATTCTCTCTGCCGCCCACTGCTTCCAGGAGAGGT | |
| | TTCCGCCCCACCACCTGACGGTGATCTTGGGCA | |
| | GAACATACCGGGTGGTCCCTGGCGAGGAGGAGCAGA | |
| | AATTTGAAGTCGAAAAATACATTGTCCATAAGGA | |
| | ATTCGATGATGACACTTACGACAATGACATTGCGCTG | |
| | CTGCAGCTGAAATCGGATTCGTCCCGCTGTGCC | |
| | CAGGAGAGCAGCGTGGTCCGCACTGTGTGCCTTCCCC | |
| | CGGCGGACCTGCAGCTGCCGGACTGGACGGAGT | |
| | GTGAGCTCTCCGGCTACGGCAAGCATGAGGCCTTGTC | |
| | TCCTTTCTATTCGGAGCGGCTGAAGGAGGCTCA | |
| | TGTCAGACTGTACCCATCCAGCCGCTGCACATCACAA | |
| | CATTTACTTAACAGAACAGTCACCGACAACATG | |
| | CTGTGTGCTGGAGACACTCGGAGCGGCGGGCCCCAG | |
| | GCAAACTTGCACGACGCCTGCCAGGGCGATTCGG | |
| | GAGGCCCCCTGGTGTGTCTGAACGATGGCCGCATGA | |
| | CTTTGGTGGGCATCATCAGCTGGGGCCTGGGCTG | |
| | TGGACAGAAGGATGTCCCGGGTGTGTACACCAAGGT | |
| | TACCAACTACCTAGACTGGATTCGTGACAACATG | |
| | CGACCGTGAGGGGGTGGAGGCTCTTATCCTTACGA | |
| | CGTGCCTGACTACGCC | |
| 29 | GACGGATCGGGAGATCTCCCGATCCCCTATGGTGCAC | pCMV3-tPA- |
| | TCTCAGTACAATCTGCTCTGATGCCGCATAGTTAAGC | HA (Plasmid |
| | CAGTATCTGCTCCCTGCTTGTGTGTTGGAGGTCGCTG | sequence for |
| | AGTAGTGCGCGAGCAAAATTTAAGCTACAACAAGGC | the tPA-HA) |
| | AAGGCTTGACCGACAATTGCATGAAGAATCTGCTTA | |
| | GGGTTAGGCGTTTTGCGCTGCTTCGCGAGTACATTTA | |
| | TATTGGCTCATGTCCAATATGACCGCCATGTTGACAT | |
| | TGATTATTGACTAGTTATTAATAGTAATCAATTACGG | |
| | GGTCATTAGTTCATAGCCCATATATGGAGTTCCGCGT | |
| | TACATAACTTACGGTAAATGGCCCGCCTGGCTGACCG | |
| | CCCAACGACCCCCGCCCATTGACGTCAATAATGACGT | |
| | ATGTTCCCATAGTAACGCCAATAGGGACTTTCCATTG | |
| | ACGTCAATGGGTGGAGTATTTACGGTAAACTGCCCAC | |
| | TTGGCAGTACATCAAGTGTATCATATGCCAAGTCCGC | |
| | CCCCTATTGACGTCAATGACGGTAAATGGCCCGCCTG | |
| | GCATTATGCCCAGTACATGACCTTACGGGACTTTCCT | |
| | ACTTGGCAGTACATCTACGTATTAGTCATCGCTATTA | |
| | CCATGGTGATGCGGTTTTGGCAGTACACCAATGGGCG | |
| | TGGATAGCGGTTTGACTCACGGGGATTTCCAAGTCTC | |
| | CACCCCATTGACGTCAATGGGAGTTTGTTTTGGCACC | |
| | AAAATCAACGGGACTTTCCAAAATGTCGTAATAACC | |
| | CCGCCCCGTTGACGCAAATGGGCGGTAGGCGTGTAC | |
| | GGTGGGAGGTCTATATAAGCAGAGCTCGTTTAGTGA | |
| | ACCGTCAGATCCTCACTCTCTTCCGCATCGCTGTCTG | |
| | CGAGGGCCAGCTGTTGGGCTCGCGGTTGAGGACAAA | |
| | CTCTTCGCGGTCTTTCCAGTACTCTTGGATCGGAAAC | |
| | CCGTCGGCCTCCGAACGGTACTCCGCCACCGAGGGA | |
| | CCTGAGCGAGTCCGCATCGACCGGATCGGAAAACCT | |
| | CTCGAGAAAGGCGTCTAACCAGTCACAGTCGCAAGG | |
| | TAGGCTGAGCACCGTGGCGGGCGGCAGCGGGTGGCG | |
| | GTCGGGGTTGTTTCTGGCGGAGGTGCTGCTGATGATG | |
| | TAATTAAAGTAGGCGGTCTTGAGACGGCGGATGGTC | |
| | GAGGTGAGGTGTGGCAGGCTTGAGATCCAGCTGTTG | |
| | GGGTGAGTACTCCCTCTCAAAAGCGGGCATTACTTCT | |
| | GCGCTAAGATTGTCAGTTTCCAAAAACGAGGAGGAT | |
| | TTGATATTCACCTGGCCCGATCTGGCCATACACTTGA | |
| | GTGACAATGACATCCACTTTGCCTTTCTCTCCACAGG | |
| | TGTCCACTCCCAGGTCCAAGTTTAAACTTTAATACGA | |
| | CTCACTATAGGGGCCGCCACCAAGCTTGGTACCatggat | |
| | gcaatgaagagagggctctgctgtgtgctgctgctgtgtggagcagtcttcgtttcgccc | |
| | agccaggaaatccatgcccgattcagaagaggagccagatcttaccaagtgatctgca | |
| | gagatgaaaaaacgcagatgatataccagcaacatcagtcatggctgcgccctgtgctc | |
| | agaagcaaccgggtggaatattgctggtgcaacagtggcagggcacagtgccactca | |
| | gtgcctgtcaaaagttgcagcgagccaaggtgtttcaacgggggcacctgccagcagg | |
| | ccctgtacttctcagatttcgtgtgccagtgccccgaaggatttgctgggaagtgctgtga | |
| | aatagataccagggccacgtgctacgaggaccagggcatcagctacaggggcacgtg | |
| | gagcacagcggagagtggcgccgagtgcaccaactggaacagcagcgcgttggccc | |
| | agaagccctacagcgggcggaggccagacgccatcaggctgggcctggggaacca | |
| | caactactgcagaaacccagatcgagactcaaagccctggtgctacgtctttaaggcgg | |
| | ggaagtacagctcagagttctgcagcacccctgcctgctctgagggaaacagtgactgc | |
| | tactttgggaatgggtcagcctaccgtggcacgcacagcctcaccgagtcgggtgcctc | |
| | ctgcctcccgtggaattccatgatcctgataggcaaggtttacacagcacagaacccca | |
| | gtgcccaggcactgggcctgggcaaacataattactgccggaatcctgatggggatgc | |
| | caagccctggtgccacgtgctgaagaaccgcaggctgacgtgggagtactgtgatgtg | |
| | ccctcctgctccacctgcggcctgagacagtacagccagcctcagtttcgcatcaaagg | |
| | agggctcttcgccgacatcgcctcccacccctggcaggctgccatctttgccaagcaca | |
| | ggaggtcgcccggagagcggttcctgtgcgggggcatactcatcagctcctgctggatt | |
| | ctctctgccgcccactgcttccaggagaggtttccgccccaccacctgacggtgatcttg | |
| | ggcagaacataccgggtggtccctggcgaggaggagcagaaatttgaagtcgaaaaa | |
| | tacattgtccataaggaattcgatgatgacacttacgacaatgacattgcgctgctgcagc | |
| | tgaaatcggattcgtcccgctgtgcccaggagagcagcgtggtccgcactgtgtgcctt | |
| | cccccggcggacctgcagctgccggactggacggagtgtgagctctccggctacggc | |
| | aagcatgaggccttgtctcctttctattcggagcggctgaaggaggctcatgtcagactgt | |
| | acccatccagccgctgcacatcacaacatttacttaacagaacagtcaccgacaacatg | |
| | ctgtgtgctggagacactcggagcggcgggccccaggcaaacttgcacgacgcctgc | |
| | cagggcgattcgggaggccccctggtgtgtctgaacgatggccgcatgactttggtgg | |
| | gcatcatcagctggggcctgggctgtggacagaaggatgtcccgggtgtgtacaccaa | |
| | ggttaccaactacctagactggattcgtgacaacatgcgaccgGGGGGTGGAG | |
| | GCTCTTATCCTTACGACGTGCCTGACTACGCCTAAAC | |
| | TCGAGTCTAGAGCGGCCGCCGAATTCGGGCCCGTTTA | |
| | AACCCGCTGATCAGCCTCGACTGTGCCTTCTAGTTGC | |
| | CAGCCATCTGTTGTTTGCCCCTCCCCCGTGCCTTCCTT | |
| | GACCCTGGAAGGTGCCACTCCCACTGTCCTTTCCTAA | |
| | TAAAATGAGGAAATTGCATCGCATTGTCTGAGTAGGT | |
| | GTCATTCTATTCTGGGGGGTGGGGTGGGGCAGGACA | |
| | GCAAGGGGGAGGATTGGGAAGACAATAGCAGGCAT | |
| | GCTGGGGATGCGGTGGGCTCTATGGCTTCTGAGGCG | |
| | GAAAGAACCAGCTGGGGCTCTAGGGGGTATCCCCAC | |
| | GCGCCCTGTAGCGGCGCATTAAGCGCGGCGGGTGTG | |
| | GTGGTTACGCGCAGCGTGACCGCTACACTTGCCAGCG | |
| | CCCTAGCGCCCGCTCCTTTCGCTTTCTTCCCTTCCTTT | |
| | CTCGCCACGTTCGCAGGCTTTCCCCGTCAAGCTCTAA | |
| | ATCGGGGGCTCCCTTTAGGGTTCCGATTTAGTGCTTT | |
| | ACGGCACCTCGACCCCAAAAAACTTGATTAGGGTGA | |
| | TGGTTCACGTAGTGGGCCATCGCCCTGATAGACGGTT | |
| | TTTCGCCCTTTGACGTTGGAGTCCACGTTCTTTAATAG | |
| | TGGACTCTTGTTCCAAACTGGAACAACACTCAACCCT | |
| | ATCTCGGTCTATTCTTTTGATTTATAAGGGATTTTGCC | |
| | GATTTCGGCCTATTGGTTAAAAAATGAGCTGATTTAA | |
| | CAAAAATTTAACGCGAATTAATTCTGTGGAATGTGTG | |
| | TCAGTTAGGGTGTGGAAAGTCCCCAGGCTCCCCAGC | |
| | AGGCAGAAGTATGCAAAGCATGCATCTCAATTAGTC | |
| | AGCAACCAGGTGTGGAAAGTCCCCAGGCTCCCCAGC | |
| | AGGCAGAAGTATGCAAAGCATGCATCTCAATTAGTC | |
| | AGCAACCATAGTCCCGCCCCTAACTCCGCCCATCCCG | |
| | CCCCTAACTCCGCCCAGTTCCGCCCATTCTCCGCCCC | |
| | ATGGCTGACTAATTTTTTTTATTTATGCAGAGGCCGA | |
| | GGCCGCCTCTGCCTCTGAGCTATTCCAGAAGTAGTGA | |
| | GGAGGCTTTTTTGGAGGCCTAGGCTTTTGCAAAAAGC | |
| | TCTCGGGAGCTTGTATATCCATTTTCGGATCTGATCA | |
| | GCACGTGATGAAAAAGCCTGAACTCACCGCGACGTC | |
| | TGTCGAGAAGTTTCTGATCGAAAAGTTCGACAGCGTC | |
| | TCCGACCTGATGCAGCTCTCGGAGGGCGAAGAATCT | |
| | CGTGCTTTCAGCTTCGATGTAGGAGGGCGTGGATATG | |
| | TCCTGCGGGTAAATAGCTGCGCCGATGGTTTCTACAA | |
| | AGATCGTTATGTTTATCGGCACTTTGCATCGGCCGCG | |
| | CTCCCGATTCCGGAAGTGCTTGACATTGGGGAATTCA | |
| | GCGAGAGCCTGACCTATTGCATCTCCCGCCGTGCACA | |
| | GGGTGTCACGTTGCAAGACCTGCCTGAAACCGAACT | |
| | GCCCGCTGTTCTGCAGCCGGTCGCGGAGGCCATGGAT | |
| | GCGATCGCTGCGGCCGATCTTAGCCAGACGAGCGGG | |
| | TTCGGCCCATTCGGACCGCAAGGAATCGGTCAATAC | |
| | ACTACATGGCGTGATTTCATATGCGCGATTGCTGATC | |
| | CCCATGTGTATCACTGGCAAACTGTGATGGACGACAC | |
| | CGTCAGTGCGTCCGTCGCGCAGGCTCTCGATGAGCTG | |
| | ATGCTTTGGGCCGAGGACTGCCCCGAAGTCCGGCAC | |
| | CTCGTGCACGCGGATTTCGGCTCCAACAATGTCCTGA | |
| | CGGACAATGGCCGCATAACAGCGGTCATTGACTGGA | |
| | GCGAGGCGATGTTCGGGGATTCCCAATACGAGGTCG | |
| | CCAACATCTTCTTCTGGAGGCCGTGGTTGGCTTGTAT | |
| | GGAGCAGCAGACGCGCTACTTCGAGCGGAGGCATCC | |
| | GGAGCTTGCAGGATCGCCGCGGCTCCGGGCGTATAT | |
| | GCTCCGCATTGGTCTTGACCAACTCTATCAGAGCTTG | |
| | GTTGACGGCAATTTCGATGATGCAGCTTGGGCGCAG | |
| | GGTCGATGCGACGCAATCGTCCGATCCGGAGCCGGG | |
| | ACTGTCGGGCGTACACAAATCGCCCGCAGAAGCGCG | |
| | GCCGTCTGGACCGATGGCTGTGTAGAAGTACTCGCCG | |
| | ATAGTGGAAACCGACGCCCCAGCACTCGTCCGAGGG | |
| | CAAAGGAATAGCACGTGCTACGAGATTTCGATTCCA | |
| | CCGCCGCCTTCTATGAAAGGTTGGGCTTCGGAATCGT | |
| | TTTCCGGGACGCTGGCTGGATGATCCTCCAGCGCGGG | |
| | GATCTCATGCTGGAGTTCTTCGCCCACCCCAACTTGT | |
| | TTATTGCAGCTTATAATGGTTACAAATAAAGCAATAG | |
| | CATCACAAATTTCACAAATAAAGCATTTTTTTCACTG | |
| | CATTCTAGTTGTGGTTTGTCCAAACTCATCAATGTAT | |
| | CTTATCATGTCTGTATACCGTCGACCTCTAGCTAGAG | |
| | CTTGGCGTAATCATGGTCATAGCTGTTTCCTGTGTGA | |
| | AATTGTTATCCGCTCACAATTCCACACAACATACGAG | |
| | CCGGAAGCATAAAGTGTAAAGCCTGGGGTGCCTAAT | |
| | GAGTGAGCTAACTCACATTAATTGCGTTGCGCTCACT | |
| | GCCCGCTTTCCAGTCGGGAAACCTGTCGTGCCAGCTG | |
| | CATTAATGAATCGGCCAACGCGCGGGGAGAGGCGGT | |
| | TTGCGTATTGGGCGCTCTTCCGCTTCCTCGCTCACTGA | |
| | CTCGCTGCGCTCGGTCGTTCGGCTGCGGCGAGCGGTA | |
| | TCAGCTCACTCAAAGGCGGTAATACGGTTATCCACAG | |
| | AATCAGGGGATAACGCAGGAAAGAACATGTGAGCAA | |
| | AAGGCCAGCAAAAGGCCAGGAACCGTAAAAAGGCC | |
| | GCGTTGCTGGCGTTTTTCCATAGGCTCCGCCCCCCTG | |
| | ACGAGCATCACAAAAATCGACGCTCAAGTCAGAGGT | |
| | GGCGAAACCCGACAGGACTATAAAGATACCAGGCGT | |
| | TTCCCCCTGGAAGCTCCCTCGTGCGCTCTCCTGTTCCG | |
| | ACCCTGCCGCTTACCGGATACCTGTCCGCCTTTCTCC | |
| | CTTCGGGAAGCGTGGCGCTTTCTCATAGCTCACGCTG | |
| | TAGGTATCTCAGTTCGGTGTAGGTCGTTCGCTCCAAG | |
| | CTGGGCTGTGTGCACGAACCCCCCGTTCAGCCCGACC | |
| | GCTGCGCCTTATCCGGTAACTATCGTCTTGAGTCCAA | |
| | CCCGGTAAGACACGACTTATCGCCACTGGCAGCAGC | |
| | CACTGGTAACAGGATTAGCAGAGCGAGGTATGTAGG | |
| | CGGTGCTACAGAGTTCTTGAAGTGGTGGCCTAACTAC | |
| | GGCTACACTAGAAGAACAGTATTTGGTATCTGCGCTC | |
| | TGCTGAAGCCAGTTACCTTCGGAAAAAGAGTTGGTA | |
| | GCTCTTGATCCGGCAAACAAACCACCGCTGGTAGCG | |
| | GTGGTTTTTTTGTTTGCAAGCAGCAGATTACGCGCAG | |
| | AAAAAAAGGATCTCAAGAAGATCCTTTGATCTTTTCT | |
| | ACGGGGTCTGAGCGCGGAACCCCTATTTGTTTATTTT | |
| | TCTAAATACATTCAAATATGTATCCGCTCATGAATTA | |
| | ATTCTTAGAAAAACTCATCGAGCATCAAATGAAACT | |
| | GCAATTTATTCATATCAGGATTATCAATACCATATTT | |
| | TTGAAAAAGCCGTTTCTGTAATGAAGGAGAAAACTC | |
| | ACCGAGGCAGTTCCATAGGATGGCAAGATCCTGGTA | |
| | TCGGTCTGCGATTCCGACTCGTCCAACATCAATACAA | |
| | CCTATTAATTTCCCCTCGTCAAAAATAAGGTTATCAA | |
| | GTGAGAAATCACCATGAGTGACGACTGAATCCGGTG | |
| | AGAATGGCAAAAGTTTATGCATTTCTTTCCAGACTTG | |
| | TTCAACAGGCCAGCCATTACGCTCGTCATCAAAATCA | |
| | CTCGCATCAACCAAACCGTTATTCATTCGTGATTGCG | |
| | CCTGAGCGAGACGAAATACGCGATCGCTGTTAAAAG | |
| | GACAATTACAAACAGGAATCGAATGCAACCGGCGCA | |
| | GGAACACTGCCAGCGCATCAACAATATTTTCACCTGA | |
| | ATCAGGATATTCTTCTAATACCTGGAATGCTGTTTTC | |
| | CCAGGGATCGCAGTGGTGAGTAACCATGCATCATCA | |
| | GGAGTACGGATAAAATGCTTGATGGTCGGAAGAGGC | |
| | ATAAATTCCGTCAGCCAGTTTAGTCTGACCATCTCAT | |
| | CTGTAACATCATTGGCAACGCTACCTTTGCCATGTTT | |
| | CAGAAACAACTCTGGCGCATCGGGCTTCCCATACAAT | |
| | CGATAGATTGTCGCACCTGATTGCCCGACATTATCGC | |
| | GAGCCCATTTATACCCATATAAATCAGCATCCATGTT | |
| | GGAATTTAATCGCGGCCTAGAGCAAGACGTTTCCCGT | |
| | TGAATATGGCTCATAACACCCCTTGTATTACTGTTTA | |
| | TGTAAGCAGACAGTTTTATTGTTCATGACCAAAATCC | |
| | CTTAACGTGAGTTTTCGTTCCACTGAGCGTCAGACCC | |
| | CGTAGAAATCCGCGCACATTTCCCCGAAAAGTGCCA | |
| | CCTGACGTC | |
| 30 | MDAMKRGLCCVLLLCGAVFVSPSQEIHARFRRGARSY | tPA heavy chain, |
| | QVICRDEKTQMIYQQHQSWLRPVLRSNRVEYCWCNSG | lacking the |
| | RAQCHSVPVKSCSEPRCFNGGTCQQALYFSDFVCQCPE | protease domain |
| | GFAGKCCEIDTRATCYEDQGISYRGTWSTAESGAECTN | (kringle 2 |
| | WNSSALAQKPYSGRRPDAIRLGLGNHNYCRNPDRDSKP | domain |
| | WCYVFKAGKYSSEFCSTPACSEGNSDCYFGNGSAYRGT | underlined), |
| | HSLTESGASCLPWNSMILIGKVYTAQNPSAQALGLGKH | amino acid |
| | NYCRNPDGDAKPWCHVLKNRRLTWEYCDVPSCSTCGL | sequence |
| | RQYSQPQFR | |
| 31 | STPACSEGNSDCYFGNGSAYRGTHSLTESGASCLPWNS | tPA-K2-HA- |
| | MILIGKVYTAQNPSAQALGLGKHNYCRNPDGDAKPWC | KDEL amino |
| | HVLKNRRLTWEYCDVPSCSTCGLRQYSQPQFRGGGGS | acid sequence |
| | YPYDVPDYAKDEL | (HA tag |
| | | sequence bold |
| | | underlined) |
| 32 | Agcaccccugccugcucugagggaaacagugacugcuacuuugggaaugggu | tPA-K2-HA- |
| | cagccuaccguggcacgcacagccucaccgagucgggugccuccugccucccgu | KDEL coding |
| | ggaauuccaugauccugauaggcaagguuuacacagcacagaaccccagugccc | sequence in |
| | aggcacugggccugggcaaacauaauuacugccggaauccugauggggaugcc | mRNA (HA tag |
| | aagcccuggugccacgugcugaagaaccgcaggcugacgugggaguacuguga | sequence bold |
| | ugugcccuccugcuccaccugcggccugagacaguacagccagccucaguuuc | underlined) |
| | gc | |
| | GGGGGUGGAGGCUCU | |
| | UAUCCUUACGACGUGCCUGACUACGCC | |
| | AAGGAUGAACUU | |
| 33 | GACGGATCGGGAGATCTCCCGATCCCCTATGGTGCAC | pCMV3-tPA- |
| | TCTCAGTACAATCTGCTCTGATGCCGCATAGTTAAGC | K2-HA-KDEL |
| | CAGTATCTGCTCCCTGCTTGTGTGTTGGAGGTCGCTG | (plasmid |
| | AGTAGTGCGCGAGCAAAATTTAAGCTACAACAAGGC | encoding tPA- |
| | AAGGCTTGACCGACAATTGCATGAAGAATCTGCTTA | K2-HA-KDEL) |
| | GGGTTAGGCGTTTTGCGCTGCTTCGCGAGTACATTTA | |
| | TATTGGCTCATGTCCAATATGACCGCCATGTTGACAT | |
| | TGATTATTGACTAGTTATTAATAGTAATCAATTACGG | |
| | GGTCATTAGTTCATAGCCCATATATGGAGTTCCGCGT | |
| | TACATAACTTACGGTAAATGGCCCGCCTGGCTGACCG | |
| | CCCAACGACCCCCGCCCATTGACGTCAATAATGACGT | |
| | ATGTTCCCATAGTAACGCCAATAGGGACTTTCCATTG | |
| | ACGTCAATGGGTGGAGTATTTACGGTAAACTGCCCAC | |
| | TTGGCAGTACATCAAGTGTATCATATGCCAAGTCCGC | |
| | CCCCTATTGACGTCAATGACGGTAAATGGCCCGCCTG | |
| | GCATTATGCCCAGTACATGACCTTACGGGACTTTCCT | |
| | ACTTGGCAGTACATCTACGTATTAGTCATCGCTATTA | |
| | CCATGGTGATGCGGTTTTGGCAGTACACCAATGGGCG | |
| | TGGATAGCGGTTTGACTCACGGGGATTTCCAAGTCTC | |
| | CACCCCATTGACGTCAATGGGAGTTTGTTTTGGCACC | |
| | AAAATCAACGGGACTTTCCAAAATGTCGTAATAACC | |
| | CCGCCCCGTTGACGCAAATGGGCGGTAGGCGTGTAC | |
| | GGTGGGAGGTCTATATAAGCAGAGCTCGTTTAGTGA | |
| | ACCGTCAGATCCTCACTCTCTTCCGCATCGCTGTCTG | |
| | CGAGGGCCAGCTGTTGGGCTCGCGGTTGAGGACAAA | |
| | CTCTTCGCGGTCTTTCCAGTACTCTTGGATCGGAAAC | |
| | CCGTCGGCCTCCGAACGGTACTCCGCCACCGAGGGA | |
| | CCTGAGCGAGTCCGCATCGACCGGATCGGAAAACCT | |
| | CTCGAGAAAGGCGTCTAACCAGTCACAGTCGCAAGG | |
| | TAGGCTGAGCACCGTGGCGGGCGGCAGCGGGTGGCG | |
| | GTCGGGGTTGTTTCTGGCGGAGGTGCTGCTGATGATG | |
| | TAATTAAAGTAGGCGGTCTTGAGACGGCGGATGGTC | |
| | GAGGTGAGGTGTGGCAGGCTTGAGATCCAGCTGTTG | |
| | GGGTGAGTACTCCCTCTCAAAAGCGGGCATTACTTCT | |
| | GCGCTAAGATTGTCAGTTTCCAAAAACGAGGAGGAT | |
| | TTGATATTCACCTGGCCCGATCTGGCCATACACTTGA | |
| | GTGACAATGACATCCACTTTGCCTTTCTCTCCACAGG | |
| | TGTCCACTCCCAGGTCCAAGTTTAAACTTTAATACGA | |
| | CTCACTATAGGGGCCGCCACCAAGCTTGGTACCatggat | |
| | gcaatgaagagagggctctgctgtgtgctgctgctgtgtggagcagtcttcgtttcgccc | |
| | agccaggaaatccatgcccgattcagaagaggagccagatcttaccaagtgatctgca | |
| | gcacccctgcctgctctgagggaaacagtgactgctactttgggaatgggtcagcctac | |
| | cgtggcacgcacagcctcaccgagtcgggtgcctcctgcctcccgtggaattccatgat | |
| | cctgataggcaaggtttacacagcacagaaccccagtgcccaggcactgggcctggg | |
| | caaacataattactgccggaatcctgatggggatgccaagccctggtgccacgtgctga | |
| | agaaccgcaggctgacgtgggagtactgtgatgtgccctcctgctccacctgcggcctg | |
| | agacagtacagccagcctcagtttcgcGGGGGTGGAGGCTCTTATCC | |
| | TTACGACGTGCCTGACTACGCCAAGGATGAACTTTAA | |
| | ACTCGAGTCTAGAGCGGCCGCCGAATTCGGGCCCGTT | |
| | TAAACCCGCTGATCAGCCTCGACTGTGCCTTCTAGTT | |
| | GCCAGCCATCTGTTGTTTGCCCCTCCCCCGTGCCTTCC | |
| | TTGACCCTGGAAGGTGCCACTCCCACTGTCCTTTCCT | |
| | AATAAAATGAGGAAATTGCATCGCATTGTCTGAGTA | |
| | GGTGTCATTCTATTCTGGGGGGTGGGGTGGGGCAGG | |
| | ACAGCAAGGGGGAGGATTGGGAAGACAATAGCAGG | |
| | CATGCTGGGGATGCGGTGGGCTCTATGGCTTCTGAGG | |
| | CGGAAAGAACCAGCTGGGGCTCTAGGGGGTATCCCC | |
| | ACGCGCCCTGTAGCGGCGCATTAAGCGCGGCGGGTG | |
| | TGGTGGTTACGCGCAGCGTGACCGCTACACTTGCCAG | |
| | CGCCCTAGCGCCCGCTCCTTTCGCTTTCTTCCCTTCCT | |
| | TTCTCGCCACGTTCGCAGGCTTTCCCCGTCAAGCTCT | |
| | AAATCGGGGGCTCCCTTTAGGGTTCCGATTTAGTGCT | |
| | TTACGGCACCTCGACCCCAAAAAACTTGATTAGGGTG | |
| | ATGGTTCACGTAGTGGGCCATCGCCCTGATAGACGGT | |
| | TTTTCGCCCTTTGACGTTGGAGTCCACGTTCTTTAATA | |
| | GTGGACTCTTGTTCCAAACTGGAACAACACTCAACCC | |
| | TATCTCGGTCTATTCTTTTGATTTATAAGGGATTTTGC | |
| | CGATTTCGGCCTATTGGTTAAAAAATGAGCTGATTTA | |
| | ACAAAAATTTAACGCGAATTAATTCTGTGGAATGTGT | |
| | GTCAGTTAGGGTGTGGAAAGTCCCCAGGCTCCCCAG | |
| | CAGGCAGAAGTATGCAAAGCATGCATCTCAATTAGT | |
| | CAGCAACCAGGTGTGGAAAGTCCCCAGGCTCCCCAG | |
| | CAGGCAGAAGTATGCAAAGCATGCATCTCAATTAGT | |
| | CAGCAACCATAGTCCCGCCCCTAACTCCGCCCATCCC | |
| | GCCCCTAACTCCGCCCAGTTCCGCCCATTCTCCGCCC | |
| | CATGGCTGACTAATTTTTTTTATTTATGCAGAGGCCG | |
| | AGGCCGCCTCTGCCTCTGAGCTATTCCAGAAGTAGTG | |
| | AGGAGGCTTTTTTGGAGGCCTAGGCTTTTGCAAAAAG | |
| | CTCTCGGGAGCTTGTATATCCATTTTCGGATCTGATC | |
| | AGCACGTGATGAAAAAGCCTGAACTCACCGCGACGT | |
| | CTGTCGAGAAGTTTCTGATCGAAAAGTTCGACAGCGT | |
| | CTCCGACCTGATGCAGCTCTCGGAGGGCGAAGAATC | |
| | TCGTGCTTTCAGCTTCGATGTAGGAGGGCGTGGATAT | |
| | GTCCTGCGGGTAAATAGCTGCGCCGATGGTTTCTACA | |
| | AAGATCGTTATGTTTATCGGCACTTTGCATCGGCCGC | |
| | GCTCCCGATTCCGGAAGTGCTTGACATTGGGGAATTC | |
| | AGCGAGAGCCTGACCTATTGCATCTCCCGCCGTGCAC | |
| | AGGGTGTCACGTTGCAAGACCTGCCTGAAACCGAAC | |
| | TGCCCGCTGTTCTGCAGCCGGTCGCGGAGGCCATGGA | |
| | TGCGATCGCTGCGGCCGATCTTAGCCAGACGAGCGG | |
| | GTTCGGCCCATTCGGACCGCAAGGAATCGGTCAATA | |
| | CACTACATGGCGTGATTTCATATGCGCGATTGCTGAT | |
| | CCCCATGTGTATCACTGGCAAACTGTGATGGACGACA | |
| | CCGTCAGTGCGTCCGTCGCGCAGGCTCTCGATGAGCT | |
| | GATGCTTTGGGCCGAGGACTGCCCCGAAGTCCGGCA | |
| | CCTCGTGCACGCGGATTTCGGCTCCAACAATGTCCTG | |
| | ACGGACAATGGCCGCATAACAGCGGTCATTGACTGG | |
| | AGCGAGGCGATGTTCGGGGATTCCCAATACGAGGTC | |
| | GCCAACATCTTCTTCTGGAGGCCGTGGTTGGCTTGTA | |
| | TGGAGCAGCAGACGCGCTACTTCGAGCGGAGGCATC | |
| | CGGAGCTTGCAGGATCGCCGCGGCTCCGGGCGTATA | |
| | TGCTCCGCATTGGTCTTGACCAACTCTATCAGAGCTT | |
| | GGTTGACGGCAATTTCGATGATGCAGCTTGGGCGCA | |
| | GGGTCGATGCGACGCAATCGTCCGATCCGGAGCCGG | |
| | GACTGTCGGGCGTACACAAATCGCCCGCAGAAGCGC | |
| | GGCCGTCTGGACCGATGGCTGTGTAGAAGTACTCGCC | |
| | GATAGTGGAAACCGACGCCCCAGCACTCGTCCGAGG | |
| | GCAAAGGAATAGCACGTGCTACGAGATTTCGATTCC | |
| | ACCGCCGCCTTCTATGAAAGGTTGGGCTTCGGAATCG | |
| | TTTTCCGGGACGCTGGCTGGATGATCCTCCAGCGCGG | |
| | GGATCTCATGCTGGAGTTCTTCGCCCACCCCAACTTG | |
| | TTTATTGCAGCTTATAATGGTTACAAATAAAGCAATA | |
| | GCATCACAAATTTCACAAATAAAGCATTTTTTTCACT | |
| | GCATTCTAGTTGTGGTTTGTCCAAACTCATCAATGTA | |
| | TCTTATCATGTCTGTATACCGTCGACCTCTAGCTAGA | |
| | GCTTGGCGTAATCATGGTCATAGCTGTTTCCTGTGTG | |
| | AAATTGTTATCCGCTCACAATTCCACACAACATACGA | |
| | GCCGGAAGCATAAAGTGTAAAGCCTGGGGTGCCTAA | |
| | TGAGTGAGCTAACTCACATTAATTGCGTTGCGCTCAC | |
| | TGCCCGCTTTCCAGTCGGGAAACCTGTCGTGCCAGCT | |
| | GCATTAATGAATCGGCCAACGCGCGGGGAGAGGCGG | |
| | TTTGCGTATTGGGCGCTCTTCCGCTTCCTCGCTCACTG | |
| | ACTCGCTGCGCTCGGTCGTTCGGCTGCGGCGAGCGGT | |
| | ATCAGCTCACTCAAAGGCGGTAATACGGTTATCCACA | |
| | GAATCAGGGGATAACGCAGGAAAGAACATGTGAGCA | |
| | AAAGGCCAGCAAAAGGCCAGGAACCGTAAAAAGGC | |
| | CGCGTTGCTGGCGTTTTTCCATAGGCTCCGCCCCCCT | |
| | GACGAGCATCACAAAAATCGACGCTCAAGTCAGAGG | |
| | TGGCGAAACCCGACAGGACTATAAAGATACCAGGCG | |
| | TTTCCCCCTGGAAGCTCCCTCGTGCGCTCTCCTGTTCC | |
| | GACCCTGCCGCTTACCGGATACCTGTCCGCCTTTCTC | |
| | CCTTCGGGAAGCGTGGCGCTTTCTCATAGCTCACGCT | |
| | GTAGGTATCTCAGTTCGGTGTAGGTCGTTCGCTCCAA | |
| | GCTGGGCTGTGTGCACGAACCCCCCGTTCAGCCCGAC | |
| | CGCTGCGCCTTATCCGGTAACTATCGTCTTGAGTCCA | |
| | ACCCGGTAAGACACGACTTATCGCCACTGGCAGCAG | |
| | CCACTGGTAACAGGATTAGCAGAGCGAGGTATGTAG | |
| | GCGGTGCTACAGAGTTCTTGAAGTGGTGGCCTAACTA | |
| | CGGCTACACTAGAAGAACAGTATTTGGTATCTGCGCT | |
| | CTGCTGAAGCCAGTTACCTTCGGAAAAAGAGTTGGT | |
| | AGCTCTTGATCCGGCAAACAAACCACCGCTGGTAGC | |
| | GGTGGTTTTTTTGTTTGCAAGCAGCAGATTACGCGCA | |
| | GAAAAAAAGGATCTCAAGAAGATCCTTTGATCTTTTC | |
| | TACGGGGTCTGAGCGCGGAACCCCTATTTGTTTATTT | |
| | TTCTAAATACATTCAAATATGTATCCGCTCATGAATT | |
| | AATTCTTAGAAAAACTCATCGAGCATCAAATGAAAC | |
| | TGCAATTTATTCATATCAGGATTATCAATACCATATT | |
| | TTTGAAAAAGCCGTTTCTGTAATGAAGGAGAAAACT | |
| | CACCGAGGCAGTTCCATAGGATGGCAAGATCCTGGT | |
| | ATCGGTCTGCGATTCCGACTCGTCCAACATCAATACA | |
| | ACCTATTAATTTCCCCTCGTCAAAAATAAGGTTATCA | |
| | AGTGAGAAATCACCATGAGTGACGACTGAATCCGGT | |
| | GAGAATGGCAAAAGTTTATGCATTTCTTTCCAGACTT | |
| | GTTCAACAGGCCAGCCATTACGCTCGTCATCAAAATC | |
| | ACTCGCATCAACCAAACCGTTATTCATTCGTGATTGC | |
| | GCCTGAGCGAGACGAAATACGCGATCGCTGTTAAAA | |
| | GGACAATTACAAACAGGAATCGAATGCAACCGGCGC | |
| | AGGAACACTGCCAGCGCATCAACAATATTTTCACCTG | |
| | AATCAGGATATTCTTCTAATACCTGGAATGCTGTTTT | |
| | CCCAGGGATCGCAGTGGTGAGTAACCATGCATCATC | |
| | AGGAGTACGGATAAAATGCTTGATGGTCGGAAGAGG | |
| | CATAAATTCCGTCAGCCAGTTTAGTCTGACCATCTCA | |
| | TCTGTAACATCATTGGCAACGCTACCTTTGCCATGTT | |
| | TCAGAAACAACTCTGGCGCATCGGGCTTCCCATACAA | |
| | TCGATAGATTGTCGCACCTGATTGCCCGACATTATCG | |
| | CGAGCCCATTTATACCCATATAAATCAGCATCCATGT | |
| | TGGAATTTAATCGCGGCCTAGAGCAAGACGTTTCCCG | |
| | TTGAATATGGCTCATAACACCCCTTGTATTACTGTTT | |
| | ATGTAAGCAGACAGTTTTATTGTTCATGACCAAAATC | |
| | CCTTAACGTGAGTTTTCGTTCCACTGAGCGTCAGACC | |
| | CCGTAGAAATCCGCGCACATTTCCCCGAAAAGTGCC | |
| | ACCTGACGTC | |
| 34 | Agcacccctgcctgctctgagggaaacagtgactgctactttgggaatgggtcagcct | tPA kringle 2 |
| | accgtggcacgcacagcctcaccgagtcgggtgcctcctgcctcccgtggaattccat | KDEL encoding |
| | gatcctgataggcaaggtttacacagcacagaaccccagtgcccaggcactgggcctg | nucleic acid |
| | ggcaaacataattactgccggaatcctgatggggatgccaagccctggtgccacgtgc | sequence |
| | tgaagaaccgcaggctgacgtgggagtactgtgatgtgccctcctgctccacctgcgg | |
| | cctgagacagtacagccagcctcagtttcgcGGGGGTGGAGGCTCTAA | |
| | GGATGAACTT | |
| 35 | MDAMKRGLCCVLLLCGAVFVSPSQEIHARFRRGARSY | tPA-S513A- |
| | QVICRDEKTQMIYQQHQSWLRPVLRSNRVEYCWCNSG | KDEL mutant |
| | RAQCHSVPVKSCSEPRCFNGGTCQQALYFSDFVCQCPE | (kringle 2 |
| | GFAGKCCEIDTRATCYEDQGISYRGTWSTAESGAECTN | domain |
| | WNSSALAQKPYSGRRPDAIRLGLGNHNYCRNPDRDSKP | underlined; |
| | WCYVFKAGKYSSEFCSTPACSEGNSDCYFGNGSAYRGT | GGGGS linker |
| | HSLTESGASCLPWNSMILIGKVYTAQNPSAQALGLGKH | in bold; S513A |
| | NYCRNPDGDAKPWCHVLKNRRLTWEYCDVPSCSTCGL | bold underlined) |
| | RQYSQPQFRIKGGLFADIASHPWQAAIFAKHRRSPGERF | |
| | LCGGILISSCWILSAAHCFQERFPPHHLTVILGRTYRVVP | |
| | GEEEQKFEVEKYIVHKEFDDDTYDNDIALLQLKSDSSRC | |
| | AQESSVVRTVCLPPADLQLPDWTECELSGYGKHEALSP | |
| | FYSERLKEAHVRLYPSSRCTSQHLLNRTVTDNMLCAGD | |
| | TRSGGPQANLHDACQGDAGGPLVCLNDGRMTLVGIIS | |
| | WGLGCGQKDVPGVYTKVTNYLDWIRDNMRP | |
| | GGGGSKDEL | |
| 36 | ATGGATGCAATGAAGAGAGGGCtctgctgtgtgctgctgctgtgtg | tPA-S513A- |
| | gagcagtcttcgtttcgcccagccaggaaatccatgcccgattcagaagaggagccag | KDEL mutant |
| | atcttaccaagtgatctgcagagatgaaaaaacgcagatgatataccagcaacatcagtc | encoding nucleic |
| | atggctgcgccctgtgctcagaagcaaccgggtggaatattgctggtgcaacagtggca | acid sequence |
| | gggcacagtgccactcagtgcctgtcaaaagttgcagcgagccaaggtgtttcaacgg | |
| | gggcacctgccagcaggccctgtacttctcagatttcgtgtgccagtgccccgaaggatt | |
| | tgctgggaagtgctgtgaaatagataccagggccacgtgctacgaggaccagggcatc | |
| | agctacaggggcacgtggagcacagcggagagtggcgccgagtgcaccaactggaa | |
| | cagcagcgcgttggcccagaagccctacagcgggcggaggccagacgccatcaggc | |
| | tgggcctggggaaccacaactactgcagaaacccagatcgagactcaaagccctggtg | |
| | ctacgtctttaaggcggggaagtacagctcagagttctgcagcacccctgcctgctctga | |
| | gggaaacagtgactgctactttgggaatgggtcagcctaccgtggcacgcacagcctca | |
| | ccgagtcgggtgcctcctgcctcccgtggaattccatgatcctgataggcaaggtttaca | |
| | cagcacagaaccccagtgcccaggcactgggcctgggcaaacataattactgccgga | |
| | atcctgatggggatgccaagccctggtgccacgtgctgaagaaccgcaggctgacgtg | |
| | ggagtactgtgatgtgccctcctgctccacctgcggcctgagacagtacagccagcctc | |
| | agtttcgcatcaaaggagggctcttcgccgacatcgcctcccacccctggcaggctgcc | |
| | atctttgccaagcacaggaggtcgcccggagagcggttcctgtgcgggggcatactcat | |
| | cagctcctgctggattctctctgccgcccactgcttccaggagaggtttccgccccacca | |
| | cctgacggtgatcttgggcagaacataccgggtggtccctggcgaggaggagcagaa | |
| | atttgaagtcgaaaaatacattgtccataaggaattcgatgatgacacttacgacaatgac | |
| | attgcgctgctgcagctgaaatcggattcgtcccgctgtgcccaggagagcagcgtggt | |
| | ccgcactgtgtgccttcccccggcggacctgcagctgccggactggacggagtgtgag | |
| | ctctccggctacggcaagcatgaggccttgtctcctttctattcggagcggctgaaggag | |
| | gctcatgtcagactgtacccatccagccgctgcacatcacaacatttacttaacagaaca | |
| | gtcaccgacaacatgctgtgtgctggagacactcggagcggcgggccccaggcaaac | |
| | ttgcacgacgcCTGCCAGGGCGATGCTGGAGGCCCCCTGGT | |
| | gtgtctgaacgatggccgcatgactttggtgggcatcatcagctggggcctgggctgtg | |
| | gacagaaggatgtcccgggtgtgtacaccaaggttaccaactacctagactggattcgt | |
| | gacaacatgcgaccg | |
| | GGGGGTGGAGGCTCTAAGGATGAACTT | |
| 37 | MDAMKRGLCCVLLLCGAVFVSPSQEIHARFRRGARSY | tPA heavy chain |
| | QVICRDEKTQMIYQQHQSWLRPVLRSNRVEYCWCNSG | (lacking the |
| | RAQCHSVPVKSCSEPRCFNGGTCQQALYFSDFVCQCPE | protease domain)- |
| | GFAGKCCEIDTRATCYEDQGISYRGTWSTAESGAECTN | KDEL, (kringle |
| | WNSSALAQKPYSGRRPDAIRLGLGNHNYCRNPDRDSKP | 2 domain |
| | WCYVFKAGKYSSEFCSTPACSEGNSDCYFGNGSAYRGT | underlined; |
| | HSLTESGASCLPWNSMILIGKVYTAQNPSAQALGLGKH | GGGGS linker |
| | NYCRNPDGDAKPWCHVLKNRRLTWEYCDVPSCSTCGL | |
| | RQYSQPQFR | in bold), amino |
| | GGGGSKDEL | acid sequence |
| 38 | ATGGATGCAATGAAGAGAGGGCTCTGCTGTGTGCTGC | tPA heavy chain |
| | TGCTGTGTGGAGCAGTCTTCGTTTCGCCCAGCC | (lacking the |
| | AGGAAATCCATGCCCGATTCAGAAGAGGAGCCAGAT | protease domain)- |
| | CTTACCAAGTGATCTGCAGAGATGAAAAAACGCA | KDEL encoding |
| | GATGATATACCAGCAACATCAGTCATGGCTGCGCCCT | nucleic sequence |
| | GTGCTCAGAAGCAACCGGGTGGAATATTGCTGG | |
| | TGCAACAGTGGCAGGGCACAGTGCCACTCAGTGCCT | |
| | GTCAAAAGTTGCAGCGAGCCAAGGTGTTTCAACG | |
| | GGGGCACCTGCCAGCAGGCCCTGTACTTCTCAGATTT | |
| | CGTGTGCCAGTGCCCCGAAGGATTTGCTGGGAA | |
| | GTGCTGTGAAATAGATACCAGGGCCACGTGCTACGA | |
| | GGACCAGGGCATCAGCTACAGGGGCACGTGGAGC | |
| | ACAGCGGAGAGTGGCGCCGAGTGCACCAACTGGAAC | |
| | AGCAGCGCGTTGGCCCAGAAGCCCTACAGCGGGC | |
| | GGAGGCCAGACGCCATCAGGCTGGGCCTGGGGAACC | |
| | ACAACTACTGCAGAAACCCAGATCGAGACTCAAA | |
| | GCCCTGGTGCTACGTCTTTAAGGCGGGGAAGTACAGC | |
| | TCAGAGTTCTGCAGCACCCCTGCCTGCTCTGAG | |
| | GGAAACAGTGACTGCTACTTTGGGAATGGGTCAGCCT | |
| | ACCGTGGCACGCACAGCCTCACCGAGTCGGGTG | |
| | CCTCCTGCCTCCCGTGGAATTCCATGATCCTGATAGG | |
| | CAAGGTTTACACAGCACAGAACCCCAGTGCCCA | |
| | GGCACTGGGCCTGGGCAAACATAATTACTGCCGGAA | |
| | TCCTGATGGGGATGCCAAGCCCTGGTGCCACGTG | |
| | CTGAAGAACCGCAGGCTGACGTGGGAGTACTGTGAT | |
| | GTGCCCTCCTGCTCCACCTGCGGCCTGAGACAGT | |
| | ACAGCCAGCCTCAGTTTCGC | |
| | GGGGGTGGAGGCTCTAAGGATGAACTT | |
| 39 | ATGGATGCAATGAAGAGAGGGCTCTGCTGTGTGCTGC | tPA heavy chain |
| | TGCTGTGTGGAGCAGTCTTCGTTTCGCCCAGCC | (lacking the |
| | AGGAAATCCATGCCCGATTCAGAAGAGGAGCCAGAT | protease domain) |
| | CTTACCAAGTGATCTGCAGAGATGAAAAAACGCA | encoding nucleic |
| | GATGATATACCAGCAACATCAGTCATGGCTGCGCCCT | sequence |
| | GTGCTCAGAAGCAACCGGGTGGAATATTGCTGG | |
| | TGCAACAGTGGCAGGGCACAGTGCCACTCAGTGCCT | |
| | GTCAAAAGTTGCAGCGAGCCAAGGTGTTTCAACG | |
| | GGGGCACCTGCCAGCAGGCCCTGTACTTCTCAGATTT | |
| | CGTGTGCCAGTGCCCCGAAGGATTTGCTGGGAA | |
| | GTGCTGTGAAATAGATACCAGGGCCACGTGCTACGA | |
| | GGACCAGGGCATCAGCTACAGGGGCACGTGGAGC | |
| | ACAGCGGAGAGTGGCGCCGAGTGCACCAACTGGAAC | |
| | AGCAGCGCGTTGGCCCAGAAGCCCTACAGCGGGC | |
| | GGAGGCCAGACGCCATCAGGCTGGGCCTGGGGAACC | |
| | ACAACTACTGCAGAAACCCAGATCGAGACTCAAA | |
| | GCCCTGGTGCTACGTCTTTAAGGCGGGGAAGTACAGC | |
| | TCAGAGTTCTGCAGCACCCCTGCCTGCTCTGAG | |
| | GGAAACAGTGACTGCTACTTTGGGAATGGGTCAGCCT | |
| | ACCGTGGCACGCACAGCCTCACCGAGTCGGGTG | |
| | CCTCCTGCCTCCCGTGGAATTCCATGATCCTGATAGG | |
| | CAAGGTTTACACAGCACAGAACCCCAGTGCCCA | |
| | GGCACTGGGCCTGGGCAAACATAATTACTGCCGGAA | |
| | TCCTGATGGGGATGCCAAGCCCTGGTGCCACGTG | |
| | CTGAAGAACCGCAGGCTGACGTGGGAGTACTGTGAT | |
| | GTGCCCTCCTGCTCCACCTGCGGCCTGAGACAGT | |
| | ACAGCCAGCCTCAGTTTCGC | |
| 40 | UAAUACGACUCACUAUAAGGACAUUUGCUUCU | tPA-K2-HA- |
| | GACACAACUGUGUUCACUAGCAACCUCAAACAG | KDEL coding |
| | ACACCCCUGCAGGCGCCGCCACCAUGGACGCAA | mRNA (full- |
| | UGAAAAGAGGACUCUGCUGCGUAUUGUUGCUC | length; sequence |
| | UGCGGAGCAGUCUUCGUAAGCCCAUCCCAAGAG | encoding for |
| | AUACACGCCAGGUUCCGACGGGGAGCAAGGUCA | tPA-K2-HA- |
| | UACCAAGUCAUAUGCUCAACACCCGCAUGCUCC | KDEL |
| | GAGGGAAACUCCGACUGCUACUUCGGGAACGGG | underlined) |
| | AGCGCCUACAGAGGGACCCACAGCCUCACAGAG | |
| | UCAGGGGCCUCCUGCCUCCCCUGGAACAGCAUG | |
| | AUCCUGAUCGGCAAGGUGUACACCGCACAGAAC | |
| | CCGAGCGCCCAGGCCCUGGGCCUGGGCAAACAC | |
| | AACUACUGCCGCAACCCGGACGGCGACGCCAAG | |
| | CCGUGGUGCCACGUGCUGAAGAACCGGAGGCUC | |
| | ACGUGGGAAUACUGCGACGUGCCCAGCUGCUCC | |
| | ACCUGCGGCCUGCGCCAGUACUCACAGCCACAG | |
| | UUCAGAGGAGGCGGCGGCAGCUACCCAUACGAC | |
| | GUGCCGGACUACGCCAAGGACGAACUGUGAGCU | |
| | CGCUUUCUUGCUGUCCAAUUUCUAUUAAAGGU | |
| | UCCUUUGUUCCCUAAGUCCAACUACUAAACUGG | |
| | GGGAUAUUAUGAAGGGCCUUGAGCAUCUGGAU | |
| | UCUGCCUAAUAAAAAACAUUUAUUUUCAUUGC | |
| | GUCGACACGCGUAAAAAAAAAAGAAGAGCGAA | |
| | UUC | |
| 41 | GGGGS | linker |
| 42 | YPYDVPDYA | HA tag |

What is claimed is:

1. An isolated polynucleotide molecule comprising a nucleotide sequence selected from the group consisting of:
(a) a nucleotide sequence set forth in SEQ ID NO: 6;
(b) a nucleotide sequence set forth in SEQ ID NO: 10;
(c) a nucleotide sequence set forth in SEQ ID NO: 15;
(d) a nucleotide sequence set forth in SEQ ID NO: 39;
(e) a nucleotide sequence set forth in SEQ ID NO: 2;
(f) a nucleotide sequence that is at least about 85% identical to the nucleotide sequences of any one of (a)-(f), and which encodes a recombinant tissue plasminogen activator kringle 2 domain (tPA-K2)-containing polypeptide that is capable of binding apolipoprotein B (apoB), and/or inhibiting apoB secretion from a hepatocyte and/or an enterocyte, and/or inhibiting apoB lipoprotein lipidation;
(g) a nucleotide sequence that is the complement of any one of (a)-(f); and
(h) an RNA sequence encoded by any one of (a)-(g);
wherein the nucleotide sequence is operably linked to a heterologous nucleic acid.

2. The polynucleotide molecule of claim 1, wherein the heterologous nucleic acid comprises an endoplasmic reticulum localization sequence.

3. The polynucleotide molecule of claim 2, wherein the endoplasmic reticulum localization sequence encodes for an amino acid having the sequence KDEL (SEQ ID NO: 11).

4. The polynucleotide molecule of claim 3, wherein the nucleotide sequence is set forth in SEQ ID NO: 8.

5. The polynucleotide molecule of 3, wherein the nucleotide sequence is set forth in SEQ ID NO: 34.

6. The polynucleotide molecule of claim 3, wherein the nucleotide sequence is set forth in SEQ ID NO: 16.

7. The polynucleotide molecule of claim 3, wherein the nucleotide sequence is set forth in SEQ ID NO: 4.

8. The polynucleotide molecule of claim 3, wherein the nucleotide sequence is set forth in SEQ ID NO: 36.

9. The polynucleotide molecule of claim 3, wherein the nucleotide sequence is set forth in SEQ ID NO: 38.

10. The polynucleotide molecule of claim 1, wherein the nucleotide sequence encodes a polypeptide having the amino acid sequence set forth in SEQ ID NO: 7.

11. The polynucleotide of claim 1, wherein the nucleotide sequence encodes a polypeptide having the amino acid sequence set forth in SEQ ID NO: 14.

12. The polynucleotide of claim 1, wherein the nucleotide sequence encodes a polypeptide having the amino acid sequence set forth in SEQ ID NO: 35.

13. The polynucleotide of claim 1, wherein the nucleotide sequence encodes a polypeptide having the amino acid sequence set forth in SEQ ID NO: 30.

14. The polynucleotide of claim 1, wherein the nucleotide sequence encodes a polypeptide having the amino acid sequence set forth in SEQ ID NO: 3.

15. The polynucleotide of claim 1, wherein the nucleotide sequence encodes a polypeptide having the amino acid sequence set forth in SEQ ID NO: 9.

16. The polynucleotide of claim 1, wherein the nucleotide sequence encodes a polypeptide having the amino acid sequence set forth in SEQ ID NO: 37.

17. An expression vector comprising the polynucleotide molecule of any one of claims 1-16, operably linked to one or more regulatory sequences suitable for directing expression in a eukaryotic cell.

18. The expression vector of claim 17, wherein the one or more regulatory sequences comprises a promoter.

19. The expression vector of claim 18, wherein the promoter is a thyroxine binding globulin (TBG) promoter or wherein the promoter comprises the nucleic acid sequence set forth in SEQ ID NO: 17.

20. A cell comprising the polynucleotide molecule of any one of claims 1-16 or the expression vector of any one of claims 17-19.

21. The cell of claim 20, wherein the cell is selected from a hepatocyte or an enterocyte.

22. An infectious particle comprising the polynucleotide molecule of any one of claims 1-16.

23. The infectious particle of claim 22, wherein the infectious particle is a virus.

24. The infectious particle of claim 23, wherein the virus in an adeno-associated virus (AAV).

25. The infectious particle of claim 24, wherein the AAV is AAV8.

26. A lipid nanoparticle comprising the polynucleotide molecule of any one of claims 1-16.

27. The lipid nanoparticle of claim 26, wherein the lipid nanoparticle is lyophilized, in a suspension, or emulsified.

28. A composition comprising the polynucleotide molecule of any one of claims B1-B16, the vector of any one of claims 17-19, the infectious particle of any one of claims B22-B25, or the lipid nanoparticle of claim 26 or claim 27, and a pharmaceutically acceptable carrier.

29. A method for lowering plasma apoB lipoproteins in a subject in need thereof, the method comprising administering a therapeutically effective amount of the composition of claim 28 to the subject.

30. The method of claim 29, wherein the plasma apoB lipoproteins are selected from very low density lipoproteins (VLDLs), intermediate density lipoproteins (IDLs), low density lipoproteins (LDLs), lipoprotein a (Lp(a)), chylomicrons, chylomicron remnants, or any combination thereof.

31. A method for treating a disease associated with elevated plasma apoB lipoprotein levels, the method comprising administering a therapeutically effective amount of the composition of claim 28 to the subject.

32. The method of claim 31, wherein the disease is selected from atherosclerotic cardiovascular disease, hypercholesterolemia, hyperlipidemia, or type 2 diabetes.

33. A method for lowering plasma triglyceride and/or cholesterol levels in a subject in need thereof, the method comprising administering a therapeutically effective amount of the composition of claim 28 to the subject.

34. The method of any one of claims 29-33, further comprising simultaneously, separately, or sequentially administering a therapeutically effective amount of a plasminogen activator inhibitor-1 (PAI-1) inhibitor to the subject.

35. The method of claim 34, wherein the PAI-1 inhibitor is selected from the group consisting of: MDI-2268, PAI-039, TM5441, TM5275 sodium, TM5441 sodium, CDE-096, Aleplasinin, Loureirin B, Diaplasinin, Toddalolactone, SK-216, Geodin, Fendosal, AZ3976, TM5007; and any combination thereof.

36. The method of any one of claims 29-35, wherein the composition is administered intravenously, intraperitoneally, subcutaneously, intrabuccally, intradermally, intrahepatically, or intramuscularly to the subject.

37. The method of any one of claims 29-30, wherein the subject is human.

38. A recombinant tissue plasminogen activator kringle 2 domain (tPA-K2)-containing polypeptide, or a pharmaceutically acceptable salt, tautomer, hydrate, and/or solvate thereof, (tPA-K2 polypeptide) comprising:
(a) an amino acid sequence selected from the group consisting of:
(i) the amino acid sequence set forth in SEQ ID NO: 5;
(ii) the amino acid sequence set forth in SEQ ID NO: 13;
(iii) the amino acid sequence set forth in SEQ ID NO: 9;
(iv) the amino acid sequence set forth in SEQ ID NO: 30;
(v) the amino acid sequence set forth in SEQ ID NO: 1;
(vi) the amino acid sequence set forth in SEQ ID NO: 37;
(vii) an amino acid sequence that is at least about 85% identical to the amino acid sequence of any one of (i)-(vi), and which is capable of binding apolipoprotein B (apoB), and/or inhibiting apoB secretion from a hepatocyte and/or an enterocyte, and/or inhibiting apoB lipoprotein lipidation; and
(b) a heterologous amino acid sequence.

39. The recombinant tPA-K2 polypeptide of claim 38, wherein the heterologous amino acid sequence comprises an endoplasmic reticulum localization motif.

40. The recombinant tPA-K2 polypeptide of claim 39, wherein the endoplasmic reticulum localization motif is KDEL.

41. The recombinant tPA-K2 polypeptide of claim 40, wherein the amino acid sequence is set forth in SEQ ID NO: 7.

42. The recombinant tPA-K2 polypeptide of claim 40, wherein the amino acid sequence is set forth in SEQ ID NO: 14.

43. The recombinant tPA-K2 polypeptide of claim 40, wherein the amino acid sequence is set forth in SEQ ID NO: 35.

44. The recombinant tPA-K2 polypeptide of claim 40, wherein the amino acid sequence is set forth in SEQ ID NO: 37.

45. The recombinant tPA-K2 polypeptide of claim 40, wherein the amino acid sequence is set forth in SEQ ID NO: 3.

46. The recombinant tPA-K2 polypeptide of any one of claims 38-45, wherein the polypeptide does not comprise serine protease function.

47. The recombinant tPA-K2 polypeptide of any one of claims 38-46, wherein the polypeptide is not fibrinolytic.

48. The recombinant polypeptide of any one of claims 38-47, wherein the polypeptide binds to apoB.

49. The recombinant polypeptide of any one of claims 38-48, wherein administration of a therapeutically effective amount of the polypeptide to a subject reduces plasma triglyceride and/or cholesterol levels in the subject.

50. The recombinant polypeptide of any one of claims 38-49, wherein administration of a therapeutically effective amount of the polypeptide to a subject reduces the plasma level of one or more apoB lipoproteins in the subject.

51. The recombinant polypeptide of claim 50, wherein the one or more apoB lipoproteins is selected from very low density lipoproteins (VLDLs), intermediate density lipoproteins (IDLs), low density lipoproteins (LDLs), lipoprotein a (Lp(a)), chylomicrons, chylomicron remnants, or any combination thereof.

52. A composition comprising the recombinant polypeptide of any one of claims 38-51 and a pharmaceutically acceptable carrier.

53. A method for lowering plasma apoB lipoproteins in a subject in need thereof, the method comprising administering a therapeutically effective amount of the recombinant polypeptide of any one of claims 38-51 or the composition of claim 52 to the subject.

54. A method for treating a disease associated with elevated plasma apoB lipoprotein levels, the method comprising administering a therapeutically effective amount of the recombinant polypeptide of any one of claims 38-51 or the composition of claim 52 to the subject.

55. The method of claim 54, wherein the disease is selected from atherosclerotic cardiovascular disease, hypercholesterolemia, hyperlipidemia, or type 2 diabetes.

56. The method of any one of claims 53-55, further comprising simultaneously, separately, or sequentially administering a therapeutically effective amount of a plasminogen activator inhibitor-1 (PAI-1) inhibitor to the subject.

57. The method of claim 56, wherein the PAI-1 inhibitor is selected from the group consisting of: MDI-2268, PAI-039, TM5441, TM5275 sodium, TM5441 sodium, CDE-096, Aleplasinin, Loureirin B, Diaplasinin, Toddalolactone, SK-216, Geodin, Fendosal, AZ3976, TM5007; and any combination thereof.

58. The method of any one of claims 53-57, wherein the recombinant polypeptide or composition is administered intravenously, intraperitoneally, subcutaneously, intrabuccally, intradermally, intrahepatically, or intramuscularly to the subject.

59. The method of any one of claims 53-58, wherein the subject is human.

60. A polynucleotide molecule comprising a nucleotide sequence set forth in SEQ ID NO: 8.

61. The polynucleotide of claim 60, wherein the polynucleotide is formulated for delivery to a subject in an infectious particle.

62. The polynucleotide of claim 61, wherein the infectious particle is an adeno-associated virus (AAV).

63. The polynucleotide of claim 60, wherein the polynucleotide is formulated for delivery to a subject in a lipid nanoparticle (LNP).

64. A composition comprising the polynucleotide of any one of claims 60-63, and a pharmaceutically acceptable carrier.

65. A method for lowering plasma apoB lipoproteins in a subject in need thereof, the method comprising administering a therapeutically effective amount of the composition of claim 64 to the subject.

66. A method for lowering plasma triglyceride and/or cholesterol levels in a subject in need thereof, the method comprising administering a therapeutically effective amount of the composition of claim 64 to the subject.

67. A polynucleotide molecule comprising a nucleotide sequence selected from the group consisting of:
(a) a nucleotide sequence set forth in SEQ ID NO: 34;
(b) a nucleotide sequence set forth in SEQ ID NO: 10; and
(c) a nucleotide sequence set forth in SEQ ID NO: 36.

68. The polynucleotide of claim 67, wherein the polynucleotide is formulated for delivery to a subject in an infectious particle.

69. The polynucleotide of claim 68, wherein the infectious particle is an adeno-associated virus (AAV).

70. The polynucleotide of claim 67, wherein the polynucleotide is formulated for delivery to a subject in a lipid nanoparticle (LNP).

71. A composition comprising the polynucleotide of any one of claims 67-70, and a pharmaceutically acceptable carrier.

72. A method for lowering plasma apoB lipoproteins in a subject in need thereof, the method comprising administering a therapeutically effective amount of the composition of claim 71 to the subject.

73. A method for lowering plasma triglyceride and/or cholesterol levels in a subject in need thereof, the method comprising administering a therapeutically effective amount of the composition of claim 71 to the subject.

74. The polynucleotide molecule of any one of claims 67-73, wherein the nucleotide sequence is set forth in SEQ ID NO: 34.

75. The polynucleotide molecule of any one of claims 67-73, wherein the nucleotide sequence is set forth in SEQ ID NO: 10.

76. The polynucleotide molecule of any one of claims 67-73, wherein the nucleotide sequence is set forth in SEQ ID NO: 36.

77. A recombinant tissue plasminogen activator kringle 2 domain (tPA-K2))-containing polypeptide, or a pharmaceutically acceptable salt, tautomer, hydrate, and/or solvate thereof, (tPA-K2 polypeptide) comprising an amino acid sequence selected from the group consisting of: the amino acid sequence set forth in SEQ ID NO: 9 and SEQ ID NO: 35.

78. A composition comprising the recombinant polypeptide of claim 77 and a pharmaceutically acceptable carrier.

79. A method for lowering plasma apoB lipoproteins in a subject in need thereof, the method comprising administering a therapeutically effective amount of the recombinant polypeptide of claim 77 or the composition of claim 78 to the subject.

80. A method for treating a disease associated with elevated plasma apoB lipoprotein levels, the method comprising administering a therapeutically effective amount of the recombinant polypeptide of claim 77 or the composition of claim 78 to the subject.

81. The method of claim 80, wherein the disease is selected from atherosclerotic cardiovascular disease, hypercholesterolemia, hyperlipidemia, or type 2 diabetes.

82. A recombinant tissue plasminogen activator kringle 2 domain (tPA-K2) polypeptide, or a fragment thereof.

83. The recombinant tPA-K2 polypeptide of claim 82, wherein the recombinant tPA-K2 polypeptide comprises a sequence with at least 80% identity to SEQ ID NO: 5.

84. The recombinant tPA-K2 polypeptide of claim 82 or 83, wherein the polypeptide does not comprise serine protease function.

85. The recombinant tPA-K2 polypeptide of claim 82 or 83, wherein the polypeptide is not fibrinolytic.

86. The recombinant tPA-K2 polypeptide of any one of claims 82-85, wherein the recombinant tPA-K2 polypeptide binds to apolipoprotein B (apoB).

87. The recombinant tPA-K2 polypeptide of any one of claims 82-86, wherein the recombinant tPA-K2 polypeptide comprises a sequence selected from SEQ ID NOs: 13-14.

88. The recombinant tPA-K2 polypeptide of claim 87, wherein the recombinant tPA-K2 polypeptide comprises SEQ ID NO: 14.

89. A method of making the recombinant tPA-K2 polypeptide of any one of claims 82-88.

90. A polynucleotide comprising a nucleotide sequence encoding the recombinant tPA-K2 polypeptide of any one of claims 82-88.

91. The polynucleotide of claim 90, wherein the nucleotide sequence encoding the recombinant tPA-K2 polypeptide comprises a nucleotide sequence selected from SEQ ID NOs: 15-16.

92. The polynucleotide of claim 91, further comprising at least one regulatory sequence operably linked to the nucleotide sequence encoding the recombinant tPA-K2 polypeptide.

93. The polynucleotide of claim 92, wherein the at least one regulatory sequence comprises a promoter, an enhancer, or both a promoter and an enhancer.

94. The polynucleotide of claim 93, wherein the at least one regulatory sequence comprises a promoter.

95. The polynucleotide of claim 94, wherein the promoter is thyroxine binding globulin (TBG) promoter or wherein the promoter comprises SEQ ID NO: 17.

96. The polynucleotide of any one of claim 90-95, wherein the polynucleotide comprises more than one nucleotide sequence encoding the recombinant tPA-K2 polypeptide.

97. A nanoparticle comprising the recombinant tPA-K2 polypeptide of any one of claims 1-6 or the polynucleotide of any one of claims 90-96.

98. An infectious particle comprising the polynucleotide of any one of claims 90-96.

99. The infectious particle of claim 98, wherein the infectious particle is a virus.

100. The infectious particle of claim 99, wherein the virus is an adeno-associated virus (AAV).

101. The infectious particle of claim 100, wherein the AAV is an AAV8.

102. A pharmaceutical composition comprising the recombinant tPA-K2 polypeptide of any one of claims 82-88.

103. A pharmaceutical composition comprising the nanoparticle of claim 97.

104. A pharmaceutical composition comprising the infectious particle of any one of claims 98-100.

105. A method comprising administering a therapeutically effective amount of the pharmaceutical composition of any one of claims 102-104 to a subject.

106. The method of claim 105, wherein the subject has hypercholesterolemia or hyperlipidemia.

107. The method of claim 105, wherein the subject has been diagnosed with a cardiovascular disease.

108. The method of claim 107, wherein the cardiovascular disease comprises atherosclerosis.

109. The method of any one of claims 105-108, wherein the subject has been diagnosed with type 2 diabetes.

110. A method of treating a cardiovascular disease in a subject in need thereof, the method comprising administering tissue plasminogen activator (tPA), or a fragment thereof, to the subject to treat the cardiovascular disease in the subject.

111. The method of claim 110, wherein the tPA fragment comprises a tPA-K2 domain.

112. A method of reducing blood cholesterol levels in a subject in need thereof, the method comprising administering tissue plasminogen activator (tPA), or a fragment thereof, to the subject to reduce blood cholesterol levels in the subject.

113. The method of claim 112, wherein the tPA fragment comprises a tPA-K2 domain.

114. The method of any one of claims 110-113, wherein administration comprises oral administration or intravenous administration.

115. The method of any one of claims 105-114, wherein the method reduces a level of intermediate density lipoproteins (IDLs), low density lipoproteins (LDLs), very low density lipoproteins (VLDLs), lipoprotein (a) [Lp(a)], chylomicron, or chylomicron remnants in the serum of the subject.

116. The method of any one of claims 105-115, wherein the tPA comprises the pharmaceutical composition of any one of claims 102-104.

117. A method for lowering plasma lipids and/or apolipoprotein B (apoB) levels in a subject in need thereof, the method comprising administering a therapeutically effective amount of a composition comprising a polynucleotide molecule comprising a nucleotide sequence selected from the group consisting of:
(a) a nucleotide sequence set forth in SEQ ID NO: 2;
(b) a nucleotide sequence that encodes for the polypeptide sequence set forth in SEQ ID NO: 1;
(c) a nucleotide sequence that is at least about 85% identical to the nucleotide sequences of any one of (a)-(b);
(d) a nucleotide sequence that is the complement of any one of (a)-(c); and
(e) an RNA sequence encoded by any one of (a)-(d);
wherein the nucleotide sequence is operably linked to a heterologous nucleic acid comprising an endoplasmic reticulum localization sequence; and
wherein the subject is suffering from or at an increased risk for atherothrombotic events.

118. The method of claim 117, wherein the endoplasmic reticulum localization sequence encodes for a polypeptide having the amino acid sequence KDEL (SEQ ID NO: 11).

119. The method of claim 118, wherein the polynucleotide molecule comprises a nucleotide sequence set forth in SEQ ID NO: 4.

120. The method of claim 119, wherein the nucleotide sequence encodes for a polypeptide sequence set forth in SEQ ID NO: 3.

121. The method of any one of claims 117-120, wherein the composition is formulated for delivery to the subject in a lipid nanoparticle or adeno-associated virus.

122. The method of any one of claims 117-121, wherein the administration of the composition lowers plasma apoB and/or lipid levels in the subject.

123. The method of any one of claims 117-122, wherein the subject is undergoing, will undergo, or has undergone thrombolytic therapy.

124. The method of claim 123, further comprising simultaneously, sequentially, or separately administering a tissue plasminogen activator to the subject.

125. The method of any one of claims 117-124, further comprising simultaneously, sequentially, or separately administering an anticoagulant to the subject.

126. The method of any one of claim 29-37, 53-59, 65-66, 72-73, 79-81, or 105-116, wherein the subject is suffering from or at an increased risk for atherothrombotic events.

127. The method of claim 126, wherein the subject is undergoing, will undergo, or has undergone treatment with a thrombolytic agent, an anticoagulant, catheter-directed thrombolysis, and/or thrombectomy.
